SEQUENCE LISTING (3 sequences)

SEQ ID NO: 1 - DNA, 33747 bp, Equus caballus
  source 1..33747: mol_type = genomic DNA
gagcgaaacgctttctcactgctctttgctccccgccctctgctgccagaaccttggggatgtgcctagactcggcgcagcgcacgtcctggccaaccgcgagcagaacaaacccttggccttcggccaggaggctccctcccagccaccgccccccgccagcgcctttttttttcccccatacaatacaagatcttccttcctcagttcccttaaatcacggcccagggaaatctcctcaaagccttcatccagccacgggccagcatgtctgggggcaaatacgtggactccgaggtaggcgtgggtgggtgcgcgccgggccccggggtgcgcacggagtgcctccttctgctatctcccactccaaatattccgactgctttccttgccccagccctctctcgaccccggagcactcctctcgcgctggcactgctgaggaatgggcctgggcggggaggtgaagaggaggcaggaatgttttatgttttcctattgggacgggggccaagggagcccctaagctagaggaacacggaaaagggggtcgtcggggtcctcaagttgggtctgccggacctaggacgcgtttctggataggtccggtgtcctggcttattgcgggacccccacggtccggctcctgccagctaggaggcgaggagctggagcgcggggtgggagatcattcacccgcaatcttagggacagtccccgcgactccccgccaggcgcctggactccccggggcggcggcgcgcattgtgtttggactttccaaagctcttgggccacctcaagaactcttcctgcatctcggcgcctagcaagggtgtttggagaggggtggacctctccttcccccaactaccaccataccttctaaccactctcgcacgctcgggctccgcctgggtgtggagacttcattttcccaaacgcagccgcggttcagccgcccgctgacagcctgggagtgacctgagggtgggtcgggggagttatacaggtgagatatcgttctaaggagtttagggggcctgacacatggctctgcccagagatgggggcgttcgggactgtgaccaccacagggctgggggaagcagtggagggacaaacagggcagagggactgtgcgggacgcgagcaggtgcgagagagactacgccaagaagcaacgaaaatgagggcactccatgagtcggggagttcaggctgaatcttaaggatgaacctggaagggattatggagagagggggagtagaggctgggacgagaaaatgaagcaggtgaagcactcggggcgagggcggcgtggcggagagagcggctggagagaagtcagagatcgggtcccctcccctgccgaggtctccgcggggtaccgcgcgcagggcgagcgcagggcgggggccggggcggaccgcgccgcggggtttgccctgacccctggcggcgggcgggggaggcaggagctccctgcctggtaccgaggggtgtggtgtcctctcctcgatcctcttaaaaagcttgctgcgccagggagtttactgcgcgcggagccaccgctgccgaagcggttagttcgattttgagcctcgaggtttgctccgccgcctggctgacttctgatcgcttgtttttcttttcgaaattttcttcccaccacccgtgctaccctccccctcctggtcgtccgctctccgcagggacatctctatacggttcccatccgggaacagggcaacatctacaagcccaacaacaaggccatggcagacgagatgaacgagaagcaagtgtacgacgcgcacaccaaggagatcgacctggtcaaccgcgaccccaagcatctcaacgacgatgtggtcaaggtaaggtgaggcgaccagcgaggaaaggatgccccttgaggggccgctgtcagggccggtacagttctcctctggcaagcagcctgtgcaggcttctctacctttgccacacacacactgttttctggccttgatgtgtgggagctcccgcggtcgtctgaaatgtcacatctcccctcccccctcctgctggttagttcagctggaatttagcaaacgagttttgtaggcgtttctataactgcacttttctcctagcaagtgaaccccctgatctcagcaacagacagacccgcacgccttggcagagtaagacggttgccctgcaggtcaccctctctccatccccaggttgaaagtctagcgaagggtggtgccccattgcacatagagttaaagcagcaagaccattttgtaaggtcttgtgctaagggtgagagccggtatacagagtgggcaggttcctccagctaaaattaagataacctgacgggatttttttttttaatgataaagtgagtggaaacccttccgagtaaagacaaagaatataatatattatatataatatgcatatgaaggatattttaactggaaaaacacgagcaaatttacacaaccaaaactaatgtgcgcctcactttctcccacagacatctctagttaggcgtgcaatgctgcttctttagtaactacctgggtcagccttccctgagctaacgggacgtttgggttggagttcctttttatactttcaagatgcattgggtgagattaaaaatagcttaattgaacgggttgttggccaaatttagatgtgtctccatggtcgggttttgattatcattatgttattcaactttgacccttttagcaaatcttgtcacaagaatttattgtctgaatccatcttgattgggaaatcatgggcctaaggcatccttggcaaaaggtcttgtttgaggggctaggagggccaaggaaagtcctggcctcaaagatgagagaatgaagacccagttctatacaaccccgcagcagccaatggggtggatgagcgatttccacccacccagcctcctatccatggatgggaggagctatagttcagagactgttcacatttatgaataatatatttcaaaagggggaacaatttaatctgtaattggaaggaaaacataactgtcagaattgactctcttggcttgctggtggaggaaaaaggaaaattggagcattagcaggtttttctcctagccagattatggaatatttcaaagcagcagcaataacaaaatagtttacaaacttgaaattagaattgctaaaaactctttactaaaaacattaccttaaagggggaaagggcatttttgcttttttcccttctcatgtagctttggacaagaaggagaggtgccaggaaaagaggcagatttcagaaagagctggctccattggagcctccctcttgttccactgcagtgagactgggattcctggagcaagtgaacctcccgggatgagtcagaggagccagtagtgtggatgtgggtctccacacatagcatgactaagttgagaaagaaaggccccactgggaaaaccaacttcaacacagatggaaaaaaggcataacaggcttagaggaaatagtagtttacagactagcatttcaaagagcaagtgtggcgatcttcatattaaagaaattaaaaggaagagttggagcaagctcctgctggcctagagaaagtaaaccccgccctcttgctcatagaactgggagcaaaacaagacagtcagaccgaaaaatccacttggagaagaatttggtaggcccacttactcagacgccctgcttagagaggacttcagagaatgtcctgcagagagagctctaaagattgtcatctgccattagggccgggaataggggcctacacgccatggagattcctattttataatattttttctttgagtccaagcacgaaggtaatgatagaaacagggaggatcctctgaatgttaccatgatagtaagtaagaaatttccctaaattttacttttgacaacatagactttttaaaataataaagacacccaaagtttcacggtcttgcatggtagcttaaaacgtcccgtgataataaagaaacggtggtccactgaattactattaaaagtaccaaatcaagtcaagaaaggtcagctctaaaggcaagcagctctataaaagatgccaattctgtccaattctctagtcaccagccactggtggctatttcagtttaaatttcaattaatttacattaaatacaatttgaaattgagttcctcagtcacactagccacatttcgagttctcaacacgtgaagctggtggctaccatattggacagcacagatctagaacatttccatcaccacagaaattgctattgggcagtgtcagactagagtgttccctgcatggtaaaaaggtctccatgccaagtttagaaaaataactggatggagaagaaatgagactctgatgtacagtgtgaggagcagcatggtctatccacacagtatccttttagtccagcaaaacagctattagggaaagaaagaaaactcagacgctgagtgtgccatgaatttctgtttcaagaaggtggagtattatcacctattccactggtgataatggcgtaaattagactctgcaatttagttctgataaaattggtttcttatcttactgaaggtttggtgccccatattatgagatatatgcaaagacatttatctaaaaaaaagagtaggttaagaatgctttttcaaatttcctctgattaaaatagtcacaatgtgagaccttctgttctccctctgagatttctatgtcttcttcttttgttcacatttcattcatgagtttattacgtacttatctggattttctagataaagaaaatggcttcaaatctatgattctgatgtcatattagccatcaatcaatttatttattaaactcttatcatatgccagactagacactatgctgcgtgttacaaagggtgattcccaaaataagtcctagtgatcacaaagattagagttagtgagacaagatgcacacagaaagtttaaaaaaatacacacacacacacacaatatcacaacagtaagtcactgccaaatggtaactaaggggtccttttttgccttgactgccagggaacatgtaactagattttgtttcattgacgcatgttacttattctcacgcacatatggttttgaattttcactttctttttcaactgggatgctatttatgaggtttctattgtaagctttctcgtcattattttggaaatagacaaaatacatgctataataaatcaaaagtaagtagtattgaatattgtgttctaaaaaggtttttaaaaagtgagtcgtattaattatttggatcttggtaatcattctgcaacgtagataaatatcaaatcatcatgatgtacactttaaatatttacagttatgtttgtcaattattcgtaaataaagtttttaaaaaagtgaatggtattgatattaagtgttagggagcccctactatgcatggggcaatagagtgagagaaggaggatgcattatagatagttcaatggagaagggcatgaaggaggtgcaagctaagttggatcataaaggacagatgggatgcaaatagctgagaaaagcatgggaaggcattctaaatgagcaaggcatggagatggggatgtctcacacagggcctgagcctggaagtttcggtagcccattttgcttggaattagagttcatgcggcagagttggtaggtcgtaaagatgaaaagactgatcatggccagacggcagagtcctgaagccggtactcatgataagttgggactttatcatcagcagtgagaagtcaggaagacttcaagcatgaggatgacatgacgaaggcgggcttttcagaagaggactggttaaggtacaggatgtaacaaaaggtatccagtctggatgagcagagaatagttatgatgtaattgcatggtggtccagacatgaaataagaaggacagaatgtagtggcagtggggataaaccatataaacacaaaatgacagaattttagaattggaagggaacatatggtttcaaggctaacctccatttcctcttttttaatattaggagagatttaagggctaaataagtttaaaatttatccatgttttcacatggttactccccaaaacatatattttgaaatgtatcatcagggacgaatagacaagattatgtgatttcatggagtattttcaccaggggaggaatgagttgagatgattctgaagttagctctgagacaaatcaggacatttgagaggagatctttgttggaaaataaaatatgatttgcaactttcttttctaaaacttgactgtaatgatccttaaatatacacagacagaaaattttctttagtagtcaagaaaagttatacgcttggtaatgagatcaattttggtttcaaaataacagcctaacgcctctgtgatccctttcaattagaaatcattctagagcgattggtgattaatatcaatacaatggtaattttcaatttgaagtttatctgatataatcaaaacaagatttaaaagatcagatttctcagagcactcagcgccatgttaatattgtcttctgttggaattggtatcagttttgcttgtttgtttaggagatagcttcacaagatggtgattaatattctgccacacatgagatcatgcatggaactcagaagccccagcttgcaaaagagcattagggtcatctttggtgcgaaattattctcacatcaatgagcaaagaacaatactttaatgggggatattattcattcttaagcatctaaaacactttcaagttcagccaacaatgatgtacatgttaaaggcttattaaaagaaagtctttttttctcctccatctcctaattgtggtcaaaaaacattgaagaattagtttaactttggttctaactaagtaaaatctgatattatgtattcaaatttaagacaagatacccaaatatcctcaaattatgcaaaagttcgtgatgtgggttattcttccaattaacagcggtgttggctatattttaaaggctcttaaagtaagaacagctgcaatagtaagcagcatctggaaagtccagcaatttcaaaaaccacctgcttatgcatcctgcccacttaagtccataaaataacagacactttcatattccaaatgaaactggtttttagtttgccctacttttcaacataggttctctatgatgaaacgcccagagagcgctggtccctaggagaatgggactgtgtgcactgacctgtggagttctggacactttcatgatcctcctggctgagtttgcagtggggaaagggcaaggctgctgttttcatgcaaagatggtttatgccaagaaacacaaactctttcttcttccgcgctctgagccttgtacatatattatcttcattactatatactaggtcctggaggaaaagatttgcatactatatttcttttaaaagaagttcctaaaaaagaagccctcagcccctagaaatatgcctaacacataggtgctcagaggcaccctgatggagtggaaagagtattgtgccaagataagaaaagatatttttgcaacttttgtaactttcttctctaagacttgaatgtaatgattcgtaaatatgcagacacaaaattcttttttttttttttgaggaagattagccctgagctaacatctgccgccgatcctcctcttttttgctgaggaagactggccctgagctaacatctgtgcccatcttcctctactttatatgtgggacacctgccacagcatggcttgacaagcagtgcataggtccacatccaggatccagactggcgaacaggccaccgaagcagaacgtgtgaatttacccactgcgccactgggccaaccccgcaaaattctttaacagtcaagaaaaggcatacacatagtaagcatatcagtttaggtttcaacataagggcctaatgcttttgtgatctctttcaactagaaagtgtctagactcagtagtgttggacaggacaccaaggaatgagaagtgaaggatgtcagggcaacagatttctcttcctcggtaggtccctgagtgtggggcttactaagagggagcaaacttgtttggctaataaaagctaaaaacaccagccctagaaatatttttggcaagctattttatgtttctatgcttcaacttctctaattataaagaaaagaatgagcttctttctcatataatagcatccccccccaaatctttcaataaatgattattatacatctaatatcatgtgctgggggctacaatatggaacaagatagatatgacgctgcccgtccagggtttatggctatctccttgcctctcttcatgtagtttcctttgaaaattgttgctgaaattcctttaccaaatgctactgtctgttaaaaccttataacctgtaggccgtagttcaaaaaatgccacatctctaatgccttgaattgttcttgagccagtaaacgaggcttgggcaaaataaggaacacatattttttaaataatttgcccctgttttggtcctataaaaacttgcaatgaaattatgggaactacttcagttcttctctaatattctctcttcccatattggtgctcaatttctaatcttcctattgaaaatgtgaaattttcaccctttttttaaatctcatagccctttacaaacccaagaggtctctcaaattcagctaggcatcataatcaccagaagaattttttaaaatacaaatgcctgggcccactacagatctattaaattagaatatttgggatggggcctaaggatctgtatttttcaaaactccctagatgactattgtgaagtcaggtatcaaagtgattgtacccaagggtgactttagatatacactctggacggttccaccctaagagaagagtggtgtcaccctgtagttgtaaatgttcttcctgccagaagacaacttgagggttgggccagtttaaaacctgccagggagttttgaaacaccacagagggttcccaaatcaacgttgctcaatggtcctccatcccttgaggctactcgggcttacagccaactataagcttagagcctcagggtgacctaggaatggggtgaccatatgttccagggtgtcccatacagaccagttaatactggttagccctgagtcactactaatagtgcccctttcatgttcagaagtgatgcagtttggaagaaaaattataggtcacaaatggattggcaaggacctgtcactgattcttgtactgggaatacactggaagtccctatgcaaactgttctttagcagattgggcaaaatttccacataccgagcatgcttttctttcctttctgctctccggtatcacctctctcctaccttcatcgcagccatcaagctgcactataatttgggagaggagcttttacatgccaatctactctaccctcagaccttagtttctttgcctgcaacagagggttaatgaaactgacatccttccatcacatattctgtcaactactcaatcaaagattaaaatgtttaaaatagttttaagtatgtttcagttagtccgggaaacataaaatggcataccttcatatgaaccattgtttcaggtagaggaagtgtttttaattttgtcttaaaggaaatctgcatgatccatgtactgtgcaactaccgagggaattaattggcagaacagacttacacagaatacatatttcctgcctctcaggggagctatgctgatgtttcagatacaaagtacatcttgttttctttattgaacttgagaagatatctcttgttggtcattatttcatcacaggggaaataagtattcctaaagacacaactgagttcccgttagccattgttgaccgtttaggtaattaagagccaggcatttttaattttgaggaggcttaaataagtttggatggtgtatgtatgtgcttagtatgctatctcacagcaatttaaaagtgaaattttaaaaaggtgcattcaatctgtaaacctttcctgagcccagcaaacattgtaatacaaatggacattgatagattctatgtaaaaaggtaaattgggtctacagattgcattgggtcatctcaaatatgttgcacgtatcaaacacaattactttttcttatctacaatgaataactggaaaagatgctggtttctggacccaaatagtgaggaagtaggagattgggaggaaaagcagcaaagataaagaaataaccataaagtcacttctccctgtcctgccagtcactctgtgctcaataggctttactcgtgccaattctcggacccccacatccgttgctttgcacaatgctgcattgtctgatgacatgatcaggctcaggcactgactaagtattaacccactgctcctgccccaggagctttagagtttagcagggaagcacacatgtaaacaaatctgagtgaggtcttgtaactaagggacaggccagttgggcacaaagcggaaagttctcagcagtctccaaagccgagcatgtgttcaccaggcactcgaggcagggaagggttcccagcagagggagcagcaggagcaaaggcccagagacctgagggcacttggcgctttctgagcaccagcggcaggtcactgtggccaaagcaagggacacaggagccagatcagcagggccttctgtgcccagccaaggcgctttgttgaccacagtgacctgcagaagcatttttaggcaaggaagtgacctgatgagatgtggtctttactaataggaagggctaactttggtgcaatggggcaaaccagggggtaccagatgtgtaggtgggagtgaaacaggaggtgggatgcttcttccagcgctgggtacaggctagcttcgtggtaaagattaccttacttctccaatgacccttcgaggtaggtgctattgtcattatcacagtcatattcattttacagataaggaaacgcaggcttagccatttactcaggattacatagttagaaagtgtagagtcagagtctgaacccagacaggtgagaccccaggacccagcttcataaccactgcactccagtgcctctccgtaataggtggtgatccaagcagaccgtggtgtcaccttagtgcagaaagagatgactgtgttctcacaggaggtgggcagcgtttgccaggggattccagatgtggtctgtgttgggagtgacaggcctgcagttctaaggtcagactctagatcctggcaggtggactggggaaggggaggggaggaccgatgtaaagactaaggtaaaaccccagatctcaagaaatgtgtgtgctgggtgagtttccaaccaggggcggagggtcaagtcccagagaccagagtggggtacaaagtaggggtgtggtctcggggattaggagtagaacacgcactccgagccagacaagctgcaatgctggcttgtgttgaatgccctgagtctgtgaagcacccagattgcccaggccagccaccgcacctggggtgctgccgatcctgccgggtgtgtggggagaggtggacgtgagagaaggaagcaggtgctgacaggcactttgcatttaacaggttcatttaaggttctcaatttgcctgttcgactccttcagatattcatgccattgcagcaatatgttgtaaaaacaggaaacaaccgagaaaaaccttagttttatgatagtgagagaaatgcccatttaatcctgaaagtagtttctctgagacttcctccctacaaattacgtatttgttctcaaaaacaaatgccagaagtaggggacagacttaatcgtgcattttacaaggcaccgttaacctctcagaacaattaaaatgttccgtcaatcatggctcactgttcgccttttctatattattaacagtaataatatatagcaggaaatggacgtaccaaaatatggcaagggaatggagttgcaatcattggatttcatcatcaattattttggttaggttgatagagctttcctccttgcttgatcctgagcgtcaggctttttagtagggagagaaattgtttttctaaaagttaatacccaccagttccccaaactattttgggtaagcccctctattacgctagttataaaggtaggaaagagagtcattctcagataaggtgctcccaagaaaggccactcaacaaacacctaccctgcaccaggcacggagcacagcatgagccctgcctacctgacagaactgcggccatcacccaggctcacccagacacccgagcctctgccagccttccctgaccgcccttgtcagaactggtcctctcttctcttggctcccaccacatttggtttcatttctatttaacacatattgaagccctctaaggattctgcttctctgtccccatgcctctctctctccttctcctcccacttcgtgcatgagtccttcagggcagaacagtggccccaggactgtggctgcccagtgcccatctcaaggccccaccctgaatctgccatcagaaaatggatgccaatttaaatccgccaaaaagaatctgaaatctattttttaagtgtataatgtcttctgtgcatgaaaaacaggagaattatattattgtcttcctttgccccttttctctataacagccacatttaaaacctcgtaaggaaaacatatccaaataattaataaagaaatccaaaaacatccactctatttactaaggtaaagagaaatatgttctaatatgtgtatatatttattattatgaatgcttttttagtctccagtttgaggaggacgcaaatacgtctctgctgagccttccagtaaggaggctgtggcacctggatgatctccgggggcctggaggataaagctaatctcctaagtgcctggcagacagggcaggcaaattcccagaatgccgagctgccatcactgctgcttggcattgccccctagggccagaagcatgaaggctgagagtggatctcatgccacatgagaactcgcctgccaagaaaggggtagagggactccctgtggaaccccctatatacccctggacaactgcaaacatcagaggaatgactaggtcaggcagcagccctgaagaaaaatccaaaaccttccacacctgaaggagacaaccggtttgagaaactcaagcctcttccttctctgtggctttactacgtctagatgttaagtacacagggctctgaaatgttactagctgggaggtgaccattaatccactattccagcagcatagaagtttctttgtacctcagaggtgatctctcatagatctcagatctcttgtcaggaaaaaaaaaaaaaaggcaaaatgcttaagtgtctctggaagggatcagacatcattcatttcattcttgatgtctgtgccaagtaaaacgaacgttagcttcttccccagatatgcttctttctctggtctcctgtgataggaggtggatttactgtaatattatataaaacttgccaatttggggcaggcctgagggcgtagcggttaagttcgcacgttccactccctgggcctggggttcaccagttaggatccgggctacagacgtggcacggcttggcaagccatgctgtggcaggtgtcccacatataaagtggaggaagatgggcacgggtgttagctcaggaccagtcttcctcagcaaaaagaggaggattggtggcagatgttagctcagggctaatcttcctcaaaaaaaaaaaaaaaaaaaaaagacttgccaacttgaccctaaatagttctctgtcaacaagctaagtagaggaaatcaggatcattattcaattaaaaataggtaggactagaaggtagtcatcaaaaagagatgatgatgtgtaaatgaattttagatgtgactcatatctaatgtacaacaaaggccacagtcctagcacatgtaatattttgtcaatgactagaacgaacaagttaagaggactgtcattatgacaccattatgacactcattatgacaccagttatgatgcccttgttaacatctatgataaaaataatatcaaggtcattttgacaaattacaagtagcccataaaaataagataaaaaccaatacaaagtgagataatgttaaacaactagaaataaaacatatagatattaagtgggaaacaaatacataaacatacgaaaaataagaaaataccagaaaaggattgtcatttctgtggaaaaccccttgtaataattccacaacccccaggggaccaagatctccaaggtaagactcgctattgtaaacacacccttaaatgaatccaccgcactccacatcccttgggcaataggggacccaaactaagtctatcaattattcctgaatattaagcatgtcaataatttaatgatactttaaaggaagcacaatgagtagaatcaaaaggacaatgcaatgcaaagaaatgtcataataaagattcgctactttactttcttttggtcagacttcttaataaggggttcctgtttgatcttataaaggaggtagagatgctggagagatcctggggaagagcgaaaggaaggagataagcggcgaagagaaacccccaggtatagctggggagggattgttttgtctggacgattttttagaagagttagaggggaacatgggatgacgactgcctttggttattgccaggtcattgggagactaaggagaaaaacactgctcagacacaatcaccattcaggacaaggagaaaggaggaaggggtatacacccagtttaaaaaaaacaaacaaacaagttttaggttacatgggagaagaactcccaagtaataagagtggttaatcccaggaatgtgtcacaagaggtggttttgtaatagttgggttaaattccttacttgtctatgtaactcctccaatccttccatctccacaattacaatttggtgtgtaacaaccttgacacccagcctcataattaagcacaggcatgtgtatgaatgcttccctctattctagcaggatacctggtttggaggcaaataggagttgatgtggcattattttaaacagtggttgagaatgcaagtggtaaacatcagaaagtagaacactgcaaaatgcatgcattgtatgtaccaaaaggtcagtatgaaacattatctttatggcaagcctgcctatccactccctcctacacattgcatattcacacaattttgaagcttgtataaacccctattgtgtatgtgatttaaactcatgagagagcgtgacttctgcaaaggatagctgttttgtgactttagaccagtgattctttaccccggctacaaatcctctgaggagtgtttaaaaatactgttgtgtacaccccatccatagaaattctgattgaactggtctatgatagggcccaggcctatgaatttttttttcaaagctccccaggtgatgctaatgtacagccagaattgagtctggtttagactttcagaatttcttaataataaaactatgtaccctgataaaactttcaggaccataagggccacagcgggggaaatgattacttctgtaggtcctcattttggggattccttagcatttttattggaatattacatttgtcacttaaattcctattacaaaaatactttctggagctctgcataggaactgcgtaagagagactttatatgtggaataaatctcatgtcacttatgtgatccagtcctgttgtgtttccctttcttttgctcattcttttattctccccaagaggaagatcagcttgttagagtgtcagaatgtcacccccagctctctcttcctgcctgagtttgtggctcaggcatatcaggaatggggctgacaacatgcacaggtttttctcctaatgatgcggtggccacaggaagcttcactggacctagggccaggcaggtgtcattcatttctccagtgtctggcacagagaaaagaccccaggtcaccaagaagtgctttactgatgccacttactcctcacagcagatggggaaactgagccttaaaggaaacctgaattccagccctctcttggacattgaccctgagcactaccttgggccttggaaaagtcacttggactgtctggatagcagtctcttcatctatagcaatcaagaagttgaactagacaatctctaagtcattttcatttctaaattctgtgatcctagttgaccactattcataaaagagtggaagcctataaaagtacctaaaagtccatatagagagaatggtggaagatccctcatcaatattattttttattcccttcttcctagattttaccaattgtcattctcactcttaacattcaggctgtggggttgaattaaatcatctttgaaatactgagctatcaacagatggcatgctgaacacagatataccacaaataaacatttgggactgaagaaaaccaagagctggacttcatttccccttcttatctgggctcagaactctgcagtcttcccttttattgcccttgcctccaaacaggctatttgtgttctgagatggatcagaaaatcacttccaaagttggctacaaactctcagtaaatttttttttttttgtaactggccaattcctcttagttcacgtaacagagaagcacataattacttgtgactttagttaaagcaatggtcttaaaaaggtctagctcaatagaaaacgttcagactttctgagtgctgacagttgtcaaattcacctagttcatgtggccacatttctgaagttcttttcccagcccatctgtgagcaatagggtcagatgactaagagcaacggggcagaagcagcgttactgtgagtcaggttttcccattgcccagccataaagagtttgtctctctctgacccatataaacttcagcatttagggccctgtgtaaatttaatcccatcacttagcaccccagctgtatgattctgaagataaacatgaagttttttgaattccaggccacctgaaccctcttaggaaactcattagctccccaaacaaagctatgtctaaatctgtaggttggagcccaaatttcgtgataaacaaaaactgaattgatctgtcttccctgtaattagtcaccatttgctcactcttttaccaaaattactgtgactgggttagaataaataaacgcaatcaatgaatacgatttactaatcctttggccacataataacacattcggaactttctttcctaaaggagattttatcaggttggtgctcttggaaataggagaaagagaggaaaataacttagcaaatggcagcattcacagattcattcacttattcaacaaatattaatttagtacctactatgttccagggatcttacagtctcgtagcagaaataatcacacacacacacacacaattataccattaaataagtgtgagataggctggatgaaaaaaagtccaactgagaaaaagggtggggaccagaaagaagtctttgaagaaagaacattcaagttaagacattaccagtaactcagagtcagccaggtcaagactggtggaaggagtgttaaaaccaggagcaggtaggaaggccgcaaagcaggaaagcgtttgggccattgtcctgggagaaatttttcaagattccagtctaaatttaagagtttactgatttggggaagggtaagaaagtgtcttcctctatgactttaaaagcaagggagcaaaccacagaaggacatcacccctctttggtaatgattcctgagcagccagaggctgtttctattttctttgcactgtccccttttcaatgacccgtagccagctcatcgaccctgtcctggctctgtttcctcacttcacttattccattctcaaaaagtaggtgcctcataaatattgaatgaatgaatgaatgaatgaactcacaaggccacagggatgcaagccaaaagtgtctgcaaaaaataagcaagcattctaggctacaaatatttctaaagttcaggtcctgtcttaccacattctgattttctagagcagcagttttctaccaaggataattttgctccctctcccagggacatttggcaaagtctggagacatttttcagttgtcacaatttaggaagtgccactggcctctagagggtagaagacagggatgctaataatcatcccacgtcatcctacagtgctcagaacagcacccctaccctcaccccatcaacaaagaattatccagcccaaaatgccaatagtgcccagatggagaaaccccatggtagaggttggggcagcatcttggcagccacataatgggctgtgattaaccttacaagaaccctagtgtatatccttttcctcactcaccaagcaatgttaaaacctgcagagatttatttcattcattttctatttgtttaataaacagactcgaagcactggagggaaaaatattccagaaatgagctaagacgaaagctattggtaaccctagtttgacttaactgaatggtaggaaacaacctctgccatcaggaagtgaactgtagaaacagacaagaagttaataatgtttaaaaagctctggttcaaacaaatgtctacagggaccaatgggtagcctgaaaatggtgaagacatttccctttcttgtgtctttgatagaatgcaaagagcttttcttttctttggtgcggtaatcattcatatttgtcccatgacacacccagaagtttaaagaataacaaactcccaagtgtaaacacagaatttcatgagcgagtgaggcctctggatgaatccttataattgcgtatttggataaaataagatttccgtccattaaacaatgcagggcttttctatttgagaagagaacactttgaagggctgtagttattacccgtcaaagatggcaaacataagtgcccccaaggtatctctactccttgttcaagttcaatgctccattttcaatgctcccatcactgacccttgtgtggcctatttttacactgttcactgttccgcatcactagtttggtgatattcaacatcagcaagtgccaccaaatcaataacagaagcttgaagattgaaaaaaaagcaatcaaaagatatgcagcctgttctgtcccgagccatccacaactagatacttttcatgataatgaaaacgtgaggccatgtggacaggagagatggttccaggttcctgaagaatgctcatggccagttggtttcaggtgcatcaaatgtggctttagttcccacctgggatctccataggaaagggtgggcagcctacacgagaaggtatgaaaatgctctggaaccccaatatcatctcatcgttgaggacagtttttagccatttcaaagagcatgtgtattcatttcctagggctgccttaaaagtatcacaaactgggtggcttaaagcaacagaaatttgtagtcttacggttctggaggccagaagtctgaaatcaagatgttagcagggccgtgctccctctgaaaactgtaggggcatctttcctcatctcttccctgtttctggtggtttgccagcaatctttgaattccttggcttgcagctgcataactccaatcacatggccttctccctgtgtgtcttcacactgccatcttcttataaggacgccagccatatggatgagggcgctcaagctactccaatatgacctcatcataattacatctgcaagactttgtctccaaatgaggtcacattttgagctactggagattaggccctcaatgtatctttttaaaggggacacaatcaatgcatagcaggatgtaaactagaaatgtatatctttgttatcaaaacagggaaatttagcttaagttttcagagtgtatacaaaccatatagtaatccatactgcctcctaagaagaacaaaagatggagaaacctgctattgccttcaaactaaaggaaaggaagaattaggcaaaaaagaattaatccgaagcaaagatggtcccatctagtcagtcttggtgccatattctgacacatccaggcactcagtcagctggggcaccttctcccgcagggcatatttggtcgctatatgcctcgtgtttataattaactccagaaggctgatgcacttgctagagacgagctaattcttcttgctggcttaatgacagaatgcaaataagtactcacctgaatgaaatgatttcaggaatgccaagaagttctccttttccaaaatatcacctccatcctcctggcactgttactaaaatctctcatatttccccacttctgctctgggcacctgggccagaagctcctgatgtagcgctgaccattgccccaggcaccatcccaaacattttgcataagttgtcatttaatacacagaacaatcctatgaagtaggtactatcataaccacattttataaatgagaaactaaagcacagagagggttattagcttatgtgacataacacagcagaactggggttcaaaactagcccccatggtatcaaccagggtaccatattgcctttcaaaagtatggtacagtaactaacctactaacataccgactcagatctgagtgtcaggttttccagatcctctcacctagctgagaacgaagcctaaagcagcttttgttttcaattttcctcctcggaccacctgcatcaagacagaaggaggagatacagatatctggagcctaccctcagaccaccaattcagaatcccagggcccaggaatctgcattttaacaaacatccccagaaaattcttgtgtgtactaatttttaagaaccactggccctaaaggaatggaataggggaaatgtacagtgttatggtaaaccaggaaaagccagagagagatgacaaggcagtgggaaccctggtaaccaatggtcagtcaaatgacccaggggtcctgaccagaggagctcggctgctggggaatgagaataatgctgagcgagacggtttgacagtaggaggaagccagccgcctcctcttgcttggatcacacgccttgtgctggtggaaatggttatttgctgagtcaggggaggcattttctattagtttggatgggaagcatgggtgcaggcaaaggagtgcaactagagcgtgtgaactcagaaacccctccatgactccacgtgagcgagcagggttacgtaagccggacaaggactttggtgcacagttaaagacagtggtgtgatgcagggagaactgaaggatgaactgagggacctaagtcctgggtttggtccagtcactgattccaacctcgctgaggctcagtctcctagtctgtaaaatgggagaaataatacctgcctcgcgggtcctcacacacaggatctgacgtcgacccactgaggcatgtagtgctccatagcataagttactgctattccaaggcctctgtaaaagtttcctgcctcaatatatctgccacacggatgcctgaagtctattaatgacacatgagggacaactggtgatgattctctgccttactctgtgcaccccactgcaccctggccaaaaaagaaaacacagtcacctgaattcattctaaagtacagattgcttgttggcgaccttttctgcactagacacatacacaatacgtgcgtgtgcacacacgtgagaaagcatgaggacaagtgaaggctcgaggcaccaaagaacaaagtcactctcctcaccctatccctcccccagcgggtgctctgctgatcccctgcagggctgggctggctgctgctcctgcttccactcctgcttcctccccgaggcccctcatcctgtgcgtgccccctgctttggggccaggtctgcacaattgcagttattcagctcattggcacaaaaccatgccttcatcaatattttctaaatcagttttccctaaaagtgaagacttctgagttttcatcgccccctccttggtaggttttccatactgcccaaggaacctaaatgatgactaaccctccacttcattttaatcagaatggtcatgatattatagagctatggtgaataagaacatcattttgggggccggcccggtggcacagcggttaagttcacacgttccgcttctcggaggcctggggttcaccagttcgaatcccaggtgcggacatagcaccacttggcacaccatgctgttcttgacatcccatgtataaagtagagaaagatgggcacgatgttagctcagggccaggcgtctctcagcaaaaagaggaggactggcagtagttagctcagggctagtcttcctcaaaaaaaaaaagaacatcattttgaataaactggaataccaaaaaagttaattaaatagatagataaaagtgcacttgagataccaagtaaagaattcgtaagagataaaatgacggttagtgaaatgttatgtatgcagacatcccttcaatggacattttacctcctttaaaatattatacttattgaagaaatatttatttgctaattaatgtgtgcatagtactaccaggtattgtggaggatatttgtgcaacagacacagacctgccctaaatcctggttcagtccaatcttttattaaagtgacctttctttagtggggaaataaaagactattttccattcttattatgaagataacctagaatttcagaagtttgtgaagagaagcacacgagtaaaacagggcactaaaaacataaaatattgttattctgaggcttctgtgatgtctgggaagcaataagtccaaagggagagacttgaaggtcccagctggaggcccaggccagggactacaggtccccttcgcactcctctccctgcacacaacctttctcttgctcacagaggtaagtaaatacccgcctttctatcctcttccagcttgacgccctgattagtcatgcagctctctcctttcctccccaagaagcaacgctgtgctcttctgcaagaggaagttgcctagtacctgccaccccagagcatgtcagcgttcccaacccaggtttacatttttcctaggactgtctgtggatcctctaggaatccaagcaatcgcaggcatgaacaacacaatccaggcctcctgttttcagtcagggctgcaagaacgctgcagccttttcccctaacagtgccagtttgtcccagtaaacacatgggtatttgcccttgtccctgagaaaacacatgggcatcagagcaaagttcctgtgttctggtaacacttaattgtgctggttctcaaccctctttacacccacacactcacacaccacatttattccattgacttcaaaggctgctcaacagttggaaatatgcatgagaacagtgaaaggagttgaccgtatgactgagtttggtacatatgttaggattttagaagtgcatatttgactctttaaaacatacatttgtttatactgggtttagaatcatgaaaatacatcaagatttctattgccagtctgtgtgtttttcataacaaataatagtcattcatccttgttcaataatacatatttataggtttaaattaggaaaagattttcagagagaatttccattcattacccacctacttccagatgccatccagcacttgaggagctcccagaaagaacaggaggcaaacgagggctctgaggcatggaggagaaagagtggcatggacaaaaaagagctgttgctaaatccaaatgggcacagcatatgctagttcagtcttttgcatgagaaaaggagagaaatcttgttaaaaatacagattccctaggccccaccccagagagtctggtctgagtctgaaatgggacctgagcatctacattttgacaagcccctccccctcactgattcttttgccacttccctgctgagaaccctgggagttgaggacccctgtgctagtgcattcaggagcactatattcaagagtccggattcggtaatcggtcttctagatggagctggcttttgttctttggtgcatatccgggtgtaaagccaagccacctgaggtatgtgagctgatagcactatcaacagcctaaaacttttctaacattctgtttgacgtgacttgtagaaagttgaaatgctattttttcttctcgtatgtttctggactttgcctcaggaccaaattccaggatatttacatacattcattgtatagatttttctataagcatgaaaatttaatcttctaatagaaaagggcaaaaaaaatagaaaataagaaatgataatcaaaataaaggtgatttttctggaattgtattagttacctatggctgcataacaaattaccccaaaatttagtcccttaaaacagcaaacacttatcatcttacagtgtgtcaagaatccaagtgtgacttagcttagtggttctggctcagggcctctcacaggctgcaatccagttgtcagggctagagtaatctcaaggcccaaatgagaggaccatttctaaactcactcacatagccaatagcaggattcagttcctcccaagccgttgaactgagacctcagtttctcaatagctgttggccagagtcctccctcagttccttccacatagacttctccataaggcaactcacaatatggcagctggctcccaacagagcaagcaagcaagagagcaagaaaagaggggagatgatgccatagtcttttgtaatctaatcttggaaatgataccccctcccccatcacttttgccatgttttattatttagaagcaaatcactaagtccagcccacacccaaggggagggcattacacaaggacacgaataccaagaagtgggggaaggggaatcattgggagtcatcttgaatgctatcacaaaatacactaatttgtttctttcatagttaatgtatcatcctatttatgttttgctgaaattaaatgttaatgtaaagcaaacattttctgtacatgttaggcagcatattctgggaatagagtctagtagtgttggaaagatacattgaggtcataaattcaatgaaggtttctaaatgaggagctgtacacctactatgccaaccaagaagaaagagtattagttactgtaccacttacgaatgttttctgctacaggtaactgaaaatatgaatagctgtggcttaaaaataagggtatgtatgtgtcacataaagagatgtctagaggtggaccactgttggcattgtttcagaagtttggtaatggcaaaagcagcatcttctattatcttggcttgttaatcatgcatgtgacctcgtggtcacaaggtgacttcccaacggtaaagcgatacatcatattctaagtaggatgaaagggcaaagggccacaccgtatgcctctgtctcttttcatagggaagataaaactttcctggaagtcctgcccagcagacttccacttagacctcattggccagaatgtctaccctaaactaatcactcaccaaagagactaacattaccatggcaagtctaaaccagccatgacttatctctgaaatcaaagggttgaaattgctattgcccaaagaaatattctgtcggcagagaagtgggactgtggatgttgggcagacaacacatgtcttctgtaaacatcttctgtgttgttatttatgtgttctatatatccaatacaatcacaattttcactgaccatctaaaaagatattttcccataaaaacagaatatgcaagcatcattgcttatgaaatcccaaacatacttaaagtttccatcttgaaaattccttattcagaaataaaatgtccagattttaagacaaaaagtgaccccatgttttttaatcctgtgtacatataaaatgtttttttaaatattgctatcagataaattaacaagatgaaacctcctaaaatttactgcattagccaagtattatcaaaacacaagacagattgcttttcctgacgtctgctgctagatttttgttaatttatgtttccaaaattgtagcatcataagtcaatgttgcacaaaggtatttcatgtcatttataaaaatccagtaatataaactgttgactccttataaagcatctattaagacacagaaatatcactgaagcatatttatgcctttgatcttgaggtctgcataatgattcttgccagaagggcagcttgttccctggattacagaacagaaaatctgttcttggaggagcagctcaacctcctctgaggtattccattctaagaaactaaatggcacctgcacgtcttggcttttgtttttgttttttaaaagcaaatctctagttcctgttctgctccaaatctaagtgagtagataaagttctgctctggacaagacctgccctcccgcagagggataagaggagtgcacccctgaggaaataggactccagagtggcagttttggtgagggataaatcaaatcgatgatgcatgtgatcgatgagcacagtgggattgtcagaactgggattgtcagaaacaggtaccatcgttcattcaacaatgttcaagttcagatgcaagcccacctctctttgctttggctgtggcatgtagcagccagactaactgaggatatgtggtgacagcagacagtaatggaagagcccaggagaattcctaagcctgaattccaaccctgtgtctgccattaagtgggaaggtatccttgatccagtcacctagcctcctgacagctttggccatcaatttctccatctcaaatgggactccatttctttaaggatcttctgctttagatctattaatttgccgaagcaactgtgctttgattgatgtggaattttgaactgcctttaaatggagtgactgattactagcagaactttgttgggttcagtgtattatgaatgcatgtcaaaatgtacagtgaggatatttttaaaaatattccctccagtttccttcaccctagatagatgtacaggaatgtccatgttcatgttgtgaaaaggacatttctcctaacctcacacagagatacaagttaagtcaatgtcttaccaagaaggcaataagagaattacaggagacccagagatctgaatagggtaggcagagaagggacgtggggggagagatgtaaattcctagggaaatagtgtggagtgcagggctggacctaggcctgggaatcaggagtcagagttctaatttcagcttttccactgattcacttgggaccttgagaatgtctggcgcattttaatcattctgtgcctcagtttcctaatctgtcaattagagtgagagtccttggctgagtgcccaggattgtgagaacatactgtgcaggaccatgaaaacacaatcagtaccaaaaatgcaaaacatcctagttactagatgtcctagcttaaagtggaaacgttaacaagagtaattcctttaataatcaaaatgtgaatgttttaggaagcatatctttctgggtgaagattaagtgggaaaactattataagggcttgtagtcctggggatacataagcaaaaagaggtcagacttgaagtcggacaaattccagctttgccacctagcatttgtgtgaccttgagtaatttcttaacctcccacggtacccttatctgtgaaatgggaataatggcatgcatctcagagggtgggggtaaagatgattacagatgggagttcttttgagccactggtggggtttgaatttacagacactaagtactttgcagtgatagtttctcttattacaattgcatattaaaaagcttagttccccagagcaaggaagtttttaatgctattatttactgagcatttactatattgcaatgctctatcttcatgctaagcagtttacatgaattatctcatgtattcctcacaaaaacctattactgttactgttgtggaaatggaggtagagagagactaagctttatttctggctccattgtgtttccttagcttgccttgctttctttttctcagctacatctaacaggctgtaattttagcattctttgaagccaccttaaattttcaagactaaagcagcaactaacaaactgaatatatttatataacatgttatttttgtcatgttgctgttcaccccttgagacctgctctaaattcacttggatatttgaggataaatcatgctcactggcagtttctggaaaacattgaaaaagcagctaccctgaaattttctaagcaacttaaaagaaagcttaagaatttccaatatgtttcttgaaaaaagcatttatctttctgcttagaaaaataaagttattatcaagaaatcttacacacattcatatgttcacagatcttcgctaaggaccaattgggtatatgatgtgtatgacttcattataacactgacctcagagaccatgctgtgggagttcttacaatcactcaaaaggttaaagtcagcaaactcttttctgttaatgtgccttttaatcaggaaatactataattgccttggagaaatttgaagaattgtttttaaagcaatgtggtattaaagaaagagtcaatatcttttagaccagatttgaaacgaaaatccatctcttatttatctggacaatttattagcatgtctgaaccatgcatcctttttccataagatggaaataatacattaagagttgctgtgagttaaatgagaacagtgtccaatcataatagccagttaacaaatgttagttactatcccttcccttggaaaagaatgtcaaggcagacatgcttaaagtctgctctgtaaagaggtgggaacctgccccagtaaagtgcttctggcacttgttaggtaatagacttgctttattttctaataaaagtgatgggccttctcccactgtacactcaaaatgtatgtatccatttcagggcattcatggcgcccattgcccatcctttgaccccatattaagaacatctgctttagtgtattaattttgacttcatcaaatcctcttacaaaacaaggtcatctttagaaaataaagttctttggatatcttgcagttaaatgaaggaaaactgcaggcctttattttcaatagctttcgaaaaatgcagtgtcagaacaaagccgtgtagtagacaattaagtaaactgcacagaagcttcattaaaggattgatctaacttgccacatattttgtgcaaccttttaaaacacacaagtaaatgttttctctgagtctagatctagatggacaaagaaaaaattagaaccacaataaatctatttaacttcgtttacataattgtggacataccgttttacatattacattattaggacattagacaatagatcaagaaacagaatgttctagaaaataaattgactattggtaagcatcatgtactttttccaaaagaagttttattgtgttgaatcaaaggtggctccttggcactgagagccttcaaggagtcagccaccaaagtcctcattccttaatcctgagcttggctggatggctgctgacagttgtcaacttgtttggatttcaaactatattagatctcttcatatagctgccactaccaagcagttgagttggctttgtcctactcttctgttggtaatttttattaatcgtatgcttcaaagtagtctataaaaatccttccagagggataaaagctactgagaattcattgtttaggtgaacatgtatcatgggaaagaatgctattgggaaaggggccaagtttgcattctgatcccatcattgctgtgtgcttgatgctagcaagtcactttcgcctctctgaatctctgtatcttcttttgtaaaataacaacttaccttcctagactggtctgaaagcccagatcacatggagatgatacacgtagaagcacttgatgactcttaagtgctacctgactgcagatcatgtgattacttcctagcggccaccacaaagggactgccacaggattcattaggtctgcaccactgctatctgcacctgtccttagtcaaaagtttactctatttgtttaattcaaaagtgaaactctccattaacattttttaactgccttcctttataattttcttcttgctgttaatgtcatctctgttctccctagcaattaaatcagaatctcaaagctcaaagctcatcttaggtttttctctctccctatctttgaactaatcgagtcctgtagattcttcctttaatgtctctggaattcattcttttcttttttttcatggactctattccaggtttggccctaccgtctcaaacttgggattcagtctttctgccttgagtctcttttcccttttccccagcctgcgcactgctgtcaaattcatcttgctaagtgaccgcattgatagtggctgcatgccctgactctgacatcttcaatagacccccactgtttgtaaaatgaaatcgagtctggcctcaacctgtctttccagctgtggccatacaggtctatctgcctgtgacgctcaccatatcatccttccttcttcctggggccttgcacacaccattcccaaggtcccttcttctttcctctccaaatcgtacctcattttccaagccctgctaagtcccacaaccctcatgaaggctttttgagtcctcacagctcacagtgatctaatttcctgaagaaattctaccaactgagtgtttgaagagtcaataatcaattatgaactaactactctttaacatcgtgtgtgtagttgtcttgaaatgcaattaacttaggttgtgtcatttacattttactgagtgccttcaattaataaatatttgataagtatttatccgtgttgctgggttttacttgaaggcaggaattttatcatgtcctttcggtttcccgtaaagcggtggttctcaacctttcacatggtaaagaaacgcatggagaacatattaaaagctctccaggattccacccccagagacgcagtaaggtccgggtggggcccaaacatctgcatttccaacaagttccaagtgatgctgattctgccttccagcatcacacacagaacctgtacgtaaaagctaatatatatgtataggtactctgtaatgagattttttttctctttttttctattttaagctcatgttctgtcacttcctccctttagattgattttgaagatgtgattgcagaaccagaaggaacacacagtttcgacggcatctggaaggccagcttcaccaccttcactgtgacaaagtactggttttaccgcttgctgtctgccctctttggcatcccaatggcgctcatctggggcatttactttgccattctctctttcctgcacatctgggcagttgtaccgtgcattaagagtttcctgattgagattcagtgcatcagccgtgtctattccatctacgtccacaccttctgtgacccactctttgaggctattggcaaaatattcagcaatgtccgcatcaacctgcagaaagaaatataaatgacatttcgaggatagaagtatacctgattctatttttttccttttaattttcttggtgccaatttcaagtttcaagttgctagtacagcaacaatatatgaatgaattttcttggttcagaacaaagaaatcactctctcagtcttcataactgttatttgtctcttctgagttatttcatctattttttttttgatggtctgaattttttaaacccattcaaaatttttttcgcacatttttatttgcatgtggatccttgtgttattggcagagatgtgaacctattgttgaaagataatttgagagaaatatggagaactgaggaggaaagaaaagaaccaacaacctcaactgcctattctaaaacgttgatcattttatggtaagggaagaattccaggccatggccattaactgtacaggtatgtgggcaggttttaagcaaactctttccccaccatctgaagcgttagtggactgctgctattcactttaataattcaatgggctccaatggtctttctcatgttagaaaacataacctgcattcacatggtccgactaatgccttacccttcttgaaattttaacctgtgatactttctgtgcctacatatttgttatatagataatgagacccaagtgccttcctgttcttcacattttcgttttcaaatagggtccaacttaactgtcaactttcattaggtcaacagcctccctgaagaccaaaattagaaaattcattaactagttctctatgctggtttctgactctgcgatccagagtcagatgaagtccacgtctgcactcgatcacacagcatctttgtccatgttgagtatggttcacatcagccccatgaaacaaattaaggtggacgaacggggttgagccctctctgagctggcaggagtggaagccaactttccctgccactcacgagctgcacgaggtcagcatgtctattcagcttcgttgattttcaagaataatcacgctttcctgactccaaactaatccatcacccgggtggtttagtggctgaacattgtgttcccctttcagctgatcagtgggcctctggggaagggctcataaaatggaggccattgtatgagtctatcagagttgttgcaaatgtgaccccttcaaagtaaagcacttgcaaccgtctgttatgctgtgacacacagcccctccccctgccaggagcttgggtctaatacaagcatcactttgctcacagagaagatggggaggaggcagtcataaaagattgaggtaattttgctggaataagttcaaattcttttgaactcaaactgaggaatttttacctgtaaacctgagtcatacagaaagctgcctggtacatccaaaagctttttattcttgttcaaattaagattctgcccttggggattttatttttaaccttcagttatgcttttatttttattttcatacacctattggaactctgcttgattttttttttcctgcctcttccagttttcctgacacttcagttatcaacctgttccctactttgattttttgcatttaaaacagacactggcatggacatagttttacttttaaactgtgtacataactgaaaatgtactatactgcgtactttttaagtgtaaagatatttttatctttatatgaggaaaatcacttagggaaatgctttgtgattcaatctgtaaactgtgtatcccaagacatgtctgttctacatagatgcttagtccctcgcgcaaatgaagcgctggtccaggagactgctaaaattttatatgcttactgatatattttacacttttttatcctgcatgtcctgtaaaggtgacaagtctgcacaataaaaatgtttaacagtt

SEQ ID NO: 2 - DNA, 13440 bp, Equus caballus
  source 1..13440: mol_type = genomic DNA
gccccagtcccgctcccgccgcccccgctgccacccgaggccctagtctcccgctccaactattccaatcatcctaggaagggtggggcactcggctcttgggtccccctcggttgccccccctttccccatcatcggtctccccttctgccccggtccctccctttctggggtggggccagccaatgggcgaggagactccggggtttggcctgggagccggggagctcacagatcccccgccccacagttctggccgctgtcccggtgcgcacggacgtggctcgagtttcctccgctctctgctctctctcgctctcccccctcccgctcttcctcttctccgagtctccagctccagctccagctccacccggccggccccgcacggctccgggtagccatggaggacacccagctccatatcatcgagcagccgcttcccgggtaccccgacgctggggacccggggtcctcccccgtgggggcgccagcggcggaggagccgtcgggggccggctctgaggagctgatcaagtccgaccaggtgaatggcgtgctggtgctgagccttctggacaaaatcatcggcgccgtcgaccagatccagctgacccaagcccagctggaggagcggcaggcagagatggagggtgccgtgcagagcatccagggcgagctgagcaagctgggcaaggcacacgccaccaccagcaacaccgtgagcaagttgctggagaaggtgcgcaaggtcagcgtcaacgtgaagaccgtgcgcggcagcctggagcgccaggcgggccaaatcaagaagctggaggtcaatgaggccgagctgctgcggcgccgcaactttaaagtcatgatctatcaggtgagctggctgggcggaccgcgcccagagctccgcgcccggcccactgtccccggcccggggcttggggtgggggtacctgcccgaggcgcgcctctcccgccagcctggcctagctgggaggacccaccctccttcctgcgagcgctgctggggtggggtggggtggagtggggcgaacggcgcattcttgcagcgcgcagaggggtgcgctctgcgacctgcacctcctcaccaagccccctccctccggcgtcaaactcttggcggaaccgagttagagggctagagtcggggacactggccctttcagacatctcccaacttggctctgtcaaagtcttacttgggggaagttttggggaaagccgggggtccctggcggccgttgcggcctgctgtaagcggacactggcggcgcggccggggtttggggcctccggcactgcccgcccgatatagcgggtgattcagggctcaggcacgcaccggcgggggctgcgccaggcgagggacgcgaccgccagcacacccctccagaccccgcgcccccgcatcagcctcggctgcctaccctgtcctctcactgcgctctgcccctggcctcccgtcactttcaggcccttggggacatcctagccggtggcgcgcttgtaccctctgcctgatggaagcaggcggggcggtctgggggccgcgagagaagtgcaagaggggctagtggaggggagtggacagcccccgcctggtcccgcgggctgggcgcagtgaatcaccctgcacgcctgcgggggtggcccaggagcaggcgcatgccaggaggaggctgctgcgccccaggctccacctaggccgcttgttgcttcttttgtacctctccccagtcctcgtgcctggataaaaatgtagtttttgtatcataacccaaaatgaacctgagaatctcagtaattctgttcctctccactccctctcccaatcccattaagggtaggtctgagctaagggcttggtgatcttttgtccggctttgcctcctctctgcttccaggaggtctggttgatcttcctaccccaagaaggtgagattgtgggggcggaggagttagtctatgctccatcttccccttccactccgtaccaaagggaatggaaagaaaaggaaggtagatttcccacactccctaggtgagaggacccctaacagcgagctgtggtttcactcactgctctggggcctggtcaagctgggttctgagtgaccacctgggatacagtgacgtccctggcttggtctctccaggggaggctaagaaagacgaagaatgggacaagtgtttgaggacagtagacacagggaggtggtgagctggcacttagctgaggcggtggtggcccagaagcggcaccctccccctctccccaatgcccagggactccccccaagaggcatttggcctttaagaagctttgcttccccccatacagcactatctggcagaaaatatttgcccagagttgtttccacttaatctaactgcgtggaggaagctggatgggggcgggagcaaaggggggggaggtgagtgagatttttaaagagatctggctagccctggggtgcgtggagggaaggagaacaagacaggggtctgggacagccagctagctctgcctgttctccagagcctgtgaggtagagggttgctatcatgggcttggatgggtgtcccagaccacagaacctgggaaagttctcagcctcaaggtaaacctccttgtaccaaagatggagattctgagagaaggacgggtatggagggagaaagacttccatgcagaggccagtggcatcccgccgttactccctcttggcagttggcagggatctgtgtctgtatattgtgcattgtgggaggggggtgtctaatcacagggtggctttgactgcatctatactaaaccagggtgcgtggaggacagagggttgaggtagaaggaatggctcaagatggggaccctgagtccccaaaccacagaggaaataactaagaactgaagtggtcttaacgaggagaggagctcagagggaaggaaaagattccaacaatggggtggggctcagggaagcagaacttccctgagctgtagagcagatgttccaggtgggggcagaggggtggggtactcagctgctggaggaaagcagaggctgctcctccctgaggtctgcaggcctgggaagagaatgaagtaaaaatagctctgtgccttggcagacctaatagggaagattgggggcggcaacatttatggtcaagtccggctgggggggtaggatgcttgggtgtccaggcagagagagatctggagaaaggcctcctgggatggtgtgagttctccagcatggggcgggggtggggaatgctatattcctcgctgcagatgtgggaggtgatgatgtggcaaggggggcagggtggaggggagaatgaaaggctgggaaggccccagctgtgggatcaggtcatctggcctcccacatcttcttcctggggaataagggagggaatcccttgtgcagccccaccccagattcagggagctgctagacataatgatttcacaggttaagtttgcatttgaccccctttccccatacacacacacacacacacacacacacacacgaaattcagtacctcctaccccaggatatcagagccctcaccttgagctcacttctgaatcattactgttcttctctggcattccctcccgtctcctaaacaaaaactagcaatttggcataccaccctgtcaggcagcacctaactaagtaacagccagtcaatgagagaagtgagttctactagtagctgggggtcagtggtcaatgatttggatgttattagagttcttttagaatccagaaagaggggctaaaatggaaataaacattttctggagcagttttattagtttgttttttaaattcctgtgacttgctttattgtgatattcactttatttatttatttattattcctttgtgttttttttgttttgttttttttttgtgaagaagattggccctgactaacatctgttgccaatcttcctctttttgctggaggaagattgtcactgagctaacatctgtgccaatcttcctctattttgtatgtgggaaccgacctcagcatggcttgatgagtggtgctaggtccgtgcccaggatccgaaactgtgaaccccgggcagccgaagcagagtgtgcgaacttagccactcggccaccaggctggcccctctggagcggttttgctgaggcagttagtaacctacacatggacccatcatacctgcttcaagtgcttgaggacaggacgggaagcacttaagggattaggtggctcataggcattaagggggaggacaccagggccggaaggagggcctgctagcttctcctccccaaatcctaacccaacccatagctccttccttgaggcaagtcagggctaccttgttccagaagaaccccaaacctttgcctcaacatattcccaacttggggctctggtccctcttacctcctaagcctggtggcctagtagagagagtgcttgccatgcccttcctccaaaaaactgagtttgggttgtttttcttggtcactggatccaaagcaggtagggtatttttaggggtattcctaagatcaaaggaaatcatgctcctccctctgtttttttttttctcttttttagtaaatacaatctgactattgtaaaggtcaaaatcagaaagggaagagatggcacagagtgggcaactgatagggagaggagccggaatacagttgctaggcaaccaacctcaaagatgagtggtggtttgttccgttttttttcaaccataccattgtgaatatttcatcacttgctcattatagactttttgagctggaaggtgttttagagataatcatcttcagctccttcattttgtttcggaggaagctgagacctaaagaggggaagtcgtttgcctattgtcactcaagtgattggaggtggctggggccagaacccaggtgtaccccttcttctgctacactgtctgctccccgcctcctccttccaatcccaggctctgcgtcagaagtcagggtagaggctagccctaatggagggttagttctggctcctccattctctgagggagctggttgcacagtcctggcactggggctgctttgtgtgtgtgtgtgggtgggtgtgcagtgttggctgagagtgcataagattgctggccattagctgctcagcctcagaggtgctgaagtagaggcagcgtgtggtcacctggtccaacgacagtgggaaggcaggtgcagggtgtaaggagtggagctccctggccagcatgggtgtcccctggcacggactccgcatctggctaggagaggaccacgttggcttctttctccttaacctccctgccactcccatcttctccctacaccccaaaggcaggttggcacctgctgctttgtgtttctgtggctgcaggcctttactcaacactgttgtcgccaaagtctggttgtgctcccaactgtccctctctcctgacttctcctgattgtccaaagcaagagtggcagctcagctcctcaagagggagagttgggagctgttgctggtgcaccaggacataggcccacccatcataaaaacagcttggtttttgatcctgatttctctgtgcatcttgatgtctgatatcccaagtgtggatggagggaggggtggaaagggaggcaggaggtggggatttggccaaagggaaatagccaggatgtccctcttccatgcctgtccctctccttatcctgggaagcacccagtgcccaccttccctggggggaatgacgagaactggaattagcagcatctggcccaagacagtgaagctgtttgccttctcttttactcatcagagcctctctggttggaatccaacaactgccctcttagaaaactcttcctctacccccagcagcctctttccccatcactccaaacccccgatagcatcatcccacaaccacagctgggtggggatagaaggtaggaggccaaggatgagacttggaagattctagagggaggaaaggagagggtttccgtttaaattgtttattaattcatgggcctgggtttgttccactcctttcagccccagggccctcttgatgaagcatttaaaatgtggtttctttctatcctaatgcattgtgtactcccttcccgtccctactcaccagccccaaatctgcgtgcccatcaaggcctctttctctcaaccccatccgcacccatcccaagtcccatttaaaccccttttcctcatcttagcccaagaagaagctgaatgcaccctctgacctggccacacccaagatcgtgccttttgaccttctgcctgagactgagccctttgacctgaagcctgagacagtgcctgagctcccaccctttgaccagacgcctagggcactgccctattacccagaacctgtgcctctgcccacccctgtgaagccgacagccggtgggggcgcttaaggtcttctgggtcccatgaggctgtgggcatggggtgtcgctccgtccaccatcagctcactgcttagtggtggtcagtactgagatgaccagcctcttccaagccactgccgcttgtgagtgggtgtgggtattgtgaaaacactcgcatcatgcagagttcaggggaatgtgatgtgtgcagtggggtgggaggtgaagggaaggggccctcctctacttcttggtttctctcttaatggtagacgctcccgacacatacttgagctgcactcagtgctgccctgcatgaaactgcgccatggcttctgactctcctgccccttttcccttctgcctcctcctcagggacaggccagtttggctgaaagcctctttggagagggccctcatgaggccaaggcctggggtcggttctgtgccctctccactccctccctgtagctggggaaaacatttacacttattttttccagttttttaagaaaatgttttgcaaagagatcccctgtgtttgccagatcctgccttgggcggggcacccaaaagctgccaactcagagaggccagaaggagggacgttttagctggctctggcttggattgtctggcctccctatagcacatttctcaacttttttttttgctgatcatccccctaaggagtccttttagacattttccccctaattgtcccctctcccatgaaattttactactacagatttactatatatctgtttatctactgggactctttggagcaccacaaactattgcagtacctaagacctctccctcccgcgagaatcgatttctgccctcttggaggcaatgtcacccctgttggggatggtgctatagcagaaaagaaggacccttctgcctttggtgtctctccccacttgtcttggcgacctaaagtagaggccccgagtgttcaagacttccttcttcacctctccctcagtctgtaccctcttctaccgtctctctgagcagctcctgggtctctgaatttcctctccggtgtcttctctggccattcccaggcagtggctggggacagagctggttagctctaagtcaagagaggctcctacccagagccatatgatacttgtcccttctccctgagaaatgtgaaatgcctgaccaaggaccaggcacatagttggtgctcaattaaaatataatataaaaatcttcttcctaagccagaggatcgtaagtttatatagcagggttggggagtggagcggaaggggctgggagttgccctggggctcctctaaattcatcctggactagtgcctgcctgggttggggagttgtgtgtgcttggtggggggcaggggaagctgtgcatgcttttgttcagacctggtcattaaaggtaactctggaaaccaagtcttttgggagtggtggttttgtttaataaaaacatgaaaacaagtgaggcccctcctttggtttaggaactctgccagggaagtaggggtgggacaagggtgcctggcccatgtgcccagggtagggggaggtactttgggattgtattggtggtggttgtagcaacaggccaactgggtgggcccaagacagcactccaaggctgtgagtcataggggaaacctggcttgaaaagggggaggaaatagaaaatccattaatggaggggaaaagctaaagtcacagtggtatgccctctccagagccagtctactggacattgtccagggtcttaagtcaaattgtttttcctctgtctcctgaccttccaccaagaggccagctcctctggtataagccatcacccttggggaagcctacagtgaatagggttatattctcaatggggttattttctggactggtgaagatgaccccaaatcccagaataaacctcccttcactgggagtgcttcctgatgtatgtgagacctttcaccaggcactaggaagatgggattggctacaagaagtctgcagctcttgccccagccctgttcacttccgagctcagcaggcaaagacctttatctaggaagccagaggccaggaaaaccaaccagctcagcctttagagggtggagtggaacaggggggagaatcagggatgctgggggtggggcagggagacaatgtggatttgttttacagcagctgggactgcttcctgactattccccacccatgactcttaatccctccctagctcatccacattgtctcagcttagtgaaaagagggctactcctctaccctctggtggagggtgggtggctttttagtttgggaggctttgaaggagagatcagagctgaaatttttttgatactgggcctgcagattaattctgggtggagatggatgaagaggtagtggagtgtttagaccctagcaaatcctaaaatggagtgtaagagaaaggactccagtaggaggcggagaaaagtgattacaaaaggatacccgaaggatacaactggaagtcaaagggaatgctaagaggggctgggacctcttacactcctaacaagaaaaacatcccttcaatgcctctgtgaatgataagaggcagaaagatggacagaataccataggatgcccatgatagtataagccagctatggcaggtggagactacaagtcccagcatgccctgtgcccaggtcaccttgcctgctgggaggtgtagttgagtaatgagagctcgaggtggcttggtgcccagtagagctggtgcccagttcaggagaagaggtggaaccagcattccctctttgtccctgctaatgctattgacagggagggaaggaagttgggagggggtgttcctagttttcaatgtaaaccaggaaactctgcagggatgtggatgggagggaagaggaaagagattgggggctggggaaggaatttctgcagcaggtggtgcagagataccaaggggccaatatatatagtacgcgtgtgtgtttatgagagaggttggctcatgttgccttatatttcttttttatttttatttatttatttttaaagatcttatttttcctttttctcccaaagcccccggggagatagttgtgtatttttagttgtgggtcctgctagttgtggtacgtgggaggccgctcagcgtggcctaatgagcagtgccatgtccgcacccaggattcgaaccggtgaaaccctgggcctctgcagtggaacgcgcgaacttaaccacttggccatggggccggccctgccttatatttctcagatgggattttcttacagttctgggtaacaatcttcagaaaacccttcagaaaaactattgtctctcattcattggtctgaaatccctgaagagagtctgggccaaagagaagatcctgtttatgttctgggttatgccccttttccctttctgtgctcagcctcccctaccttctggtgggggctgatcctgagtttttgaaatgttggcccacagtcaggatcttggaaaaccttcccagaatgcaaaacagtgctggtgctaggtcctcccacgggaagagggaagaactggctctgagctccaggatcatggggaatctgggaccaccatcctactcctggtcccttgaactccctagagaaaaaagcaagaatggaaagtattccctcccttctgttccaaggcaaggaagtcagaaccagaagaaacgttcacgttgggaagaaaacatgcccttggctgccagcaagcttcttcctcttttctccctcaatttctgcatggggagagaggagggatgaccactggtggagccaaacagtacgaggaggtcaagtagtgggggcgtgggacacagctctccttccctaagactggttctgaaacggtctgaagtctgggaaacgaactccctctaccttgccttcaagctaccctacttgggggtcgcctgatattggctgtgagctaattccccaacccccaacccctcaacttaaacaaactcctaagcgctttttggcctggttcgggcagtgtgcgacttagtgcaccagagggcgctctgctcatttccaaagttaccttgtgttccctgggacgcaaagggtaagaactgctttaatataagatgggtggatgcagggtccaaggacagggttggagggcagcagagatatttaaccgaagtgcaattacaccacgcccaatctccgacccgatgctcggttccctccttgcgttgtctatatagttaccattcattcaacacatattttttgggcgcccacgaggtgtccacctctgtgccagatgctagggatacaacggcgaacaaatcaaggccaatttcacgggtaccacgcactctccaggatcaccaccgccctgctgggttctcccagcgtccctgctcagccctccggttctgtcccccttatctcttcgctctgctttccttttaggatgaagtgaaactgccgtccaaagtgagtatcagcaagtcgctgaaagagtctgaggcactgcccgagaaggagggcgacgagctgggcgagggcgagcggcccgaggaggacgcgggggcgctcgagctgtcatcggacgaggcggtggaggtggaggaggtcatcgaggagtcgcgcgccgagcgcatcaagcgcagcggcctgcggcgagtggacgacttcaagaaggccttctccaaggagaagatggagaagaccaaggtgcgcacccgcgagaacctggagaagacccgcctcaagaccaaggagaacctggagaagacgcggcacacgctggagaagcgcatgaacaagctgggcacgcgcctggtccccgccgagcggcgcgagaagctcaagagctcgcgagacaagctgcgcaagtccttcacgcccgaccacgtggtctacgcgcgctccaagacggcagtctacaaggtgccacccttcaccttccacgtcaagaagatccgcgagggcgaggtggaggtgctcaaggccaccgagatggtggaggtggcccccgacgacgaggagggcggcgcggagcgcggcgaggccgccgacctgctgcgcgggagcagccccgacgtgcacacgctgctggagatcaccgaggagtcggacgccgtgctggtggacaagagcgacagcgactgagcgggacgcagggcccgccccgcctctccctgcccccgccccagattcctttgcctctccagactttctcttttgccttctctctcggccccagaagggctgagatgtttctaaactgagaacacacaccgtcgccccccacccccagcacccctccaagtcttacagctgttaagacgggatggggaggcagcttccacaatggacagacccaatttgggcacagtctgggtggaaatggagaggggagtgatctgtgtcctggttgtccaagttggggatcctagaagaaattgctgtcatttccatggttgggccccccttggggcaggctgccttccccccagtctccccgttacactcacccagctgctgtcattcctactcactgagctcttttttctcatcctgatccccggaagcttcaaggccaaaattacccttgcccacatgggaggccgcatactggaagcactcggaagcggtttttgggagaacctgaggcaatggcggagagagggcagagtgagccgtgaccctggatgagagtctagcacaggctgtatttgctaaggtctcaacaaggacggctggagccaagcagagccctggggaaaggggagggggcggagcacgatgatgaatacatacgtgtgggaggaagggtacacttaaatgttcatgtacctgcacacaggagctcatggccatggctttgggaaggagaacgggaaatagtagaaggtggaaatgggtgaagagaggagaattctcctcattagcagcaagagaaacagaggacaagaatcctgtcttactggggttctcagaggacagcaggaccattttcaggataaggagggaggacagtgtgagaaagcgatgatccaaagagaagagagaggtaaagctcccctccttcaactgagatctggacggtggctgcccctagggacaccccatttcttaaagcccagatacttaggggcccagaagggggcagcaccaggctgtggagagctgagaattaggagggagactgatggaaagagagcaaggtgttaatggccaggccaccaagctgagagctggaggggaggggaggagagtatagtatgcctgtgtagagagcaacgtagccttggctcgctcttggcagtttttttctcaagctctttccctagagctagtgtcaggaagcagaagaacttgccgagacctcctgcccttgtgggttcctctcctgaatctttgggggctgcgtacagtctgaaggcctgatctgtagtccactgaccaggttctcctaaggacacttcagtccctctgttcctcgctagtaaaggtggcttgtcagaagcaaggccaactcccccacccccaccccatccgcagccgaaagaggaaggctgcggccttcttttttctccccccacctcctctgcctcatcttccctgctctgctttttagaattgcagctaattattttaaggggtggagggagggagcctggggacacaaacctttttttacaatacaaagctttgcactcccccctccccttttcttggttcccttctctcttctgaatggttggagacacatcagctgagggaggatggggattgtgggacgaggtcccttggtgctgatgggctgaaggggcctgagttgtgggcagatacagtttcctgtgggctctggggagcctctcacgttgctgtgtcctggtgagcagcccgaccaataaacctgcttttctaaaa

SEQ ID NO: 3 - DNA, 347636 bp, Equus caballus
  source 1..347636: mol_type = genomic DNA
ggggggagagataagtgaggggaagaccgggaaggcagaggagagccccccaatcccgcgccgaggcggcggcggcggcggcggcggcgcgacgatgaggatgatgaatacattgcaaagtttttttggccccggcgaggggtgtcagattgagtgctctgtgcgcatgtgcgaaggtgtccaaactgacaatgctggggagatgaagatagtgtgtagctgcttctggactcaaggaggaggagagagattccgcgagccgacaccatgcgatccaaggcgagggcgaggaagctagccaaaagtaagtctcccgcgctcggccgcgccgcgccgccgggcccgggccgcggggccggggcgcccgggccaggggtgcgcgtcggggcgcggccggcgcgcctcaggctcccggcccccggatcggccgcgcggcccgggggccgctcctcctcccgcgctccggggcggccgggctcggcgcggaggctcggggcgcccgggccgcgcgctccccgaaggcgccggccccctcctcgccgaaccccctccccccccttgtcaggcgctcgggcccgggaacccgaggcgcgcggtgggggccagggaggggggcggagggggagggctgggggtggaggggagcgaaaagcgaaagttagcgaaaagttgacgaaaggggagagcaactttttcctcctcgctcgctctctccctctctctctccgagtggctctcagtcctggtcaaatcatattccgggcttttgaaatagtgggcgctagagcaccgcctttggcgtccgccagccgggcgcagaggagggagaccccgagccggggagggggcggaggagggggcgcgggccggcgcccacccggctccgcgggcgcaagggcaggggtggcgacggcaggacagccacagccactgggggagcaaaatggagacgtcttcccgggcttggaactatggtcgtgaagtttattcccaactggtgccccgcgcggcggcgggcgctcctctgtgactccggcagcccgcgcgggccgcccaggcgccggtgcctctggcaggagggtccccaccgggccgccttccgacccgccgccgccctcgggccccgttcggactgtcccgcacccgctctcctggcacagccgtgaggcccggtggggacagagctgctccggcgagactgcccggtccccggtcttactttctctttcgctcagtctccggccgagccccgggctccgcgcgcccagcctgctgccgtcccggtccccgccgccggccgggtgcgcccaccgcccgcatcaccgcccgccgccgcgccctggggccagccgtgcgccctccgcacccctgcggccggcgccatcggctgcgaggccagcgggcccggggctgtggcccgaaaggcaggcaggtggggacggcggcgccgcggcccggcgagcgcggctcccgcagtcccggccacggcccggctgcagcgaagcggcgagcaccgcgcggggcggaatcggcgcctcctgctctcgcctcccccttctccgccggccacccgcccacccgcggaagggtcaccggcccgctccccttgggattccctgcccgaccaccaggccgttccgtctgctcctccgtggcctgaccctgctcggcccggcctcacagcacccacggccgagcccctaagcgttagcacggtgggctcgaaactcgcgacagttttccgaagttctgctctcttggttcggtgctcggcccggctcggcccccaacagcagccccgcagtcgtaggccgcgcggaggcccgccgggggaaaagcagctcgcagcttctgcaggatgcccccgcactccgccggcgctgttccgggcccctccttgaggcgccccttcccagccagtctcctctgcccggtgctgacggcctcggcctccccggcctcccttgacctctggcccctgttggtacgaacaaggccccgcatttcgtggctggcaccgcggcctgtgcgtgctaccgtgcctgcccggggcgcgtgggatgtgcgtgttcgtgtgcatcttcgggtttgtgtgtgcgctgcaaggccttcccagagtctgggcctgggactgcaggctgccctggtcaggctctcgccccgaggaagtgcccgaagctggagcaagagagtggaaagcagctttggtttctcaggccttcgctgcaagcgagagaccaactcagtgtgcccagggaggccccagaaacttctgagcggcccacagccggcaggccacccctccctccagccctcagctcttatctgctccacccctgccaggtggggaaggttgccattccgggctctgcccaggacccagaacgggtgcctggagcgtcctgcagcctggcctgaaggccgaagagctggactcaaggggagggcagagcgcaggtctgcctgtcctgggagtccttcgtgacgaaaagccaacacagagctcatctgtcaggcccagacccagctggagcccacactcgaggaagcactgggaacctctcctgcgccggccccacagcggggacgcctgccccagccctccccaggaaggtggccgaggcaggcacaggacggcggccgcctgcctgacacagagcccaggatctgctgaggaatttcatttcattggacgatattttacgcagatgtggacgtccctggggacctgactccattacctgaatttgaggcagcaaggcagccccaatgctgccagcagagcccaccccaagccacagcccagcagggtcagggggcttggggaagggaaagaggtcaaggttgcaaggccagtgtgcagggctgggctgtgtctgcagccgggcatggaaggacgaccaggtcagtggatgtgcctccctgactcagagcgtggggggctgccccagagggtgtatcttgggctgtcattcattggttttacccaggagcttactcagcggggatctctcctgctgggcaggcctctggctgagctggtagaacctgtggtgctcccactgggggctcagagtggagcattccgagggcagaggccattggggcgcccaggcctgggctgcagttcagctgctggggagtcccccagctcctcaccctgagcctgggagccccggcagagggaggacagatctggtccaggggccccttcccccagcgagggcagtgctagcctgtggggaaacgcggctgtccctgccccatgctaaaaaatgaaatgcctcacaagagctgtgtgtttctgccactgttgtgagccaggtgggcaggtggggggtcagcacagacacggtgccctcttgacctctttacagcacagctggggggcccctgtgccagggtggatgtgccccaagagacactgggtccagccagggcccttatctgagctgcccctgaggcctgatccccatggagtgacagaccccacacatcccaggactggggatagtgtcatccagacaactgccatgtagcccctggggtgggtgggggtcgctctctctgctagatgtcgcactttgcgtgtcttgcgtacgttccagggtgatgtgtgtgaggaaggtgtgcctgtgacagcgtggggggagcgccgcctgcggctgagctgccagccggctccttctccgcagccgaaacaggcccggcccgggctctgctgggcctccccccaccccccggggaggctgccccttcccgcagtcctctccctggagccaggacggcagcctcctgtctggacgcctggtagggcagcagcgctaggaaaggtccggcgggagtcggcggctcgcccgcttcgggcgaaaacgcggaggccgaggtgcgggggcggctgcagggctggcggccaaggccaggtgccccgcggcggtgcgctgagcgcccgcaggcgctgcgtcgccgtcggcccggcaggtgagcctgtgggacgggaggttactaagtcctgttggtggagtgacctcccagcggggtccccgggatggccactgcctgcggcttagggtccccaacatcccgacgagccggcccggggcgccctgctccttctgtcggctgcgggggcagagcagatgcgagacgggcctccccgagtctgcctgcccccgcggctcccgcacaccacgcgtcccccttctgggcagagcgcggggcagagagtctggtcccccgtttctgttgagaggccaggtgccggcggtgtggctggccgggggttccccagagcgggcccctcgggggaggcgcgccagctcgctgcgccgccgtgggcgcgattagccccctgttttattcaattaggaggcaattagggatccttggggggctggggctgctaattaaggtgatgtataattagagtttcaacaaaaattatccaggattaaaaatcttttgaataatttcgtttaaaaatttcaaatagcgttaaatacaatttatcacgttttgaatcaaacccgtgtaacaagtcttggacgctgcagctcgcgaagcgggcgcgggaggacgaggaacagccaggcgccgtccccgtgcgggaaagaaacttcgcgaggccagcgagccggcgggcctggcggcgcgggacggcgggcggccgcgggcgcagactcgggccggcgagggctccagaccgccgggccgggccgggccggggcgcggggcctggggacccgggctgagctccccgttctcctcgtccaaagtcgcggccagctactgcgcggcgccagcggcgcccgcgtctacaccgcgcagccggttcctggagatggctgcgcccgggcgcccgggaccccagcccagcgggagcagagccgtcctccggagccgcggtccggcctcaatggtctccgcctcttggcttcctggacggagaagggcgggctccgcgctccgagtcccccgattcccgaccccgaccccaactctgcgggcgacagatctgggtcgggtttgcagtcgcttcgcggacctggggccgaagagactcgggagtgcacctgggagcctgcgcctaagccggggcggtgggccgagggggagaccgtgagggaaaaccgagctagccggagggtcggggtcgggctttccaggatcgacctccgccttcctgggcggcatcccggggcccagcttctcccggggtggtgcgtgggccttgtgcactcgctccacgcacctgtctctggggaggcctggagcgtgctgcaacctaaaccactgccccgtccctgtcgccccggcaacctccagggagaaggtcaaggtccccatagaggcactcatggtccacttgggtgatgtgaccccttcactgaccctgagaaggatgaagtgtgggtagctggcaccacttttaggggtaggaaacccaaagcacagagaggtcaagtaaatcatgggaggtcgcacagcaagttaagggcgaaagtgaatgtcacttaaataaagccctccaaggtttcttatttggcttgtaccccagccctccagcacactgcaggctgcaattaattccataggattttttttttttaagttgtcagggtttacgcgttttcccaaggccagtgctcagcttgtaacaagtctgttcttcaaaagcaaataagacatcagtgtgcttacagcttgtgaatgcaactggctgacatgcgtgcgctcagcctcctccagcccgggtgcttccctcattgggtctggacgcccccgctgctccgtcccccgcccaggttggcttccccagcttctgcagccctggctttctgcgtcctctgccaagggcaggtgccggttagtgaaagtgttgaccaggggcctccttgcctgcaccctcacttactgccatcccctttgagccaatgcaggaagtatgggacctgtccctcacctcccacccacacccccaccccaaatctggagggaaatcatcaccgcccctccccccagacagccagggagctgctgtgtgaacctgagccccggcacagagaaggagctggcccctgcacatgctgctccagcctggctcggccgctgtgcctcaagtctggccacctcacaggctgcggccagggcctcggtggccgctccgtgccggccagtactcctgaactcctgagggccccccgggcagtggaggggtggcgtcttttgctctgaccgctctccactctaggggtttcgccatcaaatcctccttggtctggaggtgcctcggctggtccaccggctgggctctcccgagactttgaggaacaatgccctggggctcacggagaggagcgtgtagatctgagatacatatttgtttaatagttaacattctgtgcaaaagaggagggaagtcagattctggtggaggctttgcttattttatatccactataactatttctgtccatgaaaattggcttaattatatcaatggaatgtttctttttcaattctgtatgcaattagtccatcttgccggctgcaatttattttaataaaaaaataaaccagtggtgtgttcagttcctcgtcccctggggacaaatgcgattggtgtgtttaatttcattaactacaatgcggcctactggaaaaatgtataaaatcagctttatacagcttaaacactggcatttgaaacaaatcattattgcattagctgtcacactggtaattctagaatctaaggaaaaaaatatattatggctgtttttataaccctttgcaaatgcactgttaaaataatttataatcattttaaaacatggctgggtcctggtgaacgttttctctcctctagatgaggccccgtattatcccatgcacattttgcatcctgacgcgtgtgctgtttcccaccgcaagcactcgttttcaggatggagacagtgtaacctgtctgtcaacagacctgatgagtacggggtcacttgttccttctgtcctgaccccgctcttgagatggaatgcttggcgcagggacagactcatctgaaatatgcctgggccagaggggaccaacaggctcctgctgcagagcccgcaggcccccggccccgtgacccctcctcgggtgggaggaggggtccataaacacgcaggctttatggtcgtgggagcagaaacatcccacggacagtgttccggactcagactcggggaagaggagcagcccttcctgctcacctccaaaactctgctctggaaagccagagagccggggctctgggacccccgctttctgaaccccacagaggattctgcagagtaaactcgcctagggaattcaccagactcgcgctgcctctcggtggtaacgcacttcccagaagggcgtgtgccctccgtctgcaccccgtcggtcggtggggtggggtgggtgggtgtgcccagtgcttgtgtggggatcagagttgtccgggaagcgcagcctgagctgacgaggcagcgggtgggggcggcaggtgacggtgaggctgctgctcctttctgccgcgggtgtgtgtctagagagactgcacggggggtcttcggggagcgggtgctgggcagagctcagggctcctctgagcctctgtgtcccagttctgagatctgctctgaggagagccgaagcccctgccgagtggcctctggctcttccccagcctggaggtggcgagggaccgtgtctggctgtgaaggagaaagagacactggctcagcctctgtgcctctggtcgttgtccccatgccggcgccttgggcttcttctaggcggtctgtcttcctctccatccctaggtagtgtcaaggggcgttaccgggaagaaaaggcctcagtcggcttggtctaaggctggggaccggggctgctgtcctgctggtgcgatgcggggaggagaggggttcccaggtctcactggggttttcttagggaattcctggaaaatagtcaggcatttctggccacgttaaggactatcccctcaccctgcccattagcctcctggctcacctggatggatttaggtagatctgattttctgcttttgacgcaagatttatattctatactgaatctcaatgtggctgatggtggggcattgttgcgggggggacacaggcgcagtggtggggccgggtggacagagcctcggccaggtctccccagatgcctggctcggggacaaggccccgttgatggacctctgtgtgccaggaagggtcctcaggcctgcggtttactttctgttcggcgtgctgctgagttgagaagtaagtggctttgtctactttcctacttgagaaggaacaacaaagctggacgcttcgtgcaaacatcaagtcctggctgaccggcgccctggacgctgctccccaggcctcacgtgggccctgcaggcagccctgccatggcacaggctgggggcatgtgcttgggagcttcctgcctcaggccacagaaaccctgcaccccgttctgcaccctctaaccgtaccgtgcacgaatcccactctgatccccatgcgccgtgactcggctctgccggcaggacacccctcccctcgctggcgccaaacctggcccttgtgaaaccagggcaagggaggttgtttgggataatgaaagtgaattggaactcagttgaagcgctgcagtgcgtgtacgcgtgtgcatctgcacctccttcttttccagtcccagcctcgagcagagaggactgcacagaaggttctggaagggtccggcatagtgtttggtggggtgctttagcctggaaaatgggggtgagtgagaacgactccttccaatccgacaggcagtcggaaccgagtttggttttatctccttggcctgcttggttggtcctcttcctcccaagcttagtcggggcattcaggggtccatagaggggcgtgcaggcctgcagggatgcaggccaggctgagggtactggccaccagctctgcagtatcgctgtagttgcataacaccctgccaccctgctggtctgccccaatccagcaaagggcccaccccatcactgcccttcagctcagtcgtgaaataaaacacaggcgcgtggtccttcattgtgattttactttcaaactgggagacacaggtgctcaggcctgtttaggctcctctgacctgcaccctggagcttgctgagctccaagggtgtcgaccccctcccgtgccttcctggcccttcagccatactgggcctttccaatccttacgtcggcctttgggggtaaacagctgttggcatctgccagctcttctcttatcagcggtctcccggttcctgcagggggtcggggcttctgtttgtttcagcagctccattccgtcgtcctttcgtctctgccccctggagtccagcacgttgtttgtaggccttctgtgtgcggggctccaagacacacctgaggggccctgggggtggctgtgggatgctcgcttgtggttggcccacccagaggaagacatggtcacaccactgggcaatgtggcgggtctggggatttgcagatgcgaattgatcctctttgatcctctcttgccagcaggtccttgtcccgggatctgggcctccgtgtccaaactgttgagaaggggatacagccagactcagaccagcaggccacctcccatctcagcatgcttggagacactcagctctcgggtatccctggccttgcctctgtgcgggcagaggggtccctggacccccaactaacagctcttcaaattagagaggccaaaaggagagggagggagggatgctggagagaaggtttacacatctgtctgcttccaggagccttttctctctctctttcaaattcggtggctcacatgatgttggtggcgtgtttctctggtccctatggcaggcgtggcaccgccggcctttcggcacagggctgagcgcctgggttggtgtgtgatgaatgggcactgaatgtgaccgggaactctttcctgtgacacagtgtaatccccgtatccatattcagaagctggcagtccatattatgccgaaatatggatttacataatagacccccaaccctccttctagttgggtccctgggaaagggacatgcattgaaaagagcacagacgtttgtatctgctcaatgggctgtaacagcctcttcagagaggccccgtcacattggtccataaaacgtttaaacaaagactgaaagtgatactcaccggaggagcccaaatggtgaccagaggacggccaccgggctcccgggcaaagtgctgggcattattctcaattccgcccccctcgcctgctccctcccagtgcagtcgggtcccgagcctctgtcactgcctggcacttacttgccctcagatgacgctccctcgtgaaggctcagaatgagactatttccccagaattcatatttttgccacgaggccactttctccctcagtcccggggaaagcaatacgattaggtctttgctgtgttgaaaatcagcatctccagcctgtgctgacctgacaaccgtttccagaccccagattcagaagcggctgttgatcacccatctcctcactcactgccctgctggcccagccctgagcatgcacgtggcatcagggttaaaggacagggtggtcctgggggatatttttggggtgtcagcatagaggagagcaccacgctgccagaagttccctggggtccctttcggaagggagcttgtgacccagagaatgtctgttgctgggtaaaaactgagcagcttcaggctgcggcaaccatcacggggtgcggcgtgtccaccaggctatgccaccttcctctttgctgccacccacttgtcctgagaggccttcccgggaaatctgggcttccttgcaaaccgctcccaggccaatgcattcttgaactgcctgaaaactggggagcattttgttggccagagccgatgctgctcagaggagacacgacccccaacacagacccctccagacgtcagtgtcattgtgctgagatgtgctgagcacatcgtgctgagatggtttccagtgagtgtgcagacgtgtccatgcatgtgcccgtgtgctgtgcatgtacacgtatgcttgcacttgtgtgcaggtctggttctgacgtctgagtgagacttaccctcaaaatgttcctttctgttttgtcagaagcatttcaccatctctgcccactcgtgctttgcaaagagcctctcactttcgctgccccctgccccacgctgctccgctctccgtggtgtctggagcagtggctgtgtggccgggcagggcctgtcctgagccgtttgcagctggcagttgtgggagaagagacgggaggagttcaaaggtggagagcaggactatgcgagcactctcctgcacgttcacaacccccgagaagaaacaaaggcagaggggtgtttagataatcctggccctggtgctcacatttagaaaaattaggccctcttgaaaaattacagaattatgctgccagtgtcaggttccgagataatgatgtgtctgtgtgcgaaaatattaaattgcaataacacgcccgtgagtacctctctgaatagggtattcagttcccagctcgggctgcacataaagctagtgagtgacaggatacaaatggtgccgtaacacacggggagttccagccggactccatctttttgatcggtttctctatctcggcccatccagccatccttttctttaatgaaacagctcgacgacgtggctaattatttatttatcacgttagcaggaggaaaagctatcagagacagatcaatttttcccttgaagtgatttgacgttactttaaaaacaagacccgctgtgatatgctgggtgctgccctcggacctcccaaagttcggttcgtcggctctgctgacaatctgtgtttagagcaccgatggcctctcctccacgggcggccccacggcctgagctgtgggtgagctcggtgaggccggctctcctctgacctggggcttatcacagatttctgctcagaggaacgtgtccgatggcttcttctgggcaaatgaaatctgagcagcggcaagtaaaatctgggacgatgggggatttttcatatttccgcagatcccggggagacctggcgtctctagtaataagccaggtgagagtcccggtccctggtactcagagctcttcttgcccgtggtccgggagctcgtgcaagtgcggggcatgtgcacgaggggagggggtggggttttggtggctgcacattcggccaagtgtccttatgaacaaaagggctaaaacagccagtggaaagtaagaggctctgaaatccaggggcagagagccggcccaggaggggctctcgtttcacggctgggaatcggaattgcaaagcaaaggggcccccagagtcttaactctctccccatcgaaggtgttaatcttaccttccaaaggttagataaagccgctcaaagctgctgtttgttattgaatctcatctgctaacgatggggggcgattccgatggaggcccatctggctgcaccgccgggcccccaccgctggcagcctggataaggatcattctttgccccagcccagcttctccctttacccagggttttaggttgcctcccgatcccgacagacgaccctcgttcaaagaggtaggggctgatggccacagcgagggtgagaagcccattacggcggccctggtcctatctgtcccctcgggagtccccctccttgacccacagactcagcatgacagagtgagggaaagggcccgcgtggaagaatgcagcctggcgtttgctgacaaagagccccgattctttattcaccaggatgcagaataatgcttggcctcacctgcaggccctgcggctggtaggggcaccaagaagtggccgcagacaagtcagctgggcctgcggtgggctctccagaacattgggacctccgggctggatatgggctcctgggaaaccgtgaccatggggtgttggtcaccaacaagtgggcacacagggcacattgaaggggcgccactggggccagcatcccgtcaacttccttcctcaaagccctgacactcgccaaatttgtcaaagcaaaacaaggccaaaccctccccgctgcctgcccagaggcagaggccccgggccagtgggcaggcctcaggcaaggaagccaaggtgcccggtgatggtggtgaacgcccgtgagtgtcacgccgggcactgcggggtcttcacctgcctccagtcctgagacacctcagagttgagtgccggcggcccccagcctcaggcttggcgcttggtctccaattcccaaattgcaaatgggacgagcggctccaccgccttagcagaccttggaacaaaccacgggaactcgatgggcctacctgcggctgagtgttagctcccggggtggaaggctggcccttggcccccatgccactgggaagagggaaggtggtccgggtggtctcggggccctggtgcaagcctgtggggcccggtgagggaggtttcctgctccctgccatcttcaggagcaggtaggtctcctgttggcacagcacctccgggccagtttgaccagtgtcagagggacaataacctgccctcaatctgggctcactcttggccacagccgagagggacaagagggttgccgatatgcagggaacctaggggtgactgaaccccaacttgctgggctccaaggaccattcccaaggaggcccccggctgaagtgggttcaggtttagagatgtggtgatggggccttggaagcacccttcacccttatcagggctgaggctgggggacacatgctagccacagccatggccccctgtccctgcaggtcagctcctggactccctgaccggtcccacctctgctgggtcaggatggagcaggcccgatggctggtgccacaaatcctcattcccgggaacttcagcctgcagggctgggggcatttctccagacaccgtgaggcagacgcagggtccagctctgcctctgtccccatgagagctggagggggtgtcttcaccctgtcggtccagaagggccagcctggtgtggctgcaagaatgaaatgagacccgtgtgctcactgagggccagcgcctatgtcccccttggctgtggcccccactttctccttcctctgctccagggcaggatgctcatcacggccccccactgatgtttctcaacggctcagatgggacacaggggaagaggcctcagttgggggagggagggctggcaccatgtatgcagggtccttggacgtggtgttgttattactgttgctgtctgtgggatcgaggctatcattgttagcaactatttcgagtttctagaactttcttgcacctgccgagggcagatttgtatgtgagtgtgtggggctgagcagtctcccaggggcctctagatgtggtctacagattacacagcttgtcccaaacgatattttgataagtggagagaaaacgatcctaggcgtgcgttcggctctccctgaagcccttgcaggccccggatgggagcatttggcccgaggagccgtgccctgcatcgtgcgcaggcctggtgggttgtctggggtgattcgagcggctgtgctatctgtgtgacccaggcaggcatctttattggatttgggggctccatccaccaagttttcgggagtgaagagctggggggctgtggcgggcctcctgctcagggctgccctgcgagcatcgggacacagtgccagccccggggagggaagccagcctcgctttggagaggttagagttctgagctggaccctgtcttttggggtctcttgcttagtttttttccagggggttttggggtgttgactggcaggtcccaaagctgaaaagaggaaatctgtttctccttgctctgctgtaatccagctgacatctgacttctgagagtgtgtctttgatgccttcggggggacatgcttaggacttgctcagggaactgcaagataagggggctttgctgtaatcagtttgcatgcactggcacacacatgcatgcacacatgtgtacaggcacataccctccacacccacataggcgcacatgcatgtatgcataccccacgcacatgtgtaaggcacatacttgcaacacacatgcacatgcatgcacgcacatgcacacacatttatacatatatatgcacaagctgtgcaatgcagtgtgtatacgtgaaacacatgcatgcaaatacacatgtgtacacacatgcagagtgctctgtgaacacagatgtgactgcatgagccagactctggttttagtgttggacggtttctgaggctgatgcgtagacatcagaccagagcacatttttggcaggtgaaggggacactcattcagggagctgtcagtgggtctctttggtcccacaggcttgtagacagagcaggaggagagggagcctgggacatgctggtctgcggggctccctgcaggagagagggccttggcatccactgggctggggacaggcctttccctcctccttcgtcggggatgacagacctcaccagccctgggatgttaggggcccttccctagggtggggcgggcatggcctccaaagcaggggcaggacgggctggatctggtgtcatgcagacccgggagcatgggcacagggtccccttggccctttcaaagggacattcgatctgaaccctggggcccagtaaggagattcctgcaaaccctgcaccgagtcagcatgctcagcgctggagaaagcactttgcagttccgggttctggcatgaggccctgggtgggatgatctgccccctgggaggcgcggaatgctgggggcgaggggccacggcaatatagagctcacgccggccgccgacaaagcaggctgctttctgcggcagctctgtcgccgaaatttgaggtgcagccccagagtggaggctgctggcccagaacatttgagccagggaggcaccggccattctgcccctgcccttgagaatttgagtggaggccatgcctgctggtcccagtacccagggctcctgcccgcttgaaaacacaacgtctggagaggcctgggcagcatcttacttcccagcctgagctgttcacgagcatccctcgtgaatcacgatgcggggcccagtggaccacagctgtcgtgggggtaggggagcaggcagggaggtgcctctctgggctgggggctcttgggtgccttttgtgagtgggcggaagcaccgggggacccacacacaccctgctgcaggtgctggccccagctttggagtgcttggggtgtgtttccacccagcatctgggctccttatccggccccctctgtcccctaatcattctcgtctttgtcagtggacgtgggtacatccacctgctctgggggctggtgcccagggcccaccctgaccctctggaaggcaagtctaggtggaatctgagcacaaagctcaacacgcagggggcttgcagcctcgaggggctgtggacgtggatgggccacctgcagtcttggtgggacgctatggacctgaaggccttctcaagcctcggtgatgggcatctctgccccgctgggtcggaatcatttcgtggcccaaaaccgaagcttcgttttgctacccagcagggcctgggcagaccacgatgtgacaccttcaactcctgagcacagctccagacagctctgggacggggaagggaggaaggcagacccagggcaggagctggacacccccttcaggggggtgggtgcatcctgactctcgagctgggtggggtgtctgtgctcacagtctgtctgtttgtggatgggcggtacaggcttggggttcagacagacccctaccgaatcctgtacccattgcctgcaagctgtgtgacccgggctcctgtcggcctctctgagcccagcgttccatctgtgaaatgggggatctgtaacttgtggggttgtggctgaatcaagaatagacttgcctgtctttcctggaaatcggcatcctgttcctgtgccagctcacctcctgcagccacagcaggccttctcggggcacctggggtgaccttcccggttcaagggcaggcagcaggaggccgtcggttgggatgtcccccaggcctgtgggaagcgcgtctggccccgagagctgcgccttatacacgtgggttgacagcgagtggtcagtcagctctctggagctctgaggctgtctgccccgaccacaaacccgctgtctgggcacgggcctggcaccttcctttagcctaactccgtgaggctgcagaacgtgtccacgggcgcccgtgaaggtcatggtggcctggggacctgggtagtcgtctctcgcaggtcccatttcttttgcacatctcaggccccttcctctcagggcggacagatgggcaaactgaggcatcacagggtacactcgtgcatgcgggggtgtatatactatacggcacctgcctggggtcgtccatgtggaggtctcacagctgggccggccagttcggacccccaggcttgtgtccgttcagcctggagttgttgctctgggaccatgtgcacctgcacttgctgtccggtgtcgggggggggcgctccgctctcccgtcaggaaggctgccccagggcaggggcccgcgcctggtgccacgtctccccacagccctgcccccgcacaacactcggagtggaaaggcctcacgccggacggcagcctggcttctctcctcgtgtcgcattttcgtctggaccctttggtcccgtttgggagcaggaggccaggagggagcctgggggctaagctcgctgagctgggcagcatcaactgcgacctgggagcgccaggcgcagagcccaggcggagcagacgctccacggccgacccctgcccgtgacctttccggtggagattctaccaagttcttgcgatgtggaggaggcgtcctcgggagctgacgaagggctcttgccttatttattaatttatttttacgaagtatgctgttaattaggcaaaagatcctcggaaatgtcctcacgcctggagatttctgcgaaaagtgagaaccctcctgaaatgtccagtagtgaagcaattaaggggcggaattgtgcaatctgaacgtggcgttgttcgcctcgaggaaagtgaagaaacagagacaagtggagtccgggtgacgggaactcccaggccaacgccgtgagtctgcagtgcacgctggagccccggaaacgcccatccttctggaggtgaaggggcagacggtcgcttcttcccgggcctcacaaaccctgacgtgtgagagcggagggccgaggggaagatggcggagaggaagcggcttccggcctgcgcatccagcctacggcagagagacccgtggttctagctccccgtgaccaccccggctcgagtgccttagaagtgtggggtggggtgcagggcgtgcccccagggctctgctgtctgggtcacttggcccctcacacggcctaagggccccagtggggagccgctgtccacattggggtcaggccaagggtccttccttgcgcttggcaggcaacccaaccccaaatgaggaaattcacatttcttaggtttgtgggagggagcttttagatcataggaggagagttaaatcagcttgcatttttttttgctttttgcaaatgagttcttagtcaagagtgtccagcatgcccattcccttggatgttatcatccgacctttccagtccggggagggagccacctttcagttgttggaatcacttctttctgaaagcagctcctgaccgcgagccatggcgatagcttccatgtctcctttctttaaacaacgttgagatgcctttggacgagcctccattgttagaaatcagttttctgggagggagacgcgtttctgtcggtggcactggggtctggcctcctgcctgtggctgagccaagattggacagaatttctcggcgagcgagttcccctcggtccccgtgacttgcctgctctctggccttcctgctacattctttttctctcctcatttcgctccgtaatgttgtttctctcacctcctaaagccagccagcagcgtggtggagatttatgaggtctaaactctaacaggatcacctttttcatacattttctctgctgttccggccgtttgacctcggccactgtgcacacgcaggggttctgttggctgagaaatctcctcgtggaagagatggaggccgggctctggagtcctggctgcattaaatcagggagctaggggcccgagtcgctgagaacaatccggaaaccaataacaacccaccccctccgatggccttttccccacatgtatccttatgtgcttgaataaactgaaagctggccccttggtcggggtgggagccagcaggggcatgaaagcgttcacagaacatctccccaccttccgcctcccaccctcccacacacccactcctcgtgatggattccccttcctccggtgagaatgcacacgggaaggtttttgcctggaagaaatggaggctgtggtcagcaatagctgcaggctggttcccagggccggggcagccggccgtgctgctctcactcaccacgaggagaaggttcgttaggattgggacgtgaagcagtggctggttcccatgtccccatgtatttcttaccttttcccatcgtcgtgggatcaatttctcgttcagaaccccagtcccacgctggaaggactaggggatgcctctcctcacatgcgtctggaagatgcaagtgtttccagggctgcactcagccacttgtcaccccactgtcctccccctggggagggctatgaccacgccatggatgggctgcaccagtagtgtcgcccctgcctggagaagatgcacgtggactccatagctctttgcacccccagctgcctgcacagtgatacccggctgagagctcagctgagcatctttttctgaaagttgtggggtccgtgggcatcccccatccagccccgggtccctgcgcactggagtggtctctgcctgggcctgcattctccacggactcggccagagaggagcccgctcacggacttggagctgaagggcgtctgcaagtgcccatgtaaggtggtgggagggggtggagagcctctgagcacagcagcctgtccatattcctgtacaggaaaaataaaacgtccgcaggccataaaatgcatatcgttcaatgggcctttttatatgacagtttccccattagaacagaagtgcccctgccgggcgcaggccagacgtcctttctcggggtgcatttgctccccgagtggcttgttcctggggcagccgccaccacagagaaaattcgaaggggccgcgtgtctggcacttgcaaattcccctttttattgtgtctcctccaggcccagcctaatcccagacgtttatccgaggccagtctgctccccatcggcatctcttttgatttggtagaaataggaacactgtgcatgttgctttgctgcaccatcccgtcacctggagagcagcttcccccaagggactgtgacctcattcattcgtacctccgctcgttcatctgtgcagtgaccatttctgggggccccactctgcctctcccgcagctgcagggagctcggtcctggccttccacgtgctcagagtccggttaggcaagtgaggtataaacatcccccaagtcaaagcccgggatgcaggactgtaaggtctgcctcctgcgtgcggcagacagttaggaacccagacctcgcatccgacgggcctgggttcgcatcccaggccctctaccctcagtgaccctccaggccctggcatccccagttctcaaggggagatattacgagtcccagctgcagggtccatgtgggaattgagaggagactggcgatgagcttcctggtacctaggaagcagcccagagatgtcacgtcgtggggtgatgcaggggctgtgggcaccgcatccccaggagctgcctgtctgctcagatggagtccagcttggagacgccgccatgtgccctccaggtcgggtcagcggcctgcccgatgctcaggcgggcttcctggagctcctctgagcctgtccatgcatttgctgacccagctccagagacggacactcggttcccctggggtggcccttggaacggcggaagctcctcaggaatcaagtggccactttcgcccaaagaagctgggtcagccccttgtgccccctccaggcatgtgtcccctcgagaggcgggccacgtgtgcacccctgctggtaggctgcttgtgcctgtggcactcatcctgggttgtgccacaactgacctgtggtgacctttggcttgggggaggctggggcctgcccaccccctgctccctctgacgtgaatggcagcttcccatcagaccccaaagcctccccactttgaactctggctgaggcatcccccatctcaaatcaaaagggtctcttctcgtaccaacgtcccctgaagagcaaacagggcatcgactcaggttttctgaagcagcagcgatgttcctgtcggatgcaagggcacggatgcacaggaccctcaggttcctagacgctcctggagctgagagcagagtcttcaggaccgaggcccacggggatgcgggctgcccatcgctctgctcccaagtccttggtgtggtctggtgagctgtaaaaccaaaagcccccggtcctgaggcgtccggaagctgggctctcggggcagccccttcctgtcctcggatgcctggggagcgagagggagagctcgtcctggccgagcgcagggcctggtcctgcatcggaaggccttgtttctccaccgatgtctgctctgcagtcacacgcttggggtcagcacggggtttctctcttctacaagcccagctgcagtcaggaacctggggagggagtggaaaggctgtgaaaaacggatcagaatcctcccctgaggccactggagcagcttaaacctgctggtcccgcaacagccgagccagacaggctctccgacacctgggatgtgtggatggggaaactgaggctgggagaggacagtgagaaacagagatcaccaaggatgttcccagcactggactttgcacggaggagcatgaccaaggtgccagcctctcctccatcttcgtaagctcatctccctggttttcctcaaggagtggaggggtcagacttttgagaactgctcagatccacgggagctggtgggaaaggctggcattggggctaaagccattgtccccctgctcacctgcctgccagcccccatgccacgtcttcccgctcacccttggttcccctgggagaggctgtctgccccctagtctccaggggatgggcaggaggctgggggtcccagcccttctgacgtccatccaggagcatggagccggctgcccatgccacgtggggtggggagcatggcgacccacagagaggtggccctgggcagaggctgcagctgacgctccctgaacagttaccagccctggaatcctgtagggcccctgcctggtcatctcaccgccctaggagaaggaggacaatgtgatcctagccatagatacatgggctgtgtggtccccaccgtagacacaggagatggtgtcgtccccaccctagacacatagggcaagagctcaggttggggccacctgttttcactggacacaggtgtatcaggtggctaccatgtaccaccactgtgtgagccatggaatcagggcagagaactaagcaagagaggaagagcccgtccttgggggcctcccctccagggtgggcacacagacaagtggtcagatatgagaactaccaagaaaatcgacagggcgataaggaggagccgtgtgggtggtccaggaaggcctcctaggcaggggacgtgggccctgacataaaacataagactgagccgaccccagatgagcaccgaccacgcagggtcctcccttggtccctgtggccaggctgcacatgcaggcatccccctcgtagacaaggacacgcaggcacagagggctccccatggtgagcgatgggtgggttccagcccccatcctgcagctctggagctgggcccagaccctaaaggggtcaggagaggtggctccacagggtgtgtggcatgaggccccccagagtgatccccaaggcagagggtgctgcgtgctccccagggttccagccaccaggagcaaatgtgccccatcctgcctgctgcacatccttaaaccttccccgtcacgtgtccacgccctgcgggcccccgaggcggccgccagcactccagggccatcagtcgggtcccaggagagggctttgctgtcactggcccagggatggtttcaggggcgtggggatgggtgtgctggggctttggtcaagagtctcctcttggtgtccgtggtaaaagatgctgtttagttaaagaagccccacagccgccatctgcggccagtgagcaggagacggaaaccgagttccatcggcaccgccgggcccccactcagggcttctcttcgaggcccaaatgagctccgtgttggatgcacctgggcacgggatcctcctctctctggcagggcagcccctgcctggctctgtgctgcgaggtgggagccgataccctgcggggtctcccgcctgctcccggctaggaacctgggctgggggtgctgcctccacctacctcgaggggccccgtgatatctcaaggcaagaactttaaactcaagggaagacggtgaaggattgcagctgtgacccgtcacagcttctctgaaggagaccatcagagtgaggcagcagccggcctggaacttctggccacgagggtctctaggattccatggagcagttttcccacctctgaaaatcactgcccgattgatggccaagaaacgataggcaactgggtcaacggcctcgctggtcacttccctgagacggctcctgcggacccggccggggcagaggagatctcacccacgcgccgctccctcccagggcccaggctagcccagtgtcagaggcagggcccaggctagcccggtatcagaggcagacagcttaggtgcctgtcgaatcgccaggtgaacatcagctcccgcctggactggagtcaagtatcagccggtgtagacgggctccaaggtacagggaaggatcgggaatgctcggggagtggggtgggttttggggtcaggccagcccacccccaacacacctaagcagagtgcaggcaggatgaggaaggaattcttggtaaatttccctgagtgctgagaattttcagaagttgccacgggcgtgtggacttctgtgattctgtggtcaggctgccgtgttccaggaagtgccaaacagaagtggtgactctggccagtgaccttggtctggggaaagcggtggccgcagccagtgtggtctgtggctgccggtgacctagaaacccacgcctgagtgctcggagcgggaggctgggtgccggccgcagcagagctggggggtaaggggaattcacggcaccagggctgggagccgcgggacacgtgggcagggcacggggcagcagggcacctgggggggagcctggaacagcgagacccagagcagcacgcacctgaggcccccaggctgcctttctgttccttcatggttctgagcatctctagggtggctgtgggagtctcagaatggaacgacttacaaataaatccacgttaagtgaactgtgggttctccaacccccccgccccgtctccaggtctcagggtcagaatccgttggttggagggccatttgggcaaatgagagcagacgaggctgggaagacagcacttcttagccccttgtaggttcggtgctggggccggggacctgcagagagcggctgtcacttccactgggtctggtgagtggtgaccgggaggtgactttcacgctggtgactgagagagcatttcacttattggtgtgaacagactggttttgtgggaaaagctgcaggccctgagctctgctcagtgcaggaatgctgatgcagccggctggtggctcctgcccgttcctgctggagtgagcagggacctggggacttgccctggtacgtgtgtcaccgtggggactccaggccatgcgcctgccccctgggccgggaagggcaggggctgcgtgtgtgctaggctgggggcttccctcctctcttcccttcagtggtgttgacgcagagggacagaacccctgcctcctggcagcccagcgcccgtggctgagagcccttctcacagccccgtggggagttttctgcatcgggcaagacaaaaccaggacgagggtgaggctgggggctgggctctccgggaccctctgcctctccaggcccctcccacatcccgccttgtcatgcaccaatggccagcccatggctctgccacccctccagccctggcaacttgatgatctcaggggttccaggggtcatccttactgggcactggcctgggtgctggggaggtgctggtggctatcggggtcttctgctgccctgtctgcctgctcctgaggagaaggggccaccctgtcctccctggtcccccagccctgccctgggtcaggggcctgaacgcccacggccaacgagggaggcgggtcccactcagaggcctgagctccccatctggggtctgtggagaacacagtacctgagaacgaggtgtatttacgtagtttggggtgtgtggaagggtgcaactgaggcctcagctgacctgggctcagaccaccctccgtgcctcagtttcctcatcattaatgcagggataatgctgttccccacacagggttggaggggtgggcttcctaggctggagaatccccagcagagtgaagcgcatgcctgtgagcacagcacccaccaccctggggaggcgggaggaccccagagtctggggaatatcgtggaatattagaaaatgtgaggccatgaggcaggcagcgagctgtgggaggggaggccgggttctgggcccacctgggcccccgggcacagcttctgcactggtgagaggacagagttggcccagacgccgccacggccccacctgctgctaactgtcaaggttccaggtcagcgggtgattctgggcaaagcctggctcagccccttgtccagatagccctcagggtccccgtgcccatttgagactcggccagccccgcaggtgctgtgggcagccttgggccagggccgggacagcaagggccgaggggacaggggtcggtattggggtgtctctgtggggccagggggactcatgggggtgccttcactgcttgggagtgtcttggggttggcgccgtctgctcaggggtgtctgtgcgttgtgagccgtctcgtacagttcctcattccatggtcccttcatctctgcagagtgggcttgatgacagacaagtgtcagcaagaaacccaggccaggggagtcgccctccgtcccctcgtctctccctgctgtatagatggcccagcaacctccgtccccagacagtcccgccccaccccgtgaagccccaggagcctgccctcccgctcagcctgggccaggatggcctacatgcgatgtcttgccccactgtgccctcgtgcccatccatttggggtggaccctgtgctgctgtgccctggagcccgggaagcccgcagtgccggagccacctcggggggacagggctttccacccccgtcctccttgggctccgcacggagccctcactgctgctcctccctcctgttgctccagccctggaccccccagcctcacgtccttctcgcgtcccccaccctgcagggctgctctgagcccctgcctgccgtcctggctgagctggacctcgagttgggaaggaatgaggtcatgggagggaggcagctgggggcatgtgcttagaaaggagagggtctgtttttagaacccacaccccacggctgaggccagaaccgctcgccaagaccgagcttcccctctcggccccgccgcttcctcttttgccagcataggtaccttccctgccggcccgaggcacagggcccgccagccgccccggcctccctcagttccattgccgcctttacattgaaagcattgtggggctggacgtcagggacctccctctcctgttcctggacaagtgaaaaaggcggcccagggcctagggctggtgaggctcggaagcctctggcagctctgcctccgagcagtggtggacggtgagtaggccattaccccaggagagcggggagggtgggtgtgctgtaaggcctcttcttcctcagtttccctgtgtcggcctgtcctgggagcagccaggagggtgaggccaggcacctccactgtaggtcctggccaggtgcccagtggggtggccgccgtggtctctaggatcagtggggccgtttgctgacgactcggtgctacattttaaggcggagcccatgccattctccccgcctccctcctcccatccctttgctccgttccatagacttgcccttggggagtctccgctccttccgagaggaacagcctgtgtgcccagggcctgcccgtgggtgcctggggtgtgcatgtccctggggaccgtgaggccatgactggggtcaaagatgccgccgcccgtttccctggtgaggactgagcaattcagagaagttgcctacgttggcaaggtcacgagaaaggtcaagggcagggcctctcaatgagccgaaggacgaggtttggctctgctgggagagctcaggattccaaaggtcccgttgttggccgtgtggcctctggcaggtttcctgactgctctggtcttgttcctcatctgcgggataggggtgaggatgtccactgcgcgggctgccaagagcagcgtctccaaggggcctggtggagcgtaatactcctccctcaatagtaacagtactagcagtaagtagtgacgaggctgccatccctgcgtcctggtgtttctcaaggttagcaacgttgggaggtgctggtcaggatccagtcactcacagaagccggcagccagcgctgtcgccagaactgcaactgaaacgatcttgaaaatgcctcgccatcccgctcaccccccatcttcaggtcccagctgcctccctccctgtgctgctgtcccgtctccagccaggagaatttatgggggtgctgggggtcctggctgctgagccaagctgctcttccttagccagggaggggctggggaggcggcagccgcaggcccagctgagaggcctgagaaggtttccccccgaccccgagaagccaggacgctgcgggggcgctgcagtgcctgagcttggaccctcgcagagacaccccggggctgcccagacccgtctctcggggtcaggaggcaggtggggaggctgaagacgtttggacagaagcccactcggagggtgcagcagctgtggcagcgtggaacgcaggaagaccgggggccacggggagttctgccacccccgccccctgtcccttcttctcagtgcccgtccctgagacgcgcagcccgggccgctgtccacagcggagttcacccctactctctaggcagccgccagctgggcacggctcttcaggccggtggtctgacctggtcttcctgggggcagcctctgcataaggactctccactcccaactccggccccaaaggtttagaaactgccctgtgcctcctctgctgcccgagtccaccctgtggctggggctccactcaagctccggtccctcctggtttgtgaaggggcacacccaggccggtgtgagggctccagggggtgttctgccctgagtcacgccgaggtcccctggccacaggaggagcagcccctgagccccccaccaaacccccttcccactgggagggatttgccaggaagaggagaggagccggcctcggggagcagagagcttccctgtgggccagatccctcacaagctgttccgacagtcacagctctgtttgggataacccacccaggcctccccctgacccaggcctggctcctctcggggctgctggacgccgtgcagagccacaggctcgaggtctctgtccagagcgaaagccgagacctctggctgcagcttcgtaaaaatgcagctgtatgcgaggagataaaccacgtcccctgaggcctgcaggagggagctgctgacctgcgaggaacaagggaccttacaggaaagtggcagggggcagataaagggacccgagtcaactcatgcggaaaggatgaatggcccagccgaaccaggtccttcagcatctgccgtttggaaggggcccctgggagctgacggggggacgggcaggctgtgggaatcaggggttctcgctccccggggtgcatggagggctcccctctgaagcctcttcctgccccggccctggccgagccggccacttcctcatgctcacagtggacgctcccagttctgaatctccacccacgcccgctgtgtggccctggggggctggggccgcatcccaccggtgccaataccgccgactccccatccaggcagctggcactcagcacacacttactgagtgagcagagcccacctaagaggggctggcagaggggaggtgctgtctccttgagtgtccccttgagcacagggtcagaacccctcactgcctggaatgcctgcctgaagggctgtgggcattgggaatgctgaggtgcaaggtgcttggggaatcgaggacaatcccacgccccgggctgcccaggggaaggcgtttggcttcaggtccatcattaaccagcctctgtagagctggggttgtcgctggtgtccatgatgcggacgcaggcccctgcaaccccgtttgcttggcagtgggtcccttgtgttggggctcaagggctggtctgggcattaaccaaaggcgggggggtgccgaggggcggcttgtgggttcctggccacgtggctggcgctgggcgtggtgtccatgccagtggctctgagtgtccctgctcccgtggagtttggcggcgctctgaaatattccctgagcaccatagagaggggtgctccccgaaccccagacttgctagagccccacgcttgaagctgggcacagagacaatccaataaattcattcagacccgggggtcacgctcactcctgcgttccagggtcagccgcgggggtcgctgcctgctgcagacgtgtcagtaaacgggtgtcagagattgtttaaaacagaaaaagaatgcccagtgccctccgaacaggcggaaaggcagccagtaagccgtgaacagctgagggggccgcctctgccccacagctccttccagggcagtggcacgaggccaggaggcacggtggaataagggtggggtggggctcggggcacacggagcatcgaggggacacctgtgaggcccggtagggcaggaggccatggcgagaggggttttcctgagtcccatgggggcttggggctgcggtcccagctctgtcctctgagggcccgggtggggccctgagcctgcccacacctcccgatgtgtccccatcgttggccgcggcatcttctcctggcagcagtgggaaggaggggggcagtgggtcatttttcttcctttttctcgagaacctgccgtgtagagagatggtggagtccctagggccataggctgtggcccctcgcgggcgggcaggtatttctcatgatcccgccgtgaacctgggtcccggcgcctccccagctcccgctccatgtcctgctggtgtcccacttgagggtgcagccaaaggcacctctgcctcctcagccgtgccccaacccgctctgtggggaaggggcatttgaacacgctcagttggcttttcgatttgatcatcttttcagccaaattctgagaactggcccgggctgggctgacgatggaaggagccctctgcaggggaggaacatctcttcctgcctctcgtggctgggggtgcattgtccttgtctcccaaagtgtgcagggtctgggtgggcaggggcggctctgaaggcccagggaatggccacacttaccccctctgccagacactccacttcatcccttcactggatgaactaaaagtcaacctcttagctggcgtttgtgggcgctcactgtgcgaggcacaagccagggtgccccaagtcttggaagtgaccagcttagtgggaaagatggacagggcccagttgttctcttggaggggagaagggggcagggggagctctgaaccccagattggggcaggaggagttcaggctcatgaggccaagccaggtctgcagggtgggagggcgttgcccagggggccagggggcagcaacgtgcccatgagtttggagcccacggtggtcgttgacagcactgccctcctctataccacaaaacctttttagtcgtaaccaggtaacactctcataatcgttgttttctccactcatttctttagtttttgaacaactcatccttaaaaaagaagaaactctgatcttcactaaaatttacgcttatcagacaaaactatccccctttgcagttcaactccgtttctcagtttttgattttaaactcgaataagacccccaggcgggcaggcagaaaggcaggtggaaggcaccccgggcgcttctcccactttgccatgtggtcagcgcagacttcccacctgtggtctgcgtgtggggagatgggatcctcgggccgcgactcgcccgcccccaaagtcccagccgctgcacctgcccttgagagggtggggaggagccgggtggcgtgtgccagcgcgaagcttccatgtgcgttactctgactgcgtcaccgtcactgcaagacttgctgaggacgtcgtggaccgatgtcagcctgtgggagggctctcggtccctgacgtggggttgccttacgtggtgatgataaaaagcagacgatgatcagctgcctttgtttttgttttaaattctctgccctgtgcctcctcttgtggggtcccagctagggccgctcctacctccctggccccaggtggtgggtggggaggggtgcctgtctgggaggtgggcgcgggctgagcgcaagtgtggggtgtcgcagcacagagcgtcagtgtgtgttgagtgaacccagggcagccatggcggctggggtgactgtgagcgatgggcggggtcatcgtctctgcagagccaggctctctggggtgacaggtcggtctagacaagaggtttctccccttcaggaaggggactgtgtgagaagaaaagggatgggaggggaagggagaaggctccatgtggcatcttggggcctgattctgaaagaccccagggcttgttggtcatgagggctgggctggtcccaggacgctaagtgtagacccctgtcctggcacctggcacatagtaggcactcaagcaacgaacagaagaggggaagtgttcctctcactccgtcctgctcctccgtgggcgctggtacctgcgaggagatgtggtagcgagcgaaccctcactgagggtccacgaacagctctgaggacctccaccctccttcagtacagggcgcctccctggcacctcccagctagtgtcccacaggcctggaaaagcgagttgggccagctcctctggagctgccacctggagccccctcttcagcccgtatgctcagggagcaccccttgtaacattcccacgagggaggagctctcccagcctctgtcgggagatcttggccgactgtgtgtctgtgaaccggcctgggtttgtcaggggagacgatggatacccctggaccccaccctcagaggggcgcctttgggaaggggtgatgcgacaggtgctcccggtaacggggctggagcccctccaagaggtaaagagcgaggctgggctgtcagatggggaggagcttgcttcagggtgacctttatcactttgtcacggtgtcacattctggcccggagcctccatgaggaggagcgggggaagcatcggccggcaccccttctaagggccaggacccgggcaggtgtccgctggggatagggctgccctgggctggacgggttgcactgtttctggggacccgatgggggcggaagctgcctcctgctcaggtgtgtccccacctggtcccgacaggaacctgaaggtcgtcttgagcagcctccgggcggtgcctctgtggttgggggaagagagatggcactgagatgagctccactttctctcctccgcctcctcccccctggccctggacgaccgcaaacactcttccttgagagatagtggcttccatccaacgcacctccgctaatcaccagccatcgtgggaaaagcccgagctgagggccgagaggcagctcattgtctgggggagattaaagcccccgcactgtattgtctgggcaggggataaatcttcgcgctgtcactcagacgctgaattaaatccccctcgtttctaaagggcttatctgtgctgaatgggcggccgcgccgcagcgtgagcggacgtgggcaccttcggagcaggagccatctggggaggggacgctgtgtgcgagaggtggcacctcccctggtggggagggtgggctccacctgaagtcccttccgccccacgggacgtgcatctcactggctaggggagagaaaggccgggaacctgtgggggtccctggctgagccgtccaaggaaggggcccagagagggctcaaacttggcggggtcagagggccagccaacggcaggagaggagcagctgggagtccccagggcaactcgaggctgagaaggaagtgacgtaagcaatctggaactttccagagaagccactccattcctgtccttttcaagccccctcagcctgggtgagccagtggtggagccctgggctggcgcaggtgcccctgctaggtgccatcctcgaagcacagccccccttggcggctggccctcgtccgccaccttcatctaaggcaggagaataggctcatgtgtcgtgggggtcgggggtccaacattttagccacaggacttctttttaacctgagcctcacacaggaccctcacttactaaacagatggtttggggggagtgagagtccttccccagcaccccagagccccctgctgcccctgggccaccacagtgcctcctcagtgctgtaggaatcccagagcgatgccccttgtacagatggggaactaaagtgtagacaggagtcagggggtagcatgtccaggacacccaggcaggcagtggcggggtgaggtgtgacgcccagtcttcccgcgtgggcttgggagggggtcggggtgggtttgagggcacacactctgaccctgggctgtcccctcccatccggtttccctagagaatgtgagctccagctgaccgcattcagacagtggccagagatgcacttgtgaaacaagcaagtgcacggagatcgagagctgatggacagaggtgcaggggttgggggtggtgcttggggctcacgggtgaagacttggttgagagtttcggtgctcagcgctcaaggcgccgtccccagccctgaccagtgggcagaggaacctctggcacctcccccagaggcaccgttgtattcggcgtaccctgcatctcagtctgcacatttggagaaagactaatcagagttttccatgctgtgtgcgaagggtcccctggttcacagatgttgatactctttgtggaaatttctggctgtgcacatacctcccatctctccttggagggacgaccccagccattgcctgaacaagccagcttgtcacagtaccagatttttagtgagctgaaagcagagccctgccctgccccccagacacctagactggtctagaatggtctggaacatgcagcttcctcctgctccctcgcctcctccctgctttagacgagctggtaccccgctggccagtgatgctattttggacattagtaatagcttgtgtttctgaacatcatggtcaaataatagcaattccatgcgtgtgtgctgagtgcatgactgccgtcagaccttactctagttagaccagaagctgtcagtgtgtctctgtggtggtgacgcaaattcgctcatctcagtctgggctgggtaggttcaacgcctcctccctgacctgccccctggggccacagcaggaaggactggcggctccggctccctcgcctgcgtctgctctggaccccaggaccgctgtcacaaagaaccacagagccagcatcttggaccatggacgtttattccctcaccatcctggaggttgacagtccaagatcgaggtgtcggcagggatggttcctcctgaggcctctctccttggcgtgcagatggccggcttctccctgtgtcctcatgtggtcgtccctctgtgtgtctgtgtcctcatctcctcttcttaggacagtcacattggattagagcccaccgtaagacctcattttacgtggtcacctctgtaaagacccatctcctgaaacagtcacattctgaggtcctggggttaggacttatcgatatgcaaattttagagggacataactgaaccccaaacaatgccctctgccatgatctgtgttgatctgaagccgcgcttcattttgactctcagagcgtattacctgtgatgatattctagcacgcccccagaaaccagattgcagatgggcccccaggaacgcccagtccctctaggggtgaggcagaagcactcgtgtgggggtgtcctgaacgcagcccggagaagggtcctagcctccgggcagccccacgaacagaagtgccgggagcgggtaagcaatctgcaattagcagaaacactcctttctgagaagcaatgagctcaaaagagaggccttgaacctcagaaacgtccatggaaatcagggcttgactggagagcacaatttttctcatttttgtggggctgagccttttcaataagatttcaagagaatgaaatcaacaggccccaggaaatttgcatacggctactcaacatcaattctgtatggttttttttaaaaaaagaaataaacatatcttcaaacttccccatccgcagggcgggactaccctgggagttaacggttcaaacctttacaattttttaaaacccaacagctttccataacaaagggcaaaattcacaaacaaattgcctgcctcctcccaagtctgctccttggccgagtgaaggcgtgttgacaacacggctccttgggtcaccgctgaatggcggatcccacggagacgggtcccccaaggtctgctgagaacagcgtggcccgggagcctgagtgcaggctctcccccttccagtcttttcagaacagcagctaacaagcagcgttgtgcggacctgggaggcagccctcagaaggcaagctggggctgtgcagaggctgcccaggaacagagctgtccttgctggaaagtgctagaaggcgctggaaggcactggaaggtgctggaaggcaatgcagctcagcagcttgctgttctcaaagcacgtgtgtgctgcaaatgaatgaagcccgtcccggggttgggagcagggtccgatggacagctctggaggggccaggacagggtgggcacctccagcaggtgggaacccacaggcaggagcctgagactcagccctgaggaccgctgtggggagaattgccttcactctccgcccgtcccacctgaccggggctgtgtgcccttctcaggggtctcagagggtgggatgcccaagctggccctccagaagccctcagtctggcccgggagagggatgcaggaacaagcaagcccgggcaggtaggtgcgaagtgcccaatatggccctcagggtccctagagctggctctgctgacccctcccacactttcaggggcacaggctcgtggtggtctcccagaccccctttcttgcctctgctcctgaatctgtggcttctcctcttccttgctgacccctcacagctcatgcccccttctgaggccttcccctgagcccatactctcccaggggtcaccctgtccctagccgtgtgtttctggggtgcagccaccccagaagaccatgaccccagggaagggatggtttttctgttctcggctcctagcagctgctggtacccagaaggccctcagtaagtgcttgcagaaggcgtggatgagagatggagcagctctcagggtgaatctgtctaaaaggaaggtactggttgtgcagaggtatgtgggacaggatggactgctctggaatggctggcacaggaggcattggcccaggagtgcgctggtgggaggactcgcaggctcacctccccaggcagccagtcctccccagcacagagaggcagggggccagcgtagggccacgggctctccaccagggattggtcctggggaggtgaccactggtgaccctgggcccagcctccagggactgcccccctccagggataaacccacgtccacgtgtttctgtgtgcccagggccccccatccattttgtcttatccccgtctctgggccattgtggctatccagctctccccgtggctggcctggagcaggggcctggctgctcacctctggcctgaacactccttattcctgctgtgcgttcaaagagagataggagcgctggtggacgacaggtcctggaaggccccaggccctctcccggccagtggctggtcaggagactcttctggaagggcagcggtggacaggatgcttggacctgactggagagggagtgggccctcccacggtcccgtctggaccccaaagggttaaggagcgagtgggggcctttgttccgggtctcgtcagtggaacccgcagcctgtccccaatcccttcctcatgaccgtgattagtccgcgatgaataggctggcgattcctatggtaaccttcaaaatgcattcctcggccgctcgcctcctcagtaattacattcctagctcggcccacaaacagcatgctaattagcccctcgagaaagaggggactgcggttccctccccgagaagagaaggggccggaggaggagacttctgtttacaacgccccttccctcctcttccttgggcaggatggagaccctgaggtgctcagggcggcctggccgagcctgaccggcaggtccaggagcgggagcagggacgggaggagggccccgagcgcctctgccccctcctgcctaggctgtccttagcgtcctcagtgcatcccctgggcgtccacctctgtcctactcagggagtgtgtcctgcaaggtcagcccaggccccgtcccctggagctgctgccctgcttcccatcccggaggaaggcccagcccagatcctcatgtagcacgtggtgatggagccctgcttggggggccaggggatgcagggtgagcagctgccagcggacagggcacctggcctgcaaaggcgtctgtgctggaggatgaccgagcatgtgcagaggacaagcggggctctttgggtgaagcaggggaaatccagtaggaccctggaggagagacaggccagggaggaggaaggtgccacagggggaaggagtgcttggccctgaggctgtcgggggctcgatgcatctgagaagctggacactggccctcagggtgccagagggggctggagggccagtgggcgtgagcagccaggctttgcttgaggttgaaggggaagctactggaaaggcttttggcggggagtaatgtgagcccagtcctggttgtcaaaggtaccccgggctccagcacagagaacagactcaggaggtgcctgagtgggcgcgagagacggctcctgccaggaccaggggacagacaaaggctgggggaggcgggggctgggaggtgcaagagtagggggcagagctggtgggaactgctgatggattgcacgtggggtgcaggaagggaggacgcccccaggtttctgtcctgaccagctgggcctacggtggtcatttggcgaggcccacatggagaggagcagatccccgctggccttcaagtccacatgggaggcagagctgtcactgagccagcaccagtggacaccagcagggctgggctgcaggtctgatatgccggccctggctcagactggcactgcccgtctgctgggcaggtgtggtaccctaagaacttgttcctgaggccagcagagccttgccagcccagccaatgggaagcagagggggcatgacttcctgtccctgctctggtcagaggggaaggatggctaaggggtcagatacgtgggccctggggcaggtcgtctggggtcaagccaagcagcgtggccttggatgggcagctcagactttgtcagggcggggacaatagggccatatgagcgttgatagacactgttgctctggcctcagggtgtgggacccagaaagcagactggccatttctccgggcagtggggagcacaggccctagttggtgtcacgtgcccatgagcaaagctgcttgtgtcagcgccagggcaccctgcagctccttgaaccccaccgctgagagtgtccccactctgctggccttgggggaccaatctgggggggttgcagactggcttcaccccagcctggccagcgggccaaggccaagggctgggagcaggcggctggccaggcgggctggcccggaggagccgctgacgcgtgattctgcgcccaggtggttcagacagtggggcttatggagggattaccggcctcgggatttggaagccagcagttccttggtgtcagtctgctcagctaattctaatgtaaacagagcaatcctttaagtatagatcatttaatgcccgacagggagcccgcagatctccttagcacccgccggctcaaatacctgctgggggacttctccggtctcagcccatttccccgacgggccgtgctgtggtaaaccctggtaggatatcacaataagtatgggaagcagcctgggtggattttcatcccctattgtcctccgccctggactaaccctcagaaccaccttcccttgctggggacacacatgatcccaggcactgtgccggcaaaccccagacacctccccggggggtcagcccccagcaggtctctgctcactctcttcccagagcatcggccaagcccagccccatgacctcgacatgtggccagcctggggctggtctctcctgggcactcaggagcagactgtgtgtgactgcacgcaggggagtgggaccggcagggacaacccgcggcagacgggcaccactgcggctgcctgcatcatggaggctggctccccaccgggccctgccaagctctttccttgctcaggagaaaccaacttcacagcctgggagcacgcccatggcacagacgcatgcaggctcacatttgaagggtgaagggagggggcacggggagccagaacgcccaggctttgctttcctccccagcgcattcgtcttagctcgtatatcctgtcccgtccacaggtcagattctcctcgacttcctgctgaaagttttgaactttgcagtctggatctaaacccttcaatccttctgtcgtgtgttgttgtgaacggcgtgaggcaggggtctggctttattctttgcggcatgcgggcagccactgtccccgcctggcagtgttgggctgtgcgatctgacacgtcaccaggtaacctgcagtgccaccctggtgacaggtcaaagttttgtgcacgtgagggtctgtttccaggccccccaccccttcccatgggtcagctggtccatgcctgggtctatgctgaaggcctgaagggagtgtcacaataagcactttaaactgcttcggcacgtcgcggggaggctgcactgacgtccaccctgagaggaggagaaggggagcgaggtcagagaggtggtgaggctcatcggggctcgggccggagccgtccatgcagcctcactctgcactcacaggtctggaagccgtggtgcctgcacgccgctgccgcaggctagacctcccagccgcagggtctctgctggaaccgaaagagggggacagtttgaacaggaaagtctgaggcagggtagcatccccttgtcactctaggatgaccgcagattgtcccaaaagacgttgtaatatttaaaaagtagagtaaaacaaagtcacgtaggttgaaaacataggtttatgcagtgtcggcccccgcgaaggctgggccttgcccgccctctgggtggagcgggctggctgtccctgtctggggctgagtgggcttctttatcgtcggattttgcaggtgggtgggctgccagcttcttggatgagactgaaagctgcggacactaacttgtgtttattaataataattaattagtagtagtttgctggtgtccaagggatggcaccatctggggagcagccacatccgtccagccccagggccgcagggctgtgatttcctgcctcctgggtaccagtgctcagcatggctgcctaaacagatgctcaaatgatcccgctgctcggcgctcccagaagtcaactatcttttaataactgtctggtacttcaacctcccgggggcccatggaagcatcacaggccattagagcgggggcttcgccaaggattaatgagcagcgattagggaccagcttcacacggggcaggaaactgtggtcatcagtgaacagcactgaattcatcccaggggagcggcgtgggggagaatttccatccccatccccgcatttgagcaaagaggacccctgagtcctgaagtgacggtggggtctcaaaagccgcactacccccagacaccctgcaaggggcaggcagctgtactgaccaatgacgggtgtgtttctggtggaagctcacctgtggtggacgctggctccggttccctccccgacacagatgcagcccacaggctcctccatgccctgggtggggcgcggtgcctgaacggccacgggctcctcctcgctcttgatctagagagcacggtaatgacgctgggtccctccaggagcctccttcgcagtgttgtctttgctcatcaccacacctgtctgccctgtgagggagaagaaacagcctgtgggagagggcgtggtgggctgctgccggcacatgcagggcagagtcaggccctgctggagcccacagatattccccatgacaccaagggcactctgctcctccttccttctggccttttcttccagctgcccttgccagggagatctcgtaaaaaggagaagccagaacttagctggtgcttagtggaggagagaggggcccacaccccaaaatctgcagcttaaactcagcctgggaaccctgaggtctcccagcctccctggccatcccccagcatggctctctgcgctcctcaaggcgtttcatggtgccgaacctgcccagtgcccagcctggggtcagcatgtgaggctctccctggtgctgtcgcccactggcgctgacccctgcttctgccccaggaaggcctgcaggaggactggcagccttggggggcctggctggcttccctttgggtctagggctgcgctggcaagccccttggcttactcacgtcaagtgaggaggaggctgcctccctgggccctgtccttttgcatcctggcttccttgagaacaccttggccaggtcccaagggcaagggggatggaggagggatgggagctccaggacacacctgtgtcctctcgaggggtttgtgctgggtgctggcgggggcggtggcaggaacctggaggccggcctcccaccgcctcgcctcttcccggggtccaaggctgtgctaactgccctgccaggagtttctctgcttgtcccacttagggctctgtcccacttagggctcttttcttttctgtctccttaatgttcaaatgagcatgttttagtgattcaagttggacctaacagaaggcttttctcaaaaagtcatgcaaatcacgtccaaagagatggcttatttctgttaggctgtcgaggttagctgagtatgagatttccttgtgctcatgtggcgtctctgagttttgggttccttcttgcctcctagagagaaggttccggacacggcaggctgagcatcgctttgacccggagcgagggctccagacagggcaggtccaggagccatcggttccagagatgggcccaggtggtgcgtggagacacttgctgggagaccagttgtgagctggtctgtggacaagaatggctgcccagtcctggccctcctgaggcttctggggagatgtacccagaggttgaccgagtctgggcagggtggtgggccgtgggctggccagggccttacatggatgtctgcgggaccctggctccgagccagcaggccctgtgtgtgtgtgcccggctaggaggacctggagggaggaagggaggcccctgtggtggctgtgcgtgcaggatcccgggtgtccgaggtcagccacctgggcctgtgtccacccccagctctgggtcccgctggccccagctggaacccggcgcccacgctggcagcctgtcctgccagatccccggaggacaggcggatggaagagaaagggctttgccctctgccagcctcagtgggggcgggtccgcacaggcctctgtgtgttccctgagctggggggtggggggcacgatggacaagattctccctcttgagtggggcactggctgggaggaatctgcacacagaggcgggattcttgtcgaaagggctgagagccaggctgtgacctgagtgcaggacctggggccggagggtgtctgcggggagggtgggggggaggatggggacggcaggcaaggagggaaaggggcaggagaaagggaggagggaaagaaggaggggggaggagtggaggagggagatgaggaggtggagagggaagaaggagagagaagggagctgggggaggagggagaggaaagaggagggaaagaggagggggagagcagtaggagtgggggggagggggaggagggaccaggagcgaggtggggagcagcggccggccagggcattgaggggcctgagcacatgcaaaggaggcgggtgaggtgcacacacccccttggggcggtgcgggtccagctttcaggtaccggccttttgtctgctgtcctgagctcgcctgtcagccccgccgtcagaggcctaatctttttttcttcacgggctgaaaaggcccagcatcacatcaggcaacacactgctgcctattctccttccccacggaacccaatcactggacagcgccgctataattcactccccagcccgccctttgaaaaggtgggccgcctcggtgctgctcgcccagcaagataaatcatgtgctcgaaattaacttccattgaaagggagggcaggaagaaaggctgatggccgggcgggcggagcggcgacggccttgataaatgtcagcgcggataatttgggggctgcgattaagatcttatctaggtgggaagtaaatagaaaagactttaataaaactccggccccatccgtgctcaccagctcgggaagttcctttatttcccttgtctttctttcctctttttcctttattttttgggtagatcctaagggaatgccagaaaggtcttcattaaaggaaaagttgggtccaggacatcacttgaaaggatcagacagacgcagagaagccaggcgtcctcctctggtgacaaagggcagaaggaaaggaatgcagtgctgcctctgtctccaaattccggcccgcccgccgcccggaagagatgctggggcttgagtgggagcgccagccctccccggcgacccaccttcccgcctccacgtttgattttctttggtcaccaacattattgcgctcagtgaaagttttcctcaaataaaatgaaaccgaacccccgcggcacacaggagtgggaggcacgggctcgtaaacgcgctcactcggcgcgctccggggtgcgcaccgccctcttttataggctcccggcacttgggtgtttatttttgctactgatgacttcagcgacctgcagcttgaaccccacgccgcagggcagggcgggggccgatgaccatccacccggtggccccccatggccatgttcctggaggcaggaccttcccatgccgctgacccctcccctgcagctgggcagctgctctgggccactggcttctgctggctgagcaggaagtcacacttgattgacgaatctatcagagggtggcaacgcccctaagtggaatctgcagggaggtgaagggaagggcagctcagcgggccggccctatggccttgcttgctccttcctagtggaccagggtggtcactggggcctcctggggggaaggggccccaggcactaggaggccacacatgcaaagggctcagcccagggcccgctgttggctccagactcagcgaactgttatgaagatgggactcgcccccagcgccatgtgggtcttccagggacggagtagtgccagccctggtcacccggcatctcaaggccacctgggaactgactcagtgcagctcccttgggtccatcactggacagcttggacccattcccagaggttggagacccccgagagagggtctgggtgtcccgacccccagagggcggaacgctctcctccgagctctgtgttccgtagtcctgccgtggctgcaggagggagggcgattcagaagagctgagtggggggcaggggtggggggtctctgagggggctgagtccctggtccactccaccacccctgcaacctgggtccgagccagagagtgactcagccctcagcacacagtagctgctcaataaagacatgcaggaaggaaggaaagaaggagggtttgagcagacacatcacacacgaggcagagagacctgggcgcaggggggagcagcccagaaggaggactaggctgcagcagggttgtcccatcgatggtgctttgttccggaaaattccttgtcttgggcactgtggatgttgagcagcatccctggcctctgtgcactgggtcgcacccccaaatgctgacaccccgatggctcccacacagcctgcgccctggtcccggcggggagggagctgcctggggcgcttgtgccctgacagcctcagagctcagggctgggtctaatttgactcgagattatttcccattccttcctctttgatcttaaacactcgggatgcacagggacaaacgttagagcagtttccttcgcctggcaatgtttttctttgattttgtttttaaacccacaagcagtgtgtggagccgtttcagcggcaaagtcgccaggttttaaaatgtagattcgcgtttagatgtttgtttgaaatgtcacggggtccttttaagagagcagcgtggccccactacacgcatacacatacacactcacacagacacgctcacatgtgctcacactcccacatacctgtagatacagactcatgcacttgcttacacacgcccacacattcacatgtatgcacgcacacagacaaatgcacacacacacttgctcacactcgtgcacacacactcaaggaaagctggctgccccagatgctggcacatgggtgtgtgcatgggggtgtgctccggtcaaggagcccacagcagtcatttgtaccctgagcctcatccaccaggaaacactccctctcccaaaacagcccccaggacggccctggagggcaataggcagtgtgcagacatcaggggtcggggaaaggaggctcctgcgggtgctccccaagtgtgagggcaggagcctccccagcgcacagctggcccagccccagctcctcccatcacacccccagccgtccaccctccagcccctgcttggcgacagcatccccggtgtctcaggacgcagcctttggaggccagggcatttggttttcaacctcagctgggcccctcctggctgtgtgacctgagccaagtgctgcccctctctgcgcctcaccgttgtcctgatgggtcgaggaggacgatgggactggcttcgctgactggttggggagacgctctctgtgggatgacatgggccatgctgggtcagtgctaatgggaccttcatgtgatggcctcagaacccgacaaatgtcctggcagcggggggcgggggtggaggggctgtgagaatgtggtggccccataaactcccattgcggaaggcattcgggggaggcttggaaagtgacagataaagcaaataccccagctcctaaacaaacccagcaggacagggcagatgacaaatgatgggattgtgtcagcttgagagcagattggactaatggcacgccggactttgcactcctaggatcaatactccatcaggcagggcttgtgcgcggagctccccaggacaaagaggcgatcggagatggtctgcgcacaccctgcccacagcgccgcccatgctgtgagcccacgtggggcgggtgggggcaggggtgccatgagagagcaaggctgcctgtccgaaggctgcacctggccgtggctatccagccctggcctgaatcctggccacgaatgcagtctcctgcggccgcaggttagcatgttagaggctcccgggctgcagatgcccagtgctagtttgtcagcttccacaaagctccacctggctcttctccagctcctcgggggccatgatgcagctctgcaggcactatggccatttccccctgtgtccggcaccagggagctgctgtccccacagcccctcgcggcccctgcagctcccgggaggctgggttcaacagcaggtgctgaccccacaccactgtggggctgggcgggcatggagggtgccaggccagggccttcttgggggccccagggggcccctgtcttccagcaggtcactcaatagaagttcacctgctacgtccattccctggtatgttcattccatccatgtgttgagtgcctactgtatacaaaacggaaaaccacagctgaggacagagcacgtgggagcagcgcaggtgggaggagccgagcacaccctgggtggctctggtggttccgtcccctggtccctgaggggagtttgcttgtaagacggcaactccacccagtgccgctgaccctgctcccaggcagcaggccctggtctgggcttgatcactcgtggacttggaccctcacctttccctggctgggcagcccatgccaggccctcttgcagccttggacagaggctgcaccgtggggccctggccccggctctgatcactggcaccgtggggcctgtccacccccggagccgagcccaggcacagtgagggtctccgaggccaggaggtaagacgcaggtcaaagctgcttcttgctggtccagttgcctggccccccaggcccctaggacagctctgccccctgaacctggctctgggattccggacctcagtgggcccctgccccccagcccccagaaaggttatcattgttttggtggatattgcacagcgcagccccagcccccctccagcagcacttcagagcaaactcgcggacgtgtgatggcggacagcaaagtaccgctcacttggccacctcacaaccaacccaggccgacgctcacggcctccagcgccctcacaccgggagtcctgggtctgcaccgtcctgagaggtgggaattgagtttgcagtgctgggtggacgaggcacggctgtcctgcaggtgtccccggggatgggggtgacgggctcccctgcacagcgatcaggccaggggctcccgttgggggatgctgttctctgtggggtagacccctgccttcacgcttttctgctctaagctttttcgccatggcttattcagtaacatcctcttagaataattcattcctggtttcccatctccacgctcacctctaggattgctgccccaggcgggcccaggtttaagggcattttaaatacaaatatggcaaccagggagccagaggccggccgtgtccccgtggccatggccacagccttccttaggagaggaaatcggattagaccgtggagggatggggccaattgaaaccaggggtcgcagagtccctgtggaggtggggggccagccagcgaggtgcagtgggaacacccccgccccccagatccccccaggacggcgcgtgagagccgtaggtctgggcagccttgtggagcccaggcccacccgtcctactttcttctgttttccagcctcttctgggtgatctgctgagtgacccagaccagaaaaggcccctgtacctcctgtgtgtccatctccagtgtgaggagcggggtgtgggggaggaggccctgggtgtccagtgcctggcttggtcccgagaaccaggccgtgtgcgtgtgttgggccgggtcgcagcctcgtgccacagtgggggcgggagccatggacccagcggtcctgcggacgtctctgagttctgttctcttcagtaggtcataaacccagggctcgctggtcttagggcagctgtgagcatagtcctgtgtctactttgaaatgaggccaggagggttggggcaccccggcccccagcccctcccattcaccggattcagtagaaagagcgtttgagccgagcttggagccaggagcctccctggcatatgtttggatggcgtaagccaagactcacggatgtccagcaaactgtcctgagcaccacagggccgactccatcgcagcctggaggaggccttgggggggggggtgggccacccaccaagggacgtgagcaggggagacccaggcctgtgtggggcggggggggctctgctgaggaatgcatgcacacgagtgtgcatgtgtgagtacgagaatgtgggtgcgtgcacacgtgtgagcatgtaccggtgtggggatacagggcaccagcattgcctcagtgccgaggtggcacgggagtcccataaagaaaggactgagtgagccccagcccagcccgcaagacctcggttctcgggactcctgacctcgaggcaggccaggggcctcagccgcaccccttggtcagcctaggacggagagggcagcgtgggcccaggatacccctccactgccctgggcctcatctgcctgctccaggtgcctctgtcaggctgagggcgtttgtggccaaacacgcccgccttgtgacttctgcctggacgcccatgggtcccagaggctcgcgtggatacccccgcccagtagagccctgctgttccccttcccagcggccagtgcctctctcagccgcagggggcttcctttcgaggggtgggtggaggcgagtgagctgcccagctcttggtgtgcatggagcgccatggcaggcctggcaccccagtggctctttgtgtggccaggcgggtgacccaatgcaggctcggccctctggggtctggttaattacctttctctggaccccagcttggaaagtcatttccctgaaaggccacggggaggggtctctccgtccagccttggccgacccggctgtctggactggtcggggggaagggatgggtcttcttagcacagcctcggcggccccaaggccttgctgcggggctggggggttttggggagcatggcgtgggtggagggtgcgggggctgcaggggcacgggggccctgggggcttgggggctgcagggctggggtgcagctcttctcgggcgtcccggggcgggagggccatgccatgtgtgctctcgggatcagccagggtgggccgggctgggaaggttgttggggagagggttctagaagcctggggcagcgttcctggctggcaggaaaccgaggcctgaggctcgggaacgtgggagagtggtgggggtgccacgttcttgtgccggcacaatgagatttcgatgactgatcctttttcttaatgtaagccgtggagttaagtgtttgaaaatcgttccagaagctgagaaaggcctgcccttctggtcacgctggattgtctcttacttagcagccgacaggcccatctgcagactgcgggggagggcgagactgggggaggggcagcaccccatccctgctgccctcagagctgcagtaaggaagggaccgtgccctgcctggtggccacagccctctgcggactctgaggaggggaggggcgcccaccttcctgccggcagctctgcctgcttgccccagggtctgagtcccacaggcggctgtgctgtggatcccttccccagggctgagggaccccggagaggccagggcgcaggcctccctgctcagggtcggccggagcctccaccacccatccttggcccaggtccacaggtggttccctgagcctctgtgggaagacggtgacgggccagcccctgccgcccggctgacttatcagttcacagcttgaaatccaagtaattaaggggatggattaaatctgtaggatgtctcctcgctggccccatatcatgttgttccacaaatatcatgtgtccccccctcccccggcccacagtctggcggccagccctccccaggcgctggaaggttctggggttgcgtctgacgcacctcggccctgtgcgtgctgccgtgttcccgtcgactcctaactgccagaaaacggacgaagcgtaaagaaagcggaagatgggtgtacaagtccaccccccgtaaagggcacacctgctcagggtgtggctggagggtggggacaggggacagacaccagctgaagggtggctgggaggaggcttgttcatgaacttgagggggctgtcactcacagagagcaccccaggtacagaccattcctttatttttagtgaccccaaagggtctgccaccaccccgttgaagcccgagacggaaacttgttgtcagctggacaggccacccagatggcctgagaggaactcaggagacctctgcccgtctgccctgctccttgtgccctgcagaccggagggaggacaaggggtggacgcctgcctgtgagcctagcggccagagctgctgaggccttcagtggtgaaagctggggtgggtattgaggggtgtccggtgaccccaggcaggtggaacgaggggaccatgggcggcgggccgagccagggccccagagaggaccgtttgaggctggcttcccacttctgctgagaactcgttctgtgtttagattagaaacaggatataataaacttgttaacatgtcagaatcttgtgttgggacaacataagtgggtgttttctcctcttgtctttctcttgtgcaggaaaatgctgcaattagtgctctggctcttgaggcaggcccgggggaggtggtggagccgcaagcagaggggctgttggactgcaggcccatccccagcctggggccggcttccccgcggtcccggagcggtccagggtgcatggggtcggatggccagggccccgggagcccaccctcccagcaaggcctgggggcagcagtggctccaacaccagcatctgcccatgaaaatgaccctaggaggtggtcgaggtcgggtgaggggaggacattatttgttttcgaaaaaggcttttactttaaaagccttatgtcctggaggaaagcaacatggagctattttcaggaatgggctcaaaaaaggatcagctcttaattaaaacaaaacacagtaagatcaagctgtagctcacaggcccccagccggaacaaaaccgtttctgtcgagaccccaggagttgtgcggccgccggcctgcaggccttgctgcccggcccgagggtggctcccagccctcagccaggacaaggaaatgtggacaaagggcgcagtgaaaaatgaccacggcctccaaatgtagagaaccagatgtgagtctgggaaggcgggggctaggcacagccacgcagcctcgggggacagtggctgggcctaggcagccgcctgctgcctggacccactctgctggccctgcaggcctgccctcagccagcaccctgtccccgaggtggctggagacccctggaatgtgagggccgggtgggacgtggaaccatggagggctgcagccacatcaggaacccgggcgatgtagtgagtgaaacatcatgaaaaatgaaagcaaatctgctctaatctccccgaacaaaggcctttcattcgggccagtgtggcgcgggcgctgggtatcgccagatcttttgtattaaatgtgcacccggcagaatgtggatcttattaggggagccgcgctcaccgcgagcccctgccccgcgcacggtgtctggggaggtcagggggccgaggccatgtcctggccgctctgcagccgcgtggcgcccaggaacacggcccggtaaggcctgcccatcgctccgccaccgccctcggcgtccggcacaggaaaaatgaaactcgccacgaaacgttcctgagatacccagctctttctttgtagataatcctctgtgtctaattgcacaatctcatgtccccggacagtcaaatatggtccccaaagtgagtgagccccaaaagggaccgatcctggagagtcaggtgtctgcagattttctgagaagggtcgaatgtctgtccaggggagacgccccctgcgcctttaggttcatcacagacttgacgcgatgtcttttcggagctctgcccctcggggcaagtggccttcctggagatgccagcccagcccaggcctccggcagttccgagccaccgtggggagcagattctggtttggacagaatcaacagaactcacaggcctgcaggggaagaaaggatcattccatgagtgaaaaaaatgaagtgtctccactcaacaaatgccaccgtcccggcttctaccagactcagccccccaccaccgaaagcagccctgggaaggaaatcgaatttaggggtttcccttaccggccagaattcttaatttaataaggggtggagagatgctgttgcattttattttgtttcgactttgaacaggttgaatcttcctgtagggctcaaggaaggcggctgccgggaatgaaaaccagccccagcctcgcaaattcaaaccaggactccgccgcatcccagcgcccggacaacccacgacccacgacccactgagggggctcgtctggaaggaggggcgaggggagagagtggggcgtctggggcctggggggcggtgcggggtggggcttggtgctctgggcctcgggtggcaaagggccgaggtgggcgggagatgcccaggtttcatcgtgtgcgaaggagaaatccacttggccactcgcacgctggcacacggtcgaggcacccggggaggctcgacagtcgggttttgcatgtcggccgagtggttgtagccgatgcaccatcgggacttccccgtcttcagtgcctcctcctctgcaaagccggagctctgccccaagcgtcaggagtcttcccttgcgcgtgggagcagggacctcaggatgtgacagtgccagcttccggcaacgtgtctgtctccacctgcagaggagcctgggaaagctggcagggtggtaatgggaaaccatcattgtctgcaggggttaagagctgtgactctcgaggcgtctgtcaaatctacaaacctgcaagcgaggtgtggaaggccaagataaaacgctttcctggcatgattggcgagtggctggagcccatgaggccttcctggaggcggtggttctctcaccctcctcttgtactgcctgtcagttcagagagcaggatcccgctcctgtgtgatggaagcccagagtcttcccaggcgacgtgggagccgctagaattggggggaccgccagtcccagccccttctctgccctcctgtgggacgtcggaggccatggtcctggtacagtggggatccgggggacgaggccagcgcagagtcatcctttcctgtccttcaacgtggttagacatcccgtttgaagtccaagctctgcatctgcggtgcaggcccggggcaggtcacctgacctctcctcatttgcgggaaagacaagaaagtcacaaccttgtcaagactgtgaagtgcccccccccgggtgtggagatgcaggccacagccatggtgacagctgtgggggcatcacgccactttgaagcaggacacacggaggcccgcgagccccaccctgaagggcgggcatggccccactcctcggctcctggcaggatcgtgtctctctggggccatggcgcccaggcggggcgaggagtctgccagcctcagatgtgagaccccagccggccccatgagctctccggggtcgtgcagctggctgcaggggcccctggaggaggtcccatttcttctgagcccgtgcaggaatctctgcactgctgttggcctggctggcctggcagtgaccttcaccgggggccgtctgcccacatgacctctaaacatgacctgctgtccagtaatggacagtggaccagggaggacggaggtggcagccacagctgggccctcaggtcaatgtcaccccctctgtctgggagtgagccccttgctgtacggcagccacctgctgcactccaggcctggcctggcctctgggccactcagccctcctgccaggctccagggaggggaaggagagtgagggctgggtcagacccaagcatgcgtcccttctggcttcatggtggcgccgtgaccccctggcattagacattaattaaggggagacattagaaggtggggtgaccccgtccccttgccggctggtgtgcccagcctgagggcctggaggcagccctccgcctggacagaccctgcagagcaggaggaccccctgaagacccctgtgcatcgtgggccctggggggtctgctggtgctagaaggaggtggcatcacatgaggcggcatcacatgtggcggcccctgtgtccccctcaacccccacccggcgctcagattccagccacgctgcaggccctcaggtcctgggtattctctgccatctttctgggtctctggagaaactccatttggccaacccaaaacaaacacgtttctgccgagtttctctttggtgacggctctgaggttttgcttcgctggggcctgtcggaggctagaggcaacgggccgggaggcgagggcatcgcacagctgcgcctcactcgagtggcctgtcctcctcccgcggcggctttgggtgcctcgatgcgtcaggcagaggccagttcttaccacacagcccaggcccggggctctgggctgagaggcccccaacagctgaccgggaggccaggcctggtgccgccgacgaggaggaaggggaggtcagttagagggtgggctgccctccctgtcatccctgggtggggtctcttcagcccctggggtctcctgtgccccctgaggtatgtgctatagtgccaggttgggcagggaaaactgaggcccagacaccaggcgactttccccaggccactcagcactgcccggcggcttggaagggcagggtagccagcccagctggactggcactcagaaaactcagggccactgaccgcgactggccctgaagaggtttcggggaacttttgagttttagggagaattgcacaggtcgctggaccaggcaggttttctttaagggtctcaagagcgagctctttccttcgctggcggtcccggtgcgtcccggcatttctgatgtgttggtcagcatgcagggcgctgcccccaccgtgccgggcccgggagcggagcgggctgtgcggcgaggtgcgggccggtggcgcggggcccagcgctggccgattcattagggtctgggctcctttgtgggccccaagccccaggcccgctgctgtcacacgggctgttacaggcataatgatgggcattagagccccagagcaattaacaggctgcttaattagcaagaggaattagagtttgagtggctgatcttcgcttaaagcctcgtcgagcgggcagttatctgattatgcattgatcgccggccggcctccttgggtcctcctgtcccctcactgcaatctgtgaatatacaactgcttaaatattccgaactgaactgtctcaatcaaccaggggagaaaggggcccattgtggcctcccttcccggcccagctcctctggccgggaggaggcggcagccggtcccagggcccttgcccggccagcctgactgtgcctgggaacaatatcctcccagctgggccccaggcgggcctggccacagctgccaacgtgtgtcctcctccactggcctctcctggctcagggtggctcagttcctcttttctgccgcaacgggctcagacaaacccattgtacagatggataaagtgaggcctgagggtccaatggcacctgcacagtgagtgacaggcccggctgcctgtgggcagcgtctgggttctctcgtctttgtcgagggaggttcggatcacgtgtgcacagccccttcaggaacaagttgatctagcaccttgcatggctttgaaagcctggggcactggctgatgccaggcagtgtcctcttgtggtttgcaagctctttgggagatggaggggtagggagtcagaccagtggccccagccctggggtctagtgtggagcctgtacgggagggaagatgcttctggagggagaggggagaggtgaggctcccagccaggcctgaggggaagggtgtacctgatgaggagaacagcacatgcaaaggcgcagaggtggggaggtgcaaggaatattccagaaatgctcctgctcctgtgtggctggtccacagggtggaggtggggcacagggttctccagtgtcaggtccagcacttgctctcggaccccagatgccctccctgaatgttctagcaaactgaggaggccaggagcttgcttcatccagactcagctgggaggccccagggtgggccccgtgaagttcccggacgcagagcgggcaggatggccattgggtcctagacacatttgctgggattctgtcccgaccccttgactcagcctggccacagtgagaagaaggccgggatgctggcttttaaatgaaaagggaaagttaaggagaaaagatggagagagagagagagaggggcccagtcggccacttgttttgcaaaactcaaaatagatttcagcaccatcctttttagagacgaatctctgggctcttctgcgatcctcgcttgacctctcgtggtttctggcaaggggcgcctgttacgatttggcatcgagcgactcaaaatgcataacagcggtgcgtggaccttgccaagagctgatctgcgtcggtttctcgagcgttgctttccttgtttgctacactgggtttcgtcttacccagacgatgccctcatgagatagacccccagcccccaactttgtgggtgcagccaacgtagatcattccactgctcgcaagacctgtgtgtctggagggaaaggacgctgcttagcggtgcggcttcacagcctcccgggcacttggggaatctttatcgagctcaaatgattagacctggtttctggtttcattttggaagaatcgctgctaagcagatccattccgcctgtaaaatgatacagacgctccgggccggtgcactccctccgtgctccaggccctgccccttctccgcagggcaagggacagcaggatctgggctgggcgggctgtccctggccaggtcccctgggcgcaggtttccctgtgtgagtgtgggagcagcgggaagggggctagcccccagcccctggtttggggaggcccatcggccctggctgctcatggaggagcctggcttgccttcctggagcagaaagctagacagaggagggggctctgagctgagagtcagctggatgcggcccttaccaggagcctcctggcatctgggacactttccccagcttcctgcttaaattgctaattcccgagaaaagggcaagcatgtttccgtccgctttggtcggtgcttgaatgaacccctgtccccctggtcacctggctgctgagggatcagctccacgtttcgtctgagaaacagtccttctgggtgttttaaaattgaaagaaagattgggtctgtcttccaggcatattctgctgtacggtgggcgaattaccttcagagaaggggaaacaaagagcgtcaggtgtgacgggcagcggcaaaaatctctgctgctcgagaccctgggtgctgtgtctgggaaaacttcagggcctggaatccgtttagattccggtttcatcccaggcagaatggacttaaatattttggggtggcttcttgattcctttttctggtcgtgaagtcacatgcccggctttggaaaatgaaagccacagtgttggagacatcgaaaaagaaccctatggatcgggatgcgtgtggccccgtcttggtgacattctctcccgcagtggcggctcgcaggctcagcccgggggaatccagctcaggagcccaaaggcaattggcagaaccgtttggggtgagcaagctgtgaaaggacgtgtcctgggagatggaagcggtcactgtgttttgaaatatgtgagttaggggcgaggttggttaaatgcctggcatcacgcgtgtatgtgtgtccctgtgcatggggacgccctgtgggcacagatagacaagccaagcccccaacacctgatgaccttccttagccaagaattgatagccagcctggaggcttgtctcaagcccggtgtttctcaccctctctatggactcgccaggtgggtgcgcgtctgcaggtgctgggctggtttctcccaggctctgcactgggtccctcagcacccagcctcttcccagctcggggcatggagagcaatccctccctcagcaggagacctgggcagggtcactgtgcgatttgaaaggctgagtgtgcaagggtgagggcgccccagcccatactcctgacccctggcctgacagtcgtcacctagcctggccaacgatggatgacctggggcctcgtgggggctgttctgcttctcccaccttgttgtcagtgaccacaccatcggtcagttggtcttttctgggtggagacagtgcctgtgccagactccgtgggaatggcctggacagactgacatcacgtctgtctgcagcccagcgagtgcccctgctgggagctggagcctgcttggcccctgcttacatcctcgaggaaatgacggccctgttctggcctgggagtctcagccattgctgccagcctggtgtcattgtctggaaagtgggaggggctgtttgaaatgagaggggaggtgggccaggggaggctggaggggtgtcagcaccctgccatttccttctccccactgcgggcactgcaggagcgtgcgggctgtctgcgagccttgacaggcagccaggagggaagcccggggtgattgcatgagtgtgggcacgtgcatgcgtgttcatgtgcagccgcaccctggtgcctgcgtgccacaagtgcagagatgcaggggtggcgtcgggcgtggttccgagcacctgtgcatttgaaacccatttctgtctctcctggatgctcctcttcagggccaggacggatgggagcccagggacctttggggcagatcctctctggatctttccagccaccgtgggaggtcccgagaggcgtcagtgttgcaggtgggaagggacacgtgcctcagtcacttggtcccaaagcctgcagagcagagactcctagaagagaaaaaaggatccatgtgggctgctcctgccccagcccaccaagttctctttctcattttgttttcctctctctctgtgttttgtgtctatgtgtatgtatatatgcacacacatgtaacgtacgtgtacacatgtatatagaatttattctcttctgctatgtataacttagttaatgtaataaattacttgcattataatttttggtagttttctcttcatgagataatggatcctccttccacagaaaacaataaaggagggaaaaatagaagcacagggtcccaccccagccttaggaccccctcgggcaagctgggctggggggtgcaggacagggaaagtcctgcaccagggggtgctggggggcgaggatctcccagtcagaggatccgcacccacccagaagtctttacagaatgatctgaagaaagattgaaggcaccgagaccccagagacaggcccactgctacgtggtcccctgcaagtctcaggctgcgtgcccatccccagggtctggggggcacagggtgacaggaccccagatctcagtgagaagtgagcagacccctccctcccaggcaggcacaacacaacacaggggcccagccggcctgcgtccccagagtctgcacgtagcccggctctccctggaggccacaaagggtccagtccaaacccacctccgtgtcccacattcctgtcaggcccctgaaaccacaaacagaaacgaagtgaaacagcaggtatcagcctcccggaggagggcgagtcctcgcgtggtcaagggccgggcgcttcctaaggcctagaaaccttgacgaaccgcctccccgagccatccggcatccggcatccagcgtccagcagtggagggggccctccactccggcttaatcgctgccacttcattgctgattcggtcactcggctgggactccggggaggggtggtgcttcccgtccgccgtccactcccctctgcccaccccggccagcccgcccctgggcaccttcctctgtagctgatggattttcaagagctcgttaaggacaagtttcacctccgttctgccgtgggcggtttgccttcttagtcgtggtgctgaatttggggtcttgttttcccgccggttggtgtttgatgctgctgcagtcagaccaccagcccttccctccaccccgcccgcctctgtgctccgctccagccgggttcccttgccgagagcgtgtaatgccgacgtccgcttcttcccagcattttcctgattcgccgggtgtattgagaacgtgccccatgggtcacgtattccgatcgcagccctaggagaggaagtttgctcgggggtcctactgaaagagcagtgccgatgggatggaggcgtcttttattctagaacattctgtcgtttggctggctctgctatgcttcctccggcctgtgcctcacctggcaactcttttattttttttgccttatctttgtaccctatatcaaatatttaaatttctggaaaagcaagcttctccttcagtgttaacatctctttttacggcggagttttactttctttcctcccttcgagaaaaactttgtcatcatatttttcgagttaaaaaaaaatctattttgtaatcttattacatttttgggttaaagttgggggaaggaaagctttatgattttgaattcttcccatttaggaacttgatctgtctctccatttagtcaaggtttttgagagaacggttttcactgcgttttgtagttccctttgcaaagtcccaacacatttctcattgagtttatttcaaaggatttttagcgtgaaaataaaaaaaaaattcaaactgaaaataattccaattcttatatccctctactgcccctagtacagaaaataaatttaatgagacaattctagaaacaataagcaaatatttgcacgccattggacaagcgacggttttctgctcctgggtctgtgccgcgctccgcgctgcaggcggtgagactgtgccctccccgtgggagcgcctcgggcagcgggagggctggccggtgctggtgcggctcctccaccagcaaatgcgccaggtggccccagccctgagcgccggcctgagggagtgactggctgccctggggtctgtgctccgctccctctcccagaaggagcggccctttttctctgccctaggctgctggggaggaggccttcctccggcccctatggcccacgtcctagataagcctgttaaattagccccataatacttaaaatgctagtcctttaatatgtaaacaatgacaatttttttccaaagggtttacgatttaaattagttttttaaaaacccaattactgccatttaattcggaggagagagttctttatgtgctaattatatagttgggaaatgagcaatcaagggcacaatggggcattaagggcactttttaaaggtttaaacttatttatctgagtttggggttgggtctgcagtgtgagcggcccggatggggggcgccgtagagggcggattttggaaccaggatgaagggcaacatggccgtgcagcaaagaaaaatacgtggtcattataatcgtcttattatttcaacaatgtttacgaagcatcagaacctggacacgggccctggtctctgcccagaatactccctggccagcaagcaaaggagaggaggctgctaggtggacggtggacaggaaacgtgggcctggtcagagcagagtcttgggcccctgaggggcctgcccacatagaacctcctggaactttccagaaccttccagagctttccagaaccttcctgaggagcgcacatgaccctagaggccgataggctgtaccgtgcatagttcactgcctcagacgttggatgctgggtaaacgagggagggggcttctccacctccccctatcatgccccccttcctccccatccatgaatctttctgaaggatttgccttccttggagccaacatgagcccagaatgttccagagccatcctgcccatgctcttgccacatgaagtcaacacctccgcttcctaagagaggaaggatggttcactttagacagaatttgtctttaaagcaaagtgacccttattttggtccagctttctctgaggaggtgcctggaggagttcggtcgggagttccctgcttcctgaagggacactggtgcttcagatttttggaagagcagcttgtggttaacactgagccgtacgtaactgaccgggatggtggacgtttgcgcaagacaccgcaacactctccagagaagcttacgcctcgcgcctgtggcaggtgcgtctgcgttggcgattggaggtcctcctgcgtccagctggggggtgagtgggtggctctgaggccccagcatggcagccctgcttgcccccctcctggcacagaccagggaacgtccttgagcggatgttggagatgaaacacacctggtgtccttcagcccttgggtgggagcagctgggtcttccccagaggaaggctttgctggcctggcggagtcctgcacagcgaccctatggtcaggaagggagccgcgcccccagaatccccacgatggaggagcttccccgggcccttccttgcccttcccagggcccccagcaggcgcccggcctctgcactcggtctaatctgcttctacaaaaagcccacgacacgtggagtttgtgactgttttcagaggggtttggggctcaccggggatcactgagcaagtgatccatcacccggggagccagaacctgggctcctgacggggcccagccccgccgatgggaagggaagtccatgcacagaatcttcccaaataatgggaattatgtggaaagggccagggaggcaagccgcggggaggggagagtgggagcccagaggttccctggaacatggcacgcatcttgattcttaaagacgaggggttggccagcagaggagctgttggcagagggtgggaacgccaggcagagggcacaggctgagccagggcggggaggtggggaggtggcatccagagtcggggctgagaggggctggctttctgcatcccaccctcccctccacgcggccttgggcgccagaaccccgggacatggaagtcacgtccacgcggggaccgactaccagggcatctggggcctgtgcagccgcctcccagagaagggggaggagaaagacccgtgaacagcggggcctgacctaatcggtgcaggaaagacgggatcggcacctcgagtgttcaaaacagtccagattaaacttaaagcgattagacaacttattttctttttcaagacattagctggtcaaaaatgacaaaaatgtccttaagccgcgatgaggccgaggcgggggacaggcggccttttgtctggtgcaggggcctgactgcggagcctgcgtggggccctcgggagcccaccttcccgcatgctggcgcaggcaatcaggccagtattactgggggattagacggccgggtggcaggggagctgccggccacccacagaccaggcccagggccccgagtctgcacgccggctgcccgctgggttgggggtggccgcgaggaagggtgtcttcctggcagggctgggtggcacatttgtttggggggctgtggagcaccccggcccccgggaagaggtgccagagctctgcctgtgaggggctgcgagtccagctgtctcccgttggcctggaggaccctggccagcaagggtcacccgggctctgtcttcccctgtgccagggctggagagcaggcgggggtccccagggggcgcaggtgggaagggtgggcccagcggggcccttcccctgcgcccagctcttcctgtcttgcagacaccgtccttgggcccgacattttctcaagaaaagaaaacacacgggcccacatttcttatctgagggcaaacaatgagatcccgccggccagccgggaaggggctttcacggccaatccagcgacgagagctggagcagccactgccaaggccagcggggcccagcctggcagcctctgcgctcggccatggcactccctccccctccgcctaggcaggtgacacgccagctgtgagcctgtaaccggagcccacctgacgcaatgcgcctggcgtggcaatgacaattagagggtctcttcaccccttccttccgggcactcacattgtggccccgtctcggcccccacccggggcccgggcgtaattatagggtcgggttaccccaagaaacaggtcttggcattgttcggcccagcgctggaccctgctgccccttccccccactttcctccccactctgggccaggtctccagtgccatccagggacccctcctcagtgcccctcgtcggcccagagaaggtggggtcagggagaggcaggggccacctctctggcgctgggtggcaagcttggggcgtgaggtgggggcgtctccttttctttttccagcagttgaaattcaaagtccaggtgcgaggagatcgttttgctccattgtccaggagccatgggacaccgggcagggggtggggcacagggctccagcaaggggcctgggagctgcccccagggaggagagggggcccttgggtggcccaggggtaggcccatcagcctcaggccttgagctgcgcccagcaagagggcgatgccaacgcactgatggatctgggggccctgtgactcagagcacagcggaaggcggaaaccactgctcgccccaggcacccagcaccccctactccagcccaggcctgttgatggtggcaaaggtgagggggaggccgtgaaggtgggtgcccagtgatgttcctgggggtctagcgtctacacagcacgctggggaggtcagctctggagtcgtccattttacagacaagaaggtggaggcccagggaggcagggtcacttcttcaagaacagacggtctcagaagtatcgaggctggggtttgaacccaagtgtggccgtgtccagggccctcgcccccactgttttgtgtcatgttttgctgcctgcagagtgaggagaagccgattcctagttttcagcaaagtgacccccacatctctctgtccacaagccccagtgggccagctctgaggacccctgagctttctgaggccgtggagcctggtttgtctggctggtagcgcccggcactgggtgtggacaggggaacgcgattctcaggcgtttcctgcggcccccatggccccacctctggctcctggcaaacacacagccctacggatggctgcaggctccgcccccacccccaacgtgcgcacccctccttcgggcagcctgcgtgagtgtcagtatcccgtcctgcccctgctgggtggtggggcagccccagggctcccctgggaactggtttcggccctcatgttggcccaagtcccacggggagctctgggtggtagctggtggccacagccaggggctgggcacaccttcctcccgccttgccctggccatgggctggggaggggctggggcacagggaggtctcagccccggggctcttcccccgcattggggcatcagacagatggggtggactgggggggtgctggtgaaggagcctcaccccatttcacagatgaggaaatatcgtcagagaggctgctctgtagtgcaggcggtgggcggccagccccagccgggtgcatgcagatcagacctggggcgcccccctgctgctctctgctgcccccagagtcccgctcccagtccaggcatccagggcaccaagggcagggccgtttctgcagcagccccctcgggtggacggccccggggccccttctcccctctctctgacctcgcagccgcctggcccaagagccagcatgtgaggtgggtcacacggcgcagatttagacgtgagcctgtcgcgtagggagcaaggaatgcagggcggcccctcccgcttcctgcaaagcctcgtcatgtgtgtgagataagttctggggacttggtggctgccagggggacgctactggtgctgccgatatccgtccaggcagggtgctgaggatgtttccctcagaggtctcagaatgttccggcagcggtgcccctccctgcagtggccagggcctgctagctacaagtcagtcgggatatggctgagcccagaggggccatcctggagggctccctggaggaggtggttttatgtggctggaggagggtagagatttccagtgacgaaaaaagaaaggcttgagaaggcttccttctcgggtgcataaaccccgagttaccttctcatccccacgcagcagccaggaatcaggtttcctacaaagcgggttcttggaaatatccctgagcctagaagaaaattcctgcttctcagaattcctctctaattttcccgccagtgagcagagccacccgcctgtggggctgcagcagctactgcttggaccctgcttgtccagggtcaggccttgtgggccaggcgctcccttccagtgtcggtgatttctagcggtttccaggccaaggcactcccccgctcccagtgctaagctgtctcaggagcccttgggggctgggacccttggctgcaacatctgcccctgggccgggtgggcaggagggatggcctgagccgggccaaccggacctccttagtactggtcacaacgatgggtttggtgttcccgggaaaggaaagggggggaacctccaaacagctctgcgtccctcccccagcccaccttctgcgccgggaaacccatcatcttggggttcagggagaaacagtgtgaggggccacatccctcctgaggaaacaacgcggctgagttcttgcgacacccaaaggatctgttgtaagttcaacgtctgacatttaagggtgttttgcctccgtctcctagagaattgaaagcaaaacccatcaattagatacaaaagccatttgctggactggccgtgtggggcaggacgcccggtatttagggtagatgtgtggtgctgtctcggggctgcagtcgtccccggggatgccgggggaccctgcatctccccagtgtcacgctgcaccctcagagccatgctgggcggcatctgcatctgctctctggggtttcactgccccgtgctcctgtgctgtgctcctcacggtggaggcagcgtcctcaccctcctctgtaagatgccctgatccgaaccagcccatgagcgcgccctcgcgtccaggcccagagctctgtgctgctctgctgtcggccgcagggccgcgtggagatgtgcagaatggggggcgaaattcgggtggaaggcaaccaacgggacctgacttcctgtcacagcccgtcctttcagaagacgccacctgcatcctaggaggtgggggacacgtggcccagaaagttccagaagcctttcctgccctggcccagcaggtgccagaccttccagccatgttgggggcacgtggccgagtggtcactccgtggcctgtgttgggaggaggtctcagaggaccgtggggatgagccggggcccggacttacagccggtggattgtgagttctctgcctttgttgctgattcatgatcctttagtaagaaggaggaggaggggtgaggaccactccctgcgctgccctcttacctccccctcggctggttttcccgggtgccgtgggcgttccactttgaccttcgggatgagataatggggcttctgttcttggggcgggctgggcgcctggagctcggagatccagtgcgtctctgtggttgatcctgctggtaaatcgaagcccagatgcctttgcccttttctgcatgacgcgtttcggaggggctggctgtccgagtggccggagaagagggatgtcccacgctggtgccctgcccagccccgtcactgcccatcctgggtcctttcctccccagcagcacgcaggaatcccccacccctgctggagcctcctcactcctctccccagcccagtgtttccatgaggaccctcggaagcagaggcctggggccgccctgggtgaccctggggtccgggaactgatgtttaaataatgaatcacgtgcccctttgcccaaagcaggcagcagattgatagcagacaacttgcctcatgaatgccggtcacccagccaggagagcgaggggacaaggtggctagcggggcgggcggggctccagctggggctccagacctgcttgacatccccccaggggcgcagtcccacagcgccgtgtcttagggacatgcagggaatggtcgggtcaccgtcatcattaataccgggagaataatcctcagtgttgtcagctgtgtttcacattctctgcctgagctgtaatgatttctctcatcgctgtgagcagagaactttctcaggacgaaaggatgccccttggaagtggcgcgtgtacccctgctctgtcgacaggaaggggagctgctcgcatcaaaggggagagagctgctgggctgagtgtggtttgggggcaggggatggacgcagggatggatggcagccgtgggctcagcaggaagcgtgccaagacgccggccgccacgcattcctcctccggggctcagcgctgtcccctcgcacagtcccctggacttgagccccgagagtccactgtcaattacgcctgccagataaatgcatcagctccgcattaccacagccggccctggcgatgagtctctccgagaagggactgccgggctctggtttttgacaaagtcattagcaagacggcgttttagagctgcacgttggcaaagaggctctgggagttggttctttcttttctcttaaacccgacatgggcagcacccaaagtggtctctctcggggcggatgcagtctccggatggccgcatcgaccatatatctttgtttaggaacctggtttttattaaatttaattgcttccttttctggccttaaatcaagggtggcctcaaagcaggggacagcaccaatttaatttaattactctgcactgcagccttccgagtagaaacgtggctaccgggggatcccacagggcccccagtttgagatgagcccggccgcctcgggagggcacaggccgaggggctgggctcggaccccaacctccccgacccccactggtctctgctgggatccgggccttggacccccgttttcactcacttaattgatgtgtttgtttttcatttgccattaaaaccgcgttggctctctaactcaggggcagttcattaagggccatttatctctgcagtgatatttcacgcgtgcggcccggctccgccctgtagacatcacgggctccaaagcagtgttggggcacccttgctgccctgcgtgccagcttgcaggtgggtggggaccacagccgagcagtcccgccgccccgcgtcccagagcggctgcagcccatccggttcctcgcctcggcttccggaagacgtgtgtggccgtgcgaagggcttggccggagcgagactgtgctggacgtggaccgccgcggagaaagcggtgctcccggcgatgcggcgggggcagctgagcctgcagccccgatgtgctttcctcccgactttcatccctctcccccacatggaaatcctgcgacaaaggcagcctcggaggccgtctcgggcatgttttattaaacgtcaccgatggcgagagagcggcaccactggttcttgcagtctggcctccccagtactccgagcaaaaacggggaaattattaagatgatttgttaaataatagaaaatgtttttcatttcccaatttccagctcttgctaaagccatcttccagctgcctgcttctcattccttaatatttagacttatgggccaatttggggatattttctctttctcccatgattaatgcttctctgaagcaatgtcatcacaagtcacctacctgccctgtcagctcggcatcaatctcgctgggctgggcggtaatttatgactggtgccggcaatttgcaaatgtaatggggagaggggctgaggcgccggctcccagctccgtctgcctgtctgcggcctcgtcagcacccccccattagcagaacaaatggtgcagagatcagacatcatctccaaggctcgataaatattttatcagccataatggaaattgtcaaattgatctaattgtacaatacgttatcagtgagatatcaaagctgggcctggcctctacccgggccctcctggccaccccggctgccgaggatgcgagtcggggagggcagagcggatcccaacttggtgtaaataatacgctggccactgcagcccgttaaggggatgataaattttgtggaaataaatggccttattctgttgtgctttgctagcccctgatttttgagagccagaggagaacagagagaaataggtggaaagacagagacggtgggacagattcagaactttcttaagacacgggatccattcccaccctcttagcttgaggggtggccgcagcccttgagaattggtcacatggatgccgagcaaagtcccagcctggccccagtcatcctgtgacggaagcacgagccactgccgaaggggacacttttttcctgaccctcaggagccagtggcccttgggcccaaccggggtgggagcatctggacgcacagcacttctttagaagtctgggtcatacagtgcatctgccacccacgggggcacacgccgtggcctgagcccaacacagaccttcagcctgcaggatctgggagcgcgttttaaaactcagctcgtgagaaactgaaaatggtcccagcgtcccctcctggggaggcattcatcgggctggtcctgcaggaaggggtgggggaggaccgcggggcgggtggcggccgggcatctttcacagccaggcaggaccttttgatttccagaacaaacagcgatgtcttagacctggagcacatttgcaaatcgagtgaaaacatctgtgaacaaaccgaggccgagctgcctggcggagcgtggggagccctccccgggcgacggggcgggggctggccttgcctgctccggccgagtcccgcgccgtgccctcgcccatggcctcacgccgctccgtggaagccgttatcccactttcaaaaccgcttctgggtctcaggggccggtcccttcttaggggatgcacgggaggacggggtgttgtcatctggtgccgacattcaaagacagatgaaaacgagcgtggctcacgggcactgaggtggcccaggggtcaggacagacaggtccgtgggtcgggtgttttcccggagtgcgtgccgcacactcgcttgggagtggatgtttaccatgatggaccccatgtcataaggagggcgggaagtttgtataaccatctgctggcggagctgcggcgagcaggcagcatccaaaaggggagaggatcacaggcagaggaaaccgcttccacagcggggccctgcacagtctctgctggtgagagttcacccagggcgccggcaccagcgtgaccggcagaaccctgagctcctgtgcagcttcccagcgggggtggcctgtggccagctcagggggccttctgggcggcacctcaaggcaggacgcagaccctcagtagcagcttttctgttagggaatcctaaacattttctcagatgcaggtgctggttatgtttctcattagatgcacgggggctggtgagcacacacgcacacacgcacagaagcctgctctctgttttatcggtggcctgtcattttaaataaaaataagaagggagagtatgttagtgtaacggaagccgcgcatcgtcgtggctggttcgtggcctccttatgaggggtcgtcaagggcccgtgtggccttcatgtggcaggctctgccttcttcccctgtttgtacgaggggcatggtgagtgctggggggccaggcagagtggatgtgagtctggactccaaaatcctgaactttcctgtccgcggtcagggcgtgaggatgggggcagcaggccccccacagctgccttctccacggagcttgagcgagctccgacgctccatttttccatctttctcccttccttcttatgggagaaaatgataacactgtttggaactatttttcattctgtttttttaaatggtcgggtccagggggaaggaccgtgtctctgcatgtctgaaacccaacgtgaggcgcagggtctgagcgcggaggagcttcccggcatccgtgcagagagggtggggggatgaactggtctgtgtccctggagctccagcccagtgcgttcatcacagcaaatactcgacagtgtgtcctcaggagggaagatgggaggggtctttctcgccagattggaagaggctgcgtgcccctcctggcccggcctgccctcccctcccattcctcttcgaccccctctcccgctctctgggaccctcccacggctctgcaggcctctgcagaccacgatcctgggcacttggacccttcctgctgcctgggccccatccctgtgggagtgacgccccagagcacgcgcccctggctccaccgctgcccatgaggagggtgctcaggcctcggtgcatcgttaccatccttgacaggggccccagccccagagcccccaggagggagcacagggccccagcctttccagacgggtgctggccaccctgtgggccgggactaacaccccgctccctctgtgaaaggtcagacaggcctgggcttggaggagaagcccctctctgtccttaccgcgtgggtgcagctctggaaaggctgctcccccagccctgtctcagtttcttcgtctcagagaggggacgacagtccctgcctcacagatcagacacaatcgctggagaggttgtccgtccccaccctgccgggacccgggcagagcggtgtctgggccagacccacaccctcccagggctccaggcccatgggagccatgccccggagtagtgcacatgggccaggaggacagctcagctggagagtggggaggaggcggccccagaagggtgatcggtggacgtgaggtgggcccaccccccagacccaaagccccctgcgaggtggcagccgtgacctccctcttacaaatgtggaaactgaggcacagcgggcttcgcagctgcccaaagccaaggctcagtgtctgcttgggacagcccccagcaaagggcttttcttttccccaaatttcaccagctcaagaagccctggggccagaaggtcaaggcaggagtgtgtctgggtccccgggtcagggtgtcccttctgctctccactcaccaggcgaaggcaggtcctcctccacctggggtggggacccggggtggacccgcgcatcctgccccgtccgcgcactctccccgacgcaataccagggtcttcccactggagacgagagccggctccccttgtcctgggagggcactggaggcgtctgcgctcagatgtctgtggagctgacccacactggtcccacctggtggctgacctcccaggggaccccgtgccacccagtctggccatgctgagggcttctccccaggtcttctccgaggtcatcctaatgccgccacattcagggctttccttcctctgggccgtgtcctcaagcgatcgaaatcccctggcaaagctcagctctgggcaagtgtcgagggccatgtgcgagtggtcagggacggatcaggggggcgttgacccccacgccccagcacttccctcccagctccctatctcccacctccacccctgcagaaaccagatcttattggcaggaccacatggtatgtctgggagctggtgtcttggggccaaaaacccactgggcagaccaggaactgggcccttgagggcacaaggcgtagtcacgtggcgaattggttcctacccacccaggcccctgacaacctcattaagaaagtgcaatctatctggcagagttccagttaatgatccaatttgtctgagtgagccccggctccctatcacccagcacacggaggtcaggctgctgtccacacggtccccaggtgccctcccaggtcaggagaatcagcaagatgtgaccaccgcctcagggccctcgcatggccttcccgccaggacaggggctctgagcgcgctgagggataggggagagggccatggttgtccgcattcctgaggggccagctgagcgaggagacgtttagacggcccagcacaccagtgtgtcagggctgtgtgtgcagcaaatcctcgggtccccatgcccttggggacgtcctgccccttcacgcaccctcccccccgccggtcagaactcctaagtggtgaccttctactgagtggggggctcgcaactagcaccccactcccccggggagggtaaactgtcacgggggttgtagaagtgcttcagggccctgtgggaagaaattcgtcaaaatggtaaaatggtctgctttaaagcgagcgtgccaccttgaagtcctttatcaaggatggcatgtgtgtgtgtgcacgtgtgtgtgtgaatgtgtgcaggcatgtgtgtggtgtgtgtatgtgcttgtgcgcagtctgtgtgtgtacgtgtaggagtgtattatatgtggcaatgtgtgtgcatttgagtgtgtgtgcagacatgtgtgtggtgtgtgtatgtatgtgatatgtgtatgtgtgcatgtatgagtatgttatgtgtgtgtgcacatgtgtgtgcaggcacatgtgtgttcacttctaagcagtgacatgaggatgtcataagtgctggatggttttccacctggagaaggaaagggcacccgtgtgcctgagtgtgtgacatgccacacctacccggtccccgctcacatccctgctgtcctttctccagcagtcagacccaggcagactcggaaacacaccaggagacctgggccaccaaccagatgctttggggtgatggtatttgtgacacagccttgccccgtggtgctgggcatgtaagggtacttgtgagctgaggccgtggaggccgagggtggccatgcagtctctctctctgtggctatttccgctgtttccttttacaggaatggggttccaagtgctagaaacggctgccattgccttgtaacatcttggtcaaggagggaccccacatcctgaccctgggctccctgggcccattgtgacagcatcgcccccaggcacagtacgggggcctccggcagctgggtcctaggcccagcaggtcagtggtgctcgggggggttctgggcaggccccctccttggtttccatccgtaagcttggggcttgggccaggagcttctgcgggtggtggggggccaagggccgacactggcctgacctgcaccagcaggagcggtgggcgtgctcactgcttaggcatcgggtatctgctttctgtttcatttcgtaaaagatttcctctattcagttgctgactctccgggccaggccatgttgaaagccccgtgccgccctcagactctctgattctgggctgacctcagcccctgttctgtgaacccattcttcttgctcaccctcagagcagcagttggggagacccagatgggcaggatttgcttccagcctttgaagtgggctctcctctccagggccctcccccctcactcctcccgcctccaaatgtgctcctgcaggccccagcgcagctcggacagctcgtgcagttgtcacatggtggagtggtgttgggcattcctcgtcaggctgccccacaaacccaggagcaggggctgccggagagctgacccacccggtgcaacactgttggcaaggcgccgaaggagctgccccgccttggaggctgggccgagatgttgggagctgctggtcaccctccccaagagcaggtggcagcccctccttgaacttcgcttcctttcagaggggcaggcctcccctggtaggccgccctagccctgaggaaccgtgctctggatttggggatccaggagatgccccatcccccttcagtgtcccggctccttcttggagccagaggatcccaccaaccccaagtctgcaggcctccggtgggctgtggtgtgtcctggccaagccagcccggtcgcagggctcctcttgccccgtcgttctggaagttcctctctggccggagtgtccttctctgcaaatggctcagcaagccgcattctctgggatgtgatgggtggtggaggggcagccactagacagggcctgctgggagaagtggacaggtgcaggcccgcacagctctgcaaggcaccgggggtcaggtcgggagccccactcggccctctctccaccccttgacagctgacaccaaggaacctgcagcctcctctatactcccttgtcctgcgtcacgcctcctaccccccccccccaccccgtggttgcgcaggaagccgactgcatgtggttaaggatgccagaaatgtacccaggcctccccaacgcacggaggaaagaggccagggagaggtgagcctgggcgggcctgccctccgtgctgtgccccggggagcatactggctgcctgctgcgcttttgtctggctttcctgctctgttcagaagctggggccaggccatgctcagacctggggatcctccaagagtgtggggatggtggcatctggtgtgtccacactcacctgcgtgatggaggaacccacagccaggtcagcggtgggctctccgtccagccctggctctacccaccgcagcctgcactgggctccctccatccctcggtttcctcagatttaagctgagtgattggactgacgtgcctcctaggagtacgattctgtgattgctggaggagtgcctctgaaccgtcacagctgcgggccgctggggatggttttgaagggcgcgttctgctgacttgagctcagggaacgctctccctctgggtcctcatctgacagagttctcgagaggtcagtgcatgtcagtccgtgggcaaagcgggaccatgagaggccgtgatgaccacgattagccggaatattcagttcaccagcgaggaacgctggaaacggccaaggcagcgccgagaggcagaaaggggcacggaggtttgtttagggtcagggttgctgagtgtgatgaccttggatgaccttgaacaggcctggcttcccttgaaaagaaaggtcactctgtctaggtcaaaggcatcactcctcagtcagggtttgccgggagcatcctgagggcgtgccccatgggtagcagagtgggcttccagaaaggggcaaccaagggtctgcagggcatccctgacagccagatgtgtatgtccccactgacagagcacggctaccggctcacttagattccttcatcctgaaacaccgtcccagcttcccagagtcctgtttccagtgggattggagcaccaagtggcctttaagaaccttccatttgggtttggtgcaagaggaaaatcagagcaggcccccagggctttctctctcttctgtggccaagcaggggggctcgtgtccctgaacgatgctgggttctgcagcagggaaaggtgacccccgagaaaggatcaagggtagcagggggtctccaccccattcttctggatttggtcccacaaagatgagaagctgacatgaggcggggctgggccaccgtgagaagttggtccgttccatctgaacacctgagtgatccccgtgggctctcgccccccgcaagaagaacggtgctgggagtgtcctcaggggctgggcctggggtctcagtggccccggttcgtgtcagtcccacagtcttcacccctcaaccctgccccatgtattggggcatccagagaggagtcacctcagagcgggaaacaccttggtgccatcttgtccaactccaccgccaagtgggcagagtctggggctttgtcctgggcagagccaggagggggctgccgtcccctcccagggccagcccctcactcctcagcatccgacgctggtccatccagctggactgtcctgccctggcacacagagggtcttgggctggacaccagctgctgggggagccatggcccctcggtcactgcttccccaagacgcaggcccacagcatcttccacatcttcaagtccattccctttagtcaccacctgcccgaagtccttctagatggccagggcacgcattattgctttctaatcagtggaggggtcttaatcccccaaaactgcagcccacccttgtggaagaaatggttgcttaggggcctgtaggaaaggactttcatcggagctctgctggcatctggaaagtccatttagccctctacaccgtgtccccttcaggccactggggcatcagctgcagcagcctccaaggtcctgtctacctgaggttccccggtctggctgatggttcccagttctggctgctctggtgtcacctgtggtcggccgtctgtgtctgtggctcccacgttacagggacagtggggaggcgggaacacagggtgttgtccccagccaatggcacggctcctctgcagggagcccggatgctcccagcctttgggagtcccatgctgtccagggtttgctctgacagccttgaaggccaggctggcatttgtagggccttaccaaccggcccagtagcacagaactccatttaatgccaaagtcttgctagattttatttaaccagaaagagagaacggacaagttcatattaattaacttttttgtccaacttaatcactcaggagggagggtctggccttgggcagcaaggctgactgtccagctctaggacagggaagcacgttctctgagatgcccccgggcctgcagtcaccctagaagccgactccagtgcccgctgggccagggacggactcccctgggagacaggccggcagccacctttggtgttgacactcagccctccaggaaggcaggagcacaggggttctcgaggctctgccgccgaaggacgatagacctgacaagtgccccgcggtaaccagggtgtgtccactgtcgacactcagtgttggggatccccctgcacggataccccaagagataggcagctcctaaaaggagacaacacactcaaacgaccaggaggggaacagacatcccttccgaatcttcgagaagccaacctgggggccttgaggagtctgtgagcctggacaccattagttacggggagggctttggcggctatgacacctcccagaacttgtgtccacgtctgtgaaaggaaggtcaccgtctggacacacagcaccaagctgctgaagaagagacgtgtctgccccaggccttacaaacacacaccggctgcctgctgcgagcacgtcctctctctggagcctgcacccctgcccgcctcccccacggaatctggccaccccctctggacctgaacttgaaccaaatgggggagaaaaaacttctcggggaagccaactcacagtcatgctgttttaactgaagccacacgttgctctcaaccctcccacctaaccctcaggaacacagaagtcctgaacgcgggcctggcctcaggtgccctcggtggggagctgtggctcctccaagtcggcccttgacttcctcttcacagcaacatcaccccagcttcattcccctgtgccgccatccagtggtccagctgcctttgtctctggatacagcaaacgaggccccagtgtgtgtgtcctctcactgggcatagggcctaggtccctggggctcccctgtctccctgtctggtctggagaaatggcagccagggtctaaacctcccaagagtccgtgcagaaggggccttacagagggttctactcggggtaagcaattaaggagcatcctacaccgactccgtctaggggtggaccccccccgcctccacaccaaggcggggcacagaggaaggctgctcctggtgccctccttgcactgcccggcccagcctggcccactctgggcagttcacgtgagtgtcttcctcctttgctacaagaggtccccaaaggcagtgtcccggttggatttccctctgcacgcggatgagtccagcaaacagtgagaagttctgtttccaaagcagtgggtctgcactgcatcccagccaggatccgggtggaggctcgtgtgggcagtgcaaccaggatcccggtggaggctggtgtagacagtgccatccacctccgcagccgtcctcgggacagaccgcggcacacactgctgctgtgaacacgttagacgagagctcggtttccccaaagtctctggtgttgttcattcctggagaagactgacggggacgcccgtctgtgtggcccggtgggattcgcccacgcagtgcacccggcttcatctcggcatcacggagagccactgtgggcgcagggtcaggcctcagagcccgctctccgtctctgcagagccccacgcagcagcctgcgacttttaaacgttagaaaagggaacaagccgtggggccggggcaccccagtcttggagcaaaagagggttgtggtgggtgggagtcccctctctcctcagcgtctctcaaaagctctgctggtcacccattcccgtggcatgttgctgcaacgatggcctcgtcctcactgaggcccgaccagaaaaccaaagcagggccgagctgagcgcagactcagtgttggagtcccctctctggcctcaagggcttctagcaataggtgttcctatgctggtgtgggtggggagggggaggggtctctgaggatccctgaatcagaatcaggggggcaaggccacttcagacccctttggaatttaagcaaacaaggcccctgaactgcaaggtggtgtttatccatttatttattcatccaactgtttatttgtccattcattcattcgttcatctgtccacccatccatccatccttttgtttatccgtccactcatcatccatccatccatccatccatcttcttgtttatccatgcactcatcatccatccatccatccatctatccatccttttctttatccatccactcatcatccatccatccatccatctatccatccttttctttatccatccactcatcatccatccatccatccatccatccactgagatgccttaacttgccaggcactgtctatgctctggcttgttaatcttctgaacgagtgctgttagatccccacctcccatagagaaacaggctcagaggagttatggagcatctgggattcatgccttgacctctctttgtagagtgggccttcatccactgtgctgcctccaagacactgagcacacttctctagcaagccgcacttccccacgaggctctaagagcaaggaccactggcctctaggacggtgctgtgttgctttccgcctgtctcctgacccgttgctcgatcaccctgcagagggctgcaaagtcaccgccaggaagacaggactcagccagagcacaggcccttggcttcagagatgctgaggccaccgtcctgaggaagggcagtgtcccgggatgtccttgccctcaagtgggacaggccttgcccagaggctcaagcttgtcttcctctgagcccctttcctgctgaggcccctctctctcccgctgagtccagggccctgggaagtggccccaggagacacctgacgtcagtgtttctagagagcccttagccataaggcatccaggaagtacagccagaggagttaaccagatggcccgaggccgatgctggcccagcagagctggctcgcgggcggtgcgcctcctgtctgagtgggagcaggggtaacccccatcgctgggccttcgctgctgggtctgccatgcccaccttccctctccacctctccagatcccagctctcctcaccagtgcgcctccctgttcccaggcaagttggctggagaggggagcacagagtgggacagagactgtgggggggctggggcaggaggggtctggaagtgggtaaggaacgtgggctgggagtggggtcggaaacagggtgggaacctgggtggacccacagtgatccctggggtcctaggttgtgtgtgcacatgtgtgcatgcgtgtgcatgtgtcagggctacactgtctgtgctgggaacatggcccgagtttagttgaatggaatgcacagccgtcacccacccagagggcagttggccctcctggatggaaagctgtgcccttgaccatgggtttgcttgcactgcagcagccaccaggctggacagagtgggtcttgagtggaaacagccgtcgtttgccgcaggaaaaacggcacgagaaagactgccacgtgtctttttggaggccaatcctcaacgctggcaacagtttctcccgctaacatttggtcccggagagccccaattcacccttccggaaatggcagtcctgtttggatcaaaaaataattaggagggctttaagtgggggtttcaagtacacatttaatttacgctgattaacagtcatatgctcagtaaacaccagctgaatattaagctcgcaccattgaggcgcgcagagcacacggtcatatggaaaactcacataggaaacacaccgtctctacaaccgccaattatgttaatcacctgcagcactcatcctgcaaaactcaggttaggacgtgcggctctgcacggagatccatcctcgggagcctgggttcacgtgctgcctgggagccgggcttcgagggctcctctttaaagaaagccacagcttctgaagcaattaggctgacgaacaaaaaaataaagtgaaatccgcctcgggattccttgccccctcctggtcccacccccggtccgtgagctctttgtctgtgaggttctgattttagagtcgctgtgtcccactgggccagcgcttctctcctgcgtgtgctattgcctcggcctcggggacctgccagggccttgcctgtgatgagtcattaggatccgccgtggccatccaccaccgtggccctaaattggtgtgggtgaatggaggttccacgtggttcgactccctcacccttcgtagaaggagactctgccccttgcccaccctggagcccccggtggggggtgggctccacgcaggtctggcaacatggggtctggagcagaaatcggcagtgagtgaggctggcggcttcctgacccctggcctcctggctcaactccaggggcccccaaatcacagggctgctgcggtccctgtgaatttcccacggatggtccctggaagagacctctggaggcccagagctggctcctaggtccccgggcggcagctgaacaggcagccaaggcaggtggggctgcagctcccttcttggagggttttaaaagaacatagcacgggggagagaaggcccgggctgcagggcttggccgtggccaccgtgctggctgctcccgattagctccggctccaaaggaccctggtttgggctcgcggctactgccggctatctacgttgtggcagcttcggcgtccccagattttctcagcggtgttctctcgaggagaagaaagggggcccgctccattgttggagtcgcacttgttcccgcgctgagcaccgtccatctgttttcccccttccttcctcccataggagccggcttcctccgccctctctggcggctcccagcctgctcccagcccgccctgtgcctcgccatcgcggctcctgaaaaatgagatcatttcaaactgcgccgtttcccactttgcaaaatgtgctttcatttggggcagcaaatgtttcgtcttttatttaaataaaacctcggtggttgaagggctttgagacctggtgagaggcagcccgacctcagctcagggatggggagctgcgttcttcctctctctgaccctcaggtgtccaagccgctcctccctgaggcttgccctcttcagagctgtttggtgtaatggacttccttaaattaataatgcgtagaaggagatttacaacacgaagctaaaatattgtctctgccttttattcatgcatttgtcactgtggcctctaatcgaggtgcccagagcagggataaataaggaagagagaaaacgctctgtcataatccaggaccatttgcaaattcatcaccgatattcccggagaaggaaagacagagcggatgacaaatgtccctcagcgctaatatatcttgttgacttcctctcccattattccgataacctacttgggccgtgatagaacgaggggctctggctgccccagcaacagatggggcccggcctgggctggccgcagggctgccggccgggcctcgggacagtcgcgatgcgcttgagtggggccgattggaatgttaatggtgatcaagttgattttcggtggctttggggaaaattgttcttgggaggaaataatcagaggtgatttctgcagcaaattgtctcaaatccctctctgcagaaaaacaagtagctgttgcttctatttttgtggggggggggggggtttgtttaggtcagtgaccgttattagaaagaattggcccaaactcagatcaatatcccccaaaagcggcctggggtgtcttctggtttggagggatttggttcgttggcggtgtctcagtcccccgggcgagggcgcgagctgtgcacacggaggctttctctctggatgggtttgaaccgatgggaactgggaaggggagagacagacctgcctggggagatttttctctgctgcaggtggggctggacccgttcagaggagccttggtctcgctctctgcacggtccaccactcgggctcctccaagcccaaggtggggcaggtgggtaagagcctcaagtcagagcccagacctgctcctgggcgtcaggcacggctttagtgcgaggtggagaccgcagcccgaaagtggcttctacaggatcctctgcaccaccctggggtctctgggaaccaagggtaggggtcagctctgggcagcctccagctccctcgtggggaccagtggtccccagagtgtccagagggagctgatggggctggggccccgacgctgtggagctcgagtgcggtgctgtgctgtaagtcagcaggcctccgggaaatgccagggctgcgggtcaggaggaggggcagccaagaggcccaagggcaaatggccacccagagctggccgtggccgtccagcctccaggctgaggtcccagacaggcgcttcggcacctacctggccagcctggtttcagagccggaagtgtgaaagggaggccagtggcactggctgagccccagcaagcggtcagagcccaggagggcggcgggatttggcagatggagaatcctggatgcccagctatgtttgaattccagataaacagcacataatctttcggtgggactacctaccgcgtacagcatgcattttactgtatttgttgtattttatttattgtttatgtacttcctctggcagccccacccccaggctccacgccctcctgtgaccccacccgaaatacagcagggtagggacggtctgattctccttgacagcagattcagctagatcccctctctggtgggacaaggtcctctgggcagaaaagggacggcagatgacgagcaagtgatcagggctcatgggttatagagaactttggggtcctaggcctgctccctggacggaatggcatggcctgaacagacttcccaaaggtccccggatttgaccagctgggatgggcgcctgtcggcagctgttcacgggaggctgtgtgtggatggctcccgttggcgctgagccgagatggcccgagacacgacaccctggtgagcagtctcatggtcggtggtggtgcgggacactcccagggtcaccgtggggccagcaccccctcctgcccagccccctcctgtggtgggcccatcacttcaccatccagaactcctcttcccagctaaacctttaacataaattatgacatatattgtaaaaattatgaaattataaaaatattgaatgctcattgtggaaaatacgggaacatgttttttaaattaagatctcacatcccacaaatctcacaaatggtagagatacaccatttggtgtatttctttcagtcttgtgtgtttgtcttttatgtgtatatatatatataaaatatatgtgtgtataaaatatatataatatatattattatatgtaaatatataacataagtgatataattatatgagtaattatatataatacatataaattaaataatatataatatatataatttaagtaatttgtgtctatataaatttgtatacagacacacacccaccaacccacatatgtaaattatctcgtatacttaacgcctggtaggaggtcgtaattccccccagccttcaacgtctgaacttgcctgtctgcgtgggcagcagccccgccaggtgcctgtcccctgggagagtgacggcctctcatttgttgactcttgttctccgtcagccaacctgatgacgtcctggcaggcaccgcgatagcagcaaagacgaaaacaaggaaacaagtgaaaggggggtcttgtcctccagggggttaccttccagaaaagggaaccagacccagttggaattttaaaacttgtcttctgttgaaaatgcaccatgattatttaaatgagaggcatttaaattttttagaaaacattttttttaacctacgcctacaccactgatgtttccggctttttgctgagtcagttgaattttaaaattgatattttctttcaaagctgagaaagctgctccagttctgaggcctggagagagctacgctgttacaatcccttcggaacaagtgtgaccagcttctgtggggaacaggagtctcaccttagggtttgttaaatattttttttttaaagttcagaaagttggccctgggctgcaggagccggatgcagggggcaccccggtgtctctggaggcggcccccaggcgtggcccgggccagccggcctgagggtccagacaaggttcaggacctactcacactgatctgcccattgtttatctgcaatttcggaccgactggggctcctgcagctttaccggctcagccgctgccctggcccggagccctcccgcgccggcctgcgtgctgctcgcccagggcgaccggcgtttccgagctcttgccgcccaggctcccggacagcacacgaggctgagaggccctcaggggtcggcccctggaaaccctggtgagaccatgggtcccgcccctgggttgtctggacggctgataaggcagcgagtgactgggcagggtgagtggctggcactggactccacaccctgcacccagattccgggcccccttgcgcagccaatctcaggggtcggcctgagatgccccgtcacgtgcctctgcctccctcactgatgaacttgaagcattcatggagcgcctgctgtgtgcacagctgctccttggcattgcagaggacgtggagggatgaggaccctggaggagcatctccccagcccccagcctgggcaagagcagcttgagggaaggagacagagaacagctacggcccagggagcagacgagtccacctttcctgtgccgaggtgaggggaggcctccgaacgtcgcctggatcaggatgtccctctcgctcaggatcttggagccatgctctccccttccggcaataacaagacagtgatggttaccgctgaccaaaggcgccaagctgagagctcggcaggaattgcccagtttggttctcccagctgcacttccagatgggataggctcagagaggtcaagcgcctgccccaggccacacagctaggaagaggcccagccgggtttgagtccctgtgctgtgtctcaggcaaaactccctgtgggtctgtggctttccttctattttctcctattcccgtgatgatgttgtccttctattttctgagaccctaaaagggagagaaaatgcagtgaaactggtagcccctcttgacgcgaagaatcttcttccccgtcaccgacagcctggaggggtgagggtgcggacttcatggtacccagacgccccccctaccctggcctcttctgtcccaagaaagacagggattcgtggggacccactgacaggtgagggaggccagcgagagcgacgcctgccaggaacgttccctaagaacaggattgttttcaaagtttctcaaaagatggcagaggaggacttcagagctccctagaaaacacacagagaaccacgctggaaagtgctccggggaggttggtggggaacttggccctggagcagcccagggctctttgtttgctggtcacagccacaccaagtacaaatcgaggtgctttcccacattgcctcaaagggcggcatttcctctctcctctctcctccggctgattagcgggtcggacgatggcgtgaggacatgttgggggtaggagaatgggggtcattttggaaaccctctttgtggatgggtcgggaacggggagaaaatgaatagttataagctcatttccatagaaactgtctcattatcatgctggctgcccgctggacgaggcagggcttcctgccagcaaaccggctccacagtgaagtgtcccctgcctgcccggctgccagagaggccggtcaggagcgacggctcccaaccccagccagcccggcacggagggatggtcaagattgcttttgtcttgaaatatcagacatctgtgtgggggttttcctcggcatctaaaaaatacgagctagttcgtccagagacgaggggcgtgctgggtgatccctgctctcatcacgagctcgtgtgtctccctctcactgcgggtttttacggatgccatgtgagcacatctcccagccccctgaagacgtttgacaaaagaaattggaaaagttaagtttaaagtcactgcctgggccgtgccctttgtgttccttgtgacatggcccggtcccctcgctggggaagcactttgtcgccgagcttcccgggcagagcagatttattctgaggctgttagagcagctgcctcctccgcacaggctccagaaaagctgcccgtggcagggagccccctgtccgccccccagaccccgcgctcacgtgcggccgactccttccggtcccgtgcagaggcacgttgtgctagattcgtcgccacgaacgcgtgtcgccaggtctggagaaggggaacaggtgtctgacctgggaaagaagcaggatttcttctgcaggagacgggcgcccccaccgcgggagggggcggtgctttgaagaagcttctccccgaggactggcctgtttgttattttcaattgtcgtaaatggatatcagatggtatttgtcatttaaccattcgtaagtgtgcagttaagtggcatttcatatattcgcatgctcagtaaccatcaccgccgtctattcccaacactttttcatcgtctccaacagaaactctgtcccattagacgataaatccccgaccccctcctccagcccctggcgacctctgttctgttctctgtctttgtggatttgctggctcatgtttttaagagggaacagcttatcctggggaagcgcagattatctgagctcctccgaggccgcgggaccaggcgcccctttagagcagcctggcgcttgcaggggtcgttgtctctacgcatttgttaatcgcccgtttaccctggttttgtgagagggctgttcagacagaaatgaccctgcccctgggaagagaagaatttgggattgacagcagtgagccggcaaacaggaagacgcacgccgactggagagaatgagccgccacgggagtgtggggccgaccacgcctcgggaagtagcccaacaacacggcagggctgagctgcatccgctgtcctttaaccttcagagtcaccctacgagctggggctaaggggtcccgttttacaggctcggacacccaggcagcaggaggttaagggtgaagggtcagtacggcacgttccaggagcagcagcagggcctcgggacctgtaaacacagcttctagaatgctttaggagtgggggcacatgtggaagcccccggcacgtcctcggcagatgtttttgagaaatattagctctgcttagcccctgctggccagtcactcagcacaaccgtgtccaaactgtgtgtccaacccaatggcctggatgagcccctttggcggtctcgcacacagtagtgctcaatcaagagttatacagcataatctgtcaaattaacagatgcatcagtgtcctggggccaccacaacgtgcaccacagccggggcttaaacaacaggagtccatcctctccccgtccggaggccggaggtccgaggtccaggtgcgggcagggccggctccctctgagctgagagggagagccagttccaggcctcccgcagctcctggtggcctgctggcatcctcaacttgtagatgcctcacttgatctctgcttcgtcttcacagggtgttcccccgtgtacgtgtctgtgtccaaatttccccctttcataaggacgccagtcatatgcggttagggcccaccctctgacctcaccttaacttgcccaattgcatctgcaatgactctgtttacagatgaagccacactctgagatgctggggggttaggacttcagcatatgtagttggggggacacaattcaacaacagtggacgttgcctgacttcatcccatacgggactgtctctcccgggggcctctggggcagggctccctcccagtgcttggctcctgccgcctgggggcccgtgggggcctggacgctctccccgccctctgtgcagctgcctcgcaatcccctctgaaacaactgtgggtgcaaatcactgtggttgtgacactcgcctgcagtaccccaaaacctgtgccttctgcagaccccagaggcctccgtcccgtgaattctgtgcccctcagacagccggccaaaagcttaagtataatgtgtgataaacacgggagaccattctgtttttgtggttttctgaggtttgaaacgtgatttagaataattttaggttgtcctggggttggcggacagtgtgacagccaatttgagaagacagccttctttaaacagacggattttgcttccttgtttctaatttttaggaacatgaccaatgtcagccggcaaggtcgggccaccagctttgcacagcaaaaatcaggtaatgattgtctcgcacgcagccgtcggggctcgcaggttagggcgtcaaatcgaagccatggccgctgtgctgagggagaaacgcttccttcttaaaattagaaacctgctccctgtctcccttcggccaactggtaatcccctaagcaatctgctgatcagacagaaaacttttctgctgtgtagccggctcggtgtgtctggaaaggatggtttgaagcgtgaggaattgctcgctaagtgctggtggcagccggggggcctgaaaggggcatgggtgagcgaggtgacccaccagagaaatgaggttgtggctgtgtttcggtggcgctcagcgcgggccttgctgtgttgacaggatgagacaccagctcgggaagcaattccagaggagggcagtgttgggaggccctgggaaacgggccatgggctctacctgcggagcctgggggtgccttcgccctccaacgggccgcccagctgttgggtggggtggggcacagactgacgcctggacatctgccatctcgggaagcctaatgggccctgctccgagcccctgtccccccaccccgtgttttggggcacatgcatgtgtctgtcctgagctgtgcagaggggggtctctccagaagcctcgactgcccctgggttcagtcgtccttggttgttcaattggcccttgtatctgtgacaccaggaggaagagtttgtgactctgtgccaggtggccgagcaggatgctcagatgtggctcgagtcctgttgggtcaatgacagaagctccagggcccccactttcctcagcccttcccagagggacctcgtcttataggcgggagatctcattgagtgagtgcccacctgggctcagagatacgggctcacctgggcttacggatgagtcacacctggactcggggatgggactcagggatggggctcacctggactcagggatgggactcagggatggggctcacctggactcggggatggcggctcacctggactcagggatggggctcacctggactcggggatgggggttcatctggactcggggatggggatcacctgaacacggggatgggggctcacctggactcagggtttggagctcacctggacttggggttgggggctcacctggacttggggatggggctcacctggacttggggatgggctcacctgggctcggggatggggctcacctggactcggggatgggggctcacctggactcggggatgggctcacctgggctaggaatatggacctgtcctcctttcccagtggctgcaccatgtgtgttgttgaaagctcagcaaaatgcaagttgagaagtgccttggaaaggggcttccgtgggcctgcgaggggggctggaatggcggatggagccccctcttagcccctggtccttcctgtgtctggatctgagtgctacactgcaacctcaccttgctttccgtttggctgaaattcattaactgccaggaaatcatggatttccaaacccagcttttccacagcctccacttagcagagaaaatcaaattgatgcaagcgagtaacgagaaacagctcccgaagaactctgctgagaggccaggctgtggctttcccggcggacggtgctcaccctgcaggggacccagggtccacgccaggtgcgggggccgggctctgaccgtggaggacacactccagttttctgagggggccttcagataattctaaaaaggagaatgagttcccccagacccacagtggcccggctcaaatgtgctgccaggtgcccagctgcctctgtaagcccacttggtttgggctcctgctaaatggagccttcttggggacgcgcccctagggcatttcatgggtagcctgtggccttgtccttaaaagagaaggtgctcgtgttgtggaagcccctgtgcctggccaggaactggtggccctactcctggctgggcactggccggaacctcctgtgccccggggttgaacccagcactctcccccaacacattcagaaggttgggagagaaaggcagggcccagcgggaggtgggcagaagcgagaggtacgtccagccgggttcttggcctctctgagtgacctcaggtctcgtctgtgaagtggcacaatcactaggccaactgttttgggtgctatgtgcctcgtgtgcttagaaacctcgcccacgttagctcatttcatcctcttggcagccctgcaaagcagatgccgttgttaccctgctgtatagatgctgccacaaagcccagagaccacaaggcactagcctgggtacacacagccgcacacagaagaagaaggttgaacaccctcctctaaagcactgctccaccagcctctctcctcctcccgcttggaaggactcctggggtttcgccttgagcagatgggacgtagtgagagctcagtcagcgctggtctacactgcaccgctactaccaccagcagtgagcttgagaaaatgttgttctgaggatgtcatcaagaggaaaataatgagggggagaaagctctcactgacagcccagggtgaaggcctctgtccccagggcctccagcctgcgtgagggacccccagccacccttcatgtgagaggagtctctgaaaaccaccccatccagagctagcccgccagttggcgatcggctgaactgaagcctcccggtgctcccggagtccaggcccggatgcggattcaggccgggtcctgtgtgggcgagcaggtttaggagcagtgtggggcaggggctggaggtagaaggacaccagtcaggtgactgtcaccaaaggggtggcattgatgctgaggggtggcacgtcgcaagccctgacggaggcacggatggcgccacctcccaccacccctttgcacctctgggaaccgcccctgcccaccccccaccccgctcgcttgctgggaaacgaggcgccagcatcacaccagtgacaagtgtctctttccagtctcccaacaaccacgatgacaggaacaggtggaaagattgtggggacttgtaaattagtcgctaatgaggaaaattaggaaatttgctgaggcataaaaaatgagaagtccagagaaagaagacaaatgggtcgggagttattccctcccctgcgccccggcccccgcccccacgctttcaaactcctcgtttcgaccagactgaaatacagatctgatgaccgggttaatgggaagaaattggtttaaaatgtagcgatgattaattggattaatgctcaaaaaaatggttaaaatggtgtcagggagtgagcagggagtggggccacctgtccggccccctggcaagtgctctgcagacgccacatcactcgtcccacaaggactccagcaggtgggccacggggcgccctcagagaggggaagccctggctcaggaagctccataagcaggctggtcctccagccagctcggagccccggggtgctgcccacaccatacactcaccccctcccggccgctgacccctcgggttcaagcacctggaacaggaccgtttcaggggagcatgagcctgctgcccgtgggtctagagtcgcaggagctgagctgttctcatagaatggaaattcttaggaggggtctgtgacgtccagctgatggggggcctgcagcgaccacatgccccagccctcggacacgtcctctccgagcctcagctgtccctcacatttaatggggccggctcgtgtgctgtgtgggaattttctgctcctccttatcaacaaacccaccgtgtgtgacctcaaaagaaagtgaacatcgtgaaaatcgggtttccacaatccgcccgcggagacgccattctgagggtgcgagttagaagatactcttgtgaagctcattgggagccgaaggaaacacctcgttaatgtcacggaagccgaccccacggcagcttcctggggtccttggggtccgggagacgttcctgggggtctggagccgagagcagagacagccctggatctgagtccctgcgccgcccctggccagatgacgacccagggcaggtcctggcttctccgtgcctcagtttcctgatccatatagtgggcgtagtcatgggcctcactcctgggtgtgtctcagggagcaagtggcttattggtatgaactccccggctggcacatcagggttggtcgccaccttgacagggagagcgaaggaatgaggcagacaacctggcagggaaaccatggagcccacggcccaccacccgcccggcggcccctctggccggcaccaggctcttgggggcaggaccgggtgcctgttcccgccctggcgcctccggagccgcgcggcctctcctccggcagcggggccgtggccgcgcccgggtcagcagccagctcgcgcctttgtcagggtgattaatagtggtggggattgccgttaattcactgcctaatgactgcggccgccgcgctccgagtaatcgggtgatgtatgtggggagcgcccacctcatggcagaggtgaaaatcatttcgacaagtcaaatattgtcacagggaacaaatggctctccctgttaataaaaattaatgaattttttttctttctttctttttttttttctcgccactggcctgctaaatgaagctgcagagcggctgagcagggggatgaacgtggcggagtcgagcactttatttgacggttaaaactataaacgtctttattagggcaccaagcagccagagggctggtgtggccgcggggccggtgggagccgaagctggaggccgctctggggcgtgggggaactgggagcaggtgcctcggcagtgcagcaggagggtctttgtggccccccgagagctgagacacaaggaggcccttccccgtggggcactggctggccctgagtccccagtcctagctacacagagatggggacatcgtgccaccaaggggctcagcctgtgtgactctgcgcccctggtttggtggggagccagggctgggcagggagccggctggctttgagggacaaggaaggggagcggaggacgctcgcccacacctccagcagcaagacaccactagcccccagctccaggtctggaggcaaagggacacggtgaccctcagtgcaaaggccccattctcatcctgagcagccagagctccggggagcgccacggaggctgcagagcagaagctcaggaggagagcccttccctcccagacctctggctgacggtgtctctaagcagggccgtttctcctcccctgcccaccctgctcaggccctggcccctcccgcctgtggtccccaagagaaggccgagcggggccccagtacccccagggagccgggccttctagcacaaggggaaggctcttggggtctgctttctccctctttagatttttatttcggagaaacatttgtttacttgaggagcgacttgctccggcctgtgaatggcagccgcaggcccggtcggtaggaactgaagagataaatatgtctataaatagtttggtggcggttgtcgggggcgtttgctcagtgttaacccattcctcctctgcagggctgcgcagacagagagggagggcaaagcagagacagaaatcgccccgcacatcctcttcgctctgcgagcccagctgagcgtgtttcatccccggggcattttgccctcggcgggcagctcccggggccggtgcagagccccctgcagtgacctcggcctcgtgaccccaaacccactgcaccctggtgaccagcagcaagcaggcatggctgtccggggacaggagaccatccccaccttgccccatccccttggcacctgaggggatctgactttgcagaaacagtgggtttgtgggtgttgggggtcttggcctggccgtgtgctgagccagctcctgccctccgtagctttagcaagcagagtcccttctgggttcctccttccatttggaaggatccagaactccacagtgccctagagtcttgcagcctcagcaaatctggctgggcatgagaggaacctgaggaccctccagcgaaggaagtaccctccatctccgcagccaccaggtcctgccaccaggacaacctcccaaagcccagcttgaatgagggcctctcaaaatccaaaatccgcagctgcccccccagccccagggagcaaagtcccgttccttggggatcctccagggccctctacctccgtctaatgggagcttccatccagcagaatggggtctccgttgttcaaggagtgtgtgtggccacagtgggatacagagggatgcttgatgaggtcacagcctccttggtgctgtgggcccggcgggccgctggtgcagcccgcggccggtgtgcaggccgcctgatgatcacgcagtcacagtgccaggaacagtgtccccacgtgctgggtgcaccgttgctctgaaccccccaacaggatggatgtacccctcttctgcagagtggggactaaggtccagagagcatttgtgggcaagctggaaggagagcctgctcgtgcttataggacctgagaaccagccaggatggtgagggagagagcctggggtctggatgaggatgctgataacaggagcctaatttcactttgggggaacagcctaacagcacactgtggaccccagcacacctgtggtccacttcctgatccacaatcaccaaaaagtagaagcaacccaagtgtccattgatgatgaatggataagcaaaatgtggtccatccacactgtggaatattattcagctgtaaaaagggagggaattctgacacttgctacaacgtggatgaaccttgaggacattatgctcagtgaaataaaccagtcacaaaaggacaaatgtcctatgattccactcatctgaggtccctagaggagtcaaattcacagagacagaaagtggaacagcgggtgccaggggctggggcggggaaaggggagagagtgttcaatgggcagagttttagtttggggagatgaagaagttctggagatggatggtggtgatggttgtatgacagtgtgaatgtgcttaatgccgctgagctgtacactcaaaaatggttcagctggtaaattttatgttatgtctattttaccacaattttttaaaaaaagcaccctgtggtgttgcctgacgtgaggacacgactggtatctggagccaacctcccttgatatttgaggggtctttggggcctggggcccccgctggcaagttaacaacacaggaggtgaagggtccagggacatcccctgctcgggccgccagctcacgtggtgcttagcggggccagggagagtcgcagtgattgagaagaaaaacagtgagatttgagcaatccgcgggctccctggctgccccggggctcggggaagtggtatgttaaccaaggaacaaaggagaggaagaaaaaaggccagatcacatctctgaaacaaagaggcttttaccaggaacaagccttgataaaaattacagtggcatcacaggccattcagcgcctgcctggggcgatggaggcagcgaggaaatagggatgcggggcctgaaataggaacactgcgtcccaggagcattgcggataaataatcgtcttagaataatgcgctggctttaagtagggaccaggtggtctgggctgccggtcagagccccccgcggccctgtgtcagctcagctggacgggggacccaggggctggggaggggtatccatgggcagtgggctcccagagccccatcttggcaggcagtcctttggcagcggggcctggctgggggtgggagctgaggagggcagagggaccccagggggatgtggtggtggcctgtaaagtgcgcccggggcccacggcctgcctcacctgagtggagggggcaggagggcggagcagctcctcggttccctgctggattcacgggttcacggttctcagagcctcaggctggcccctggagaggtgaatacgtgcttgagtgggctgtgctgtggcagcagcggccagccagcgacttggatgtcccaggctcccctcccagcaaaggggctcacggggaccctttatcctctccatgtatcagtctcctcgtctgtgacgtgggtggagccatcacctgcttcacagcgtttacgggcagataagaacagagggcaggcagggagctcttagtggggggccctgggaggggtgacagtgacccctgggccacattccgtggccagttcgggccctccagcctggctctttgcatttgctccctggggctactgtaacacagtaccataaattggggggcttcaaacaacaggcatctgtcctctcgcagtccaagtcacggtggccagggcgtgctccctcagagcctcttgggaaggtccttcctgccccttccagcaccaggcggccccagcctcctcgggtacagacacaaccttcaggtctctttcccccgtgtctccctgcgtctctgtctccctcccctagggacacagtcgtatcacagtagggcccaccctaacgccctcatcttaacgtgatcacatctgcggagaccccatctccaaatcggtcgcgttcacaggcgccaggcactaggacatcaatgtatcttttagggggacacaatgtgacccgtcacagtcttgggagctgcacctgccgtgctgggtgacaggaggcagtgggggccagtggagcgcttcaccaagtcaggaggcctggatctgccactgccagctccacgtctgagacaaaagaagcacttgcatgtgacagtggccaggtcaggtggttgccatctctgggatcaggcagaggagggaggaggcccggcctctctgtctggaggtttccacccctcagcccctcgcaggagtgcctgggaccggcagggcatgagacccgaggccagcgccggcctgcgtcttcccaggctctcccaggcctaatgacaatccagctccatcagccctgcagctggggctggtcgcccctagtccctgtgggcaaggtcccaggcgtgggtttgggaatataaacggcaattgattattattaatatttagcatgttgtaggaacgtcttttctcggagctattaatctgccgtgttaataacagccgagctctgttcccggctcagaagctcagcggctgctcggcgaggggaatgccaatgagcgcggtgccgcagcggctggggcctgggcagctggtcaggagggcaaccccgggtggcagcggctccctgcaggatgttcctggagtcatgactgctgtgatcttgccttaaagtccaggtcccgggctggggggagcttgctacgtttgaacaatagcaagaccgaaccccctcagttcctgggtccctttctgccaaatggtttccagaagtgggtctgtaggatgcacgtctgcagaaccaaggacccctccgtggaaggggggcgcctgtcctcacctggttgcactcaacagaaacgtagctgcacagaaatcctcggttagaaatgaaaaaccgtatctcaccattttatcctagccagactgagtgtgcttctctctccgtctctcctctccctcccccaggcagcgggcgagatggtcctcatgactgtcattgcccgtgtggactcataagctctggggagggggacttctggggtctcttaggggcccattacacagcatcctcccttcccagtttgagaagtagaagaagagtgagaagatgggaaccaaagctcctccttgtttccccaaaagactcagtgagagggccgggcgagcctgtggctgagccccagaatcacagacatggccaccccacccttagaatgctggagcaggcaggccaccccacgtggtggaggaagggtgttcccgatggagcagcagagaaacctcaccccctgctaagtggtgggggctatgctgagcaaaccccggtaccctctcaaattaaccagtcagtaaggagagtagccaaggtgtcctgttaatgggacttgtcctcagggaaggaaagtcaagagcgaggagtaagggttcctgcctcccacctttcctgataagaattgttctctcatctatctgcccatccattcgtccatccacccatccacccatctgttcatcccatccacccatccattccacacattcatccatccgtccatccatgaattcacccatacacccatccacccatccattccacactctcatccatccatccatgaattcatccatccatccatccatccatctacccacccatatttccatccattcacctacccacccattcattcagccgtctattcatccactcactcatccatctatatttccatgtagccatacactcatctgctcatccatccatcagtttgtccatccatttcctgtgttatcttttgctggacatagggaaggccagcagaaatgccaggacactttttcaggtgctgtctcactacagtgagaatattggggtcgcgtagcacagctctgagagttttagaatcaagaccatctgaagggctcccagctgggccctgaccagctgtgtggtcctgagcaagtcatttgtcctggaactcccttgccctgatgtaccaaacgggtgaagtcacaaatgtgcctcccttacggggctgtggtgagaactagacccaacggcagtgctggccgtgtgctccacaggcctagttagcgcccagcagcccacgaggctccacagggcgagctgacttagtgctaatggtttcgaccagccacccacaggccagcagccccgagaagtcagtgttgaccaggcctacctgcagtcactcgattgctttcaaagtcacacttggaggttccagaattgaatggccatgtggccaactggggacctgcctccccagagtttcagagctgtgactgaagccttgtctgttctctccttcacctcccctctgccaccacattccccacaagttgaacagaagaggaacttctgaagaccacttttgagaggcctcgtgaaaagtcacgctgcccgagccacagaaccttgaattggccttgggggagcggctgctccatgttcctcctgcctttcctgggaggccgtggtctggggtcattgcttttggttgataagatggggacgtcaggcctgggcccacaccaatcctggagagggggacacgtccagaaggcccacggcccaccgtgtctggcagcctctgtgtaggaggcttcagtggaaaatatttgagaggcctggccacgcctggttttactggagacacgacggagtgctgctgcatgctgtggggtccctgttctgctgggatgctggggaaggaggaacacctggagatgctgtcacggctgtggtcccctttcacccaaaacacctctgttaacaaaaacgagcagcttggggggtctgagcgatgcctttggaggctcctctgcccacggggcaccgaccttcggaagtccttggccaccttcctgccatcacttacgcgctgatggctgtccgtgtttctgcccggcctggccaggcgtggtggtacagtgaaccggggcgggaaccagctctgccggatgcagctttattatggaagcatcttcctttagcgcttctgacgggaaataacctggggagccgtttcccttctctcggagacctgacagggttatcggaagcgcaggactgtcgtttttctcctgctaagtggaaaagtgcggacttactggaaatgggccagggctgcagggatgggtgctggcctagatttctagggggaccttttggtcgtttaaggagacttggcttcgtttgcgttcaaccctgtgtccccaggacatgctcttgtcatttcggcacttctgaccccggctgccccacccccacagctcgctggagcctcggccgtggttctcacgctctaggggtcttgtgggagcgcagagcgcctcgcccgcccgccgctgtctcaggttgagcaggtctagaactgtccggggaccccacgctgagaacccctgagctggaagggcagatgtccccaaaatatccaatgaggctgggtctgccccgcttccaaaccctcggcagcagggcctgctcgctcagcacgctcctcaccaggagcccccaggtgcagacacgcggccctgctctgcttctgtgtccatcagtgggcgtgtggaagtgtggacggcccgtccctcctgcccagggtgacactcttggggcctgggcagctcatctgcagcccccaggcacaggctctggctctgagtacggggctcctcaggcaccttggcctgacccagctgcatagcacaccgtgtggtctcgtctccagcggcatctcaggtcggccgttgtctgctgcttcctgggcttctccagaccagggaccgcacctcaggtcagttcccagttcagttcaacacttccagtcttattgtttgactcgaggtgtgtctgctcagcctccagcccccacatgtcctgtgtgtagcacagtcgggcctggggttacggctttgccatacaggctggtgagtctcccacaagaaaccaccagccctcggagaggtgtaggtgtgaattctctgggttcagaggtcacggggtgcacaatggccaagggagagctcctggccaagggcctacctctgggcctcatgagccccaggaagtgttcaagagtggccctgactggggagggtgtccataacagttgtcagccccagatgttttcttggagagaggttggggtggccgggagctttggacgcccaccaagctcaggcctggccgtgacaccacccacctcgggcgtcaccgtcactgcccagggagggtgtgttggttccagtttacatggagaagacgtcagctccagggcccacctgcttctagtgtccccagatgatgagtggaggggctggcctaggccaggctgccaggggtcctcctctgagcacgaacacagaagggggctctgtcggctgtgagggctgagcaaacgatgaggcctttcgggactcagttctgggaggtgactgggaccgtgtcccttcgtgggctcctcgaggcccataagccagctgcagagtgcagggtggtccttagatggctgccagggagccagcagccactcagggcgggcctgtggtcagcctggtgtcctcactggacagggcatgcccccccaggcctgcagggagtgggtgaaggctgtgggcagcgagctgacctcaggcctgggcggacttggctgcgcctccgcatcagagctccatcagcaaaccgagtcccgggtccggtacagccaccactgcggatagagcagcaggtcccccaggtgcccttgctgagcagcccggctcacggggagccacggggtccctcttctcctctctcccctctgatctctcctcccctatgcggggacagtagtgccccccgagggtcagcatcgaatcaggtgagaacaattgcttttgacccacagaccccaagacggtcgccagtcaggctctccttgttgcgcgggggggggggggggggggggggggggggcacccgggaccacaggtcaagctggctcgcgccctcgggctggggcaccctcgggctggggcaccctcgggccagcccgtcgtcatgttagaccctcctcagggcatctcccctcttctgaagtgtggcacgtccgcttctgtttctaaaccttaatgatgagggtgaaatacaatttaaccagaaaagaaaaccagacagtgaagcattttcagttttaccccaatgggtggcccgttcattttgtttctgaatcaaggggtgtttctccctcaggggaggaaaggtggcctctaatagtgagcgtgcgtgtgtgcgtgtgtgcgcgtgagcgtgagcctgggggaaggagcctctatgctggtgtttgtggagtgaaacccagccaacatctggcctagtcagaactcacctgttgggcccattttttttttatgcttttttggtgaggaagattggccctgaactaacatctgttgccaatcttcctctttttttccttctccccaaagctccagtgcatgttgtgtaccctagctctaagtcattctggttcttctatgtgggatgctgcctcagcatggcttgatgagcagagtgtaggtcctgtaggtccatgcccgggatctgaaccggtgaaccctgggccaacaaagcggagtgcgtgaacttaaccactcaggcacggggccaccccggggcccagttttgaaactgaccagatggcttcctccgagaagcacgttctctcccgttcctggcattcttgactccgtggctgaggggtgacgtgtggcactgctctgccagacagggcagctccccctgggacgagggcagcctggcggggccgggagttggggtctggcaccagggccccaaaacagggctgggacgggtggttgggtacacagagcatggcggctccaggtatgggtgccagcctgctctgtcgctcactgcgggactgtgggcaagctgcctcaccccgccgtgcctctgtttcaccatctgtaaaatgggcagctctccagcgaggaggtggcagcccaggtgggggtgggcatccaccctcctcccagtgctcatggggcccagaggatggcagcccagggctagtctcagggcagcgacatgggggcagacaggcacatgtcactggtccctgttacatttccaacaggccccagcgaggccctgcccatgctcttcgtctgcaccctgggtgttggcctctctcaggactgtagctggcccctcctctccaccctgggccctcctgaggttctgcatggtgacctccatcagcatttgaggctccaaggagcccagccaggagcctctcaggaaagctccaggtttggagtggagcaagcgaccacctttagcccccactccctgccagggtctgtcacccctcagccacctccctgaaggtggatcagcgctcacaacctgaactgaaatagcagcatcacacgagtttttaggaagtgacattttcattccattgtagcatcttattctaacacgcccagtcttcagagaggcgtgtgatgttacatgcatcttaacccctgcaaacaggctgacatgcatccgttgggtcacaggcttccagcacatctgttcatgagagtctagcagactgatagactgataaagaagttaactgatgacaatgggacccaaagtatttgctgcaaattaccccctggagctttctacctctcctccctaacgcttttggcctttgtttcctaaaacctgaaatttcaaggagctactgtgtgtagcaggtggttccctggggtttctcaggtttatcgtgaacgtgggttgtcaccctcagagacaggtgagactaattcaatgcattcttacgtaaactatcagagctgaggagctgcgagtttccgcctacacaggtggagtcaagaactgtggcttttcaaagtaggtgcttttccaatcgaaattttaactgaaataattgtagatacacacacagttgcaagaaataacagagattcccgtgcactttgctcatttccccagggctgacagtatgcaggatattgataagcacacagtctatggtgttgataccatgtgatacaatgtatctccttcaggggcaggtttgacttgtgctgtgtgcgagtttagctctatgagctcaggcaggtctgggcatccaccaccgcagtcaggatgcagagcctcccccacactgtccccagatgcctcacctggtcctcttgtagccacccgcctcctgttccccatcccacaactcatcatctgctgaggagcatcttcacccggccccagtggcaccccaagtacgaagttggctcctctattcaggaattctccctcatcctcctctccagccctgactgattccaggagcaagatcataaaggtggctgggagcagctggcacctggtgcctgctttggtgtggctgagcccttctcccctcagcgcctggcagggtggacgtgcagaggcctggccggcggctctttgggccacggcacaaaggatggaggcttgaggacccagggggcctgtgtccagtggccaccctctccccatgagcacatgcccttgggcaggccgtgcactaggtgcccagaggctcgagccaggggcagaatcctcatgtagtggagtcgaggtgtcctgggagggcagcgagcctgaggagaagcttccttggtgttgggggagagctggtccccgagagggcatgtgggcagggccgagttctccatggtccctccactgtcccctaggaagatgtcgcaggccagaattgggatgcccctgagggatcgtctctgcccgtcatgggggtcagcccccggcagccgagcgccatcccactgcctcgtgcttacaggaagaagagcaaggcggcccctggggttggcttctggtgcccgtcagagggactctctttctcccttcttctcggggtcaccaggaggagggcagcgacgtcctcccacccaaagggccacatggaggcaggaggttggctgaggttgcttggacgtgttttcagggttcctgaccatccctcccctcacacgctctccttcacactcacacactcacattcacacacatgttcacactaacacacagctgctcacccccggaggccctggtctccctccagctccggctccggctcccgggccgccgtccacgtggtgcagttttcctgcggtggccgcagggccagcaggtttagtggcttctccttaaagtcccgccacacaataaaggaggaaccggagcaaaccgcatattgactcagagaaattccctcacgctcccccggccgcggatcaaggcttgaatctgccaaattcagcccggtgacgagcagccgggagctggagcttcggggaaggcccgtggtctttgcaccgctagtcattgccctcccgggacgccgcttatgaggccacggccggcagccagccagccccacgggggccgcgcgggagggaccctctgcgccggctggttgagcacaacagccctaatttggggccccttctctctccagaccctgaaatcctggctgtgggtttgaggtttccacggggctctctggggagacggtgggggcggcttctgtttggggattatttgctatgatctgggtgagagagggcaggaggctcccgctctgaggccggaagatacccagccctaaactaagggttcggagtggaacaggggcagggtaacggcggaggacacagtggctcccctccctcactgtccccagcctggggagcagctgtgtttcttgacttcggtgtgtgagaaacctccaagtggggtttcagttcaccaagaacaccctgaccccgctgggacccggcgcctgcggcaggtgttccctggcctgatctcggcggccggggctcgggctggctcggctcacgggcatccaggccctctttcccctcctcctgtcacccccgcgcccctccccaccactctcctccctgcttttgtcattttggtaggtctctccaaagaatttttaggaatgatggactcagtcacaaactccgagcctgaatctaccgcaaaacccaagcaagggacctcaggtctcctgacgagtaaacaacacaccgcagcagagcctgtctctggggttgtaagctcatcttggtgctgcattccggccttcgagagcgcgccggctggaagtgctccggcgggcaggggcctgggcggcagggtcggggagacgcacccactccctggaaatgcacggccagtgtggggcggggggctcgggcccgggccggccgtctcagggggcatcctggctgcagcccggggccgaggaagggtgaccttcccagccagcctctccggttgctcggggccctgggagctgggcagtttgtgctcggcccgccccgcaggccctggccgcctgcagcagctttctgtttctccagggcctcgggggctgctcacggcacctcattttctccaaatgttttctgcacactaattaggccttgtctcatccacctttgaaagaagcgctctctgtccgccagcagtgggggcagcgggttggagactcacttgggttttgctggtggcttcgggcaatcacttaacccctgtgaccgccgggggtgggaggccgggctgatgcacagccttcctttaatcagcaggaaacaccacctttttctcctaagctgtttgctcggccagtaaattaaacatgctttgaaagcgttgattttcctccagtaggtgaggagttttgggaagtagaattcaaacagaaaaaagaacctgaaaaagagaccggcgatgaatgtgcggggctgggggcacttgcccgctttctgcgcagcagagctcacgcacacagcagctctggctcctcctcgtcctgcaaggcatcgcatcgcgcctggaggagttcaggaggcgcgttggggggccctgcggcttccaacctgattctgtttgcgagctctgcgcagttaccagctccagatcataaggtggcacgttaattacccccccttctaatttacctttatgccgcgcgcgcagtccccgcgaaggtttgtgggggtgttctttcaaacctaaacaacttcagatctcgagggcggagggaacaacagccacagctctgttgttgcatctctgaaaggatcccacgagtcaattaaaaaggaaccgttggccacttaaaacatagtaattacttctgtgaatgctgctaaattaaataagtgttatccccatgatttttttttttcaagactttggtttcttttgaaagaaaacattttgttgcttctcaagcggatctgtttgcaaccagcatgaggaacctgaagtcatttttctcgtacttaaaacttcccgtctccctggagacgtccatctccccagccgacgtcagggagcagggagggggcctcgagacaagcgcccaagtgaccacagctttcgggtcactttcgcaggttttcaagcctgcgcagaaggaggggacatgagcagaaatgtctagatcaggggtccgcaggctccaagcagcagagtaagcccagctcccacccctcctgctccccccatccgcccttctgcgcctctctgactttttctttattgaggtgaagatcacataacgtaaaaattagccattttaaagtgtataattcagtggcgtttagcacattcaccatgttgtgtaaccaacacctccgtctagtttcagaacattttcatcccccaggagcccggtgcccatcagtcggtcggtcagtccccaccctctccgcccccagccactgcccacgtgaattccgccactgcagatttgcccattttggacgctctataaatggagccatacggacgtggcctttcgtgtctgctcccctcactcagcgccgggtttttcaggctcctccacgttgtagcgtgagtcagtgcttcagtccttttggaggctgagtcgttctgctgtgtggagccacgattggtctatctgctcatctgctctctccccctttgggggattgtggctgacgctgccgtgagcagtcacctgtctgagcccctgtttttagttctttccggtcaacacgtaggagtggacttgctttcctgtttgtcctccttcttgctcttcctgtcccttctccagggacaggaagttttaccatctcctcggggcctcaggccaccctctgctgcatgtccaggtggggctatcttggcaccaactatctcttaggacagatcccaaagagaggcccacacgagacacacagctggggtcgctcttgttcagtcccgtgacctgcagccccttgagtcccgtgttcatgacctcagcaggctggtgtcctacctgcgatgacgtcatcggggacttcaggaagcctccccagcgcccgccgggccccccgagccagccagagggcccaggtgtgggggcacatttcacagcagcccagattctgagtcctcccaagaatgggatgtgtctccattcccgaggctaccgacacgaactggagcggttccagaatgactatttcaccatcagccctgggttgggtgcccagtgtgaccgcttcctcactgtccccaaacttgtagcgaatgggcagctcctttcccggccgtcctaaagcagaacaggcagacgtgtcccaggtcgctcatgggaccgccgtgagactcagggagaaagtgcactgtggggtttccacaggctggttcctggagcaggctggggtacgcgcatccctgggcgcctctcacgcggagtgcttgggtcagagccgaggtgatgtctcccaggcagtgggggtagggagcagccaagtggcttctgggccccttggagacctcggttcctattccgtcccatgtcccagtggtccctgagcctcacacactggggacgcgcgtgctacagagcttgacactgcattgtttccttcaggtgacggcgacattgtaaataatatgtatgagcctgacccggacctgctggctggcgagagtgccgaggacgagaccgaggagagcatcatgtcccccatccccatggggccgccgtcccccttccccaccagcgaggacttcacccccaaggagggctcgccgtacgaggcccctgtctacattcctgaggacattcccatccctccagactttgagctccgagagtcctccatcccaggggccggcctggggatctgggccaagaagaagatggaaatcggggagaggtttggcccttacatggtggcgccccaggccacgttgaaggaggcaggctttggatgggaggtgagcattctcgtgtgagcgtgattgatcacggccatttgtcttgtgttccatctatttataaagccgggctgagcagccgctgcccgaggcgggaggctgggtccattcgaatgttacatttctcaggcttattttgccctggagcttgcatgacgcgactcggaaaacaccagaggtcctcgtgggcgcccgtcagcccccacaggcccgcctgctctcttcccggaaatgtgtctttaatgagctcatgtcttaaaacaccccatgcttcccgtctggatcttaggtgctaaggctgcaatctcggcagaacagcgtagattaatggaatgtcccacagcaagagtctctactccggggaaagccgcagatggggtcctagatcctggggtgagcagcatgggcggctcatagagctcgtctggtggctgtgcagcccaggcctcctgtgcacaccaacgtccaggcctcctgtcccctgtgccaggtcagccagctcactaagaggggcccccactgcccagattcgtgggggcctaagtctcattctgccagagagaagcatcttctggtttagattcccctaaggatttgggcctggctcgacagtccgggagcccgtgtactgtccggaaaggggaggcctgttaggtgaggggatgctgaccgccccaaaggtgcccctccttccagcagtgtcctgctcaggccacgagggatgctgggccccattttgggggttttcaaggtggtctcacgaggaccacaagcctgcaagcatttccccgctggtctcggcctttctgccggccccacactgccagttttcagtggtatttttggcagggaccctgcctgtgtggaaagttcagatacaaacccagattgcttctgggtgtttcacaactcccaaagggttgcatggaggtcagctctagaaagggcgcacagttcaaagaagggagaggaccctgtgtggccgggcagccgtgtcccctcacgtcagggccctggggagttagggttgggggagtcacctctcccaacctctcggtggctctggacctgtccccacagtgttcaatgttctctgccgagtgagggaagaattcaggccttctgtgcgcgctctctctctctcggctttatggtttgtgaacgttgtgcttcatgcctccctggctccctggagattgcgacccacaagtgcaggccctccctccctttgggaagctccgggtccagcatccttgagcacccccctgggccatgagcacttacagccctccctaaggctctcagggaggctgggtgtgctgacgcacatgtcgctggcagaggaggctgcccacagcctggcagagctgaggccatcacgcccttcaccaggagacgaggtggctgtgcccctacacactgagactttccagcaggcggaggagtctggtccatggttgcaatggggtgtggaccccggggacacagaagctccctgcccgaagcttaaagccccacgctcttcagagggctttgtatgtttagctgggaggagggttggccagatgacaccggaattcaccacagtccaaaacagggggagctgcagttcagcgagctggggagcgtgagccgggcgtgcacctgccgtcccacagtggcagacacagcgtgtgctgctcgaggtcagggcccccttcaggacagaccggcccggggcctgggagcaaggtgaggcaggaagccagggcatctggaaacgcagccctcgcgtgggtgcctgggccctctttcccggcctgtggggtcttcccttatgggaccagggagagcactttcagacaacgggacgggtaactgttctgaggtggctttgtgcagactcagcctgcctgaccctccctgcctggctgtctggcagaggaacgtaagggtgtttgtctgatgagctcagaaaggtctgggctgttcagtcagtaggggcatctggatgttggacagtgaacacaccctccaggtggcacgttggatgaccttgaggtgactttgtccttgcctgctctgatacaagtttctgtctgctcgtttttctaaagaaacgctccctatcgccgccgctcggggccggcctggtacaggctgtggctattatctgccccgtgcctgttgactttgcgatactggtccttctccacggacgtgagggagcgttctcgctccgcgtgcgcctcccgtttctgcagcgggctttgttccgaggcctgacctgacagcgcctttcatgccagcacttggaaatatttgccctgagtccgacgtcctcaaatagtgttccagacgcgcccgaccgtatctcgtccgcccttgttttccttgcgagctcaggcaccgattacccgctccgtgatccgtttttgggcttgtggccgaaatccgaggggcgggggcagttgtttgcgtccttgcgattgagagccaatgacaggtttggatgggaagccaccgcctcccaccgtgaaggtgcgagcgggacgtgcccccaaacattcagggcgagcccatttctgcctgaaataagtcacgagctcgaggaggtttctttggctctatggaccgcagttgggatccttggggaagaaaaccaatcgtcttccctcataagcagcctttctggaatttcccacatggatcctgaaaccggttcttgtcgattcttggtttttttcccgccttcctctaggatgatgggcggctcgggagagagaagggacgtgtgccccttcagataggcccgtccttccttggctctgtctgcagacagcgcctgaggcggggagcagaaaacccaggcgccagagcccggatctttctgtgcttctggacgggagaggggcctctagtgaagcccgagtggctaaagcagacaaggcggcctgggcaccccaccgtcctgtccaggctcctgcaaagacgctgtccctgggctgcagtgagctcggcccaaagcaggtgggagacaaagcaaacagctagagtcgtgcgcgcaaaggcatcgtatcagcagggcgagccgggccaggctgggtaccggcagaggccgcccagggacagcagacgaacctgcatgatcttacgcgggtcctgaattttacatgtgtaattattcactcccactctggtaattaaatcttataaagcagaataatgtggttttcctacactttacatggtattcacttactaaaacagaacaggtagtgacattttagcaaagatgacagaggaaatcgaggaggacccagtgtcattcagactcccagggggatggccggcatggttttgtggcccgttccagctccagagggtggtttctcctgttgggtcagatccaggagtcgcacagagggagaaaccagcggggtctcccataggcacgggcaggtgaccccgggaggactggcttaaaatcactgtcaaagagactacgcttactgtcatttggggcactgtgggaacaattttctttctactctgagaacaaaattcttgttctgggtccttccattcaagtaaaatccaaatggtctggtttccttttctctttcgtggtggacacaacatctaaccagcacacagtttctgggcctccaccactccagcaaaggccctagcctggcacactggggcccacatggatgacccacgacccttggggtcaaggtggcgggggcatccctgtttcatgcaggaatttttcttcagccaacgggaactcggggtggccgggagctgagccggcccctcccactgttcactcactgtgggtccttccgccctccctgcgctgaccggcggagcccctccccccctcctcccatgttggcaccaggagcgctcagcgactcctctgtgggcacaccaggaagtgagtccccgtcattctggaagaagatacaccccgtgtctctgtgtccctccacaccccaggcctggcccaccctgtctggagggacaggtgggagcgtggggtgtgtggaaatggccccctgggctccatctcaaaggggggtggtctgtccccgaccgaggaggatgccccaactcccagccccctccttccagacgcagcggcggctgaggcagccaccactcacgctgcccaggataaatcacccctgattgcattttattgccctggggtctgaaaaacaatgattttgatcatttgtggagtaaatcatgacatttatcatcgtgctgtttattttgctaattaagagatgactgccagtgacagccctgagggaggccggctgtggaagcaggctcctcagaagtgccacctgccccctgggcagggccctacagctgtgtacccggggcttgcggctacggggcctgcagggtggtgggaatactgtgcaggacccccagggaaggcggaggagcagtttcacactgagagtgtctgtgctctgctaggctcgcggttgagcgcgtgcctgacccagaaagtcagcattgcccagcctgtaacctgcgctcagacatgatctgttgaataaatgtgtctgcctctgtttaagaagctggtggaaaatgttaacgtgaattgatggggtttcaattatctgaagaatttactcatgtggattacctcatccccttggctttagttcctgaatattaatacacatgttcgtttattcagtcaacaaacagtctgtgtctgggccagggcctgtgggaagttccaggatcacaggggaaacaatctgaggcctcaggctgccttgattctccgatgctacaattatgtggtccctaagcctgctcctctggggtccccggggccccctgggagattttggtgagggccgggacccgctgcccccacctctactccccagggctccccacctccaaggacaggaggcatctgtgtctacctgcctgggtttggcagctttggggacctggatcagtgagtggatgctgagtgactgtggggacacctctcgtgtgagtcccggcccgatttctgagccagagtttcctggagagaagcttccaggctctggactaattccaggctttgggcgttagaggaagcccgaggaaccagctcttccctccagacctgtgtctttcctgcaccgagagcatcccttgattaggagcttcgggctcctgcccgcagttctgagtgggtggtctctctttttttttaatttaatttttattgagttaatgataggctacaatcttgtgaaatttcagttgtacattattatttgtcagtcgtgttgtcggtgcaccccttcaccctttgtgcccaccccccactgcccctttctcctggtagccactaatctgttctctttgtctgcatttttaaattcctcatatgagtggagtcatacagagatcgtccttctctatctggcttatttcacttaacgtaattccctcaaggtccatccatgttgttgcgaatgggacgattttatccttttttatggctgagtagtattccaatgtatatatataccacatcttctttatccaacatcagttgatgggcacttaggttgcttccatgtcttggctattgtaaataatgctgcaatgaacattggggtgcacaggacttttgggattgctgacttcaagctctttggataaatacccagtagtgggatggctggatcgtatggtatttctatttttaattttttgaggaatccccatactgttttccatagtggccgcaccagtttgcattcccaccagcagtgtagagggttcctttttctccacaacctctccaacatttgttactatttgttttggttatttttgtcattctaaggggtgtaaggtggtatcttagtggagttttgatttgcatttcctgagtgggtggtctctaatctcgagggttgggtctgggctgtgggtgaagcccatggggcgggacgtcagatgcgaggctcgccttctccacagccataacggcagcaggtttgtgatgttgctcaggagctggaccaacaccccttcgtgtcctgggctggagagcctgggccagctgtgttgtgagtctgagctcaggctggagggcaccttttgtgaccaagcactggcctccctcctgggccaggattccctgggctcttgggaatctgcttcagtcccttttagtccctcctgcatgatgaccaaggaataaatctcggtcaatctggagagagacatgtggtcgacacgtcgccataaagaccctcgatgcatccatggcccccgccagagtgagccgttttccccagcaccctgcctgctctattgacctgctggggctgtgggctcctgtccagctgcaaggggcacccaccacagccaggcttggccatgcccaggacatgagcaggtagccttgtggatccaggattcaggaaggcattgctcatcttccaggagagtcgccagctgctggtccgtacccacggtgttccacttggccagcagcctctgtggaagcagagtccgtggggccggcaggcaggggcagctcctctgcaccctggagctgtcagagccacaggacctcaagcgcttgcagccccagcccgccggaaggacagcttttccagaaaggaaagcttgaccggggagcctactggtgctccaggtgactgagaagcgagttcacccaggctggggagggggtgccgaatttgcagagaaaaatgtgaaacttggtccttgaatgtcagcactgttcttcagatttgtcccggggttctgagcccactttggaatctcccggagtgttagcagtgcaggtgcctgggccccaacccacgaacgtcaggctctggccagagagagggaccaaccgtccctgtctcccacgactgttctgctttagaagcaaaagtaccgacagacctgtctgggggctgagtttgtgaaagctcaagaaatctgaacgtgcatctggggtcgaagcagccgctctggagacaggagaagggagaatcctcccgacttccagcagctgcgggagggctgacggagccgaggttttctgaaacgaccaccacccccttttccagcatgttctgtccactatggagtccagagaggcgtgagagggacaatttgggatgggccgttgggggcgttcggttactcggcagacggtgctgctgcgtgatcagctcgtcctgggagctgccccagtggccctgtgccatgtctgtgctccatgccgcacatgcacaccctggctgcactctctcttccacgtgcatccccgtgcaagaatcacactccagcctctgctgtgtgtccttcaccctggagttgatggccctgtgctcttggtggagtgcccaggggatggggagggcactcattgttatttagcccctagactgtcctactgtgttttattcttgtagttctgacagtgggggaaagagctgaggcccctgccggtgtggggaagggaagccggggcgtctagactgtaagagttgatctgtggtttcctactggtctgatagccgcatgggttagggcttggcgttctcggtcccaaatcttggacgtgcactcgggaccatcctgccagcatccctcccccgccccccgccgggactggagtctcaggatctctctttgctttgccctcctctgcccggctggctgagtcaccccaacgcggctcactctccgtctgggttgcctggagttttacttttggcctagaaacatcgtggggctttttcccccactaatgattttctgaatgcgtttgggaggcataacagacattcggagggggccccaaatcgtaagcgtgtggctcagtggatgttcacaccccgtggaaacaacacctgggcaagaatgtgtctgggcacctcctcccaggccgtcaactaggcatctggattggaggcgggaggctgtcctgtgggagcagagcctcactggaggcggtctgggcgtgcttacacagaggggctgctgacggagaagcccacagctgtctcgcttctgcgggaaccactcagcacagtaaaggaacgagattgcaagccctctgccgtctcccccaagcaccgtgacatcaggtccagacgtttttagcccaagaatgccatgaagtcctgcactatcttaggcccagctccgaggcgcgggtatataaggaattttagaaaaaaggatagagaatgactaagaatacctcctgatggttctccttcatttccttaaggggtaataactcagccacaagccagcagagggtactttctttaaatagagcattctgattcacaggcgaaaaaggaaataaatattttcttttcatattcgagagacgtccatgaataccaacttcttaaatggtattctaccctccgcatcacacatacacacacgcacacacgcacaaacacacatgccagtgacaaccagacccacccttaccccggagcgggttgcttatgaaccagagatgtggaaaacagcatcgggctatcagaaggaggatgagcaggagggaggatggctgcttctcagggtggcagctcagctcttccgttggctcctgcgactgcctgcgaggacagggctctctgtcacctccctcctctgccctccctccttccttctttcctcctccctccctcctttctctctttaaggaaaataggatttacacctaaggaactatgacaaaggcacctggtgtctctgtgctgggcaggactggcgaagtcagggccagggcaggctggccaaggagtatggacaagaggaaaggcctggcgttaactgggcacctactgtgtgctgacgttaactggattcctactgtgcgccacacacagtccacccgtggctctttctactccatggtccccattctgccgtttgtgaatgggtcccatgccccagccccgtggacgcagcagggcctggcaggacgttagagctcacccttgggtgctacttacaatagagaattctcccgaccccagagagagacgttactgtgctgaagtcagggtttttgtttttgttttttataagattttatttttttcctttttctccccaaagccccccggtacatagttgtatattcttagttgcgggtccttctagttgtggcatgtgggacgctgcctcaacatggcctgatgagccgtgacggcctgggaggaggtgcctaggttctcgcccaggattggaaccggtgaaaccctgggctgccgcagcggagcgcatgaacttaaccactctgccacggggccggcccctgaagtcagggttttgtggtggccccaaggtgcccaaggcccgcctcagggagagatgggttcaggcaggaagcacccaggcccctggcccaggcggccaccctgggaggcacaaagttgagaccttcccaccaagtccaagggagaatggtttccacctgctcaccttggccttgggtcccatggagctgcccaggggcacattcgaaatgcagcttcaggtccccgcccccacctactggattagaattcctggcaggggtcggggtggggtcccaggaatctgcattctgatttcagtggaagactgggcacttggaggaccggctctggggtggaagcttaccttcagtttcctgcccccgcctccgggcccctcctcgcccctccctgggagcccagagacatttccctgcaggtgggacccacagggagcagcagggccttcctgaacgcaggcattgtcaggggcaggtggagagaactggaaagggggagggggcgcaaccgagaaggctgtccccatggggagggggaaaaggtggagctgagggtggcaagagcagcctggccgccccactgtcgtcccgctggagctcgggagcagaggaggctaggccagggactcctgaaggctctaaccccccatacggctggacgtcagcactgctgtcctcgggtgggtggcccctggagactgaccagccgtgtgacccctccctgcgggctggcggtgacggctgaggtcaggcggtgtcccaggcaccgattgtcatgcctggaaaccgctcaatgcatcaccacttctccccctcgctgtgccgggggcaccctgagccccagagaaggaggggtcacacagggtcacttggcaggttggtgcaaagggagccaccagacttgggaccctaggagcatctttctgctgtgtgcccctgcagtccctgagcagatgcttgctgagagcagggaccaggcaggccccagagccccccgtgccccctaggcatccaccctccgctgacatgtcctgtgtgggtggtggcctggccttcagctcctccaggggaacggggaagataaggcgtggagcgacacagacgtggaactcggcctcctctgtctgcgccctcagtgccatcccagcgtggctggcgctctgtgtagcctgtgcggctggctggcccctgctctgggctgatcctgcctaccagcggcaggaagcgcatggtgggcacccacctgctgagactgagcatctgcaagaccggtcatttatatggaaatatttctattttgagtactttcccagcagcaagcccagcctggcagaacggccaggagggcggcccctcctgagggctggcagctactggccaggcggctccctagggaccccagcctggccttggccatcccgcctccagcccctccatgggcctccctcccactgggacggacaagagtcccagcattgcaggccataaaaggtctccccgggtcatgggacaagggcgtgaagctccaggccaggcctcgcctgcttatcagggcttctctcaagttgggaatttttgtgggctggtcacccccgggttaggaatgtgttctcagcgtgtcacaggccacctcagccccagccaaaaacgagggtcacgcagctgctcacaggcccgctgaggggcagtggccactgaagggtctccctgcccgcagccctttcctgctctgctgggggctggcccagggcaccgtgcaccctctcaggtccccccccggcactgatcaaggggctgggtcagcccagggcggcctctccaggacaagagaggttaacctgggtgtgtggcacaggcccttcggggaatggctgaaagccatgtccttgcacgccatcacgggtgtgttccacatggcccctaccctgccattctagttgtttagaacatagtgaatgaaatggcctgaaggtgactttttaaatcagccaacagcgtgcacgctggtatatctggtggtgtctcctcctctgaaaagggagatgctaagaccagggtggtcaaatcttatcgtcacaggagccagagcgttggtccctgctcacctgtgtgcccggcctctgggcaggatggcgcctgaccccggccacctgtgacatacacgtgtgactgtgtttcctggggagttggcctgaagaacggggctgcctccgtcggtggtgccccaggggcaatcgtgggtccagccttgtccttctgtcccacctgcagcccccgcctggcgggctggccagccagggcaggcgcagggattaccagccccaagccgggcgcgggagccacctttgtctggctttcagactgcgtttcttccttctgggttaatgcccagtttaattgcacatcccattgtgtcatccctgttcaatcgtgttcaaaaatacctacatccactcctttcccatttagatctttctgaagcccattctggcttatcagcacagtcatttcacaagcggagctaagatttctgccatttaggccccaggcattgtctctccctgcggcagctaagataaaccctcacccagaacaaagtcgcagtgaccttttacaagtagagcggccttgtttccctctagactaatttatcagccttcctttcagcctggcgctcagcggaggccccgagagccctgccaagcacgggcggacgcggattaggagggctggctgggtcctcgttccatgcacaccttggggtccgcggggtgggaactgaacgggctcacccgggaggcacagccaggtgttggccaaagagggtgggggcctgcagatagacgagccccctcctggcctccttcggggccagaatgaaaccttggtgccaagggtgagccagactggagatgaatacaaatactgataatgaaacggcctacaggccccggagccccccagaggggcctcagatcccagaagacagctcacgggtttgccctacagagcccctggccagctctgccctcccctcctgccccgtcctggaaccagggtccctcatcaggaagcagggaggagaggcaactcctaggccagacctcatgcaagtcaacacaagagcacgtttctagaaggttccaattgctctggctgttggagcttggttcatgctggaacatcccacagagtggacattgaataagcctcatgtgcaagaagaagcattgggagccaagaagcatgtgaatggcctcaggtgtgccccttctcgtggccagatgaactggctgggccccaggagcactccagggccctccggttgagcagagagccagaagacacccccctgctggccctccatggctgcccatgctgccaccgccctgcgctgtcctctcaggggaccacggtgggccctttctcaagtctgagctgtcagaggtgactctaacccttggtgctcaccttgcatcctctctctctcttcctgggaaatttggaagttctgggcctcccaggttcagcaggggaatgagctctcgtgtctccttggggcacacagcagaccgtcgggtgtcagactgccccccgaattaaagatggggccatcttgagtttatctcttcaccgaggttgacggctcatctctgtctttccagcagggtgacctcacagtgaccactgcccagcctctgaggggtccctttgttgccagccgcacaaccagacatccccagggggcctcatttttagggaaagtgagccaggcctgctcttcacagcacatgagtcagagcctcgagaggagagccggggggcctggcccagcccggatccatcagggccctgtgtcacaggcttccctttagactcgaggtaaactacgaagtgaccttggaggcgtccatgggatctgaaggatgagcttgggtctccctgacattctcgctgaagatgccgcctgcaggcttcagcacagactttcagtcctgaccccatggtgggggagaaagggcaggagatgagcgagggggcagtggagagggggtaagagggcgacgtggggggactacaagctgtagatccggggtcacgccccaagcccgagtgacgactccaggaatgcagctgccctgggtgactgcgtgtctctgggagctgggtgacatggggctctgcgccgcggggagtggaagccgcatttccttcgtttgaggagggggatggaaaaggggtctgttgtgcatttccataaaagtcttcaaactccgtgaatattataggagcggccatgctgaaaaaatctattccctttgtctgtcaagtttgcgttgtcgtgggggcttcctttcatccagctgacttggaagctcgtccagggatgggggtgtgtgcagaccagcgagtgagaggcccacctcctcgcttcacggaattggcctcagattcaggagttcagcccagcctgggacccagcctccgccctccgccccgttggagggacccagtggccggctaagctccaggccgccgggtgtctcagcacctcctgccccacccaagtcggcagtccttggggagtagcccctgacgagatctggactccatcatcgtgtcagaggctgctggtcagcactgaggatgtgggatagctacttggagcagctggccccagaaggtcctctcaggacccgagggtgttagtctctgctagttcccaactccctggcatcagccacagacccacggccagaaaatgagctgtgggccggcccagtggtgcagcggttaagtgcgcatgttgtgcttcggcggcccggggtttgccgtttcggatcccgggtggggacatggcaccgctcatcaagccatgctgtggtaggcgtcccacatataaagtagaggaagatgggcatggatgtgagctcagggccagtcttcttcagcaaaaagaggaggattggcagcagttagctcagggctaatcttcctcaaaaagaaagaaaagaaaatgagccgtgtttggagatatgtggcccattcacagcccgaaagaagacaccagaatcctcgactctttggggttcgtctgtcttggattgactcccttgttcaaccctacttggaatttaaagtcggttaaagatagggtaacccaaaacagtggcctgtcagacccagggggccactgactcggggctcccactccccaccgaaggctacacatttggtacagccccttccagcaccccaggcaggtcctcttccctggatgctggaggcaaattctttagctgcagccgactaggatcaagaacgccctgctcccagcatggcatctcccaccctcttttgcgtctgcccattgtgggtcgtttccgggatccgatgagctgatgtgctaattgcagaggatcccgcctcagtggggccgggagtcgagctgtcgagcgcgggtccagctccggagtctcctcactttgcggcatctcttcacttcccccgcttccagtaagcgatgcgtccctcccgtggggagatgctgggagaatccagaagacttgggagatgctgagaaggctgccctgctggttccctggcaccgtgggagtcagggttcagctcctggatcctcgatcttaaagtgccttcaaagttctaggaaaacaaagacctaatatgtcgggccttccgcacgtgcatcgtcaggacgccccccccaccccatgaatgcagagcagacgaggggcaggatggccattctagccttccttcgtttgttccttttaagcatcagagagttttacgaggttttctaagatcaacttacaggttattaagatattcgtgtcacagaggcggggacgctgtgctgaggaagaggagatggctcgggctcatgaaattcagctgagccctgagcaaggtgtccccatctgtgggcctcgctttccccacttgttaagcaaggggatggaaacgaatgatcctgaaggcaccgcccacctctagccggtcagtcctcggcggcgcctcccgagccctcggccgcagtctctcccacacggagctgtgttctcctggacgggcatccttgtctccggtgagctaaccgccctgaggtcaccgagatgaagtggagctaatgcaccccgggcccccgtggcatcctcttggctgacggggaaaagccgctcttgggagctgtgctcctggtttgtccacagtttttcccgggactcacacagaacggggagcgccttgggcctcaacactgaccctcagacgctggattcagtgaaggatgaggcacctagaagaaacaggctcatgccaaaacagtcccggtgagttcttgctctacagcgattttcaaatcaaccgatttgaggggaaaaagtcagctaacggataaggaaaagtattgagattcagcgcaggtgacagtaggaagatggctcctgcttccgaagttgtggcctggcactggcccctagggcaaagcaccagagaccggtggcctgacccagacgttgattctctcccaggctggaaaccggaggtcccagctccctccagacttcaggtttggctgaaatcttcgtcttgtagacgtgtcaccccaagatttctgccatcatcttcatacagcactctcctcgtgtgcgcatcacgttgctttctgtaaggacacccgtcaggctggattcgtcacccaggaggacctcatcttaactaacgacattgcaatgaccgtattccaccttaggtcacattcagaggtcctggggcttggcatttcaacctatggatttgaggagacacaattcaacccataacacaccccttagagatcaggcacctgggccaggacattcccaggtgccatcggtgacagaagacggtgcaccgtgctcctggctgtccctgcatggactgctggtgccaagacctcctggccgggtctcctcctgggacaccaagaggagccgtgtgtggactgggagggtggggagtagagacccgggacacctttagttagtggcagccatgctaacccgtctaacagtgagaaacttcgggtcagtgttcttagccaattccaacattctccatttgaagataatagcagtgagacaaacatcggaattggcttggccactcacagacacccccttcatgctctgcacacgctgccctgtctgcacacgtgtggatgtgtgtgcacgcacatggtgaacacacaacggaaaacactccagtgaatgtcgattccacctgctctctgcaggcctttgaaaacttttcttcaagcagaagaaattggttttggggtgaaaaggtgtagtgtcaacacttcagagggatgtggttgcacgaccaaagcagttaaagatcccaaaccaaccgctccagctccgggcagtcggctccagaagaaacctgacgggacagaccctccgaaacgccaggagcatctggactccttcggaatcacccgtcccgagaaagcagactgctccctttcagatccggtgtgtggggggcatcaaaggcgtgctcctcttccggccttgcttctcaattttctgtgcgactctggaagggtgcagaccagcccgtgggatgcctgagcctccgccttcgctctgcctgacccgggtgccctgaccccggcatcgggggcgcgccccccggaaggccctccggagtccagctcaaaaggaaagggttaaaatgggcgaggggcaggcagggagtggaaagggacagaggcccagcggttggctaattggagagcaggaccggcttggggggaggctgcgcgcccagacaatgtcctcccgagctgggagctgggaaggcccggcgggattgttatcgaattagttgaagtcagaacccccctgaatgaggtaaagaagggaaggggaagtgggggccccattcactgcccgtgaaaaggccctgcttccttcctggggcaggggggctggctcacttttgtttggctggggatatacctacctacagcccgtaacaggaggaggacgggaaaatggggcgttgaaagtaatccccagtgtggccgtggggtggcagggacacaggcgtggagcacacaccaggagcactgcccagggctgcatccccactcccccatttcccagggggctgtgtcaccccttcatcagagcttgcccaccaggagggctgacccccgatctgccgtcaggtcattcgctttatccatctccacctaaaatttggagatgtaatatgtgcttatgtggccaattctgtatgaaggaaatatatcttaatagttgaactagagaacattatcggaagtagaatagatcagccagaaacctgactggccaaaaaaaaaaaaaaaacagagggaaaatacagattacataaaaaataaatacttaacaaactgccaaactgccaagctcagtaaatatttgttgaattcgtacattaattaattttccagatgagattttcctaatccattttttaaaggaaacaaggattaaggacaaggcggaatctgattattaggctggcccggaggagatgaaaggagcgcggtgtgtggggtttgggggccatggcctcgatttgctgttcaaagtccacccgcagggcgggcgccgtgcccctcgccacatcccacccccggggcacgtgcagcatgtgctggatttaggggccagactctccacatctggatggtcacccctctcgtgggcatcccaaagccacggggattcaaaggccatgaccccaggctttcgcatagggtggcagggaggtgcatgccccacaccccctgcctggtccagctctgccctcaccagctgcttagtggtcgttgagccctgagtcaggaccgggtaacagcgccccatctcgtccccattacccacggcccgaggtggcaccgtggtcctggttttcctagcaagggcacttgtcgggctcactgagctggtgcgtggtggccttcaagagcccactgaccacactgaccagggcccggcacgcccctctcctgtgggccactctccacggggctgtcgtgagaacgtgaggaggtcccattgtgcaaactgcctggcgagtgagcatccgaaatggaagggggctttgctggcgccgtggttttgtttcggggtgtcattggggagtctggctctgccactgcctgctgggggccgcatgtccctctgtccgcacaggccggggttcagtcctgccaggtgccctggcgggagcaaagcctgtgtagacagcgagtcctgggcactttcgaggcgtcgtggggcaaagcgttgcattccctgttctgaaacctttgagagccgctcagtagcgagaggtggcagagctccgagcatttcaatcgatggtctgtgattttaccgtgtagatttatccagatgttggagggagaacgtgcgtgcgtgcgtgcgtgcggggccgaggaggggccgtttccaagagagaccaggcgcaggtctgcgtccttggggaggagcgagcacacatttctttgcagggggagtctccttcggggcacccgagcgtcccatgggcgtgttgggcgtggggaggcagatgacctcctggccgagcagccggtgcgagaccgcggggcggagacctgagtgttttctgaaatggcaggtgtacaatttactttttctgcgaaccgtaaatctgtttcctcagcagaagtaaggaaattactcccgggaggcgaagcgactggagcaggggaataggccccggggccggatgagagaacacgccttcacaagggcctccaggaaccgtgtgctgctgagggaacttgaggcacattctggtctcacgcgagccccagtcccagacgaggaggaagaagggaagatgcagagtctccttgctggggctcctctggtccaaggagccaggccagcacaccaggacccaaggacccgtctgggagcctgagcagcccacagggtgcacagaggggatccacgccacctgcgtgtcccctgagtgcctgcttccaggccagcggatcgcaaaggagaggaaatggcccaaggctggaggcagacctggaaggatggtcacggcggaggaggcctccaggcggtggacgtgtccgcatgggggcccgtgtgccagcccggccagcttatagtctccaaaagaactgcccagagagagctggagcccgcacggtttatcctcattttatgacttggaaagaaattcgcaactacaaaaggcaaggtcatttgattaattccaccacgccaagtgtccctactgagcaacttctccggacgcaggggcccaaccgggttcctttctaacagcgagttttacgatctcggtgccgctggtgtccgctcatggcaaagacgcgccaagttttgcttcccggtgaggccggtgtgtttccatcgacagtttcaaagaggaagcacaatctgagtgtcactccagctgctgagcaggcgctcgggcaggagatacgctgtttgcagggtcgccctcagcctcagctgcagtttgttccgcaggcagcagcagctggaggtccctctgcccggtgctctggcctcacggcggggtggtcccgagacccatgctgcagccctagattggaaccccggctctgccatccgttagcttgggggccctggaaatgttaccccggcccgccgtgcctcggtgtgccagtctgggaagcggtaccagcccctcgggctgttgccatgattgagtgacactgccgggtaacggccaccagggcctgcccagtgtgctgtgctcggatggccggcttcttctcagtgcctcaaatgtctttgctctctgccatgaccaggtgcccacagggagccagccaggcctgcaggctcccacgggcccaggtgccggctgagtccggtccgccctgccatggtacccgatccctctagagatctacgcgtgtgcgtcccttgcccctcagccagcaccaggagcggaacctgtgctgagcaactggcccctctaagggaggatctaggcggtgacgccatcagcgggctccggaccaggctggtgggctcattggcgtagtctctggcctgcatggcctgttctttattcagacaatctaatctgaagtctgggtttggggcccctcacgtttgtcctgaggctgcagtggatgttctaggagacgccaggtgagagctggggagagcgaatgagagactttctgccacgctgtgccccttcctgccttcctctgtcttctgtcttctgtccagcactgaatagggatgagcatgggatccacccgctggatggccaggcgggttgcaggcccttgagataaagctccagcccccatccaaacctggggcctgtgacaaggccaggaggcaggagagggggatcagagagcacgaggggacacagcagtcctgggcctgctggacaccgctgtgggttccccccaggaccccgcgttggggtggaaggtcttggcgagcagaaggggaagctggcgtggtctctcacagccccggctctcctccacgtgaactggcattgatgttcttacgcgtgcgagggtcgtcatttccgcggtcagagtgctgacttattccagcacccctgcacccaccgtctgcttggacgtaactctgggactcgggctgacaagggtcttgggaattctgagcaaactcaaaaggaacattctgtgctacggccgctggggtagggcagacatcagacagcctttgaatgtcttaatcaaaaatatgggaaagttctggttccagtgacaggaaagattaatcattgatcaggtagaggctcatacctgagcacaggaacgagaagataaggacatgaactacatcttagattgggacttgccaagcatggtcacacagaggccacgggagtccagaaaatgtcctgcattcaacagcagagactgcggctccgtgtggcccatccccaggtcctggcaaggttgtcctgactgccacccctgccccatcgtgacctcggacagactggagccagcccaggctcccctttcctggtgaggtttgtgacggtcactttcatgcacgcatgtgctcccctgggcaagccggccaggcagccagagcctggccgggaagctttgctgggaggcattttggctggaagctctgcaggtgcgggtggaaggggagcagacgagccaatgtcctaggaccccaggtcgggagcctttgtcacattgatttaccacaaacccacctgctgctttgggggttgtgctggtgatcgaccaggagcctcaggcccgcctcccccgggtggggccctccctgatggctgtgtctcctggactcctgagcaccacgaccaagggctatctccagaattttccaaactggtggaataagaggctctggctctggctggaattctagagaagcctctgtaactggcaccttccagccctctgggctaagtggggcgctccagaagctgctctgtcacttatcctcacaccagtccattcgcaggttctttgttcacgtggaaaatatcgtagccctgtcaaaagacatttgcaagaggacctctgacattttgaagccacttttctttctcagctacagaaaatcagtgactattagtataaccttattttgaaagttagccaggcatgaatgtctagaacgttaatatattgaataaatacctgcgtttagaattatctcaccagaaaacagtttcctacgtgccacgtatattccatcagttcagtgttattgcggtggactcgtggggcgggacgcaccgcctctgacggcccgcccaacttttaggcagtgtctgtgccacccgtccagctctgagctgttgcaatgaggaatgctttccctgtgacagatggggaaactgaggctcaggaagctggtgcatcctgcagacccatgaaggggcagatgcaggctcagaatctgggccccacacagagcccagggctgtaggcgtcctcgtctccggccttacagacgattctcagaggtgtatgtgtgtgtgcatgtgcgtgcgtgtgcatgtgtgtgttactttcagttccaattacagtagcttacgctacacgtaaaggctgctaaaatatttccaagacagactctgaagaacagattccgtattctaggttgaagggagcaaaattaaagaagatttgcagtgaaatgtctgttctggattcacagggctggaaagggctcattcttgacttttagatagatatgtgtttatgcaccattagatattcatttattcattcattaggtacttattattcgcccattacatttatatgtttattcattcattagatatatatttatttattcagccactatatgtatttatttattaattcattagatatttatatcatttattagatatatatttgtttattcacttagatatttagctgtttatttattcactcattaggtatatatttctttattttttcatttaggtatttgtttctttttttgtttttggtgaggacaattggttttgagctaaaatctgttgccaatcttcctctttttgcttgagaagattggccctgagctgacatctgtgccaatcttcctctattttatgtgggatgtcaccacagtgtggcttgacaagcagtgtataggtccgcacacgggattcaaacctgtgaaccccgggccaccgaagcgaagcacatgaactcaaccactacaccaccaggccggcccccagatatttgttgattaattagatatatatttgtttattcacacattagacacttatttgtttgtctattagacacacacttgaggactcactgactcctcagacagatggactgtctctagcctgccgtctgcaggtgctcgtgggccgcccccgagccaccctggcccgttgtccggcgctgccttcccttctctaagcaggccaatcgaaacgagcgcgagtcccttgctgggacgtcgtccacttcaaatgtgagatgaaccaaggctggacttgactgtgcgtttctcccaagggcctagtgcaccagtttgaaaagccgtgacttgatctacttagaaacgagccgtgggtgggatcccgagaaaacgcggttcggctgaaactggtgtctctggggctgagctgggggcttcagtggattcgcttcttgagtttggtttggctggtcctcatttgccgcttccctgattgcagggaccttgtccccaagggcctggccctgtcctgcgttttcgagggagctgtgtgaggccatctgcccagtgacgggaggcgccggccgcccctctccgcgcgcttcccccacacggcctcctgcttcggcgtctccgcgcccagccagccccggttcttggagaacaacacatgtgcaggaacggcccaggcacctccgaggctcgggtgcgagcgtgcgtgtgcgagcgtgcatgtgtgcgtgccacgcctggcagcggagcctgagcgtgcgagcgggagagggtgtgtgtcgccgcccggttccccttggaatgcctcgccgcagtgggcaaaatggacgaaacatgaaagaggggacaataaatagtttcctaccttgctccaataatgggcgagctctttgcatggattaatcctcgcgttgcctttggacgtcagtttgcgagtgacagtaacaagcctcctgagggctccgctaatttgcactccattcaccgtcccctgaaaacccatcagcgctgaggaaggaccgctcggcgctcgaccccattcaaagggggagtgagttgctgggaaaggggccagttttcacaaagcccaaaagagcaaattggcggaagctttgttcctcctttttcctcctgtcccttttatgaggtcctgcactagttaagctcctcatttgtccccgccgggggctcagggcttggtcaccctcgggctgccttttcaggtgatctggacggccccctggccccggctcgagtggaaggtgggggcagggcggcctgaggtggggacactgcaggaattctgggagtccaggcctgtatgggagcgggcgctggaccccggtgggggacaacagaggtggccggaagtacacatttccctccaccctgggtcttggggcctgtccgaggtcctggctgggaatccctgtggtccgggaatcttgaaagatggagcaaaggttcccgcatgttctctggcccccacttcactgctgccttacttgccacaatatatagactcaacacaaattaaaattaacgaggcagtgtccctctggctcctatccaatgcatgctgttcccctgctccctacgcccggccctgtccctctgtccttcagcccctgctttctgtcctctccctgaatgtcagcatcgcaggagcagtttgtaagtgccccattcgggaaggagccctcctccccaccctcaggacgggccctgagcatttgctttactgcgtttctcagggacccctcaagcaccctacaaacagagctgggtctggggaacctggagggagcttcctggacgaggacgggtgtcacctgaggacagaaggggtgattcctcaccacttgtcacgtgcttccttggaaagcctcgctcacggaagccgccgagaatcacatcctacatatgctggcagcctctcatttgccggaaagaaaacaccaccgccaacagcaccaacacgcacagattcacggattccccagattagccaataaataccgctcgctcagtgatcgggagggaaaaaagaacactgtcccaaatctttatgaattaaacatggcgagtccatcaagtcatagatacagccatttttgcttaatttgctgacacaaataaccccgcctgtttcgttgaggggagggtttcccgggggggatgacgggaccccagttgtctcggctccgccgttgtaattatgaaattgtgaggactgtaatttgccactcaggcttgtccctcgctgggaagccacacgggccttgccggggggtgccctgcgtctcgcctttgggggttcgatcttgtgggttctgttgtgcttgggagtctgtttttccttccctggggcgcagactcctgcacgcggtcagccgtgggcacctttcctctgaggaatggctcctgccggtcggagcgtgaactcagagatgccgattttgttcacaagatgctccaggcacctcctgttctggcctaacctctcctcagggggctgagcagcaccaacagcatctgcgctctccaggcgaggaagcggggtttccaggatgctgtgtccacctaaggtcacccacaaacaaggtcacagccagggctgtagaccgaggacttgcgaggccacgtgggtgccctcccatgaggcctcactgctcccattaatcctgcaattccagatcattatcgaggccgcccggcccgctccattaattcggaagcacacggggcagagggagtggccagggaaccaaacagagcggccgagttagctatgcctgagcctcacgtgctcaccgctcagacgcgtctgcaaccacggctgttggtctgtcaccgcccccactttcctggagggtgcagagtggcccgaggtcttcaacgtagcacagcaaagatgtgtttccccgtgaaaagattattacaaaaggcaaaagtaataaggaaggaaaatggcttccctgtgcagacagctgtcttttcatttcgttctcctgttgcacataacccagagacttctagacctgcacacgggcctgacacaccagcaactccccacccgacccctctccttctagcaaaaataaagcctttttcaccagcggcgcagggagccgaggacggtggcggcttttaggagctggtttccaaagggagagcgccagtcagacctgccatactggaaccgcaggcctgtggcgggagttcctacgaaatcgcctcgaaggggcgagaagtggcggcatctcctggctctttcttcgtcccgcatggcgggtgggcctgccgggcgcttggccctgggccggcattgtccacgtcagacaagaggtgcccatagctggggaccttgggcacggggagctgtggcccaatcatgctctgtcggtggatgcaggccctttgacggggcgggtcctgagatcctggtgtgacgcactgggcatccaccccctaaaccttcccttcagggccgtccgatctcggaggccagcaggagcaggcctgacactgggggcctgtgtagatggattatgtggtcagttgatgaaggtattcatttcagccaacggggcagtcatcacaaggccattcacagacacagtccatccgctgacccgtctctgccatcagtcctgatggtcagtcctgggctctgggttgaggattcagtgccaaccaaaggaatatcccaaggtgttctttcctgttaccaaaaatcagtaagtctaatagagcagaatgggctggaacagaacagagcagactcatttctggattggcaggtagcatttggaccacatccagaagagtgtctgtcccataagcactgtggcaaggccctagtgacaggcgacgtccgctgtggcccctcaggaggatcatgctgggccagccccctttagacctttaggggggagggtgtttgagaggggggctgcggctgccacgggcaggttgtctgcacagccagtgcccccggcacagaggccgcagctcgttctccctctgccgagtggatgcccaacgggcagatgggcaccagcccggggacctggaccccctcccggtgccccgtccttggaggacccgtcctggcgcccattcctccgtgctggtggcttcagcactgagctcccctctgccctttctcactgggcatggagggatggccgtggccttctccctgccagcccctgccctccgcctcggggtgccctcgggcctctgatttctccttggctctcacagattctgcccgtgtccgcaccctcattaatcaatgtcaagcacggcaccagcaacaagggctgcattcttcggcggcccctcagcctcatacatcccattgttccgggccgattcagatggcaagaaaacagcctatgcagcacataaaagagatgagacaaaaatgaaaagaatcgagggagaaaaaagaggaggcatttttccccctgaaacactcgaataaatactcagcaatgataatttaaattattcatacatttatgaaaacatccattgaaaagtgaagtgtatgatgctctttgtctgttttgcttgtctcttgtttgggattaatctttactgtaatccgggctggccgtgccccctcacaaaggccacactctctttccatgggaaaaatgtttcggcacatcccagtgaaaagttggtggggggacgtttgtgctgatgtggcagttgccaggccctcaggatgctggacgggccacgtccaaatagtgtccgatgaacaaaaaacaccgagtggttctcacgtctgtgtcagcgcagtgacacatcagcgccacagggccagggagccggacgggtgccgtggccgcaccgagcatgtgtgccaatccctgcagcccctggcccccctctgaggggcgagcatcaaagtgctgtccacgtgacagcccgccggggagggtcctgcatgggcacgctccaacccagggacccgggggtccagaggggtcatgtggcccaccggagcttctgggtggctgtcacctgctggacccggcctagatgggcagaccctgtgccggtcgtaggataagccagtggccgtgtctgcgtctcctcagacctggaggaggagcctcgagccaccctgtccttaggctgggtctggcctcatcagtgaaggtctgggcccaaacctgaggccgaagacattgaaatcgtcctcgatatccagagggcaggactgtggccagcagctgtcaggagcgagctggggctgacagatcgctagcagtcagtccggggacgtgtgcccactgtccgcaccgccacccccacccacaccgcctcccagcccctccccaccccttcctggggcactgctgtccccgacagcccgaagccaccgcccagcatcgtcccggattcctccatgtaattaagctcggcgtccagctctgctcctggttggaacgctgcaacgaggagaaagggcgtccagccccaggccagaaacgtcgtttttatgtaacgtcctccctgcagtgctccctcgtccaagttttggtatttccagagttcattccaacacaggataaagaggccccaagtggaattcccccccaacacgggatttcactaggaagctgcggttttcggtggtgtgtgaattgtcgggcagcgactccttctcactggaaatgacccagaatggccctggctcagcagccagcgtccttccagttggggttttgtgggtgcagtggtggggacgcccaggcggctcagagagtttctcagttcagaaaacctgcagtggctgttccccgcctcctccagacgtatttggtctggaacgggagctgggaccccacagggacagccctcctggttggatttatggccgctctggtttgggccaggtcagaaccccagaggaaactgctcctttctgcttcttactcgagatggaagtgaatcccaggggtggctggaagcacaaacgagccactggagaacctggcaagggtgtggatggtgccagtgctggtccagggagggcgaggatgcaggatgctccagacctctgcagctgggccatgacttcccagagcaaacggttttcacccaagtcattgctcctctgtcaccctcctccagcacccacccccacccccgggccccaggcagctcattctgattaatcgttttttttttttttttttttttttaacgtgggaacattcttgagggactcagcctggtttcctcaagttctctgccaggtccattttgcactggctgggttgccctctcttctgcctggaagtttctgggctgctgcctgcttgctgcctcgctccccactgctagcctccgaacaaggtgttggggggccccttggctacgcagccctgtccctccaccccctcgccttcctccctcctccgtcactggtattggggtctgggaaggtagccaaatgccaccgaggacactggagccataaatcacccctcgtcccacatctgcaggccctgggggctagcccctcgctgctgctcatctccccaccccgtccccctcggacaaaacacctattgtcgtggccgagtgaagaagactcgtttccacttacaaagggcctctgaagccaaggcgtggactgccagaggagatgcctggctctcggcgggaagcccacagcacactctttcttcctcctggttcatacttttcctctctcgtgggggtcccccaaagcctttcattccggcttcatattcacataaagacatcaagaaaaaaaacccagcagaaagaattattaggcatggctgtcaaatgctttatcagaaaaacagggagggggaacagggtaaccctttacgcgctggcctgacagccctgactcctgagagaccccaggcgtcctgggggggccggggccgcctcggagcccagggacccgtcactggtctgtagcacacatgcatgcgccggcttggacacttacacacgcacaccctgcacacgcatgcacacccgggtttgtgcacaggtgggtgtgcactggtctatttcggagcagatttccaaatcactgctcgtgatctaaaggtccggcccactttcctcaggaagagatgctctggggcttccagtggctctttctttcttgtttcagccagcaaaaggggcttcgttctcttccgtactccagcaggggcctctgtccccttagacctgggcactgggctcttttccacccttggaactaaggagcagctggaactagaagtcccctggaggccagaggaccttccttccccaggtgcaggctgggacaagggtgggcaatcctagcacactgattcagaattcagataaccctgctggctttttcggagagcgttctatgcttagggaagggtgggctgcccttcaccgttaaccctcgattttctacttccctcctcctggcacggggctttctcaccagctcactcgacagttatgccacctgcccaatcctcttggctttctgaaggggcacggtggccccagggctcagctctcccagaggaggggtcctgggactcgagagatgcccaggtctgaggtgagctgtgagggcggggggtgcctggcacactgccaccatgggaccccttagaatttgtgctgaggggcaccccagggtgggcagcgcctcgggacgcgttttcatcgcagcctcctggtgcacagaaaccgaaaaccgcatgccgacaaacggctccctcggcaaacatttttatttccaattagcaaaagaaaaattgctaatttgctgcagttggaccgggctgacgtctcaatctgcggagtgtcagtgcagtgaaagaaaatgtttcccactgagccggcgagagcgagcctctctaaagagggtcagggcagcgggcccagaaaccagattgactggccacagtgctaaattactcacacaagacacacattgtctgccgtgccagctcccggaggacctgctttctcattccttccccttggagctggggcgggggaaggggtccgagcgtgggaggagaggccaggggctggcgtgatgcggtgagggcaggatcgtcttcgggaacagagaggggcagaagctgggttaatgattaccccggcctgtctgcggtgttcaggaacaggacgataaaattctcccagagcccggccgcagctctgcacccgggagattcagtgcgagataatgtccaacgggagttaataactgaacaggctggggagaggggccagccctgacattgctctcaacgcaaccgtagccgacggccctcgccgctcacccggctgcggcccgagcagctgatatgcgtgtgcgtgtgcgtgtgcaaatctcgggcaccgtctccccaccgtggccgagcctcaggtgggtctcccagaagggctggatctcctctgggcttggtgacctcgggatccacagccggcgtagaaaaggatcgcactgaggggctccccgaggcagccccgctggccacaggtgtttggcgctgaggcaataacgagcctctcctgagaacccttgaccacgcagcctcagagaagagcctcgggtgagctcggaggggaatttgcctccgtctgcagagcagcctcgttgagctggggaggaaagtgcctggttctctgagctcagcccacgccaggtttccatctgcagggccttcctccgtgtgtttcaggttctggatcctgcccatcctcccggccagtccacacagggcctttcccaagacatgtccccaacactgtggagtcatagcatctgcaccttagcaatcagccagggcagccccaagggcctgtggtctgccccggctcagatgcacctgcagggccggtccaggaggcacctgcaggcctctcctgtgtcctctgtctctggtcagctctctgggcttaaaccaggtcacaaagggcctgggacagctcagggagcagcactgggactgagcaagctgggacctggtgcaggtgcagggcctgaggccccagaagcacatagccccaccatccttgggtcctgagacccacctaggctgcctgtggttctcttcttcctgaaaggcaaggagtcagtgacctttggaatgacctagaagggcccaggctcagaagcagttcctgtcggtgccaggtggggaggctgctcagttccttggcagcccctggatgtctgggcccaggggacgtgctgtgatgctggcagcggacgcagttctgcagctgccgggcaaacggggatgcgaggacgagagcagtctgcaggcacggaggcagcctgctccctcatccaaggctgacattccactctgagagagaaggtccttgccgtgacccctgagagacaggatgcctgggcaggaagggatggctagctcagcctgacacagtcctgggatggccaccatgaccccagcaatgccagcatttggtcagccactcaaccttcagtgccaggaccctcgagacctatgatggctcaggcaggggccctggaattccagctggccactgctgatgtctgctttagacccgcgatgtgcagctctggcatagaagatgttggagaatgttccagcagggccccactgcctctgtctcactctgatctttaaggaaatagcctctagactaagaaaacagaggcctggacttggaccctccagatactggtgcacagtctattgctgtgtgacatcagacaaatgacttaacctctcggagcctctgcctcatgatataagggagaccatcaaaccagataatgtatgtgggactttttttgcaagatgtgaagagctgcctggatgcgttgtctttgtgcaaataagggaagaatgtctgagcctgtattgtgaggaccctttgtcctgagtggacccagtctatgagcactgtattttcttcaggaaaaatagagttactgcccgagccatcaatcatggaaaagtctggcctcagatgtacaatgcacacggcactgttcccagaaacaggagctggatgagcgttttgcattcggcctcttctcctcccagcttctccccgcccggccctgagctatccacagcctgagcaccatgggacacacgtacatgtgtacacacacacacacatgcacacatgcaggtgcaccagcacggacacattgtgtccacacaaaacacatgcatgcactcacatgcacgcacagagacacatggtgcacacatgcatgcatatttgtgtgcacacatttgtgcatgccagcatggatgcatatgcatgtgcacacatacacacaaaatacatgcatgcaccgacatgcacacaagtcacgcacataactgtgtgctttcataattccctcctggagaattcactcttccctcattcagaggcctggaacccaggagcccctcttggaggccgcccgatgtggagaaagctctgccgggaaggcctgaaagggtggaatgtggtccaggcaccctcatgaggccatcactcttctgttccccagaccccagaggccctgctgctccagacagacgccctcggacctgatgctggtgccgtgggaggggaaggaagggctctcacagatgcatccccagggcccggcctcagcggcaggtgccgtgaccttgagctcaggcccgtctggcccatccccctacagagcgaatggcccaggtggaagagctaatggcaacctgtctgagtgcccgggggtgaggtgccgccagctggccagcccctgctggggaggcccaggccatgcgatgcattcaccgaggctctgtgcaggcccaagcgctggggttctctttttccggggggaagtgccctctccttgctctgccgtgaggtggtgggctctcagccatcgtgagctgtccctcttttcttagtgggcttcaggtgctggaaccttctgggtcctgcctgtggatggtgctggcacgttccagagccctgagaagcaggcctcactccccagctgccccccagggcagggcctgctccccgccctggctgagctccaggatcacccaggagattccacaaacactgaagcccgggcccaggcccgcatcactgactggtggtgggcccggtgtaggcactttgcaaagccaggaccgagaacaaaaaccaaagcttttgccctcagcactgtggcttcaccagctctgcgccttttctgcgtccacctgcgattctccagctgattgtttccaaactctgaagttggggcattttgcatccaaaggggcctagagaaagtgcacagcctgcccgcctggccatcatggcctggggctggcggctcttcggacgggccaagactcgcttgaggggcattcaatcgaccattgaagccacaagtccaggaactcccagaagtgagtacgtacctaggccatctgccatgagaccacctgcccacaggtgccccatgtctgcagaatgcccggatgaggccaagacccttgctgcttctgccgccaccaaaataaaggcctctttctactgtttgagaattatcacacggacacagcctgggctaattgtcaccagggtacagctaatcacagtgctgattatggagtcactatcgattttcatggcgagcaaacgtttctggagtttataacgcctgttatcttttgactaatagacgcgaccctgatgagccgaagttcatcctttgtctcggctcctactccagccccatagggtggcacccagaaacaggtgtgcgcacaccgcactctcctcctgtccacactcattttttttcaatggtgacaaaagggagaaagacctttttgtccgtcaaaatatgcggtattaaatccaggataaaattcttgggttttttttttctttaggtcaaattaaactcatccaaagcctgctttccctctcgtggttgcagggaaatacccataaatctgaatattcggactgtataccaataatcacgacctggggagcaaatttctcagtaaatcacagacctggcgcacagcatcacccggcaccagcaccccgtgcactcagctgtggcgacgggacgacagacctgcaaccagaatgaggaataacctgattggggagcccgctcagcctcccggctcggtttctcagctcgttgttctcaacttcaaatcctatgaacggatgaaagcgaatgccagcttcccagctccgtcggtggcctttcctcctctctcaaatattaagataaacgggtgtcacttctgaagattttaagtacaatagaaaatccattgctccgatcaagagatttgtctctcagagaagctctggtgtcaggaaaaaggcaacgatccgtcagccggcgctgcccctcaaatagataatcaatttcccgggaagcatcaataatcagagtggacatttattgcccctcggatggtttatctttggggctggggacagcagggccgtgatgaataccaaatctgccagggaacacaattaaatgctgtgaggataaagggttttatatcgtgcacaaaaagcaatcaaattcataattgaactccattgcatgaaatcggccagaaataaatagggcaccccaaagcgcaccagagtccccttgttataggtcagacgacagtgtcaccaattctgccttcgttcatccctctggagccgtagcctgccgttcctgcatgcctcagcctgcgagcaggactcggtccttctcccaccccacatgtgctgcggccccctctttcctctcttggtggagagaaagcctttacctggtctgcatcggcttaaattgatgctgggtctgcgtgtgctcacctggccacctgcattacacagtcccaaggggacaggggggcagaggatttgggccaatgggtgtgatgaaagacacctcgggctccacaggatgagtagggggccccactgcccctccccagaagaccccctcttccttcgggcaggcgggggcctcaccctgaggtgacccgagcctctccagcaagagctctgggagagactgtgaaatctcagctgtggggattctcgtctccagaaaatctgcccgttcccatcagacacagcgtctcaggggctccagttgttggggggcgagtggacaccctactcgggtcggagcaaaaggccggtggggccgccagcattggggtttctcctggggctgggggcaccgtcagctccgttctagtctgttcgcacttcgtgaacttggttttcaaaagagccttgaaaatgttagcgtttaaggtcatatacacttggcaaaatatggcttttagtttggaagggaaaaaaggcataaagggcattttggacacaaaatagctctttaaaacagcattttgatgagttaagtataaataaatcagaaacacacggatttcatttcctttaaaatacccgcgggctcaaacactagcgggaaaagtgcctggcgagcggcggtttgggattttgacaaacccaaatattttgaagagctttctgaagtgtttgcacccaggtctcctcgtctgccctgccctccgtgagctcccggtgcactcacccctctgcctgcggcggacaggtcgtcctgcgtgtggctgtgagtgtgagtgtgtgtgtgtgtgtgtgtgtgtctgaatatgtgcatgtgcactcaagccagcacgcagaaggcaataaaagcaaggaatatcagcaaagagatgaagtttggagacttaaagaattgtcactttattttcatgtggaatgggagagaaaaccaatgagaaccctacttgcaggagttgagcaaaggctccgtctaaacccaccatagggatgaaatctggggagtctgcctgccgccccaatatctgggtcacgagcggaaaacccagctccgccctcagaagtgtgggctcaccaggacagagccccggaggacgagccgctgggacccagacatgtgggcgcagtacagacaccgccgtcgcccccaatcccggcgccgggcgctgagggtgatcttctcccttctgtgggaacaaattgaaggcggtgagggtggcaggcagcccgagtcactgcagtggccacttccagggccgggcgtggccggggagtgtgggcggcgcccggcaggcaggccggttcagggacactttggtgaaacatcagctcccgtcttcgggtggctcagggaccaggaaggaaggtggaggacagcttccttcctgtccagaactagggggtggccagcaggtgggtatgtcagggagcccctggtgatgagtttgctgggactcctaatgaatgaaatgaatacaaatgaaacaagagccagtcctgtctgctgaccgaggagtgcctttgaagtcgctcggattcacagagcttgctctgccgttccacaaactaataaaatctggggcatccatggggagcgcagaagggccccggcactctgccctgcggtgtttccacctcaggctctcttggtgagcctcccgcggcctgatcattcgggcgggatcgtagtccggttcttcagtccagagtgctgctcatccacgagacctgcggcccgaagctcttatctagaacatctggccccatctcggccgtctccgtgtctctgaaacccaactgctcgctcagagacgtaaacagagacgctgcctggccaaggcccgccccgcgacgtcttccctgcaatgttcctcttggcatctgattagaaaatgaaacttgcaatcaatactcaaagttggattttgagattttctgcagaattgggctcagagcgtttaataacaaggtttcttaccgcccggcctcaaaatttataataaaaacagatatttccccttcctgatcttgaaagccatgccaacggcggagaaggggcttggatgcaagaggaagaaactggatgggcttgaaggccacaaacatcagcccctgcacatcggcatggggccgggcaccccgatgggaccccaggggtcgagagtgttcccagctgatcctcaaccacttctcttaattgtacagtgttatgctaatcagcatgcgatgccttttctctttaagaccaagacagaacaaagcctcagattactaattacacaaatacagtgtcttttgctaattggattggttaatgttagttaataaagcgctttaaagataggcatgtggtatgaaaggggcaattcgtacgtttcataacaggctaataactgtcagcgatgaataatcagccgctgtcgcacgcacccgccatcggactctccttccaggtccttggcggtctgttacaaattaacgccttcgcgggagccagcggtgctgccgcccatctccccgcggctgcgtcccttcccgcccgccagggttggcagcgcacgcggcccacagccttggtggctgtttggggagttcatcctctaaagaaaaatgaagccctgttatttctattatttatgaaagagcctttggttaagcgattaggaacaggaaccggagtcccgcatccttgtcctgaggccagaggcttagctgttttactcagtgaccccgggcaagctccttgaccctcacaggcctcagtcaactcagctggaaaattgggtaaaaagattgtggtgtctctcaggcatgagctgctgagcggaggcgcgcagggcggcagaattgtgacagctgagggctcggggacgcgaccgtggccgggggtctgccgctcactctgtcggctggtgccaaaagaaaatcaggacctttgggcccaaaatgggaccctggaacctagggcccagatccaggacctggccgggtgaagcacaaggaccgaattccctggccggccctgagagctcctcccgggggctgtgtcgggagcgggccttgctccctgagcagagctgcgtgcagacaggtcctcggtgggcctgggggcggggtccctccctgctgcagcactcacaggcccccgcaccggccccggcggtcaagtttattcctcgtccactgctctgttttgagatgtggcctccttctgcctccccagttttatcgttttatccaaatcaacctttttccgttagggaaaccaagacggaaaagcaccgccagccacgaggctgcccctgacagctggcagctcggattcagtccacaggatctccttcgaaagagagtcggaggggggatcccaccacgtcctcgccccctcccggggcgcagcctgggggagcacagcccttctggggaagggaagtaaaccaatggtgacacgggaatatgtttgctttcgtaaacaaaaggcctcatcttggagtcagaaggggaaaaatgacccccaggtgttctgctttaatccacacagtgactctgccaacccgggcagcccccacggagaggaatctgcttgttggaccagacattagaccagacagaacgaccaaaatggaggccataggagaaagcgctttcagctcctccctgagtcactgtccatttctgcggccgccccggccaagaggggagtccaggatgggcaaaatgtcagccggaatggggtctgttcccactgtctgtcccaagtgctcaggagtggaagctggctgggcagtacagccacaggccaagtggaggcccccagacgtccaggcgtgaatctcgtccttcatgcactcagagtgtccttgcccaggagcctgcccctcggccgcacagtcccacggatttctgcttcttagaccctgctctctcgcttatcccagctctccagggcccctgcaatgttggggtctctctctccctcctcccctctctctctccctcctctccggctctctttctgggtacaggacacgctgggccacccccacccctccaggccgcgctttcacagaccctcgtgtggctcagcctggcgacctcagctctcttctcgatctttggaaagagcaaataaattatgctgctgaacaaaatctcccagctccaagaatagagctttaaaaaatccagctatgtccaagaatatttctgaaaccacggtcagtgttttcccgaggctgtttttgcccatgcatgggctgtgtggacaagttcccctggaagagctgatggcccaaagacccgctcaggagccaggggaggccctgcaggcccagcacagagatgcctgcagcgtgcatgtgccctgggggcctccccgtgtctcgccgctggccctggggaggcacattcactctgtgtcccaccctttctcctgcactaatggggatcatttctctgagtccatcccgaagacagatggtgaccttgtcgtgttaaactgacaggcatgccacagggccgaccagcatcggagtgccacctcccaccccaccgtcagggcttccatgcggcgggtctcccgctaatcagagtgtcctcccatccagctgactccagggtctctgagcttggaggctccgtccagctgccccttcttatttctggccgtcgcaggagagccaggaacgcactgaagaggctcgtgctttgatttcaggactacaaagagcaaggagcaagcaggtgtgtgcactttccctgaggccctgaggagagcagtgggatgccgacctcgggcctcagaacacgggcaggaggtgggcagtcactccccttagagccgagatgcgccggccggtggggagacagtgaggaaaggtgtctccggatagaatctcgccctgggcagacggcaggacaataagcaggtgcttctggcatctgtgtcttgctggtgttcacattctgtgcacttctgatggtgtcgccatggctcagggcgggccatgcgtgtctccagcagctgtcgggaatcccccagtcatcgtcatagagtgagttcgctggggcacagcttcgggcacccagctctccagctgagggcttcaagacacagccgcctccgctgccgacttcccgatccattcctgacgtgccgggggctctccaaaaggcagtggcgaaggcgcctgttggaacggcattccagatccccagcgtcacgaggactgactgaggcatctgtggacgaggactcgcccccgaccctcacctccaggatgagccccctcccgccccccccgggctggcgtaggggagtctatggcattccgcagcgcccctggggcagaagctcgggagcagggcgctcccctggcagggacccctcccctcaggggaggccaaacatccctggaagtcgagcggcttgaggtagaaattcaaacaggcgtttcgagatttctttcgtgacaattttaagctcttcttgttggaaaacaaacaggaaaaaaaaagggaggaaaacgcacagagtggtttcaaactgcttccaatttcacaccgccttttcctggcgggaggctacagctgggaatgtgctcccgcggggccggctgggcgtggctgcagctgtggtgggatcaggtgttcctgggaactctgcctgcggctgtggatgctctcccacctgcctggaggccaccatactgtggctgcagacctgcagcatttgaagtgacaaccttgcggcatttcttctgtttggggttctgctttagctgctgtgtgttggctggcagtgcgggcaaagccagcctgggacgaggagccagccctgcccacctgagcctcccacgcctgtgggatgtgcaggaagcctccacctgcatctgcggcttgcagctgcaccggctgcccgaggacacggaggcctcccgcctccatcctccagaaactgtgtctgctttgggccagaggtccacacgcttcttctgtaaagagccagataggacctacttcaggctttttgtgccacatgcggtctctgtgtcatagtctttatttttatgaccctttaaaaatgtaacagccgtcttagctcgaaggccacaccaaaacaggccccaggctgggtttggctggcggacccccagagaccagggcggcccagtggggctgggtctctgttgccggccttcaggcaccattgttgcggcttaccgcctacaagaggattttgctctgggcgcctggacagcctcagatatgggtagtggcatctttagcggcagtatcacttgtgttcttgttgtggaataaaagagagttggctgggccctctctgctcttttcatggctggggctgccgtggacggctgtggggcccttcggatgcgtttgcacccatttcacagacgagggagcccgagctgtccggttagcacacggcaccgagatgcgctgcggggctcagaggggaagagccagctgagtccaggttctttgtgctgttcctgcggcctcgcccgctcggccaaggctcctgaaggaagttagccaggaagcccctaaaggcgtccccagggctggcaggcatagcgtgtgctgatcgagtctgggaagtcctccagcattgcctccgaagcgccgtcggtgggcctgaccctgcgcaggagatgcagaaggggccccgcgggatttgctgggaatccgaggcagagtgggggtgggtgggccggccaagccttgctggttcagcctgactcccaaagagccaactgggccgggggctcccggctgcagcggcagttgcatggatgcggcggattccagaggctgcccattcttcacagcggatcagcgcggaattcctccacacagctgtcgctgtaattgggtcccattaggaaagtgtgcggtcttgtttggcggctctctgcctcccgccgcccccacgcacggtggtctcgagttagcacggcagctgtgaccaaaagccaggcactctgggctctgccgcgctgtctcctccccacagattggaaacattcctgtgggagagaaggcacagctccttttcgtcatctctccctcccccccgccaccacccccccaccatttctccaaatggaaccagttctgcagaggccacagaagcgaatggctcagggcacagcctgattggaactccagctctgccacttcccagctgtgtgacattggacaaattactcaacctctctgagctcagtatccccaggggcaaatggggattactagaggatcattgtgagcattaaataagaacacactgacgcctttcgcagagctggcgtgttagctattagtagtatcattgctattttattacgacctggaatggaaacagttgtacttctggcttcactggcttgcaagtttgcaaaacccaaaagtgagagagtccacaaatatttgccgagtgaaggagtaagtggaataattaaagagtgaggattagatgaggccagaatttgatcagggaaaggccttgtgtcccctaggtcactgccccgaggcggcggtgacaactaggggctgctgcttcttgttgcatcgtctccctgtcccgggaaggcactgcctgtgttcttaaaggggattaaaattcaactctgcttagaaacgtggggagatgacctctgactttcccatctttaggtaattaaaggtgactccaaaccccaataagtggggtgggaacttacggcccaagaagaccccgaatcctgggggacaccctgaggggggttgcgtttgagcatcgcccagttgcatccccctggacctcgggtctcatgactcaaactcgagtgtgtcagaggagccatggccgggagatctatgaccccttcttattcacttatgattattaaggatcatcgtaaccaacaagttatcaataacagagtcgcgttgaagtgcattaatatgatcctgtcaatatctaggttatttctggaactagaaagggttgagagtaaaagacgaatgtgtgccccgagccgacagttgaccccgtgatgtatttccccctcgggaggcgccgaggcgcacagtctacccggggcggcagccaggtctcggaggccgtggctccgggtggctgtcgtggaggtgcgggcagaggtctgtgtaggggacagagaaggtgggacggcccgcagtggtcccaacatgttttttgaatgtgagttggaacctctggtttgcgatcggcagagctccgtgtcggatgaacgacgtcctggggctgtgcagggaccttgggctttcgggacgtgaaagaacactccagaaaatccagtgaggttatgttaggaccgtcctctcccgggtctctgagaggctgctggagaagggggacacgtacctggcaggcccgcctttatctgtcttggcagagcctggagccctcggctctttgaagcttttgcaagcgctctcattaagctggtgggcttccagtgtctgcagttaatgtatgccagggtcccggaggcgctggactttgggagtgacccaggaacttggaacccaggcgtgtgtgtggctgctgtgtcgattttctctttggggatctcatcccttagtgcatccccacctcccagggggccttggtgcctcccaagcagggtgaccaaactgaggggctcctgggatggggggctgtcagtgatccaacccagacagttccaggcaatgcggatgctggtccccctgcatcctgggcgctggtgcaggacacacacagagggcctggccttccggtgtcaggggtcaggagagacggccttcaccctggagcagttcaaatctggcctgggctccctaagctgttctcctctaggccacatgaccccaagggagccgggccctgtgcccttcggaccttggtgtctgatctccagccagttgccccgacctccagcccagcaaggcaaggtgcctgcccccacgcactacagggagagccccaagcacgtctccagggtggcccgagaattgtcttgggtgcagggccgggccaccctcagcctggacaacccagccccactctgacctcagccatctcctccccaaaacagggtgggctcccggtgagtgtggccaggacagccgctcagtgaacacttggagcagggatctttattcactcatttactcaccatttattattcggtcaatgccgtctggggtctacagctctgtgtccagcctggaggagccacaatggtgcctcgggcccagctcttggcctcaggaggcgtccggtccagtagcagagggcagactggccagaaaggctaggagagctctgaaataggcatgttcctgtccacgtgcagcagtgacgttcacggagaataaaggacagggcggggggacgacgggtgaggccaccccagagtgtcactagaatcaccaactttcgacaccaagtctgcagcatggagactgtgagtttcctcggggtgggccccccgggcgactgagccagagcagccccgccccctggggcgccttctgcagcatcgggggcctcccaaggccccacggctccttctagctctgtgctcttctcctctcgaacccaaaccctcagaaggaagctctggcaccagttccaggaacaaacggctctgcagaactttctccccgtttccggggcacgcgtgtccagttcctggagccgcggctgcaaggcccctggacgcaccagtgcttccctttctagcactgaaagttatggcttgtcatgagtcattgattggaagtgccacacgtcacccgcctcttatgatttacgagcacttcctctggggacatcaatcttccggaagcaggaggaggaagctgtccaccagggcccggtgcgatgctggacaccatctcccttgggacaagggcctgtggggaccgtcctgggctcggccctctgctcaccgcctggctgagaaggggttcgggagcctgcgcctcagctgctggcgggatgtggacgtgtcggacttctcctgccagcccctcctggtcagtgccctcgccagagctcctgcgcctgcttgggccatcagcatgttctctccatgcccaggggctcagccgacacagcccccaggtagcagctctgcgaaagagctcctgccaaggccagtaggacctggaggtctgacctgctccaggggtctctgggctgggtcacccaggccgccccattcggaaggatgtgacagaaggagctgagcagcatctgcagggtgggctccgcggtccagagctgcccagtgtgtggaggagcccgtctttgccctggcggaggaggcaaagccttgggtgcaatggctcgagcgctccagaggcttgggatgtcctgagctgggggccccctgcactctgtcctctccggaaaagggtagctttgggggggcgggccagagagagcaggaatccttgcaaatctcccccgaggtcagctcgagccctgccccccgacggcactgcccacgtccccatggcatgaaactcaggggacaaaacaaggtcatggctggggcaggccgagggggccaccactgtgcctgcttgtctcctgcaacacagccctgggagcctggggggaggtgctggaggggcctaggagggctgccgcacaggccaggagctggcaggagtgagaggcaggacacccccccacccaccttggggcctcgtgactctgggtccagggccacagacttgctgtctgattggggagcatctgcgtgagatcactctgctaaggagctgagcggttaacaaggtggcttgcgagttggaaggacctggggagctcccggctggtggcatggcctgtatttctccgggagctttatcaggaagatacacagttgtagtgggaaccatgctctgttggatgcccgggaacctaggatcagaggccagcctggtccacaccggtgctgcctgcagtcttcatcgagaaaggggttccactgtcctgcccaccgaccaggccctgccccacgggcgtgctcatcttgctcttgcagggccaacagctttggcttgactcctgtgtccaccctcaccagctgtgcacatgtgggaaggttcctctgcctctctgagcctcttccttatctgtaaggtttggtgccggcagccatttcttggaggcttgttctggtttttatgtgttagtgtgagccaagcacccaccatggcgcctggcccacctgaccctctgtcctcctctatctcaccgttgtccatttggtcagcctccaacaggtaccagctgatgctggggaactgggggcccagtgggctggtctctggcccctggaggccaccgtggcaccccaccaggcagctgaacagggaagatgcgcacgtgctccatggaggactacgaccacttaggggccagggcctacgaggccgctctcccctgccccaccctgcagaccccttcctctccttctctccgccctgtacctgtctgtctgtcttgccagcatcagtgtccacattcaccctcagaggcaaatcgaaaccccactcgcactcaaggacgcccccgcacagccccaaggcctttccaaacctggctgcctcttccatcttctgggcgtcctcttttgcccccgccccgcctgtgcctgtggcgcccccccccccccaccatggccccctcccctcctctgcccacctccacctgccacctcctgcttcagaattcccactcctgaaggccttgtgcccgccagcccctcactcaggactcctccgcagcagtctgggggcaccttccagggtcttcatgtccgtcctgttgaaggagcagatgggtttgacccattgccgctgccccaaggtagctcatactggaatccatgctgttttgagactttttaaatgtctccaagtcacttttcatggggcagtgtagacaaaaggtgctcactggctgcttacgaaatgagggtctgaaggatgttgtccctctgccctcaggtgacctcggggggactagaggggactagaccatctaacctggagagaggacccagggacggggattcattcattctatcagtcattcgtcctgcagaaaggaagatgcaggcaccagagtgcccgcaggcagggcagggtacccagtagaggggcgcgtgcccatggacggagagctgccagcgaacagagtgttctcggcacgtctgacatcaggcaaaccgtgcccagaggttctgggtctgtgcaaagccgtgctggccaggaaaacagtgtccgccccatcgttagctcttgcagccagagcgaagcagaaggtcccaccacactccagagaggtcaagcgagaacgcccctgtgcccagccgaggtctcagcggggatacccctggggaaagcgggccggggtgggtccagggcctggctccctgtgcagggcctagaggcagaggagcccaggttttcttgttggccacaaaaagggtttgcaaggaggaccctgctgtccttcaggcaggtgggacactgctgtgggctctcagtaaggggtcctgggacaaacccctgacttcaggctcccaagtgggaggctggggaaatgcctttgggcggggggaggcgcaggaacctcagctgggaatgtaaacagccgcctggagatctatatgcatttcggggatgggctctctgaaggagcctgggtcctttttcgtgccgtacttgactgtgtaaatatttttcttaggagtaaagagagaggctccattcattgggcagatctcacttgaaagaactgagaagtatctgtttaggcataaaagctaaaaacggtgtccaaggcgcctttcaaaaaaatcatttctgcagctcaaacatggccaagctgatttttttttaatgcaaaaaataaataaatatcaccccgctcccagttgggagctcaagagcttgtgtcaacacccctggagcctctgctgtgggagccagagcgggtcggccagtgactgaccgcccgccacggttcctgtcgcagctgccaatgggacacgacccagctcggctccagcttgcacggccgctggcctggccgaggggagggcctgctgggggtgaggggacctggccggggttcaggtgcgagtctcgtcggaagggtcggtgtgtggcttctttccgccagatgcaaggcccaagggccggctgctcctccagagcagaagcccgggatccgggacggtcggagcgcagactctaacacttccaggctatctgtcctttgccctgtgctccaaccccccatttcctgtctgtaaaagccaaggcagggtagtaacagctcccacccggccgggattgctgtgagaagcaccttcgaggctcctgggacaggcccccacacctcgggctcccagagagactgcccgctgccgtcgccatcccgccaccacccggctgtggctgctgtcacgacgtcgatgcagcatttacaccaagggaggtggcagccatgttaggactgcgatttggacaggaaattaaggatgagaatgttcttgaaaaagtccgacttcccagaaagcaggaagcaccagggaggcggtggcctccttgccgaggaagagaggagcgtggaacttggctgtcctgtgagcgccgatggtcagctcgtcgggcccattgcgagggcacggcttaaatcactgctaagagggcaggccctgtgagtccgcagcagctgaccacagagccacgtcctgacccccctgctagactgtgtccccagcaggactccgagcccctggggccctcagtctccccgagggcagcagagtgaggctcagagctgggacaatgtgagccccagtgcttccctgtggccaccccacgcagtgagctgtgcaggcagcccagagtctgcccgtggctgctgcttcgaggggaggcctcttggaatcagtttttagccacttctagacccagactcacgtctttgggcacaaacaagcgacttcagcaatggacaagaaagaaacaggtcagagagagaggtctgagctcaaaggccaagaacaagaggtcagtgaacgagagcgtttggtgacctcttgggcggcaggccagactggcggggcttcactcacatctccctggctttggaagccgtgtggctggggctccgtcagggtttcctcctgttggtgtgttttcagatactttaataacagattattttgaagggagagaaaccttctgagatccatttgtgacaccgaccagaaagcttagcccccctcgggggacatgccagagctccggagctgtgagctacgagatgtctgcgcgtgcgtctgctctgcgctgactttttggagaacagagggtatttattttaagagtcaaaaaagatgttttgaccaaccccacttagctcagtgctggcactgatacccgcaagagaagggaaacgccagagggcaaacctcgcttttctggctaaaatgcttaatcaaaacacacctaatacttttgccgggtgcagagatggggctgagagcagcgacagccactgccaacagccgaccagccagcgacgcgtgtgctcagggctcaccagcaaaataaagaggttgttcagcaaagcggctgccttggcaggggagagggccggggggggaggctccagaggggctgcgtccggcgggtgttcaccgaggtcagcggtgggtgaccagcagaggtaatgacgtgcattcctgccaagacaggctgtgaaactggggcaggccagagaagctgccggaacagggcagttctggaaccgactcagacctccctctgtaggagggccctcctttcttcaaggtcatcggcaatggcttcctctgggggggcaccactcaggttgggcttaatgcccccacctctctctgcatcatgtgactgggtggggcttgtcctggccccgtgtggtcctacagccaggcttcagggcacccacaccctgcgcatgacatgacgggaggggaggaccaggctccagggagcagaggagaccctgcccccagccccgccctgcacggatgcactccttccatctggcgtctctggcttggtgtgttgtgtgtagcatcttgacctgttaaggacggtgtggtttaaaggtccgtctgtgtgtccccatctgaccacaggctcccaggagccatcaccgggcactgatctcgccggactctccctgatagaaacaggggcctgaagtggtgacaccaatggatttcctgggatgcttcatcccatttgcccactgtcagcctctcacgaaggtggttcttgccaccctcagccctaggcctgtgagacacagtcgggcctggacagcagccagctgtgttaagggacagcaaagatctgggaaaaagggggtgtggtgctcactctgtgccccatcttcccagtgtggttgagtgaccgatcctggaccgcgcacgggggtgctggccagctctccttggaaagtctctcatgctggttttgaccaagggtttctggatgaattaggagtggtccacgtggtgcctcagatgcaggtcagggagcctgtaggaaccaggctgcagaaagggccaccgcgggttccccacctcggggtcagggcctgtgccgtcatcagagagagaggcagccgtgtgctgtctggagcccagagcaggcaggcctccagctgcctctctcccaaggctgggcctcctgtcccaggacactctccacgtcccgcaggcgcggcttagcttcagtccagcagcagcattaactccctagggctgtgagacatgtttccaggacggcctccggatcttcagaggagaataaacccactcctgcgctttctggctgacctcaagcccaccactggcctgagggaaccccgaccccccaaggaaataaccatgagccatattccctggggacactccccagagcttacagccaaggtgtatgtgatgtgccttgcaaacttcagaacctgcttggttatcacagctgccccctctccatgaaggggtcatggggagcggggagaaaggaaggtcagggccaccaaaaaccgaagtgaagacagagctgaccaaaacgcagcgggagtggtgtgcactccaacagaggggccagcaggcagagggtgggcggctgcaccctggggcccagcatggagtgtgttggagaccggagggctccggaggtagggatgcagggctgaggggtctcccctggctggcccttcctcaaagtgatggggggacttcgctgctcttgccaagtgctccgtgatctgcacactgggacggagagagcttgaagccctggcaacgcaccttctcctcctccgggaagaggaggggcccgcctgccagtgtccaaccagaggccagcaaaggcccactcccaggcaacacccagcactccctggctccaggatgggggcgtgagtggcgagggtgtgcaggctctagcccctgggcctgcgaggcgcggtcccatcaacgccggcgcctgtgcaggcggcactgacttctctggggacgccagagagacaacagtgtctccttcaatgatgtgctcccaaggcaggcagggccgggaaggcttggttctggtttcagccccagcctttgactcaccacacagtgcacagacacttccaatgctggaggaggctcggtgacagcacgaactgcggggagcacacccagccatccccttactctcggggaaccccagggccaccatgggatgggggcaccctttcaccagacctgggcctctgccctggactggggccgggggctggagccaaaccaggaggcattccacccgagacagctcagcacttggcctccggcaccgccttgggtgccgcgggcaggtcacagccccgaacctctctcctcttctaaaggcggtttcacagggcggcgggaaccaatgctggtgttcccagtcactaagtgcaaggcccacgtgggaatgtgatcgccaggcctggctgcagctggagaaggccaggctgtaaactgtgcctcaaaaagagtcacatgtacccttgacacgttcacatgccctctggggctttgcaaggtgaccgggtgtccgtacaggaggcccctgcagcctcttccctagaagggaccccagcagcggcaggggaccagagtggcctccggcctggttgaggggcccaggcttcctccatggcggttccgtagggtaggggctccttcgactaactcagtggggtgaaaggggaatgtgaggcgcaggcaccaccaggccccaggtggccgcctcctggtttagctccgcgtctgcgcctgagtcacagggacaggctctgggctcaagcagagctcccaagaagggaggtcactccggacaggggtgccccgctcatggtttcatcctgtgccctggaggctgcgctccgtagaaaaattatgttcttctcctggttcgagaccccccccgcccccacaaggggcagggtgaggaggccagagctctgcccggctcaggggccccgtgacccgagcatggagcccccacttctgcacgatgctcacggtggtctcagtctctgtgggtgaccgcgtccagggtgggcctgggtgccctgcagcaggaccgcagtccgactctttcctccagggctccgggaggcggccccgggatggcctctctggtttggggattggtgagcacagccctttcctgaagccgcagacttttctgagatccaggcggtcagcctcgctctctctctcagcattgaccaagcctctggccggccgtcttggctctggattcgggggtgaaaagcagacagacaggagggcagttaaggcctgcccgctggccgagctcagcccccagcatctgaggctgctctccccagggcctgctcagccccaggcccagtggggacagagcattcggtgacctggtctcttgctgattctcctgggggggaccttcccagggtctccgttggggtgttcacctttttctgtctcagtctttttttccacttatagccgctttccaaaccacaatttcagaaaagagaaatataaaatccccaattggttagaattctcaacatttaaaaatacgacttactgttattacagctgagcccgggcagtttcgtgagcgttttggaacgtggcatcctggtcctctcgcctggcctgcatgggcattgcgggcactctgggccccacgcttgtaatttgatagggcaatagtaattttaagggaaggggaaataaaaatgcctgaatttaacaccatttctcattttttttcaaatattagtttcaatcttggtctctattcttatggaaatgacattccaatttatacatgtgactctgtgcggctcttgtgacccagaagggaaaatcccatttatacagctaatttgtcagtttgcttagcttagaaagcgtcctggggcctgtcactgagatgctgcccctctcgggaagggaacctggtggtttttcagaggagacgccggccaggcaaaacgctaagctccccagcactgggctggggacagctctaagtcgaaaccgtcaggcagaagaaagacgcggaatggctggcactttctctcctcattgtcccaagcctgtgtccaccggcagagggaagggcaggagtgcccggcactgcccacagcccactgctggaggaggggtcgtccagggccagcccagggtgcgtctgtgacagtgggcaccactgcttttctgccttagcctgattccctctgagaagcttccaccaaaagtcatggagaaacatcccactcccaggagtccgggacaaagagcaagctaagatgctgcccctctgatctgccaaacaggcaggagagagatggcatcagatcccctttccgaggcctctagtagcagtcaggccctgctgacctctgccgacctctgccctctgccctctgctcttgcctcctccacagcggctctccttggggcatggcttgaccaagtcccaggacgaatctggttgctcctctgagctctgcctccccagctagagtgcaagccccattgggccagacggcacctcctcctgctcacccggcctgggctgcccacataccggcatcctcctggatacagtgcctggctgctggtagaactcagaggaaacgggagagtcggttccacatttcagcatgtcctcgtgccccattgaactgttcctaataatatgtgctggccttgggacccctctgcaggggtctgtgcccagttcctaaaggaccagggtgagccacacagattggccctgtccctgtaaggacccagctgcctttccaaggaggtgacatcccgggggttccttaccgttctcaacaaaccgggagacactttcgggataaaggaccccgttctcagcagagagctttcttgcccctggagaagccaggaggtctcacccttactgtttcaggaacacctagtgtgccccacgatttatagcccctgtagcttgagaacggtgtttgatcagaaactaacaacccctttcgaattgttttgcagcaaatgctgactgatgcggaagtgtcgacacaggagggctgcatccaaaaggtaggccagctggttctgcgccttcttgcgcgtccccatgggttcctgtgctaatgcgcatataggcggtcatggcgggccggcgtctttccatgagtgatgtgtcgtctgtggccctaggctcgtgcacagaaccgtgatctcgacgtaattaggacggatgcttattttcattctgtaagcaggtagggggtcacttcactgaactgttctaattcccgagaatgaggagagccctgtacctgagggaaggcctcaggccacgcagatgcatgaaattaaataaacgcgggctgtgtttatttaactatcttcactgggtactcacatgaaaagaatatggaggggaaaagaaacacgattggctgcagattcaacaaagccttttgctgtcctcagcctcaaagagggctcggtgggcacagtgttaaataagcgaaccatcgggcgagagagggggtgtccggtgggcatcatcggccactcggccgctgtgatggatgatggctggctgagaaacgctgccgttaaaagatccagagagccaacgaggccgcaaaaaacgtacatccccagaggaaagcacaggcgcacaggtgggcgcacagctgcgtgtgcacgctgcaggctaggctgccggtggaaggggtcagatcccatccagatctccaaaacctgcaagccgttcgtcagctggagctcagcttccatcgagttctggtcatcccagaattgacacatgcatacacagacacacacatacaaacacacgatacacacaccaacacacacaagcacacagacactcacacatggtagcccatctagcatttggggtttcccaggagatttagtgttctctcccttagcatctttgcttcaaagtccctgggcaaagcagagagttacatccacacctcccgcacagacggccttggtgcacggcgcgggtgtggagagaggagtgggtgcggggtgatccagcccctctcgagtgctgagctcttactgccacagtcacagagtatgtgcttgcggggttctgtcaaccgctcgtgggagagcctgaagccgtgacttgagctgagcattcgaggtggagtaaaggccccctcctcggctctgaggtgaacagctctgccactttaccaagttctctgcgagctcggctcgatggttagagccggggcatctgggcagtgttccataacggacagggggtggaggacctgccatctgggcagacctatctgtaagggacaggcgacctgccccgtctatccaggttcaaccccagaggcattgattttggagaaaacgttgcagtgggagacctcctctgaccctgcgacgacctcctgaaggacctctggcaggggcgacctttttacctcgttcatccgcataactcacggtctgtgtccgtccccaagggactgtggtctgagcagacgctgggacaaggtgtcggctccctgtcccactgcccgggggtggcctgaggccttccttcctccaaacgctctcattgcgccatgaggtcaatcccgacagcaccctttcccttttcctgcctctctgactgaattcgtgtttcctgaaagaatggaagaccttacgcaagagaccttacacagctctcccgcttctcaaggaattgaggtattttcacttcccacgaaaaggcacctacagccaactccagcctcagctcctggagagaccgtgagaacgtcacttgtcccactgccacgtaactgaccgtctggcagggctgtctccggacaacagggtggcctggcagaggtcagaggcgggatgagtagtcggttgccgggccagggtacaagggtccaggcagaggtccgaagggtcaagtgtgatgttgaggctgtcagaaggcaagtgctggatgcctcagagccagaacgagatgggacgggacaggaacaagggttgcgcgtcctctgtgccctggggcggcgctgtttgctgagtggcctctctcatttataaaccgtggggtcactgcaccttggctaacgtggcccagaggtccctcctgagagatgctcacaggatgggtgcaaggtgccatgcagccaggacccgctgacaactgcccagcttggtgcaggagtcccgtggcatgcaggcccgtcggagagggaccaggttccctgtccacacagaatcggaacagacctgacccccatggtggcccgagagtcagggaggcattgggcaccagagccagagaagacttggtggccctctatttcggaacaggcctgtgactgccagctcgggcccctccctgcaggctcccgtctgagggggctgcctggagggggctttgcccacccctcccgcacccctccccccactctgggaagctgccgcagggccctccactgtccactgaatcccaggatttttccattccgcaccaaacttagacatcatggacatccagggtgtgcgagtgtgagcggatgggtgcccaagctcacgccggtgggatggctacaaacctacacctggtgtttagagctggacccatccggtcatgtcgctccaggagggaccaagggaggaggctggactgagagagagacctgagctaaaacgcggaggcaaatggctcctagaggggccggaaagacaaggaaacacagtgtttgtttgaccttcttcccaaacggaaggtcccacctgacgaatagatgtgctcctcacagctgtggctgtatgtatgtttatcacataactccacatgcctcagtgtgctagtttacagactaacgtagtggattccttggtaatgggacttagactccagtttatcagttttcagtctgggatttttgtgtctggctgaacagggcctctcctccccaaagacatgcacatttgggagccttaaaaatatgatggggcgctgtgcggccttgggtcggtcagttgacatctccaagcttcagttatccatctttaggacgattatacctcctgttctttatctgcatcagtgaaaggtaacgaccggtaattaatatagtcagtggcttgacacacagatgcaggaaaggcggtattttgtgggtggcggcagtcacacaatctcagcactagagagccaggccaccgaaggggctggaagaacccattagttcagagtgtaccgccgaggctgcccgggccccgggaggctggcggcgcccacacccgcctgcagcgcggcggggtctgcggtgcggggcggggctcccgtcggagtgacagtggctgcagcctgtgagcagcgtcagcattggccccgcctccagagggggcggagccattcctggtttggcgggcggccccgcccctgggtaccctccatagcgcctccccgcctgcgcccctctgttcggtcctctccatccctcgcgtcccagtctcgcgcgtcccttggtctcctccgtccccccaagtctgccgtgtgccccctgcggtctcctgcgtccgtctccatccctctcatccccaaattcttgcatcctcctccgtctcccactgccccctgtccccagggcgcctcctcgggccgcgcagcccggaggtttggtgctgatgagactcaggcccgtgctcgccccggcccggtgccctcgcatcctcgcgcccgccccggtgtgcggccagggcgcgtctcccgcgcttgccccaaagtcactttggccgcaggtcgggctgtgtcggcttttctagcggcggtgccgggacctctgggactgctccctctggccgagtctgtttagcctctgtttacttagagggaagccccgggctccgggagagcaaacagctgcgggagataatctagctgttgtccatccatcactcgggtcgcccggctccctggagagcccgccagagtctgtaataaaaaggccgggcgaagaggctttggggacgaataaagagccgtgttaagggcccgctgaaaatggatggaagacattattaaggctcaatggccgtcttatcaaaatccaataggatagacatttggcaaaaggcttaaatttattctttgactttctggaaggcttgaatttatccgttaacaagagtgtagtccacccggggagatggtatcagctaaaaagaattttgattcaatctcattggtgatatgaaaaggggaaacacaaggcccccaacagccttccctttgtcccactttatcttgttttctttgaccatcttgaaaaggaaggacgagttaatacggtacaaccggggagaaaatgcaaacaaagggaagtgacctttttctttatttttatttttcttctcttccccttgtccactgccccccccccgccccccaaaaaataagtaaatcgggaatttgcatcagtagggctaggacggagcactgacaaagtgcctctcccgaagcttggaagactagccaccccagccagtgcgctcggggacgggtggggagggccgaggcttagaaggagagtcaaaaataaatggagctaataaagtcctacggctggaagagggaaggtgatgggtccataaatctctccccgttattacgtacaaaatgctacaggtcataaaagtcatctgatagcccaggctggcattgtccaaacagggtgattgattgataatgatgataatttgtgaaatgattatttggatgttaggagtgtggcctgtcccactgggcggccgtgtgtggacgtttccacagccttgcccgtgcggaatgcctctgctaacgggctcagaggtctcctgggtccctcccaggaggacctgggggctagtgaaggaggactccggagcctggaaggagagctcagggagcactgggaagctgggagtggagccggacaggccgctgctaagtgtcttttgtgcagcaattacgtgagaaggccacacaccccgaggtctaagccgtgacttggcagttggtggctggttcttggccttgctgggccatgtcagctcagcacagcgctgtgcggccaggtgtttgtgctcagtgtcgcgttagtggtttatttcgtttgcattgttcttctcttggaggaagcagaggtattttcccagtggctgaggggttcgggggtgcagagagagaaagggatgcggctggaaggagatggggagacccggaaacaccgaagcccccgactgcggtgctgatgtgcggcgctgggctccgctggtccgactgggaccagagtaagctgggctctccctgctgcgctttgcctgtgctggccaggttccgttttagaaatccttttctctttcctctttctctcccagccagcaacactcaggagcccccaaagcaggtgcccccactcaactgcagtttacctacaaatgcactttcttacaaaaaaaaatcacagcggagccccaagcccgaagaaccccgcaatcgcaattattaatttgcatgtgggatcagttatgtctttatgaaatcaaagtgtgtttagctgccacagagacagccccgagttgattttgataatctcgggctgaatagactggcttcgtcgcagtaagcccgggacaaaaggctggacagtggctgaaagccctttgacgacgcgtctctctcctttgttcgggagtcgctgataaaagagccgggagtccattaattcagtgaatgaacatatagtaaagtcagtcccctcttgagaacaataatgttgaggtcagcttcggttgccatggaaactaagtactcctaagaagtttgcatccttgattttgctgaatcaaaggccgcaaagcggagaggtgtaatggggccaagatgcagaccgtcggaaggggccgcctccgctggtctccttcttgagacagttctgggccagtggccccaaggaccctccgcctccctggggacgacctgtgtctggctccttgcccctccccctcccgggaggcctccgttttcacaactgttgacacttatctgaagaggcagcttgggcgtgactttggtggctctggtgcccgggggcagaaaatcaggctgggaggggccgcagccaggaagctccggttgcctcctccgtggctgccccaagtggactctgaccacctctcggcatctgaacttgcccccctgggcgggggcgggggtggggtggggcggtggcactgtggccccttcccccacagataagggtgattaatgggctggttgtgtttctgagggtgagttatgggcggctccagctgggtgatgacgttataaatgtcacctttttgcattttgtccccctctaaattgagttgattgcacttccccccaccccaaaataagaatctctgaaatctgcttaaaccatcagatgaggctggggagaggtgcaggtaattaatttccttcgcagtaatttattacctgctacgggacctgtgtctgagaggcctcgtctcagctccaccggccctcacctggggatgcgagggctccaagcaggccggaggggcgtggttcggacagacctggcccaagcccaggggtcccatggcgggggagtggccagaggaatgagaatttaagttttcctctctttttgctagtgagaaagcgatgaactgggttttctctctggccctggagggtccctcatgaagggcttcctggctatagcctgggacacaggtcctcgaggagggagccggcaggcagcccctggcaggtgtgtgagagcaggtgacttggggacccctctctgaggccaggcccacttccacccctctgctgagatttttctgcccaaatttaaatctcaggggcagataaaacatcaccagtttcaaaggacacactcaacacttgaaaatgcgcattaccctggaaagtaggtgaaaggactgtctcctttctgctggacgcccagggaggctgggccgggtctgtcccacccccaccccaccctttgaacatggctctgccctggacatcgtggccccaggccttgaacctggatgctctgcgtttccatttcctcctctgcaaactggagataatcagtactgagcaccctgcacgggcattaagttcggtaacaagatagcgttggacgcagtgcggggctctggggcctgctgaatggaccttagggcctgcggctgctgttgctgactaggggtaagggggctggtgaggccttgagtgtcaggacacctgcctccgaggcgtcgtcatcacactgcaggggtcagagttcacagggacgcacagacacctcagggtgctggggcggcccttgcaggagggcgctgggagaggcggctgggagcaggatgctcgctggccccgggtaatgaagcggatttattatctacattaccggctttataggagccagatggtggtgtcaataaactcagtaatcttcaggtggatacaatagtgagttaattaaatcgtgccgcactattaggagagaaaaatacatccgctctgggcctatccagtgtctcccgccttaaatgcacagatacacacctcggcgaagttgggcgtcattggtttgggtggggcatcgtctcctcccagcacaacctctgctttcaaaggggaagaccgcaggggtcccctcggctctaagggccctgcagggtctccttagtgggattattatgattgtgaatataatgccgacatctgcttttacacaaacatactgtcaaggtggcctgtccgtctccagggaaaaaggcagcagtgtgagtaggggctctggaccctggggcatgagtggggccctccaacagcctggtcacttcgagctgtgccccctctctctgggggcgcaggtcgagcgccccaggcctccagggagggccagcctcccgaggtcccctcgtgcctcagatggtgtcccgcgtagaggtcttgggggcccaggctgtgcagctgctcccctcagagcccaggggcccacacctgctgtgggcacgcagtttccaggtgggaccctgggcaggcccttggtcctgagagtagggagcagaaccttccgtcacttcccaggtctgcctggagcttctggaagccgcctgcttgcagcgcctcacctcccagctgcatatgggggtctccgaggcctgcctggtgagtgggaactctcggaggaggcagggcccctcagggaaggctgtgtcctccccatgctgtgtaagaagacacctgctgccagaaggagcgagtgggcgcctgccccagcctggggacgggcagatccatcagcatcccagctcgccttccacgcaccgacccctctgaggtgcagcactggcttcctgcatcagacgctgacaccacaggggtggtatgatgggggtccccggtggggccaggcagcagcaggcaccccacctccagcctctgccctgctccgacagcaccttctaggtcgtgctgctgtggggctgaccatgccaccctgtgcttaggcttgggggccgggacgggtgtgaggggggaggcaggagagcagacaggtgcacggggtcgaggagaagtggcattgggccatgcctgcccctggcgagcctgtcctctacagagcacagccctgtgggccaggcactgcaaacccgcttgctgctgctgtacttattattattattgatgttgtcattgttgtttatagaatgcttgcgttacaccagcaactttatgcgcatcaactcatttaccccagtcacctcgtacgagtattgctatccccacctctctgtgtaggagactaaggcacggagagtttaggtgacttgcctgaggccatacgattccgtggcatggcagcctctctgatgaatataatctattgtatataatatatatatgtatgatacccacaataaccattcagaatagctactatgaatctatcaatcatacatagtaggaaattaaaattcagagagacaaagacacttgcctgaggtcacgtagctagcaaagagcagaattgggatttcaaataataaagtttgtttctggagcacaagtgctgtgctggtgcgtctccatctgtaactgcatctggctggagggaagctcacaaacttcaggagagaatgtgtcacaaggagagtacatttggagaaggactctgcagatcaaagaaagggtcaaaggtgaactgaaaaagccattccgtgcttctcttttcctgccggaaaatgaaaggatatcagccaagaaataaatcgcatgtgtaagtgacatttggtgtcatggccctttgaacggcttcaagtgcagccggcaggactccaaagcaagtgctcttgagtgctgtgctcccctgtctgcctgctgtctgctctcagacacagagccggagcgaatctggacactccaggaatcgagttcagagtacgctgaactgtggatacagttgagttcagagaaaggcaggactggattggcctggagtaattggaagtaggtgtatcggtcagctgttgccacaataatgctgtgtaacaaacactaccagctcttaaacaagactctctgattctccctcacacgagtgtgagttgtctgatctaggctgggctcagcggggcttgggtccgagctgatggttgggtccagttggctctgaacagtctgtcgtcctccttggaccactgggctgtctggggcgtgaggtttctgtggtgagggcagaagaagcaggagaaggcatgcaatcatttaggcctcggctcaaaggggtacactgttgctgttgcccacatcagccactgctgaccgtgtggccactgccaacctcatggggtgggaaaatgagctctggctctcccgggagggatggggtcctgtggcagaggggatggaggtggggacagtaatgcagtctactgcagcaggcttcctggaggaggtggcactggagctgacacatggccggatcccaggtggcaagctaaagtgggagaatcacttgaggtgtgtgatagagaatggagctcgtgggaaggcaaggaaagtgcgagcatagaggaaaaaaggaggataacataaaaatacaggccctccctcttgcagccgtgtctagcgggcacagcttccccagcctcacccatttccatcgatgtgcccttgagaatgaccctctcctccttggagggaggctctgatctttggttgacagttgccagggacaggcggtgccaggtgggtcgtgggaggtgtcctggttcctttgttgggcaggattcgggagaccctgcatccgggatgagactgaagaatggcccggggcagcccttcctttgggtgacgggtagtgaggtggaccaggacagtggaggagggccaggcttgaggagggcgaagccacccagcccaacctctagcactttttagggctttgctctcaggccacagaagaaaatggaggaaacttaaatacacgttattaagtgaaagaacaattgttcatctgaaaggctatacaactccgtctgatgccaactacaacatcctggaaaaggcaaaacgatgaagaaagtgaaaagatcagtggttacggggggtggggcatggatgaataggcggggagcagaggattgtcagggcagtgcggatactctgtatgatggtacaatggtggacacctgacctgacacatttgtccaaacccatagaaggcagaccaccgagagtgaccccgacatcaggtgtggagttcagttagtgatggcgtatcgatactggctcatcagttgtaacaaacgagcgcactaatgcgggatgttaataatgggagaaaccgggagggcggcggggagagagggcatgcatgggagctctacttctcacttatttttctgtaaacctgaaactgctctaaaaaataaaggctatcaattttcttaaaagatggttttcaaaaagtggcatatcgcatgcacgtgtacactccttataataaaatacgcatgtgagggcatacatatatgaaaagattggaaggaagtgtacagaaataataatgtcactttgctgtgatataagggatgatttttttaaattaatgtctctgtgtggtattttggtcatgacgtatattgacttacatgaagtgctggacgctgcctggcagatcactggctggaagcgcggaagagctgaacgctgcagtgggaatggccaggccacgagagagccgtgtttgttagattacaaactccatgtggtccaggtagccccatcctactttcatgtggacgcccactgtgctgtccacccggacatctggctctgcttccgggacgaggaggaggggtcttctgcccacctgtgggtggatgagccgccaggccagtggcggtgagggacgcaggccccgtcacctctaggcttagcagctggttgccagtgagactaccctccagagctctctgccctccaagcagcagccagaaagttctggatagaccccaggtgatttccgtaatcagagccaccttgtgaatcacaccagacttgcaggagaaatacgacttttcttcaggcttcccagttttggaggctgtttgtggttacagcgggacctcactcaccaggactgattcgctttcggggtgtgcctggtgatctgcccagttctgaaaagtcacaaaacgccctccattcattcattcactcactgttcgacgaatattcaccaagcacctactgtgtgcgggacgctgtcccaggtgctggaggcacgaggttgaattccaccaggtctctgcccgaccacccgcggttcgggctcttctgagacccaggtcacccgcgcggtggcctgcacagcctttgttctcgtgcccctggtcccctggggatgcctcgctgcaacgcttctgccagtttctgctcctcctcgtgggatgagcttccaagagcctcttgggctggttgctctgctcactccaggcagaaatgccccgtcttgccatgtgggcttggccccgtccctgcaggttggcgattcgaaggaggcatttttctggacaattcctccttttcatcccctatgacttacagggggaaagtgtgaggataacaaattgattttggccagaaagggtgggattcttaatttaacagattcagtgtgagtgtaccgctttcttacttaataatatttccctctttgaataatgcgctccttcagcaccagcctctctgtatgccaacgggactcattagtccacctgacacccacctccatcagccagggctgcgtcggtgaacggtttcttcttacccagttaacaagatgtttctccagtatagtgttgataaattaagtaataaaaggcaagacttattattttatgttggaaccttgcctctgttatttctgagcaatggccaagattcagagtattagccgcagaaatgtttgcgttgccagggatggaaaattatcacatgaaaagagtctgatgtgttcatcacggatcagctgagaacaaggggagagattcaaaggctctaagaaacaatcgctgtcataaaaaccggcttctccagactcggctaggcgtgaaggggacgttgtcagtgtttccttgaggacaccatgtgtgccacaaccaggccagcactttcttcttgcctcccagacctgaggcgctgggttcccaaggccccgaaagggcagataattatcggaagagaatctgaagcgtgggcaccccagagagctgcaaaaataaaatgagggggaggcattttgagtacatttcctccctttgtatttctgttttggacaagaagtgcagaggcaggagctcagggagctcagccctcagggaggcctggcatggcggccgccctggctaggggaggctggagggaccaggggcatctttgtcccgtgtctgcccacgtgcagccctccaggaggcagaacccacccggggagaccagtgtctctccccactcacccaggtcaacgcaggcactggccggaaggtcccaggataggggtttacatgaggtgccacgatgcggggagggcagtgccagtgccgagggcacgatgactgtgtgttttgtgtgcagtcaggccatgttaggccaggacccagggaggcagaggccacatctgcaagctccctgccatgtccccaagcctggcggaggtgtcgggcttaggtcccatccggcgggtgctgggggcctcacggcagccctgtgaagcagctgctcttctcccctggggttcggacaggtgaacccacccacctaaggtcctgcagacagtaagtgacaaacttggccctcggaaaacaccccagaccaggctcttagacgggaggccgtgtcctgggggcccctgcaaggcattttgcaggatggccagccagctcaccctccgcggtcctctacttttccaggtgtttgaaggggaagtgtgtgctccccagaaccaggcgcctcccctgggcctgggctagcgcccaccacctctccagctgggtttcactggttgtctggctgtgtcccgtcttgcctcccattagcacagcaaatgctacccatgtgtcccttattaaaagcccagggtttttctttcatgatcaccttttggctctaaaagtgtcactaaaatggtcctcaagacctcgcccttctttgggcccctggtttcccaacatgacacatcatttgcctccttggccgtcctccatgatgcggtgggcggggaactttgtgtctccccggcactcccctcacggcccccattctgcatcctcactctcaaggggagactctctctcctcttcaaatgggataaaacagccttggctgcatgaacctggcaagggccctgccccaccgcatccaggggagcgcttcctgagcgtccccgattcctgtggctctgagggctgggccccgcacacagcttcagaggattcgggggcctgctcagccgtaaccccacaggctgcctctccacttggcctcctctgagccgctccagcagcccctgaaggcccgtctgcaccacttcctgcggacccgggagatctggtgtaggaaagggctgcttggaggcagaagactctgtcccaggacttctttatcctcaggtaccttgatttcctcatctgtaaaatggagataatagtagctcctgcctcaaagttgcagctagtgattgcatgagttacaatgcataagggacccttaaaatagcacctggcacatgcttggggctcacagtgagaccaaggtgatggtggtgatggtggtgatgatggtgatgatggtgatgatggtgatgatgatgatggtggtgatggtaatgatggtggtgatgatggtggcaatggtgatggtggtgatggtgatgatgatggtgatgatggtgatggtggtgatgatggtggtgaaggtggtgatggtgatgatgatggtgatggtggtaatggtgatgatgatggttgtggtgatggtgatggtgatggtggtgatggtgatgatgatgatggttgtggtgatggtgatgttgatgatggttgtggtgatggtggtgatggtggtgatggtgatggtggtaatggtgatgatgatggtgatggtggtaatggtgatgatgatgatggtggtgatgatgatgatggttatggtgatggtgatgatggtggtgatgatggcaatggtgacaatgataatgttggcaatgatgatagtgatggtgataagccctgggggtcccattcccattacatggggaactgaagagcccaggagaggacgtccacagatgggtcagaacaccacaggaactccagtcttggctccaccatcaactggtgtgacctggggcaagtcacttaacctctcggggccttggtttccgacctggcaacacagggcagacagtctgtcccccactccagttcctggtctgggaggaccctggagataaggcccagagccgcccggtggccaacacctcacaaacatgaggctgcactaaaagaaaaaacaacaggaagagacggaaagaaaactcataaatgtccctcaccgctggccctctgagatgacctctcacacagagaagcaggccttggggagttaataagaaatacaggacttcctgcgagatagcaagtgaattgtgagccgcgaattacttttttcctttgaaaaatgatgtagtcaggtcaaaatatttttgcatttcaaacacttgagttcctttgtccttggggctgacatgacttggtttccatgcagcctcacaatgcgtggaaacatcttttaactgccagacggcccttccgataagcccgccaattcataatgtgagtctggaaagctggaggaggtgagaaagcagggcctttgcaaaatctgtttgcacagcagcaagaggccttttgctccctctggtaaacagggcccggagtgcaggctattgagcgcggggggcctgggtgggggactggtacacatgacatgacagccgcctgcccacccacactctgcacccagccagacctgcccaggcgcctctggagccagactcccaggtgatgcgtctgcccctccagtgacacggtgggggctcggttctgtgacagcgtgggctgcctgtcaccgaggcagagtctgagagaactggcatctccttcatagcctgtctcaccctccaccctggtcccagctcagaaatgtccccggtggggcgggtttgcccagctgatggtgggtctggactgtggcctgcttggctccttggtcaaagggggcccgttcggctgctgttctgggccacaaggtggcaggatagaagggacagtgctatagacccctgtgaccccttccctttccaagccacatgccctgctgcccggaagctgaagggaaggctcccctgcaggtgttctctggggggaaagactgagaatgaatctgctcacctatgtggctgtttctttgggcacttgcaccagctgcagagctacagagctgtaagggaagaggctggctgtggggccacaccctagatcaatcagtgcccgttctacaggtgaggaagtgaggcacagggaggctaagtaacctgcccggtgtcacacagctgtgagaggtagagccacagtttgaacccagcttgtctggctctgtcctcttagcaatatgacgtgatgcttctcaacaagtgtctgtttgctcaacaaatatgcgtttgctgacccaggtgggcccctagaccctgaagacccagggttcccttcttcttccatgagactcaatgttattctgttagcacgtgggaatcggggggtcttgcaaggcgctgacagttcatggaatcagaggtgacgtggatccgcatggcgtggttctgagcctccccgggtgccccgcccccatctgtctccactcctgggcacgagccgctcctgaccacgctgtccccaaaagcgggtcataagctggaggctaacctccctgccgagggctcacgaaaggagcagagcttgacaagagccacagagacaagtcccacggacacatgtgctctccggggcgcagcaattcagtgaagccctaaagtgataatgacggccccataataataatgatgatgtaacagataccagagtgcactgggcttggtcctgtgtcaagggccgctcagtgggaagttggcctcactggggctccgtgggctacggccgccgagttgttggcgaggcgtggaacgggttttgaagctctttgccctccttgagaatgagccactggccccatggagtgcacacttggtgggggcataagaggacggtctgtctgggtatgggagagctcaccaggccggcagaggcagagcgggcgcactggctcctctgggcgcagggcctctgctcgggggtcgatctgcacacactcaagggtctcctcggaccctgacgttgctctacaccgcacgtcaccacttctcccaccctgggtatgtctcccagcaccggaagctgcttctgtttggtttccagttgcgttgaaggcccagggacgggaagtcccttctgagaacccagttggatgacgtggaacaccatgacgatcgattgtgtttgaggctcagcctcccctctcctgcccgactttgttgctggccttgccccaggtgtgggaaaagcaggtccagccaaattctggtgcaaatatgtagaggtggctggtctcctgcaaggccccatcccggccggtccagacgggaccacctgctgggtggtgggaattgcgttcccatggcgctaggccacctgcacagagtgcgcatgccacatggccgctgggcccctgtcccctgggcctgagggcctgagcttcctcacctggcttccgccaacggagcccccagaagaaaccagccagagtgcagccaccagtgggtccccaagcacttgcatcccgtcggagaattcactttgggacccctgggcagcacagcctgtgtgtctgagctcagctccctgctggctctgggtcacctgccccgcggacactggctccagccagcttcctctggggacagcagccgtttcactggcaaacccagtgctttgctccagagagagcctgccaggacgagatggatattgaaggaagtatcgccttaagtaatttaaatatgaaacacagagaagtcttcaacatcctttggacaagtgacactggtcaagtggcagaggctgctgggatatcagcattcattcattcattcattcatccactcattggttcagtggacaaatcccacaaagccccctgtgccagcagagagggagtgagcccacctgagaggcccttcctggtctgggggccctgtgacaccttgctccgaacccttgccctggggggcggatcatcctgggagggtgagactgggagctgcagatggttaaggggagactacggtgaaccctggctagggggacgcacagtctgaccagcccagcgcactttgtggtcagtgcccactgaggccccagggacccgtggccagtgtgggcctggggccttccccctcggtgcccccctcgccacggcgtggaggcccaccctcgtgctgcgcccacctggctcagcagccacggcccctgctcctccccacactgcgctccaggccgttgctccagcaggccaggccccaaggtcactcctttcattaggtccccattgtcttgagtccagccagggaggcgcgcggcccgttcgcttagtcagcgagaactgccctaagctccaggcgtcataaatgcatatttaataaccattttcttcttgctgcttcagaagttcagaaacgagcctggcccttcagctggtaatgagaagttaataaaagtgggattttgtgcagcactggccctctgacatcttaatctttattaaagagacgtgaacttgcagggaacgggcgggcctgcccggcacagccggacaccgggagagcggggcagaggtggcccgaggacgccagcaccccgaatccaccagaacgatgtaacgggagcttctcttccctggcgtcgctccccttacctcagctgcgtgaactcactgtcctccacgaggctcggagcggccggcatgagcggagccgtcccgtgcagagggaagctgatggctcgctggccgcacacgagctgggctgcatggggcgtttgggggccgcctcccgctttctcttttttattgaggtgaaattcacaaacatacaaatgagccattttaaagtgaaactttccatgacatttagtacatttgtgacgtgcagccaccagctctgccttcctagtcccgggacgttttcattaaccccaaggagacccgtgcccactggtagtcactccccagccactgcagccgcccctctgggtccgtctccatctgtgggtttgcctgctcttggcacgtcgcgtaaacgcagtcacagcgcatggccttgtgtgtctgaatgtcttcactcagcatcctgtttttgaggttcatccactttgtagctcacgtcagggcttaattcctttttgtgcccaagtaatattccatggtgtagaccgcccacaatttgttcatccctccgtccgctgagggacgtgcaggcggtttccactttcgctgttgtgaatagtgctgctatgtatatttgtgttcaagtattggtttgagtctctgttttcagttcccttgggaatgtacccaggagtggaccgctgggtcctacgggggctctgtggttaacttttgggggatctgctggctgtttccgcagcagctgctccattttactttcccgggcatccaggcccttgactcagagttgctcaaaaacaagcagggcttcacagagtggctaaagaattcgcccccactaagctgggtgacctgaacccctgctcctccttctgtaagttggggtggcgcaggagaccgccctctgcatcagtgaggagggggcacagacacagagggccctgctcggcctacagggagccccactcatctggaggatgaaggaccttgacgccaagtcagaagcaaacatccttacaaggaggccagactgtcgtctcaggtcctgcttagactggcaccgggtttgcctgcagtttactgacggtgtccctgaaaaccagctggcccctgacctaaggatcagccagcaggtgcccatagccctgctggcctgggccctaggccctcaggtcatgaccaaccatggctcccgtttgcacacacgtgtgatcacgccgtgagcagaccctcagtgtgagcccacagccccactaccacgtgtccccaggaaccctagggcttgagccagccgccccgaacaccaaaccccaagaaggagccgtgtgacgccatggatgcagcatcggaggtggggcacacaggcgcgtgtgtttgtcatccgctaatccaccaagagaaggagaagaggagagtctttgtcagtctccaagcaattaggataattaaaaaagaaagcttattgggtaaacatgctctcagattgtgaccccttaattaatggactacggggagaaattacagcagtggcaagataattaacacatgcatttagaagagaacggttagacggtggtgtcagaggacaggacgcccaggctgtgcagggcaggctgtggtggtgaaggagtcctgccagggacacgtgactcgcgggggccagcttgggccggcaggcagatccgttggccccggccacctgggcaggtccgagaaagcctggcccttcatttccgaagtcggctttaagaaaacaaaaacccatagtcccagcagaaaccacagccgccgcggtgctcggggtagagaaagggagtgtgaaccagagggtgctcctcagtgggcctcctgcctcccaccctagtccgagagcccactggcgccactagaatgttctaacattcctagaacatcagaggggcagggctggcagtgatgacctggcccggggcccccagccccacccctagcctcaggggaggggagtagggtgcagccatccgctctccatgcaggtccaattctggctgctctgttttcatctcggttccacccggggttccgccctggggctggagggggacattgcctcctctgctgggagggaaatgaagaccaccaactacttcgacctcctccataggggcaccagcccggggggccgggccctgccctgtgaagtgcagaggccctgaatcttcgggccccaggacctgccaaaggttcccatccaccagcaggcctttctcgcgaccagccacacctggaccctgctgggcactgcagatgggggctcggagatgggtctcagacgtgtgtctcgctgtctgggcagggaacagaagacacacaagtgcacatttgggtgagctgtaggaggaggcccatgggggcacgcagatggccggctgctgggcttgtcaaaggtcacgactgagcttgggacccacagggccagggaggctgtgggccgagggccttgatggacaggcctgggggcagagggcatgcggggtgcctgcccggccaccccgggtgccggaaggacaaggctggaggagttcttctaatttgtaagggaggccgcagggcatcctggcccgcgtgggtcctgggagcctcgaggtcagaattaaaaacccagtcctctcctaccagaggaaggggatgaaaacgttccccaagttgtggaggccgccccctacggctagacagagccccgggggggagggctcctcctgaggtcgtagctgacgccggccggtccctggagccgtgcaggagaggattcgcccagacggcctcttgggtattcatggaagacccttctgctcctagcagagcccccaggggtctctcacctcagagaacagagaggaagacagtggctaaggaaagtttggggtaaagagttcactctcagttcactgccaagcaaatccaagcacatacataaaatggtggcccgtctgagctcagcagcgagaagctgtggtcactaaggacccatagctggcccagggcagagccaggcccgacccctgcaggtacaggagagttgacctcagaactgcccttgggccataagtcagtttactgcccattgactgggccagaggactgaggccgggagggtggagccgcctgcccgggattacacagaacctggggtgctggcacagagccaggggcgtatctttcagctctgggtctggggcaaggccaggctgtctgtccaggtcagctggaggtggttctgggtggcagggaaagcctgctcctgcctcagaggccgaggacaccctggcttaaccttggcctgagtctgcccatctctggccctcaggagtcctagggcactggcgcccgcagccgcactgctttctcggacccctccacaaacactccgccctcagcctcctgtcctgccagtgggataggcttcactaacggtctcctaggtcctgccccagtcacacgacagccgctgccacgggacgtggttcctgcatgagaaggactctcctggccccaaaggcacccctgcctgcgggtcagctcttccaggggcagctgtccaaccaggggctgcctaggttggggcacagtacctttgtgggttgcaggaggacagcagcttccagaagcttccagaagcttctgaagatggccagtgcctaggaggtgctcagacccaggggaagtctggctcacagtttcttcagggtgaattccatgaggactgaggttccgaggtccgggatcctgttgtcgtcttctgtctgcaggactctgcagggccgggctggaagtgggaatgagggatccgagtgcacgcactgcaggttccgtcggtggaggtggcagctctgccaggcgacacggtctctgaggggctggacctgtattcacttgcggttctcaggacacagaggaataaatgctgcccagggtgctgggctgtcccctgagcacgtgccggtgtcaagcggccagtgccgttggtctccctggggccctgctgctccgtccacactgggtctggcccgtggggggagcaggcagggcactgggctcatggcaggtgcttgggacacaccgcctcccggggctcagctccctggggccccccgatattcacgcttcacctgagactcgggtcggattttggaagcactgataggtttgctgcgcgtcctagttttgcatcagttgggggagatttagaagctcccaggtgatgctggatctgtgggagacaccaagctggggccccacggccgctgcgtccccatgggagtgaaggccggcattccaggcctggaacaagatgggcagggccgcggcaccctccctgccctgtgcccgctaagggcaaggaatggcttttcctgctccccatgccctctgggtggtggctgtgtgacatgagctgatgaaggggacagtgggacccatggagtcaccactctgccgctcccaggcctggcccctggggcgttgggccaccctgaacctcaggcttcccacctgccgaatagcggagaccgtgctgtggagctgtgccacttcgtctttgtggcttgtgccgaggaatgacagagcacacagtcagtgcacaaacgggaagccctcacagtggggccaggacctgcgtttcccctgagagcgtcagtgtctctcctccacgccttatcccccaaccggacctgcagggtggcccctgccccaacgcaggggaccccacacacagggtaagacagacttacagtcaacagggcgtactcagcagcgagaggggagaccctcagtgttcctgggataagccttgtggccccgccctctagactgagtccagggtgagacaaggtctccctgcatccgtccgtctgtctacttgcttattgaccacacctcgcatgaggcctgctggcccgtgtggccctgccctctccgtctggaggttcacactaaatcagatgatgtagcaagagctcaccggtgctggagggcaaagagggagcaaccaggccatttgaagggaggcgtggggcaggggttgcagaagtcttcctggtggggaaggtttgggcaggatctaaaaggtggagtagaaaaccatcagttattgtccaaggaagggcgtttccaaagagggaacagaatgtgcaaaggcccagcagcatggaatagcatgaggaacagcattcaggaagttgcaggctgttctgtctgtctgaggcttagaggagggcagagcaatgggaaatgcagcaagagacatggtcaggggccctaaatgtaggcagtcggtgctggtgaagagtttataagtgggggacccctgcactgagcacgttgtggcattcccagcagccatctgacctcatgtgttctcatgcgacctcatgtgtgtgtgcacgtgcatgtgtgcacatgtaagtgtgtgcacatgtgcatatgtgtatgggtgcatacatacatgagtgtggatgggtagatgtgtgtgtatgtgtgtgcccctgtatacatgtgcatatatgagtgtgtatgcatgtgtgtgcgcacatgtctgcatgtgcgtgtgggtacatgcatacgtgagtgtgggtgggtagatgtgtgcgcatgtgcatgcccctgtggacatatgtgtgtatgagtgggtgtgcatgtgtgtgtacgcacatgtgggttgggtgcgcgtatgtggggtgtcatgagtgtgggtgagtgaatgtgtccatgtgtgcacatgtgtgcgccccgtgcacatatgagtgggtgtgtatgcatgtgtgtacacatgtgtgcgtgtgcgtgggtgtgtgtccaagtgcagcccctcgccgtggactcagcgtgcggagctgaggggaaacagtcacgtcaatctctgattcatgaattaggatccaaatttttaactaattgagtacattaaatagaaagtccctaaaaaagaaagaaagaaagactaatacaagacacttttcaaagcttctttaagaaaaagtgcattgggctttgaccgtaatggctatcctcgcttgccaaaacctgacgccctggcaggtaactttcatcacgcgtatctgaaatggtacctaaatgaattagcaatgaaacggactcaggccgagcagtggggacctccattaatctcgggttaatcactaaagaggtgccattcacttttcttaagaagtcacatttttctccccttgctggcgaccagatttcataaagtaacatatttctaatgaaaggtctctgcttgaattaaaccctgaaggtttatgcagattttgcgtttgattactgcttggcatgggcccgtagccggctgtggaaggaaggccctctctgaaagggccccagcgtgcagttcataatattctttgaaataaaatagtgcacttgtgcttagtggctttaatcagcgctgacttttgatagtcaaacaatagctaattacagtagcacctgccttccaggccctgtcacgtcggctcctgccggagaggggccttgtctccgagctgcgtgattattaccgactccctgcacaaagtcactataactgacactcagcaagtttggtagccattaggagaggtttattttgctgccgcgttgctggcttttccattacattcttctcctttgtattttggggagtggtgatggcgagggcagggaggagagagaaggcgcatcagggcccggggcccaggaaagccagctgcaggacaaacggtgcagaattcacaaaaatgtggtcagtgtgctgctttgggcttgagaatttggggcgttctgaatgctggagagcccagagcgtgccctgtgtagccggcgaggcggggacccgcacgcctgggcccgggcgccgccctgtcttccctgggcctggacggagtgtagtttcaaatgaaatgcctcgcgatgcgtggggagcgggagacgggccgagtggaagtcataaattattatctcgtttaagtctttcttttctcattaattttgcctggcaaatgagcccgaggtttccagaacccggccaagtgaggaacgtcagcctgtcagccttgggcttgacagaccctgtaacaaggtcctggaaacggacgatgggacgtgggtgatggagaagtggtgggcgtcccttctcacagctgcctgttttgggtgcccccgccccccgccctttgacaggacccccaaagaaaaacagagacagagcgaggaggaggtctggctgagcccgtcggtggaggcaggtacagactagcggtcattcattatgcccccagcgggcgtgccccttgtgacgtgggccccccatcgccccgtcctggcacagacagcgctcctctttctggggacagttttgggcacagcgggtggaccaaatgccagagttggccctgcccactttcagtttcctcagggactccatccggtccccttgaggaaggatgctgccagggctcctgggggccttcctgcagccctctgggtgacctttcactggggtgacatgagagctgggacgcagagccaggagggcgggaggggagtcgccccaaactaggagagccctgcacagccggcgtcgcagttctggccgctcccagcgaggcccgccgtcttccttgcccagggatcagagcttccgaggtgctcctgagccatctgcgcctcagccaagacaaagttctcttctgcctgcggttgggtggcgcccgcggagtgggcgtgtgcacacgcgagcacaggcgtgcctgcagggccgcgtcccgggtccctctcatgctgtgggtgccacttccgttctgctggctttggtctctggacactgtaccgtgttgacaattacccacagccaggacttgccaaaacctggagttgaaaaggcttcaaggaggaaaaaaacaactggaaagctgttttctgttaaggcaggtttatttttccttgcaattatgtctgactaaaacgtccttgtgctgaggcacttccctaagaaaggtctgattcatcctgccctggatgcgggagtggcagccccctcccagccccccacccgccctcggcctcatgttcctcgccaggagctctctgcggtttggcccacgatgggagacctggacccagagcagccaccccgtgatggcccagggaccaggctccaggtggagggagtgcctgcccagaaagggggccgcccaaagggtcgggaactcaggagtgaaggcaggctcgagcccggccagcccagagtcagccctgccgcaggagtggccttgcacagtctgccacacgaagctgacatcagccagtgaccccgactcaccttggcaggaaggctcgccaccccgactctttcgacgtctgccctgtgccatctccagcgcgagtctatgagaaactcaaagcccgggtcccagagaagctgcagagcctcgtcagggcggtcggtgacggccccagcacctgtccccctgtttggccgaagtctgtgaacacagccattgcagagaaaggccctggtttctcatttctcccttgagtggaacgagccccctacccagcgacccctctgacctgccgatctgatattttccatcccccaggctgaggccccctccgctgtcccagaggccggcctgggaatgggaggagaatgtgtgccctgacgggcctggaccatggtcaagttcagcatttagtataccatccttccctgtcagcccttgccccacccccagcactgacctcggcccctgaggctggcgtctgcctctcgtctctctgtgtttgccatcactgatcagtttgattgatcatctctgcggccacaagttggtaataaagctgttttgagagcctctcacatttcccccgggaccccattggcatgacaggacttgacatcagccattcgtcacctgctgcctgtggtcactgtgtgcaatgaccagcagacagacaggaaccaggccaggacgaggcttcaggagggccggcctgcagccagggtggcgtgggacagatgcatgtctgcctgccatcagaccccccggaagggacggatgggcttccagcctctgggccaggtgccacctggggatatcaccaagcaccatgagctttcctctccccatgggcacacagacgtccaccctgcccatctcagagggtggcttagaggccaagaggagcgttgactgggaaggtctgtgaggggccccaggagccccagctgcttgggtctccccgcccacggggcgcactcctaaggcgggtcctcctcaacaggtggctccccgggcgcatccagacaaaggagcacagtgaaggaaagcctctgccagctctgaccgttccttcttggggagaagaaaatgggtttatgatatgaatctgctgatgcccctgcagactacgcagggacgggtcagtgtcagcgtctcagacactgggacctgggtgcaacggtgcattgaccccaaatgaatccacagggtgcccaggccagggggttgagtccttgtttgcattacaccccttcacgcatgcatgtgcataggcacacacacgaacacatgtgacaccctccaaagatggtgcagcctatgcttctgagagctgtaggtgagcggttattgctggtgggccagggcatcctccgacccaaaaaggagagaggctggagcaggcgtttgagggtgcagatccaggactttctaacatatgtcaagatgctcctccagtcatcacagcatttccccggccccatccaggtgcccagagacccctgtctagccgatctttccatcctcatggtccttgcctggttctcaaccctccccacctgtggcagagaaaacctcaaggcctcacttggtctccctcgacacctcgattcctcccatggatcatctctccctggagatatcctttcccggggacggcctggtcctgcccccttgggccagcaccctgagcccaatgctgtttggctgacatttgggggataaagtgaacttccaccggcaggaccccatcagtagcccgcagagcgtggggggctgcgtgactgcacatctgaacaggacgttccccagcagggctgtggctgaccgcggcacctgggcggcgctgggaaaccggcctgccatgcacacctacaggaagcactctgggcagcgaaggaagcgcgtcgctgggacgtccctgtgctgggcagcaaacaccggcccgcggacctcgaggtcacatcaggcatccgccgcaagccaggacgcccagcggggactgacggccccccggctctggagctcatggccagatgttgctggagatgccgtgaggtgaggcctacccttaaggtccccaccctcagcgacccgtagaaaactgaccaccaagatcttttccaattgcgttgacaaattgactgatggtgcattttgggaaatgtgtctaagttcagaagaccactgctggtctgaaaacattcataggagggatggtgaaactgacaaagcacaaccccccccccatgatgcccccctctcaatggccaggctcccccagagcccttgggcggtgattattcagagtcccccccagttgcctctccaatccccccagtatttcctgccccagcggctggagctgcagctggtcctggagagaaactgggtgcacgtctgtcccccagaacgctgggtgtatggcatgctttctgtctgtcatcattggcctctcacgaccgttagggccccaggaggggctggcctgccccaaattctctctgtgctgcctttgcaaagaaagcgggtgtggaagctgctgccagagcagttccggacccctgaggcacggaggggggtgcagtggaaacctcccccccgcaagggagatggtcaccaggggccccgtaggcctgcctgtctgccccgaaggaccagggcgtctcagtggagtggagcagggcctcccccggccagcctgggctgaggggaagggcagggccctctcttgggcagatcagggtgcatggagcatgaatgagaggcaggctgaggccaggcccaggacacagctggatttgggggtaaatgtgctggtgtcagatccttaaagtgagaggggaaaggtagggcactcagggggcctgagctgggcaggcttcgccaggccagtgggagctacccccacgcagcagcccctgcacacccaccctcacgccctcatcctcgcacgcccatcctcgcatgcgcggccactggctggcttcttctgagctgctcggaccacagccgcctctcattcagggcttcctctctggccagttggagggaagagacacaagtaggagcctctgagaaagaggcctgaggtctgtcggctgcgcccttccctccactgagggcgagggaagaggcggctgccccagagtcgcgcttagactgggcagctgagagagcaggtcctgcaggagctggggagagcagagcaaaggggccagtgcaggaaggggcagggacatggagggagggcagggcaagcgggcctttgccccctgcccaggcccccacagaggggagcctgcccagaggcccatacactgcctggagctaaggtcagacatgggggcctccaagggaggtgagagggaagtcagcctgtgccctgaatgtccccgagcaacgtcacccggaaggccagccgctgcaggatcgacaggccgcctcctgccacccaccgctgatgggcggaagctttctccatgacgtcaccaggaaagcaggagctcaggacaaacctcgtgggatcccgagtctcttaaaggaaatattgatttatcttccaatcatgatagcactatatgttctttgtgaaacaatcaggggaaaaaacaagatggttgaaataagaaaatcaaaattacccatgattcctctgctaatagatagagacacgagttatcaggggagagacacacactccccatcagcaccaagctagtgcgcggccgcctcgcatggctgcatgtgtgtccgtgttcaggtgtgtgcttatgtgtacacaggtgttttatgtacacatgtgtgtttgagggcagtacagctgtgtacacacacgtgtacttatctgtctcgcacactcaccctcgcaatcacacttagatgtcgtgtcagatccacctggtctgcttacctcctcctggctggtgatgccctatggcattgcctgttcctgcgttttaatttctgcacggccatcggtggtccagcatcactggacagctggctggaatgatggccctctcaagagtcgtcacctgtccactggggctccctgtctcacctgggcagctgagtgtgggagggcgggaaggtctctgtttcaggctcatcccttgggcacagagggagtggttttccctctcgttctccccgcaccttcgctgccagcgtccagccccacatcagggagctcccatcatccccctctgtctcagggctgcgggaggctgggaggcccccatctctcaccctggctcccaccaagggccatggcttcaagaaggcctgaggtctgcaaacaggtggttagctttaaaaaaataaatactaataaaaggaaccttgggcaggactgttcctgggttaagtaggaagcaggtcgaccatcacaggggtctcggtgctcctctcagagcaggaggtctgcgagaagccagggctgggttccccggcgcacggggagtcaggaaaggtctgctcagggactcaggcgggattggaggaaatggatgggaaacaatgaagaggttcgagggtgtggctcaaatctccatagctggtttcttcccaaattctggctcccggaggccgagggccctcagagcgcagagcagagcggctggcgccgggctgtctccccagttcttctctcactggcagtttcatgttgggcgttgggccctaagcctctccaagcctcagccccctcgtctgtgaagtgggggcagtgctgtcagcctccgggctgccatgaagggggagtgagatggcggtgtagaacctggcacagggggcgggggtcctggagaatcgggcgcgtttggtgtcccgttgccagaaggatgctacaaggtgcctggctttaactgtgacctttgccgttctgccccactgactccccactgcctctggctgaggacgtgcttgtggccattgtgacccaggaggagggaccaggcagtgtagacggaggtgtgaggcctctctcctgagaaggttccctccccaccaactgccagtgtttagaggccttgaccggagctctcagccccactggccggagactcagtcttcctgggccgcggtcacagatcccagtgtctatctatgaaatgacgtagaagcgggagcgccctctgggccaaccagtaaaacactactttccttgggagaagtggcctcgaaaccttatcagccgtttatgccttggcatgatccgattggcgagttatgatctgattgattgcttatgagaggattgatcgattagctgtgattggacatatggattagctataacctgattgattagctctgttttcgggaagaggaagtcagggagaaggggtataaggattctgtcttcccctccagggagccaaagatgcccaaggaggaggcctggtttccctggggctggcatggagccaggctgggctcctgaggacgtggagaagtgagggggtcccagagggagccctggcccaggctgcagtcaagtcatgcacaaacccagtaggacacagtaggacacatggcatgcacagggacccctgagagcctcgtgattctgacatgggctccgtgacaggcagaacctggcgctactgatccccagagcacagcgggccacatcatggtgctcggcacatattttctgaaccactgcctagagccgtgaggaggaggggcggacacctcgggagagtgatcctggtgcaggttctccagcagagcatctctcggtggctccggaggcccctcaggacgcagatctgggcctcacacggggttcatgcagctctcgcaccttctgcgtggctggagtccctcggaggcagcccgagactctgttcacgcaggggcactgctttctgaggggcgccatgtgcgttccgtcatgtgaatttcatcgaccctgcgttttgccctggtccccggtccaggccctggggtggagctgccagctctctcctttaccaaggaagcccctttggacttgcaaatgaaaagcatctgaagctggtatccaacttggctccactttcctacctataaacaggcctgattcgttagttctcagaatgttctggaagaggaatgtgtttggggctggctttggcatgcagacatttccatggagaggccggtaagtgccaggctctggcactgaccctggaaacgcaggctggcctcgccagctgggtcagttcgggcgcctctgtgagctgggcggaggctccgggccccttgctcagcccccacacctcctcgacccggagacctgcccccggctctggggctcagggctgttgactggccacacccagcagtctggaccccgggatgccttgtggcccatggccggctctgccaggggtctctggtttgggaagtgccccaccccggtcctgccctgcgcagcatcctccacggctgaggcatcagtgtccctcgcaggggctgcttgacaagattcagcccacagcgaggagggcagccttctaaggagatggggagaacttacagctgagtggccgagaagcttggccaagggcgttgaggtccccctggatcccggccttgtgtctggaaaacacttgcagaggaggtgggccagcccgtgtgcgtctgagactgggaaatctgctgaccacgagcatggtcctcactgttttcttgctacctgtgtaattctcggtaaacctggacagatcccctcgttggggaggggacacgttctctccatctgaagcacaggggaggaggaggccacaggactgggggtcccggctgagaggtcggctcgggggctctgagactgagatggcccaagtgggggtcgtggtcacatcagggcactcgaatcgtggtgacttccctttcttttgtcctgtattttccttgatatcgggtcgtctttcagtgagcctagcaaagggaacaggtgacacgctgtagaccgagttagagggagagccttgtctgcgcacgtgggttttactctgtccctcccaacactgcgttttgcaggaacctggcccagtggtcgaacattgctgcctgttagcccctcagtgcctccaggggaaacgtctgtccaacggcgttgctgggcctgcggtggccggtggagcctggggccagaggtggacacatccccatgcccagcgcttctagttggtgcttcagtccctccctccttcttcccttcccagaccctggagggacacccacgcacgcacggagccccacgtgtgagtcaggatgagcggggagagctccatgcaaacccgcctcccctgggcacacccctacccactgcccgaggtagtagaagcacttccctcgagacaggggccccgaagtgctggggcagcacgggggagacagagggccctggggggcacggagggttagggggtgggtgccgccaggccaggcagccaccaagttctgagggagagcgagagcaggtttcccacctttctcccctagaaccttccagcaaagccctgtcaatggaaggccatcagctgagctggcaaggccaggagggtaccacatctgcagcaggcaggtgtggcagcaggagttaagttctgtgggtgacgctgggggcaggaaaccggctctctgcctggtagacacaggagcatctcaggactgaagctcgcagggcccggctggtggtagctggctggctccgcatggcaggcggctggtggccccaggggaccggagatgctgatcagtttgcctccctaggcctgggcttgggcaggggctgctgccaggcctcccggaagctggggtggaggcgccctctatcgcctgctcgggggacgtcttccccccactgcatgcccatcagccctggagcctttgcaaactgacatcaactccccgtgtctccccgacagaatctgccttttgggaacctttaaaataacgagtcaagcgtttccagctcccgtctgacttttaatataaaccgtgtggttcttgaagcgtgagtattttttgaagcatttaagtggcagtatttacctggagagggagggagagagaggcaaatggccattctcccgtggagtcagttttcatagaaaagcaatcgaagatatggccaaaatgccgagcttgtttcccagtggcctggaattctgtctagagtaaaagcatttcagaaatctccagaagcaacagagattgggggcgtgccacccctcccctccccgggggagctttcccttgcactgccttggtgaagcctttggaagcagggccaggggtcctcgggcctcttccgatggtggcagggcagccgggccggggtagagcagacggactccgggctcccaggcatttcccaggcagggctgcaaagcccagcctgacggttttccttcccatgttccagcctggggagaaaacttttcctcccaggttcaaaactgcctttgtgtgttcaagcatcatatttaatcagcatctttaaatgctttacggagggtttttttttttttcagtttctgaaatgtcagttttccacctgggggcaaatggatgatttttttttcctccacagcaatttgtttatcgttctagtgactcaccgtggggacagttatgccctggagcgtcgctctggttttcctgactaggcaggtggatgctggcgagacctacacatgggtctcgcccggcagaccagccgtgtgggcagcgctggggccccaagggcttgggaggggaggccgggtgccagggatgaattcacacagaggacccctcccaaggggtgggcggcgccgaggtccttcaccagcaggacagcgtgggagaaacaggttggaccggggccgagggagagggaagcatcagtgctaaaccggcccggtactcctcgtaacaacgagcctttcctgaaattctagattgggttctggagaaagcattgcgggaggcctcccagcttccaaggatgtcaagatgttgtcagacaagtcctgcatttgcggtcacctccgagcggctcaaacctccctttaaaactgcggatcccgccaacctccgggtgcaccaggatgaggccctgggctgacaaacagcagcacctctcaacccaaactgtttagatttatagcagcactggaaacatttccaagcacgtgggatcagtgttgttggctaagattttatgagtcccacacttgggaattaatttacagaggacggccgccgctgttgtgatgtggcagagaggagaccagcccgggtgcacctgaggctggcgttgggaggttgtggagtgctgtgctgttgtccaagtcttcatgggcgtgtcggtccctttccccttctggtgacaagcaactgggtgcagcaccttgttcttcacacagcttgggttccacggagagcccctcccctgagagaatggggactggatatggaccaatggtctgtagacagaatttgtggccttgtgatgggaaatcggatggtccatgctgtgccagggagggagtattgcaaacctaggcagaggggtctctgtgtgacccctcaaaagggcctttcccctagagaacatcagaggccacacatgggcctggggcagtgtcctgcccagggccaggggcaaagcgggaaagggaccagagcacgagcctggcacaagcagtccagtctgcagctgctcaaggaagcttctaggcctggtgcacgcaggtggcacgtgtgccttgccatgctggcccctgcccagccgtggaccccagggtgacatgctcctggacggatcttgaactttggcaggtggatgctggcagattcctccagccagtgcagtccctgagcacggagctggaagcccgggcgtacaagagtgccttggacggtgccggaggaaggcgagctggtgctctgtggcctccccagttctgggccaacccagagcatttcctggtgagatgctcccagctccagccggaacttctccccatggcccctggtgaggtttttggagcccactggacaaactctggggtcccggggtgggtgtagggtgtgtgagctgcagccatccagcaaagtctggctcagagcatcctgtcccctgtccccctggccccagccccacagggggcaagcacctctcccccagctggccacctggacgttttgattacctctccaggtgtcgctgggagcgcagctctaccctcctgcctgcaccccattgttctgcaggctctcttttaggtcattttacattttcacctgatccctgtaattctgcaggacgttcccaggccacaaagtgaccctgctgtagctgatgtctcgtccccactcacaaagctcagaaggaaatttctgctgctgtggcgctggccctgggcacgggtgggtctctggccaccgggataggacagccactcgtgccctggccttctcctccacaccacccccaatgtcagcgtcagatgtggtccccgcctgcccagcccagtgggtgcctgacctcctcccgcagtgctcctggggcagtgggtgcgacgtcgcaggagggtgtgccgagtggctgatggcaaaggcagggtgcccccacagacgtgggggaaggggtgcctgcctgccgcaggcgaagtcaggcatgcttccacccggaagagggtcagagcccactgcaggcgggttgggagttttctctgtgcccactcccagcaaatcaaagatccgagcagagagcccctgcagcatgacgccaggtaaaataaatatcaaaggaacatttgaagagctcaataaattgatcatgttccagtgaaccctcaacctcataaccatgggaccatttaaatgctaatgtcattttttaaaaagtcaatgacaggagttttgaaggaatggagtcagcatctccgcctagcctctgtccttgcgggcttcctcccccggcccaggcacgggagcacaggcccttccctgcagggaatactgctcaccctcaccgtgatcccaggaatcaaatccaatcaaggcgatacagactccagaaatatcccaagtcgtgtgggtaacggaaaacacgtgttgataaagcaagcagcatgtgtggatccagggcagctgcagccctgcggagggcacgacctcaccccaccccatggaagagatgggggcgggcaacacaggagccttccagaacgttccggagatgggtgccaggtttcccatcaggccctgctgtgcccccctccctcctcggtgggcttcctcgccgagccctctgggttccccagtgactgactggttcccgtgtggtggggaacggagaggggggcggtgccggccaccccgagggccctgggctcccgtctgagggctctgagtgtagcatcccccgcaggggcctggggggtctcgagagcatgctcacacctcccaacctcagttcctccaccagccatgccggtgacagccacctgatccatttccatatcggcagccatctgtgtgtgctggacacgtaactgtctgcaggcggaaggaaacaggcacaggggctccctggcccacacactagcgagtagaaattgctggaattcaaagccggtttgccaagggctgtgggagccacacgagcaggaggggatgcctggaagaccccaagtgtccagtctggtagccatgacaacctgagtccccagggaaccagctggtctgaggctccaggaggatgtgatgacctttccagcttttcgagggcttggcgtcgagggctgacctgtggctctctttccctcaggccagcgcttgggcagaacgagcaggccctggcggtcagctctgagcaggtctctggacatcccacgtggcccaggctccagggcaggtcaggagcagccgggatgcatttctgctcctttttcttgctcttctccaccactgtggcttccaaaagaaggcatcctccaggctctgctccccaagttggcagggaaagcccaccccttgggggtccccagtctgctttctgaaccccagggtcctctctgagaaagtcgaggtgaccgtcccatgcagggggttggagctgagcccgccaggctccctggtgcaggtgccagtgcccggagctcccccagtggcagtggtgagactgtgccaccaccactcggtccctctgctctcctgctgctggtgctgcacggcgttgagcggttggagccctcgtggtgtgggtgagagagcccagaggggcccctcccttcaggagtcaggtcagagaagccaggtcagccctccccaccttccttccccgggcacgtgtgctgggaaggagcacaggccaggttggcgtgcagggtccaggatgcccctcctcggctgacgctgcaggagaagtacagggctctcttcctgccctgggccactgcccagcacatttgggacttttcaagcttcagaagttccattgcggggaggccctgatcagggagagctgacatgctatccctggggccacctccttgcagggcctcagggcagggtactcaactggcctgccgcctggctaaggaccccaatccctgcaggggtctggtgctgcagcagggtccttctccctcatttacgggtcactcgctgcagggctcaggtgggggaatcccccaaagggaagggaggcggcccgttcggtcatgggggaggaacctgactgggtcacaaggtggggaggggcggggatcctgggagtgtgtccccgagccccctgccacaggacccaggagtacgcattaaacacactgcatcctgtgcactgtgtgtgtgagcgagggtcagctggagaatctgcaggttagcgagccccccggagtcctaagaccctccacagtctgaggcccagctgcctcgagggagactgcaggcagacagaccccaaggacacccagcttttgggcctcgttttaggcatctgtaagatgggtcacataccgtgtggcagtcagagtgctaacgcctgtgcacctgaaatgcctatagatgctgtttacagactcacaggtgcgtgcccatgttgtgcctccacctgcaacgtggacccctggcccccacctgtctctctgtttggtgcttcctgaactggccctgccttcctcccacaccacggtccacgctgcaccggggctgggggctgagagtgagggacacctgtcctggcgccatagtcagcattcctatgacgccaacggggcgccgggctcctgagcttggcccccagcactcgtgcagggctccccaagagggcggggctcctggcatctgagagtagaaataggaacgaataaataaaagatcaagcctgccagccccgcacggcttccagctccctgcagtcttccttgtgatggcctgacacgagagggggtctcagcatccccatttcacaggtgaagaaaccgaggcaaagagaggccagaacctgcacgggttcacacagagccagcccaaaccccagcgttcccagacctgatcgtggcctcagcccctcactcctgctcttcatgccgtttccgagcctgaaccccactgagtccagcagcacctctgtgacggagacagaaggacagcctctagacagccttggggtgaccgtcactaaagaaggcgtgtggatcccaccccgtcccctgaacgctgtcccttctgatgacaaaccgctggacccgagtctgctcggcactggctgaatggaacaccatccccacagccacagagaagaattgggccccaggaggactcacggcagaggaagaaacccaagatgggggccgaggatcgcaaaatattgcaaaattttccgaggcaggccctattagatccctgaaatgggatacgcaacctgaacttggccacataatacggctctgtctgggacgggccgggagcgccggggacccaagcagtctgtcaaagagcgatagttacggaaattagtggtgcagaaatccgtccccatcattcatcaggggccttattaactgtcctccgacgtgataggagcggcctggtctcctgacttcaggggcctgggagaggccgtccttgtcctcagctatgaggagaggtgcgggaggaagcccaggaagcgctggccgcatgcacccctggggtcctggcagtgcaggtcgcagggtcgtgttgtgagcattggaagtcatgaggcagggcatggacgagcgcgtctgcggcagagcatgacccatgcagccttgttggggcagtgataacacagcgccccagagaggaggacagccccgggggcaggcaccccagtggtcagggtgtcccctcggaagggcaaactgaaggccacagggagccctggagaggccgggtcacccgctcacctgctggggggggtcccacctccttgaacctcagtatcttcattggcactgtgggaattcccatgcaggtgaaaggagttaacaggaaagacccttgaccctggcaggacacccactcaacatcatccgctttgtcatccttccagcagccggcacacctgggggctggccccagggtccacaggctcctcccctgatgccccatctcctcatgtagacggctcaccagccagcgtgggacaccgcgtccccagccacctgctgcccattaatggaatgagggcccattttaaaaagttgtcagagctgaaagcaccaaataaatgccgtgcaggtgacgggtcgaggctgcagctctgggcgtcacctgggaacttgtgagaagtgcagattcttggggtccagcccagaccttctgcgtggagccctcgagggtggccagcatacccttaacaagccacgcagttgtcccatgcagtgtgagaccctccgcaggggacacagaagacaaggctggttccagctcctgttttccaaggcttttctctgggggggggaacaaaggcctcccactgctcgggacgcaccagggagctctcagcgcccctcctgggccgtctccccaccccacagcgggcacgttcccaccggggctcccatggggcctgggccccccagcatgcccctgtgtcccgccggtgtccggccggcggcgggagcccggtgtgaaggcgcttgctcatttcagaaggactagctgagaggccaaagtcttgggccgaggaagccttggcaggatgcttgcctttcaaggtcgtgctggtgaccttccgcccatgacggatttattgaggggaaaacatgttgagttgttttgtccaacagtatcgccagaaggaatctcccgtggccaggcctcaggcctattactcaacactcgcattattaccccaaaaagtcagacaagagagacagagagagggagagagagagggagcccgcggaggtgataactcatcggggagggggcgcgggtttccaaatctgttcctttttagcccctgtgaatcaaagaagcgtccggctctcgggggcctttgatcctcctcccgagcccgcagtgtctccacctacggagaaacagccggcgtggagagataaacggcacttgagcgtcgcgctcgcccgggccgcaccgctatctgcttccccgagtgttgaaaagagctctcttggcgagtagcgcgcacgtaaccttcgcccttgaccgcgctgcctggtggcagataaaaagttgggggtgtcgagaagggaaaaggaagggaaaggcaatctgcccaccccctaaaagaaagatgaggctggccggcgtgggaaggctgcgggcttgctgcagagctgggggcccctaggaggcaggaatcggggggttggtgcagagggaaagacgggagctcgtctgcacgtctcccaccaaaacatcacttgggggatcaggaaacaaaccccacaatcaagcgatgaaaaatcgtaccccggagtgtcttcagaagggccgtggtgggcctccagcagcggaggggcgatgcagaggaggggttgaggttttccggctcttcttgcttgaactgtcccggacggaggcgtccggctctgctgcatgaactcggcctttggcacagcctcagttcgactgtctttccacagttaattaccaggacaagggacgcatctgtgtcaaggctttcagaagagcaattcgccccatgaggatggggaccggatcctattgaccccttaccctactctagtgcttggcacatggtcggggctgaaaggcatttgaacgaacagcattcatgcttgaacagaatcgccatcggagaaggttcctgtctggggagggaggagccctcaggggtgttccctagaaagccaggtgtcccgctgcagggaaccaggttccctctccacctgagaagcgggctctggggatggaattatggtaatcagggcccacatttgcatctccatcccaggcgcatccgctgcgttggaatgaaacctttgaaaatcgccatggacactggttttgataataatatgtgggtaaaggttgtattgctctctgagcggtgattcatccatctgtgaagcaggacaaggcggtaataggaaggagccgatcgccgggggcaaagttggaatgcaaactcatcagaggcagggaactcggcccgcgctgttaaatagaattccattattgtttcataaatagagagattgaagtgcccaggccaggaaccgatgatcataggggattgcgttttagctaaactcgcgactagtatttgttttgagtaagtggttgtctcggctaatagtagggtatttcattgttaagagcttgttgaatgggatttatggaagagtcctgtgcggctgtttgtgcaggatgctcggcgctcgctgggagatggggccttggctccgacgttgctccatctctcgcggtttctcaccatctcttggttaatgtcaccccttgaaaacgcgacagcgtggtccggcccctgcaggtcctggagtatctgaagggcacggcccacagcgcaagggcagctcccagtaggtgctcaaaaagtgtgtgcgtgcatgtgtgtgagtgtgcacgtgtgtgtgtgcatgctcgtgtgatggggtgtgggtttctggtttctcttcgagagcctctgctgggccgcttctctgcttcccttgggaccgggtgtccctcctgtgtgcccacctgagggaggcagcaggtgacgagatccagggaaacaggtgaagcgccatcaggtggaaagagcttccctttcagggcaaagcctggctcagaggcgtccccagagtggagagggctgtgcacccccagccttctccacgaggccccccacagagcttcttgtgcacgggggaagcaggcgacaggctggatccctctctgtgcctgtctggaactggctgtgatttcagcatctccagaggcctacaattcgggtgtcccctcagctcacctaaggaccatggtggacggggaccccagaaggcctccgagttatccccaaatagccaatgaggcccagtgtggcagcgccagcccctctgctctgacatcagaggtgtcgctgggcttctttgaggtgctctcctgcctgtggggcccaggctggcctttaaaaagccgcctctcgagggactccagcttccggccgaccccaccacaagtcccgtatccggccgtctttgcattcatgggagctgctgatgcgggagggctggcaagccccagtgtataggagaggccaggcttctgctgccagtaagacagttacaagatggtacccaggacagacgtcagacagcgggcaggagggccgggctgtgggcttaacccttctggcctcagtttcccctcttcacatgggctagtctgagaagagggaggaaggtgttgtggtggtaagagaaagggggaaagggtccctttggacccctcacattgcctgtcccttgggcctgcccatgatgagctcgtggaagtgactctcatctccggtcccttgctgacctctggcgcccccagctccgaaccctgggagcgtcagcgccgtgggagatgggaagatggcctcctgtgatgcaggtgtgcagcccccactctcattaaaggtagccgtgcacgtgtgccggggaacaaagggccccttcactgtcacctccctgggggccggcgctggcccccgcctgtaaccgaggagccgcgctgcctccactccctccgcaccgtccggctcccgaggccgtacgccacagctcgcccactgcaggctttgtttagtctgggtttctgggcgttgggctctttttttctccaaattcttccaaaattctgtaggctgaagacaccactcatgcagcccaaggggagagtaaatagcccttctcggtggagaagttcctgtctcccccgagggcaatcatgggtctggagagcggggggaactggagcatgaccccccgcccccccacaggaaggcatggagcctgcatctgtgctccccaatcaagccagaaactcttgggggggcggtgttcccaagggcccaggagcaggaggaagcgctcaggcagctcccctgtgatcctgaactcagggttccccaaagggagggagtctcttcaaaccacgcctgacttgcgtctgctcgaatccttgtccaagggcctccctccctttgaccctgtgtcaaaaccgtggctgcatggtcctggtgggggcttcctcagtggggacagttctgctcttggttgactggctccggtggtttcaaggaccttgtctgacgcccctgttgagaggccctccagcctggccccatgcggagacacaggcgccctcctccgcctgaagtgatgtggtccaagtcactgtggtcccagcggcggctttcccgttaagttaggcgggatgaggtcaatgctcccgttcacccaacaaatacctgtgcatcttccactctgggcagaactctgggctccaggaggaagcggcagccttgccttcgaggaacctgtaccagaggcgggctgggagggacacaaaaggtgacttagcgcctcatcttattttacgcgcaaggaggaaaggggccaggagtggcttccaacagaggcgggcctgctgtggggcctcacggacagggaagggggccaggtgtccttgttggggggtgggggagaagggccaaaatattgaaagctatgggctgggaaggagggccagcttcttttcaagaagaatcttttcatgaatccctcgtgtactgtttccaagcacaagaaaaggagagaacagctgagtgggtcagatttgggactgacaactcaaccgccctcaaattcacatctttcagcccgcgacccatccaccctccacccgagcggtgctcccgggggtggcctgctttccatctcccctcgcaagcctggtggtccgttccgatctccccgccctgcagtgggcactgggggctgtgggaccggtcctgctccatttgcagtgctctcccagcacctggcacagtgtgctgggcccggggatcccttgtccatgtctgtgaggggttggagggtacagggtggtgtaggggcagcaggcagaaaggatcagggcggggggcgagtctcctggcatcaaaccagacctagcgcctgcggacggctgcccttagcgccaccgcctgggagcgcagcaggggaacagaggggagcctgagaaaggcgcccccaggtaggagggagccgagaacgtggcccagagaccagaggagaaaggaaatccagcgtccagccctgcaggagggagctaggcccagagggaaccacagccaaactgcagctcaaccttcccagggcgctgcaggggcacgaggggcacgaggagagggagacaccggcagggccgccttagggacggttcgtgcagccgaacatctggggcttgtgaatgagtgtagagtgactgagcatctgcctccctcaataagtgggttcgagtcagctgtcaatcagaccgtgagccgcaggagacgttcccactgcaatgcccccaggggtctgagttctgctctcgaggttctgagtgtccagcgtccgcgtcccatcgccttgcaggcgtgacccctagagcagaggtttgagccacagcttgtctgtgacgattgacgtgatccagaaatgctctgtcctctgcaggagccaggaatccaatgcagccactgagccgtgaaacgtggctggtagaactgaggcactgaagtttcattttaattattgttagtttaaatggcgccccagtggacaggacagcccaggattgtgggtctttgcttcctaacagcactttgtaggccctccacaaatgaccgtggcagtggcctcgttgtgtggcttccatgtcgcaaatcgcgcaagacagctccttccagtccgagctccagagaggcagctgaagctgaccatggagggctgctccggcaagacgcatgtccacaagagactctctttcttacactgatgccgttctgtcgactggggtctggggaccggcctggaccccggctctgggggttgggtagaaaggggcacaggaaccccgtggacacgccagctggagctgttggaaccacggagaggaacctgacgctgctctggttacatttgggccaaaagtcgttctctcttttcactttcgctttgctgcaagcctttccctcgagcttcaaggggtctgtgcccaatggagctctgagaccaccgagatgaggtgcatgttatctttgcagtccagccagggccacctggagtgaaagcgccagacgccgtcctcccgaagtcatgttccggcacctcccaccacggcactgactctgccgttggggagaatatcttattgtggctactggtgtttccagccctttcctccttttccggtttgtgaacatgtgtcctcttccagcaatgactggttcacagtgtctgaggaacgattgtgtgatgagggcgcgtggatcgtgggtgaggcttggttgccccacgtctggttctgccaaggtctgtgcatgatccttagcatttcctttggtttccattcctgttgttattagtttttctgtcttgtgctgcatgccccatgcgtgtgaaataccttaatccatctactgagcgagagatgggcagcttccctcagcagcttaaatctcctccagacctcggtccttccttccacactgcacgtgaggactgtgcccgagcagaggacatccactcacagtggcatctgtttgctttctgcgcttatcacccacaagtgtaactgcctgttccttgtgcccagcccccctcacccaagcgagtttctcagagacaaggctatagccgtggaccccggagcctgaccccgacagggttcacgggatacctgccaaggaatgaacagatgagaaaggagtcagtgtcttcgaggctggggacacgagcttcagagacaaaggggagtttggctgcagtcactttatgtggccacttgatcaccaagccccgactgtctctgtattttcgaaagaagcatctcggtggccacatgcttggcccaacgtggcttggcttgacgctgagggcaggaggcagctcgcagtgacatggtctccctaagcctcagccgtggactgcagccgggctgcgagatgtagagctggtcaggacagcgtgtttgccgatcgacagagcggacaggaggcgcggccagaggacagctccgttctttgtcgggtgtgtcattacttaccccaagcaaagccactgtcccaaactggcctggtgtcccaggcactccagctgttcacagtcacacctgaaccacactccttgtctcccggctgccttcttagcctgccagctgagtgtccttgagaccagttattccagctgtcaccaactcagtgcgcttcccaccagccaagtagaggcctcagaggaacccatggattctgcagaaacgcctcccagcttctccgggtgagagcacgtctggcacgtgctgggccattgggtgcaacaggtgcagttcaggccaaatttggagagcccacgggctgagcgagaacacgggcggctccgtttgtagacgcacagttgattgtcattattcgtgttaagttctgtaaagctgccccggacgctgaatcagtgaacactgaccccctgctcctagggcacacagcgggctaggttcctgcagcctctggtcacgtctttgtcagctgatcaatacacaaccctgtctcgtgtgtgtttctgtttaaagacaccttatttaatacatgctgttgactcgttcgttccaaactcacgaaaccagcactgcaactcgcgccggcacagagctcatctcgcaggtgttttctctgcgaggcgtgtcacagcatccctgtgcttaggacacgaggcagcacttctgcaccaggctggggccattttaaacagagaaatcagcaacgaaacccacaaaaatgcaaaaatcacgacactaaatagactgtgaatgggacacttggttacagcccgagagctggaacaagaaggcagagctttgccccgctcaactcccctggcagcgggcacgacagagacaccagtgctcccgccgtgcgtgctcaggaacgacggcgaatgcaccacggttgttggttttggacttacaaacacattttagcaagtgggtgaattggcaaatacagaatctgtgaataatgaggatcagctgtatataatattgtgtataaatatcagatttattgaggtataactcacacaccataaactttacccttttgaagtgcacaatttggtggtttccagtacattcacagagtggtgcaaccgtcgccagcatctaaacttagaacattttcatcaccccaaaaagaaccccgtcccccttagcctcactcccaattcctccccctcccccagcccctggaacccacttgtccactttctgtctctgaacttacctgtactggacatttcaagtaaacggggtcgtacagtaggtggcgtaacgtctggcttctttccctgagcttcgtggttttgaggttggtatacgttgtagcaggtgtcagggcttcagtccttgttaaggctgaataatactccactgcggggacacgccacgtttgtttacccattcatctgtagatagacttttgggttgtttccacgttttggcacccgtgaacagagctgctaagatgtttgtgtacaagcttccacgtggactgtgttttcgtttctctcggtgtcccctgggagcgggcttgctgggtcacgtggtgactctatgtccaactttttgagggactaccagccgttttccatagcagctgcccatgttacccccccaccggcagtgtccaagggtccgtccatcttcccctcttcctcgccggcactgggcatcgtctgcctcctggataggagccgcccgctggatgtgacatgtgcctcacgtgtgaggcgtgttttgagaggcagcgctaggacttgtgggaggagaccagctttggggagctcggggacagcgaggtgggagcccgccgagacttggtggggtggtcacctctgcgagagtggtgctgcctctgaaatcacagggagaaaagaaggcgttcctccctggaactttctctggagatgtcttagtctgtcgaagccaaattgtcactcaagacaaatgcccaccccattttgtgcaaacaaagggagccttgtcatcctcggcatggcagagccaccggcttgcctgactctctgagcgggggtgcgagacggagcccgtgactggccacacctccaggcgggcttctgcgggcatttacgacggggcgaataagctggtaccttaggacctggtgtgatggtgcccagcaatcgctcacggtccccttccctcctccaagtgtcccacaagctccgaactctccagacagggcaggggtggcctcagagcacagggtcacccccgaggcttcctcttcctcagctgctgtgccgtggctgtcaggggagcctggggaggcagctttggcccccaggtcctccatggcccagccagaggacccacagaatggactgtgtggagaagggctgtccagcccagtgtcactggagggtcaacaagcacgcacacatcgcaggtgccataggccggacagggtgcgaagaggggaggagcctgccttcccagcagcagagaagcatttggtcctctggtgcaagaacatttccattaacattctcagaggacccactccagaagacgttcccacggacgctttgaaataacagaattccagcagcacagctgcagtggctgctggaaaccgcgtacacactgcaccatgccagccaggcccaggatgcaggaagcgctgcaccgtccccagggacgagccttggtcctccggaggagggggccaggcctgccctctggtctctgccgcaggctggtctctggtttcaaagtccagggcccaatgggtgcacatccagctacacaatgaggtacaagtagattttcccttctttctctctctctgctgctgcttactcatagcctcttacatgtagttgatttgggctggagttcaccactcagtacggtcctttggccgagcatcgcagcagcaaagcagcgtgacacacaggtcccagagcccacaggcagggcagaactcgaggctggaaagccgtgaggaggtgcgaatcaggcaggattgctggacctggtgccgtggctcttgtgtcccccaccagccacgctgcacacagtgctttgtctatgcagaacgccctcgactggcggccacacctgctggggcagagctgacatccaacacaggaggaggaacccctcctgcatcagaaaatgcacatgccaggccagggttttgttttccccattggtgtatttgcgagcgctgtgctgctcacccgtctgtgccaggtgctgggttacgcgccgggagatgccctctacatccttttcaagtacacgtgtgccttcagagccaggcacaggtcaccaacgactgagaagcgtgtcagagtccccgggcagaggcgtcctggctccttcacctccgggaagggcctcgctcagagctggcttccctccaggctgcaggcacttctaaccagcgtggttttccgaggtctgcgcctccagcaaaccggcagcggaaggcaagcgctcaccacagacagccaggctgcagcgagagcgagagccaccatgcgtcccaacagcagggagaccccaaaactgcaagcaagctggttctaggaaccactggggtacgtttggagtggggcagctcactgtcacatgtagaataaccaaagttttaatgacggccacacagcaagtgcttttcttatcttcttattcaggaaacaaggcacacgttttagaaatttttcgtatagaagaactgagcgattgaaaggaacagacgctgtcttacctggaagacctcgcggtccagaacagtgccagcaccaatgggttctggaagggtgaactttctcgatccctggacacagacgtggagacgtatgatgtcataggtgcggatcgctctgggaagatccaccgtgcggcccgttcctgcacgaggggtaggatgttctggagaagttttaagcattccacagaaaaatatgaatgctgaaaagtatttgcaccatgcaataattgcatttttaaaatggctttattgagatatagttcacataccaaacaatttgcccagtcaaaatgtaccatctgggaaaggccatcttgggttggaattactaggccttgcactatcatcctctcacagtcgtccttgctctctgttctgctgggtgaccttctccgagggtgggctcgcctgcagaaggctctccagtggagggagaatcagagtgccgcatggcctccctcacaagctctcggggctctgacatcactgacctgatccccgctcaccttggctcctgggggccatatcttgggatcccgcgtgtctttccagtgagtgtcaggcagtgtagtccaccctgtggcatctttcctcctccagcccaacgctgctcattcagagagtacaccactatgtgtggcttcgtagggcaaccaaccatcccagtttgcccatttctccccctattagcactgaaagtcccatgtcccagaaaacccctcagtctcctgcaaaccaggactgttggtccccctacagatcagcagcgtctgcagaccgggcgctggtggctgcagggatcctggtggcccaccccctgccagttcctcctgctcccccaccaggaggtcagtaactgggagcacgagtgtctgttggggaaggacccagcttctatagcacaaatcaccacgcccaccgtctcaggtttgcccgtgtgtcctcatgcccggttttctgggagagcagcgcgcctgtagctcccaagcttgggcagggctttgccgtatcttcttgcttctctgagaaagaagcagactcagggcccggatgctgcccggcgaggggtcggctacggatctgggctttagctagacggctggctcgtcttccgatttcgctacttcctggccgcgtgatcagggcggatgacgtaatatctctgtgcctcggtttcttctctcagaaatgctgagaacagtgcctgggcgatgtggtgtcgtgtaacgcgcggtgaggatgagggatggtggcggatgcactgtgcccagcggggaagctcacatgaggtcggccttccctgtggtccaggagcagcccgtcccggtgaacctgggttcacttctctacgggtttaattttccacgtcttctccttggaaaatacccagcaactcccaaccccgattccccaaatcctgtctgtaccatctccagcccctcactcgtccccattccaggttttcattccaaggatggagagaaactgaggacaccaggctttctgccctcctccccctctctctctgcctcctcagtgcctggttcccattctggaaggccttggacttccgctgcctgtggacaggaactcccgcaggcgaactctcgcctgcccacagtgcgtctgctgtctgcgtcctcggagcaatgatggcgggagggtcggtggggagaaccctgactctgacagtcagaaaatatcccctgtgcactgatgtgaaattctgctcagggcgcagggccccagcccgacacctccctcccatcacagtgcaggagggacctggccgctgggttaccagacgtggggaggatttccatggcttcatggagccttcatcccagctccgttgcctcgggcaactttcttgacttctctgggcttcatttgtcatctataaaatatcatagagacataatgaggactaagagtaaaaagacagagctagtaaccgtgctggcctggagaagatgcccagtaaatgtaagccacaactcttattattactcttgttagacttatcgcgtccctccaggggcagggcgcctggttcttggggtcagccctccagtgtgtctgctgtgactctggaggctaaggcccctgcccccgagtggaggccttctggaagcacgctcttctccacagcacccagcgcagtcaggggcagggcagggacggggcctcggggaggaaggagtggaggccacggcagagcagcggggtctgagagggccaggcgcctgtaccgcagcccctctgaacgacgtgtagtacacagcaccatgttcagaccacagattccaggaaagccccagagcagccccgatccatgcaggggtcagagtgagcctgacctccagccagaggagccccagacccccaactcaccaagtgtagtatgcacgtgtgtgcatcacatggggtgtatttcaatcactgtccacaaggcaggtgcattttgtaattcaaagctatcagacggtcccattcgcacatctgagttggtttgagactttacagcagagcgcggtgccgggccactcaggcaccaggaccgcaggctggggctctgcccttgccatgcccagctgagggcccagggatgggtcacgccactgtgcctcagttccctcctctgtaaaaggggcgtcagaatgatgctacctcagtagttacggtaatgggggattaactgagataatacgtggaaagcctcctcggatgggaacccaaaggcaggaagcccacagtgagcgagagcatctccccatccctcagcctttggagttcagtgtccctccacccatcgccccaaaaagaccggggaccccagccacactcctgcacactctctcctcagctaagtgaggtgacattgggcacccgccctccttcccaggctctgacctgggggcctccgagctgtccaggggtcccagccaagcccagccgggggggaggtcaggctcccaaggcacagagccgtgaaccacttgagtcacttggtcgccaaggtccgtggctgctcctcctccacaagctgggccagtaagcccttctcaccgcaggagtcgtggtcctgaccccagccccgccccagactccctgggcccctcatcaccgctctcactccttccctcctctaccccactctctcttgcccttcagttattttggcgactttgcagaacctcagatgaggtggcccctgggaggagggtccctgtgtgtgcatgcctgcgtgggcacacgtgtgcacgtgcatggtcgagcagtgtgaattgccgttggcagcggggtgcagatggtcctcggctggacctggcccaaggccccacgtctcggggtgcccgtcgtgtgtgggtgagggtgtcgaggcggggaaggggttaacctgtgcgggcgggcgccgagtcctgtgcagaccgagccgccgtcactttctgctaatgaaaaccagacccgcctgcaacaatcgcagcagtgttagcgcggctcccaggacgcatctctgctgttgctcccagagagcaggagcggcgtgcccacctctgcggcctcgctctctttcgcttttggaaaccaataggaggagccaagtccagagagagattctttatttcttgcccaaattgcctggaaacctgcccctctgtggtccccacctcgctgcaccctgtgttcaccgctccaaggcacggtcctggctccaaggaagcttccaggccatgcagccctggcccagtgggcaggtctcaggaagtcagaagtgcagtggaagaaagagaagaagttatcaagggtcacttacaggccaaggaccaggccaaacatgtgttcatggaaccctcactccgaccttctaaccgatggggaacctgaggcacagagaggtccagaagcttgcttaggtggcacagccaggaatggtgagagcaagagtccaggtctgcctgagcttcacggcggctggtctctcccccaggggaggatcctgattccaggccacctcccgcccagggaagatgggccagacatctttctaacaagcctctggatctcaggagccctgcctagcagacagaaccagaaggccagcagcctcgctgcccctgcccccgggtcccgagaggctgccaaaccaccagtgtctgtgcagtccctcccagattcacaggtcagctcctggccagcaatgcccaggcctcagaggctggccacaggttcagatcccagcttgcccgtccctggagagatgccctgggctctggtcctgacttccgtctcccctggtgtgaatcatgtgatagtcaggacccctggtgtggtcaggacggaatgagatcaagcaagaaagggccgtgcctagcctgctccatctcagcacccggtaggcgacagcggcgccatcggcagcttcacccctgtggggctcatctctgcttattcacatgctcagccagtgcccaagcctgtggtcagtgtggggaccaagaggaagcagtccttgcactcaggagcccgcagcctggccgggttggggagcagcaggcgtgtccaaattggccagtgtgctgagcagaaggactggcctgggtgctggactgcaggcccctgtggggcaagaggccactgccccaccctcctccgaacctggtcctggaggatggccgccacccctgccatctgcgccctgatagccccgcttcccctggcctgtgtctgatcagcatcacgccctccgtggaaagtgttatgagcccatccgggagcccggggccagcgggtctgggccaccacgtggacgagctcagcaaggactttcaggagtggaccctccagccttcatgaagcccaccgacagccaagccgctctgctctgaaggctggaatttcaaacttacacaaatattccaatgcttttcaagcatgttccaaatagacatttatgttggctgaggcagtattttccttttattgctgttatgagattcaacatttttccagaaataacctctgaaaagtgcgcctggattttgaacacgtgtgatcctgacatacagggagagaggtttttcaaaacacacacacacacgcatgtgtaccagtgtgcatgctgagggcacccggggcgtgcattttaaaaagcactgcacgtgtttgtcagctggagagcctgtaggagggtctgagttttcgtgtccaggggccacgtttgcgtgtggcactgcaggcgtgggcttgtggctgtgttccacacggcatcccccgggtgagccagcagacctgagatgctctgccctgtcataaatgcccttccgagtgagtggatgatgaagaggatgtggtttctgccgcctcagccacggccttctgcccacgaagcgagagccacggttgaaaaggaatttaactgctagctgtcttcgaaacacgtaccgagacccactctgccctctggaaccaccagagctggctccccattcgctgtaacgattagtcatcaccaaagtcgtgactcagaaagggagagcacagcgtcccagcacggcctctgagctgaatttaccagaaagctttgtgaggaaagagcctggccgcttatggaatcttgaacagctcccggtgggtcggagaaaggaggagcggaggcctggtggattacaatgccgtgttctgccactcactccgctggtcccacccctgcccagtgtccaggtgggagaaaggaagtggaaagaaaaattaaaatggaaatggaaactcttttccaaaattagacatcgtccattttggcctgctgtgccatggccatccaggctccccccgcgaagaccctcaccccggcctctggaggctgcgtgggcccctccccgcacgccccactctgaggctggaatcgcctcccaggggcttctgagcgctgcaatctgctgaccccaggcaaacctcgggctcccgcttgcaccggcgagtctgggatttgcgaatgtgtgaaggtattagctgccagttataactggaacgcagcctagaggcctcgcttctccggcaggaaagcttgtaatgcagcgaatctggacctggcccagcattcagaaacaggaagaattaataggagtaaatgtgagcactgttagcactccggctacgatttattgccgatggtgggggaaaacatgggctggttgcagagttttgtctatttgaatataaattgtaattaatgagttaccgagtgtgggaccccaggaaataaggtccaccgacagccatttacagaggcatgtgaccacctctcggtgacacgcccttcctcccacagcgggctgggggtcgcgaaacagacccagccaccccaccgtcccttaaacctaaggaaagaaagcctggtcccggggtggcccgggaagccccgggtgcagttgttctggggagagtgatgagcgtgcactgtgtggtgggagggcacagacatttggggacaggggacagtgtgtccgggctgtggttggtgtgggctaggccactcagcaacactctcctggctcttctccactctgtcatgcccaagactgtttttaaatggccctggagagacgagagggagagagagcgtgtccttctgggggtgaagggctcgtttgcaccggggctgatcagatgacggagagagcccgcaggtcagagaacgtgaggacaggccaagggagcgtctggaccctccagcctggtgaagtcccccggctagcccgggcccgggtatctgctgccccacgggagcgaacttcatctgcctgctctcctctctccctccgttaatgaagggaggttggggttttgtgttgttttttcccctaatcgtcttcgcccacggagccagtttgttctcagaaaagggccccctcctcctcttctcctgagctctttgatcttctggttcgggttcagttgcttcgttttttgcaattaccagcctggagggaggatgcccctgacctgcagccgcagtctggcagacagatgggacaggaaacgggagtttatttcagccttcgcccctattcatcttggatagattacaacagtagcagctccaaatgtcttcatagcccatttatgataaagcgatgctaacatcacacaagaagagaaatgtgcagattagcgatatccgggaaaagatggattgactgagtgcaggggattgctccgcactggccccctccccttgctctcaggaaagccccctccatggggcacgatgggaacccccacagggcaccatcagagacacttgctgcagcctctttctctccctccctgtctctccttctctctctctccccaccctctttctttccttccttccctctttccctctccctgcattctttgcctttccaagtttttgaaagtctgcagggcaactctgcagcagcccaaataaatcaagctgccagggttacttctcagaaggctgggggtttatatttgagtttcaggaagaggggcaaatccatcaccctgaaggctgcgtctatagattttaaagtttgcttgaattatcctggggtgggggttgcagtggaagggaagtttggaagaaaaccaaggaggactggccgaggaagagcttctagctgaaggacacgggcgcatttataagccatcaataagaaccattaagacccgggctttctctttgttctttctaagaggaaacacttaacaagttgttgttctttgctgtacttttttaatgagccaagagaacattgctgctgtccccccatgccccctggaaaggaggggccccaagctttgtcttctgcgttttgcatctttttcctttacatgcaaactgacaagaaatccccccgtgaaggaaagccgatctgcccaggctggtttcaaagagctccaatttagagcatccccagcgacttcatttctaatcatgtttttactttcctggtctcccaatcggagacccccccaggtcactctggtttctattgattagagcctgcccggctccgaggtgaggcccgggatgaatgggggcgggggagtgatgcgctggcgagtggcggtggccctgcctagagaaggtatctgaggatttgaatgacttctcaggggacagtatcaggcttccaaattcctttcatccatcagatgagagaacaagccgtggctttcagtgggacagggctggggctctctcctggagtaaatgggctcgggccacacagataaagagcttctcctgttccctttaaactgtttcaggcaggagtcgtctgtcgctgactgagggcgggtgggggcagggagagacctttcagctctgattaaagtagcaagttcagggccacagtggagctttcgaaaatctttccagcccgtttcccgctttacttactggtttaccccacaccacggggaggggggcagctagccttgcataccaaaggaaaccttccttttccaaggcagggatcaggtctagcctgaaagagctccaggtccctctgctcccaattgcacatccctccagcttccagggaagaatgagtgcttattaaataccaacagtatacatcactgggaggaaaccaggaaggtgaggaactgggcagagggagttgacggtgtggcaggatgcagtgacacagtgtggcgggatgcagtggcacagtgtggcgggatgcagtggcacagtgtggcgggatgcagtggcacagtgtggcgggatgcagtggcacggtgtggcgggatgcagtgacacggtgtggcgggatgcagtgacacgatgtggcgggatgcagtgacacagtgtggcgggatgcagtgacacagtgtggcgggatgcagtggcacagtgtggcgggatgcagtggcacggtgtggcgggatgcagtggcacggtgtggcgggatgcagtgacacggtgtggcgggatgcagtggcacggtgtggcgggatgcagtggcacagtgtggcgggatgcagtggcacagtgtggcgggatgcagtgacacggtgtggcgggatgcagtgacacggtgtggcgggatgcagggacacggtgtggcgggaagcagggacacggtgtggcgggatgcagtgacacggtgtggcgggatgcagtgacacggtgtggcgggaagcagggacacggtgtggcgggatgcagtggcacagtgtggcgggatgcagtggcacagtgtggcgggatgcagtggcacagtgtggcgggatgcagtgacacggtgtggcgggatgcagtgacacggtgtggcgggatgcagggacacggtgtggcgggaagcagggacacggtgtggcgggatgcagtgacacggtgtggcgggatgcagtgacacggtgtggcgggatgcagtgacacggtgtggcgggaagcagggaggtagtgggacaggaagcagggacacagtgtggcgggaagaagggatacagtgtagccaggaagcagtgacgtatataatgacccagacacaggcccgaggctctccacccctggcgcacagaaagcactgagtaaaccttcgctcctggccccttatggtttggcagcatggccagttggaggaccatcgagatctctcgccacttagtgcaaggaatgtttgcccccttggctgcctgcttttatgacgtaagcaggcatcccaggggacatttcaggcacttacctggggagtgacaaggatgagatgaggagaaggacagtcctagtcagaccctcattgaccctgtgggggatcccagatgacgaatgtggacaaccaggtgcatgttctctctgggtgcccatttctggcttcatttctaagctgggtgcgggcctgggccacaggctcctggcattaaggtgcggggccatggaaagcctccggggctgggtagggctgggctacctgcggcatcccgcttccatccagatccgacttccccttgaggcccagacttctacggtgaaactaccatggaggtgggtcctgctgccactcagctcctcaccaggacctctgaccagctctggcctccaggcccagccactcaggggaagctctctgagggcaggcccagcggggagagtgtcaccggccccaagcctgcccacacaccccagcctcttcctctggacacccccagtgccgcaagggatgcacattgcttataggattatttccttttaataaagacccaaaaaagtttccaaaggcaaaatgacaggctggttaactttatatcccaactccagaaagatacattttcctctttgtaatgcactagttagtggttactggcacattacattaactaatttgagatcgaattatcaaatttgctcattgctgctagcgctaatttgaaaccttacaataaagaagggtttgagaggcacatgaaaggccgccacccaccgcggctccgaggcccgtccccggagcatcacagcgaggctcgcaaagggttcggggtaattgcagtcatgagccggagttgattgaagaaaattataccccagttttttactacaagtttttttttgtgtggcttttcttaaattatgcggtgaaattcaattacaagggaagacacatataggacttttaataactgtattaagagcgtctttcgagctcctttctgctccatctttttgggttgttaacctacagctttctcaaacttgtttgtattcttgaggagtttgggtgttgtgtgtttttttttttttaagaaaattatctggttcaaattcacttttttccatgtcattttttcttcttattcttctacaatttaaaccagaaaatgtgggaaaggaagcttcagcgcatcatatatatgtgtgcgtgtgtatatatacacacacatacacacacatgtgtataatttctttttaaacctggggttcttggctgcatccctgccagacaaactctcacggccttgaacttccctggagggtgagggatctcagcaaccacatttccaaattgcaagtcacaaaagtaaatttataaattctgagcatcatctggatatttaaggcatgccaaaaagcaatggagggtgttttttccccacttattgaaagatttgtaacaatagaaagtcatcaaaacgccgagggagctgtgcctgctgccagcgccatacccactttcctcggaaaaatgaaactgggaatgaaaaataaaggcccctgagctctgtaattaattcatttacatttttaatgctgcaaatggctacaaacatggcgtgacattagaagtgacacctgggaattctgacccagtgcgtcccgcttcgactgtctgtcggggaagttggaagagagtgttcctcagaacccagggagcaaaggcactctgggaggcgtctagcgtgcacggggtctgccatggtcaggggtccagcctgctccaaaggccaccgccgcaacctccctgtgtccagcaggagcgggggagcgaggaggacagcccggagcagggctgggacgtgtggctcggtcagtctggagtatggggtccttcagctttctctgaaagttgagggaaagattcggagctggcactaaggctcaacatttgaagggtgcatcaaaaatggtcaccattctcttctgacatgtttgggcacttattctgctcagtccagcgttctctgccttgggaccattagcctgtggagcaggaggggaacgatgtggggcgattcccacagagcgaaggagaagcccaaggccctcgaaccaggcagggagagagtgttgggcagacgggagagaaggcctcccagggtgggggtcggcatcttgctggctggcccctggctccagggacagaaagagggtggggagggttctgagcacttgtccgatctcccacccttgctgcccagttggggaaactgaggcccaggcataataatgacttccccagctcaggggcagagctgaggctcagcctagctctgcgggctctgatctggggggtcttcttctctctgtgccaccccagcccacccacatggcactagaaatcattgtccatgggcaggtatgtccttctcccatggcttttgacggcttttccatagagctttggcatctccggcccacacttgcaagaagacaaggcaggaccagccaggttaaacttatggaccttccaggctctgggcagcacaactgggtgcccggggacagccattgtcccagtcagcaaaacgttggccacatgtggcaggtgactctgaagagagagagtaacaaggctgtcccttagaggaaggtgcttagggcttactgcccaccccgacggccaggtaccgcctcttgagaggcctgcttcctttttcctagatgcagagctatgcaaagtgtacacccacgccctctactgtgtgtgagctgtactgcagccagaataaaatgcacaggtgtgccaaatgaaaagaaatataaattggcagaggcaggagataaagtgccgttagggaccagaccctacaggtccccaggaatttgagtgtggggcattggcctctgcaaggtccaactcaatggtggctggggttctgtgtgtgtttgtgcgtgcgtgtggatgtgcacgtgcacatgtggttgggtgtcttcacacgtatgggatgagcgtgtgtgtatgtgtgggggtgagtgtgtatctgtgtgtgaggtgcgtgcgagcatggggtgggtctctgtaaaggctgagggctgggctgcctctggggcacagccacaggaagcaggggttccagtgcctgtgggccacacacctgcatcctggagaccaggctcctttcccaggcggcccaggtcattgttattatcgtctcaaagcacaaagtctgtgcctgtccaccttgaccctggaggggaagagagaagggatcagtaaggggacctgcccatctggggagcaccctggtctgtggaagcccactgcccttcctgccggagccgtgtccccgggcaccgcacaaaggtcctcccgagataagacaaagttctttgatctccatcgatccctgcagaggcttttcccgcagccggggtgactcagaggccccgtctgtcccagcacagctgccatcgtgacagaagagcttccaagctattcctgctcctcctctgctccagccaggccctctgagaggagatagacgaggccaggccctggggagggccacaggttccagggggcattgtgcatgcctccctgccctgcgtcctttgtccaccaccccagctgggcagtaccctgccgtgcccctgggggagatggccaactgctcctccccggggtgcactgagtcccagcaccaagagggacgaaggccggacacactgtgctttgccctcattgcacaccatagggggcagcttggggccgtgtgcacaccacaggattgtcccgaaggaagagaattcagggaaatcagaagagggaagggtgtgagggtggaagtgcgtggcagtgtccccccacaaagcctgcaagcaggccaacctgagcaggggtgcggggagactgtgcgcatgtgtgcacatgcgtgtgcacatgtgtatatgcctgcatgtgtgtgtgtgtgcatggggggagtatgtgtgagcaggatgggcgtggttttctgtaaaagccccttgctcaccgagctgcctcccgagctcagagacggagaggaagcagggcctggagtaaacggtctgttgattgactcaagttacatcattttcttcccctcaagagcctataaatctcacgcttcttgtaaaaacagaacttaaaaacaattgagactcttaggcagcctggctccagcccctgtggcctgtggacagggccagtgagcgctcacaggcaggccgccctcaggccagggggcccccaccgaaccccactctgaggccggcagcccggggtcccttgccattttgtgggtgggcccagacgtcatagacaggcactctgcggccaccagccccacaggcccgggagctgggacaatgcctctaccccaactccgccctttgatgggatggaaaccggtccattagcatcttccttttccctccacactgctgaggactcgccacccctcccaattccctggggagaaaggaaagacaattccatcttattgttgaagtgggagtcttgctgttctggactttctgctttccacctgtggttggttagtggcgctcctgcatttccatctcttgtgagaaacagctcacacttcaggccgcagtgggaacgacaggccatgggtgcaaagcgcggggccccaggctggcgagtgatgggtggagcgccgtccctgggagccatcagctctccagaaactttactgtcaccccagcatggccagcggggatgcctcacgtgttggcttcagggcagcctcgagtcacacagggaccggtccctggttgtcggtggatttctcatcagcctgccctgcatgtttggggggcagaaataagatgccatcgtcatcttttgagacttggggttccgtgaagtccccgtccatgagcagccctgcccccgccgtatgggtcgcagggaacagtgaggttggaaacaagacatccaccgtccccaaggcggctttgaaagtcggatgccctggattcggaagaacttggaaaagattagcctgctcaggccaggcggcgggtgggccgagttcccgagccctgaccttcgtgccgcgggcagccagccgcgggcgggaattgctgggcaagctgacagaggcagcacctgtgctgtgacagggagaacacctccctcccacccggggagcgggctgctgtcttgggacccccagggggcccagggagatggatcagctgaaggagaagggagtttgcggggaccaggggggactccaccagctgtgggtggacgcctgtgagcgccagccaggggatccccggcccccgcccgggctggaaaccaaacatgcagagtcatgaaagcaccgtcgagggcgtggtgtcctggccgtactttccagaaggcctgattgcccctaatccagagcaataaaactgcggggccttgcatcccagaagctcatcaacaaagagggcttctccccgagtgtttacagcttctggcgcagtggcaacgacgtggtcgcttatgtaatgatgtcaaagagcccttctgcgcccagagagcctctgacgatcgctggctcccacatcctgcgtctccctgggcggctgggacagcggaggggggcccagcacaggacactccaggagcagcttctgccctggaaatggaaaggaaagggcgtgctggggggcggggagaggtcaccgtctccagtcaaaagcccacatggggctggccgctcccagacacttggccagagaaacaagtcctggggtggacttagtgtggacaggatgggtcatggtggccccagggacccctgtgcctcctccccagccacttgaaaaagggggtacagggcaggactcccacagtgggcctggggggaactaggaaccccaaggaaggggacttgctttgcaggactgccccacctgtctgctcgccctcctctctgaggccaccacggccctgtcctcccacccccgcctgtggtcctggctgtctggccccccaccatcactttcctagactcttgtaacaacctcccaacaacttccctatgcgccctccacgttgctgctgggttggggggagcttgtaagacacaaatcttacaggtcattcattcattcattcattccccacagaaagactatgctcctctccacctaggacaaaaactcagcagcctcagctgtgccactgggccactcccggggcactggcctctcagctgggcatgcagccgccacccgagcactgaggccctggcaccagccctgggacactgtgcagggggcaagcagggtctgcatccacggggtttaaaacccaagcagaattgtcccccttgaatctgtcatccccgctttgcctgccttccactctcagctcggtgagggatcaggtctggacgcggcgcgttgtcagcccacaggcagcacatgtgacaccaagcccaggcctggccctcagctgtgcccgaggcctgggccccagacgagctgggaaaagtgccacagcccccggggaaggatgtatgacagggtctggcagagcctccggctgcctggtaggggcacgctgccccccccccagaaccctggcctctgcacgcccaccccgcacacagagcaaacccttcccttgaggtggttccccaaaaccctagagcctgttggaggggtgcaggctgcatctcctgggaagtctctcatagactaagagctgcaaagcccgccttgtgatgaatagccccacagagcaggacaaggcggtaagaggacacctggccaggaggtgggccctggctctcaggctacccaccccgggcgggcacagctgcaggctagctcggtcctcctgggttagcactctcctcgggagacaaaagcaggggatgctcctcttggggggggggggcactgtccctgcatggaggtggagggcctgactgtgcccccagagtaaaggacacacaagtcctgctggccagtccccaccatgccctgaccacaggccagtatgggtttcccagcaaggggcttgagggttgcctctcagggactgccttagtgtcctggggctgcagtaacaaagtaccacaaaccaggtgatttaaaacacatttattctcacagttctggaagctggaagtccaagatgaaggcatgccaaggctggttccttccgggagctctgagggagaacccgtgccaggcccgtctcccagctcccagtgctgccagccttttctggggtttctgggcttgtagacgtatcgtcctgacccctggccccttcgtcacgtggccttggtgttagaataccagtcgttggatttggggcccattctaacccattatgacgtcatcttaaccaattacacctgcaaagaccctgtttccagataaggtcacattctgcggttctgggtggatgtaagtgttaggaggatgcggttgaacccacttcaggggccacagtgagctctagggatgggtgagtgctgccagggttttccctggagtctctgccctggcccagaggccagtcaaacaagggcagtggccaggggtctcccctcccttttgtgcagcccctacacgctgaaccccgggaaccgccgtcaccaccctcacccctgcatccccacctccaacacgtgcgcggcccccacccagcactacccagaggagagggacgaagaggccctcactgcctgggagccagagcccagggctgaggaagggttcccggcagaccccagcaaaggccgggcgtggccaactgtcctgcgcaccagacaggcagcagctttcaggccattggacagttgtcactcctggagcatcctcctgagctctgtgggacctgacagcaggggaggccagtttgttccgggctgggactgagaagccagtcttctgctcctgccgggccaagcctggcctcactgggaccaacatctcctcggtttcagagagaaaattgctctctgagcccccaggctgctgcagacaacctgctccgggacgagagggccacgcacatggggctgggtgccgggcctggccgggcagggcaccccgaggctccctggagagcagcgggaaggtcctggcccgggggtgccgcaggcaggccgtgagagtctctaaaagcaaacatttggattcaaaatcctttgtgtctacaccctcctttttgtttctgaattgtcactcagcaaggaatcgtctccaaaccactcgccacggccccgtgggatttcaggggacacaatggtccgaacctcagtgtcgcttcgccccgctcagactctgagcttgagatctttgctcagggagcaagtgacgccttgggctggttacagaggccgtttcagaggagaaacccggagttctgaggggcagaagctcctactgccggcctccacgggggccccccgtcccttgtctgcctccccatctccatggacattcgtccactccgtcccttcacctgtcctcctgaattgtgcacccaggagtcatctttcaatgccgccgtccacacggtcgagacccacaaacgcggcgggtcccaggcaggaattctgttgatcccgacaatgagttatttgacccgggcgctgatccgattacggataccgagggtatcagctctccgccgggccgccgctgtgcacggctgccgcttgcagacatccggggcagaaggtgggcctctgggaggcggcgacacaggcagggaggacatgtagggggcccagcggtggcactaaggtccccagatacgctgccctcgagggagggacagcaagagggtcagggtaggcccaggtggggtctcagagcttcaagagggaaccctgcttcttggcagatcctgccggcagcccctcgcggcgcccggtcccggcagcaggggctccgatctccccacttcactgacgaggagttggaggcccagggcacccagggcttgggatcccagcccactgggcagaaacaccctttcatggaagaaaaccagctggaggcccgagggtttctcactcactccctgggaacaggagtagggatctcggaggactaggacttccagcaggacactgagcctgggggctgtcccggcgcctgagctgttgacccctctctgggtccacatcttattcctcagggctgggtttctgggcacaggggtccagggaggtttggcccctactgggctgagcggtgagctgacggccctggagaagctccgagaagccccccacatgccccctaggctgcagctgctgtggccgtgggcctctgagtgtttccttgtcaggatttcccaacccccaagctgctgatatttggggtcagatcctctgttgtggggggctgtcctgtgcagcgtgggatgtttacagcctccctggcctccccctgccagatgaccatgacaagctgaactacccagacctgccaagtgtccctaggggaggcaagtcgccccctgctgagggccctggctgagatggcaggcctgccatgcgggccctgccttcctcgggggccagcagcacacccggagctccctcccgcaaagcccctggcagctggggctcttggaaggtgggttggaaacatgcagagaccctccggttcagagggctgcaggctgtggtgtcccacctggtgcccatgaaatacgctgtggatgcccatgtccccaggggcctgccacacagtccagctcgtcctctgcggcaaagaatgtcctgggcagaaaaaggttcatgctgagaagttttatcagatgctaggatgggtccccccatccagagaatcaggctcgggcatcggcgtcaggcaaggactctggcgcacccagaaccgttcacacaccagctctgcagctgggtgggatcttgggtgtgtgatctgaccctccgtgcctcagtttccctacagtaaaaaggcaaggagcttggagcttatcccccagcttcgtgttggacccactgaggcagtggagtagggcacagggtaaacccccgtgctagcgggctccccaccctgccagcccctcccctttctccaaggcaccgaagggcaccttcccactgcgcccatttcctgatgggaaaatgaggaagagtttgcagcgcccactcctttcggctggctctccccccagagggtctcccaagctgtccacccaaggcaccaaaacatattcccttaaagccaggtgtgggtccagtcttgggcagagccaacaccgggggaccccagactggatgtgggtggtctgctctgggagggaactggggtacccctgggcctcaggctctaaggagcccacactgcacagcttaagaggcagctcctgggttcctcctcacccaggggccccgtgccgggacctgtggcattcccggtggcggtgtgcccgcgcagtgccggccgctcgtggcgggggccagtttggggctggattgcagcatttccgggggaggtgggagaacgcagttctgtgctgcaggcgggcgagccacagtcgtggccgtggggccgtcctggactggggctccgggctaggaggccgatgtgctgtgagtctgagaagggagctttcctgttgtttcttcatcaaaaggggtgagcaacagagcaggagccctctgaccccccaacccagctcccccccgcccctccggctgcatcaccctgtctggatttgggcatctggtttcgtgagaattgagtcagtgggcgccctgcatggtgagttggaaggccaggaccttagctggtttttggaggctgcaccgtcacacagggcctggcagggagcaaggaggctcgccacggactttgagctgagtgtggccaaggtgcaggactcaccgtctagaatcatcctgaccgcaaagtaaaataaaaacgggagtgaggagggggccgtgggctcaccaggaggtggcccttgcaggatgcccgtctgccaggtgctatttagtcctgcacaggcgggggaggctccgagcaggccggccaccccgtccagcccagcccagcctgcccagaccccttagagaagcacagaaggcagagcagggctccctgtggatcaggcccgaggcaagataaatgctcaccagcccaggggggccctcgccccttccagctaccaggcctggtggcttgggagtgattctggctgtaggaagataagcatccacgggaatagaggtagctccagtctgtgtgggtcccagaaaggcacagccttggtcagtgtcagccgttcttactgctccgaccctgagcccttcgtgccccgccccaccccgcaccccacccggcaggcgctctttagggtttgcgttgggctgagttgagttcctctctctgaggagaaacgcagcccggcactgagcgcgggaagcataagagatcccgcaggaggactgcgttggacgcactgtccctttaagaatgcaaacccgtctggctgccagcatctctgattcccatgacaagtgatgatgggagagtttatccggagaacaacagctagatttctcagccaagaggggttaaggatgcctttgagacttctggccacccacttcaccttggacttgtcaagtcacactacagcgattaactcctgcctggccagagctgcaggccacaaagctaggggtctgcagacttcaatacgcctgaaccctgtgatggggaccttgagcaagagcacacattccacctcgcagatgccctggtcttctcctcacttggtcccttctcctctcatctcttcttctcatctgcctcttgctccccaggcacacccaccagcccaaatttcctgtaactgaagtgtgtcgcttcctccatccaccctgatggtggtcattgtccttatcctggagcctcggacacggccttgtccccttagccagcccccagatccaagccccgagtcagaggccccccccaaggacccaattttcctgccgtgcatagcggatcagataaagtgtcagctggctgcaccaggccagcaatcagtgtccaatcagagaaaacaaatcccctggggaggcagtgtttagagaagctcgtaaggaagagtcggagctcagatccttcgccagcatgggggcgccccctctgcctcctggggctcagcgggcgccccctcctctccctgcgccccagcccggcagccagagactttctcacctgctgctctggggcagaaatcggagccactcgtgtgcctgcccggggcaggggtgaggagataagagccggccctttgtgccgggagcttttcagcacttctgctcacaggaagtgggcctgaatgcagagaagggcgctcggtgggagcagtgaaacagcagcccgattgcatctgataagagaggacactgagcccacgctgactggccccacggcccacccgccgggctcccgggcctggaggggctgtggccactccaccacacaggggctcctgcctgggatggcccagaatgaggacccttccccggagcggcttctccccgccctgacctccacgccccttacctgggtgggctttggccctgtgaatttcaagaggccaccgggcaacacctcccgttgtcccttcccccttgcaggctggaggggcacattccagagccgcccagaagctggaaggaggctgcttctccctccgaggacgaggctgggcagaggccaaggctgaccgggctgtgcggacacccgctttgtccccgggtctatggattaatacaaccggccacctcctcagaggacatggcctgtcagcggatcccccagatcgagaccccacccacagaggtgcttaaagcaccttcgttgaacgaatgaatgaatgaacaagtgatggctgttccgggctaagcaacgactgagaaggtgacacagctggcacggaggcgcggggaggggctcctctgggcctgttccttaagaaggaagccaaatcctggaggaaaaacatgtttgtccaaatctgctcattagtggccgagcgggatcctggtgaccgggacaccaacagcccctgtggtgggaacgggcagagacagcctttctctctgttgcccggcaccctgggccgaggggttggcaagaggctgaggagccgtcacccccggtcccctgtcctgggctcttggtggctctgttctggactaattggcccctcattagcagtctcccctgctcgggtggcagatcagtgagctgtcgcccggctcccttttcgtccgttctgtgcacagccctagagggcctctggactccaggcttggggtctttctttttacttgacgcagatgtccccaagtctcaaaggatgaggggggtcgcccccacgctcgggcagtggctcaacagcatccctgaaaaataaagcctggccctagagctgggcaacgccccctggggcagtggaccagcctgggctccccggcccggggccctgtccccagcaagcccacctgctcaggagcaccagcccctgccctctgtccaggcttgcggaccccaccccacagctcagcccaccttcctgcttaggtggctgctgtctgacggaatatgccagaatgtgctctcgctccttcccaacatgcaacctgactggggaggggtgtgcacttgggaattgtaattagattctgcctcacgttgtgtactagttcccaagaaaatcatctattaattgattccaattactgttaattgtttctcatacatttcaattaggcggccctattgttacaggcgggagctctcggtccgacaagcaatcatcgtgcgtctagtggcaggggctcggggggagccccaccgaggggtgtgggctgcagacgcggcccaggccagccctcggctcccgcgcccggtgcccagagcagaacacagagccctgtcctcccccacgaggacctggggtctcctgcctccacctcaggccgggcggggagggcagcttgtcagggtccctcctgcccagcctgggtggcccctggcgtgaaatgcccacagggagtgggagaaagtcagccgagagggtcaggtcctgaagctgctgagacttcgttgggttttttttgttttttcttttctaaatcaatcttaagtatcaaggattgactttttaattgcggtaaaatatgtaacataaatttactattttgaccatttaaaatgtatagttcaatggcatttagcacagccacacgttgtgcaaacatcaccattctctagctccagagcattttcaacaccccaccaggaagacccgtgcccaccagcagtcaccccaccccctctccagctcctagcacccactaatccactttctgtgtctatggatttccctactctggacattccgtacaaacggatcacacagtacgcagaccgccgtgtctggctcgctttcacgtagcgcggtgttttcgaggctcatctttgttgctgtacgtgtcagtgcctccttccttttatggctgaggaatattccactgtgtggatggggctcgttttgtctatctgttcgtctgttgatagacatctggattgtgtctgccttctggtcattgtgaataacgctgctgtgagcacctgtgtcagctttctgtttgaacacctgttttcagttcttctgggtgtgtatctaggaattgccggagcatatggtctacctactagtcttatcatgacagagtgtaagcaattcggccgcagaggtggtggacggagggctgaggtgtcctggaaagtgggtgcttgtccctctggggaggctgggggactctcgccatgggagttgggcaggagcaccagggacccccccctcgacagggcagaggtctggctggggaagccctgggccctgcgtgctcacagcatgagcggagggccggctccccatagcaggtgggctcgctgggccggggcagcctcctgaggaggagctcggggccccagggctgcttctgagctcgtggattccagaacccagactggagcatttcccaacttctccagctcgtctcaggattgtgggaatgggggctcagacaggagtgacagaagtagggaggagagacctgcccctgaatgcacagaggaggccgcccctcccaggagtaaagcaccttcctccaaggcttctggaatcttctctctgccaaaaggcatcgtggtccacgtcacagacttggagtccaactgactcggtgcatgttttattcataaactatgtggttggggctggtccttgaagccctgagcatccgctccttcagctgtcgaatgaagacaggctcacaaagcagccctttgcctgaggacgggagtctacgtggccccacgcccgacaccatgtgaagctcaggagacagcaacgacagagcaaacacgcggagcacggtgtggcccagaagcttcccaggtgaggagccacaggcccggaccccagcccaggaccttcctcttgggaaggccgcaagcacaagagagacaccaagggcggtgagtaagcccgaagcccggccctgccattcccaaaagggtgccctagatgccactcgggtcatttccaaagtcgagtgtggccggggccaggtcctccagttctggtcaccgctgtgtctcagaggtttccagcatcaccatcaactattagcccacccgacctcacacctccagctttctggagccacccatcaactcccatgttgagaaagaaagcaggacacggaacaaaccccagaggccaaggccaccactctccatcccccagggagatggccaagaaccacccaggagagccctagagagcacctgggctccgttctccagatgccttcatccccaacttcagccacctccagcttcacccgacttctccaaactccgctttccattctgcccatcgacaaaggtggcagagccgttgccaggaagtgagccgatggcttctctcgctcagctaatgctcctaaccaaacccgctttacacgtggggaaacacggcttccaaggtggtcagccgggatccacggggatctgcacccgctgctggcgccttccatccccccaccccataagatagtgcctgcaccaacagcacaaacatcgccctgtgtgctcaggaccccagccccaccgagacacgggcacagccagatcgggaggagacccaggcggtcgcccagagaggaccacagactggctccgaggagtcggccccgacccggaaggagacccagggcgtgaagtccggagcgttctgtgctgagtcctgagcggaggccgcattcctggagctgcaaagggaggcggcgggcaggtggctgcacagggccatgtccctgcaggaggaggcacgcctcgcaggcagccctttcacggaccttctgcttcagaaaatgaatgaaaggatgttattgacccggggggagagcagtgtggaaataccgggccagttagctccctggaggacccggggaacccctcccgcgtgacactcagagagacctgttcttcgcccagtcacaaccagcatggcacgtgtccgaaagagcgcttaacaagaccctaagcagggaaaatttgtgtgttttaacttttgactggccaagccaaacaaataaataggacttcaaacaagcagagcaggtcggaggacatctgaaatctaacctctggctgaggtttaaaaaaacacaccctcagacataaatctgcaaataaactttccgaagaaaagcatctcctttcagaatctacccgtattccgtgttccgtgccgcatcctggtagaaggtgccagcctggcacacgtgaggtgagccggtggagcgaggccgctaatgggtccgcctgcccgctccgcactgggcgccctgcagcctccagcccgcttcacagggctcgtggggggccacggtggccgggccactgctctcaggctgggggaggatgggctgacgtgggcctggtgggaattccagatgcagatcctgtcaggcagcccacagacacggctgtgatgtctccatcgacagataccccgagtgttactaaacctatgacgtgtcagcccagtgccggcagctgtgctctcgaggattgtggagggagggggagaacgtagggcccaactgcccggccgagccatcgtgtgcgggcttcagacatgctcagaccgtggggcgtgtgtgtatgtgtgtacacgtgtgcatgtgtatgtgtgtgtgcatgtgtgcatgcatatgtgtgtgtgcacgcccggctgtgaaagtctctttcaggagaagggagaggaagggagtgcgtgggacaagtgtgctttctccccgacgttcgagcgttccaaacgcccccagtgctgacagcgtctggttgctgcttctctcggctgacagcttcccttctcctggggagaacagtccacatttagttcttaggaggaccccgatttctgaagctgcagtcttgggagcttgagcaagcccccaggaccgcgctgggtcccaaggcagcacccacagcgtcattatttctgcctgcaggaccctttttgcttgaggagctgccagagcgaggctaaacgccagcacaggctgacaaagcagtaaaggccgctttgtggggcccactcccggggctggggcctgggcactgcagcagccgtgggcgcaccccgcacagccctggcagctccaagctgtcggactctcctttgcatgatttgggacagtttttcattggctctctctgcttggggatgccaggtgcattccactcccctctcatctggtgatgtggagcccaacaacccaagatttgatgctggaaagaaggaggggaaagagacaaaacacacacacaggcccacatgcacacgcagacacgtgtacatgcatgcatcagacacatctgcacatgtatgcacatactcatgcttatgtgcacgaacgcacaccacacagagacacacatgtatacacaagacacatgcacacaccagatgcatatgcacacaatacacatgtttatatgcacaggcacacacaggtgcatgcatgcactgacacgtgcacacacaacacacgtgcacacacaccaggcacacatgtatacacacgacagatgcacagacagacatgcacatatgtgcaggcacatgcacacagacccacaggcacacacagacccacatgtgtacacacgcacagaccacagagagcctccagttctgtgagggtccggccacagtgttcattttttcaatccttatccttctagagaaaaaaataagccgaattcttttgagggggctgtgagtgacctgacctggcacttccaagggagacctcatctgaggagagagggggatctgaccatcaagggggaaataggacatgccccccgcccccaccacaatgccagggcttccgtgcgctgccagaaattccgtgcccttggatgagaggtgctgtcagggtcgggatctcccctccctgctctgggccggggaagagaggccccctggacactcggcctttgggcttatcacgacgcactgtcggcccaggacgctccattcacccctgttatctgagcaacaaaccaagaagcaagaacctcagagccctgggcccccaacccgacttcctgattgtagaggctcctttcaaggtgggggccgggagtgctgcccgtctgtctgtctgcccagggcaggcagctgatgagtatctgcctaggtttggctctggtgctgataaccagggctgcaccgctcggctggctggggaaactcctggaacccaggggctcgtggcccaataccctcctggggttccagatgccaataaggtagaaggtttctgagcatggatggggccaggtccctctctaggaggacataccaccacaccatgtgccacgttttcccggcgccgagctgggagctggcgagtctggagccgaccagggcagacagagcctgcaaagatgggtggccgagggagcactgtgcaacagcagccgctagaccatttctgtccctctcgggggagctgtgctgagctcctcaaactcgagatagcaggacagactcaattctgagggtgacgagcagccttggagctcttctctggggggattcaaagcttccaggctatgaacagggagcttctgaaccatttactccactctgcccttcctaggtcctgtcttccagggactcagaggcgtcccctgacctttcgcctcctggagcagtttcctggccactctgtcctccagccactggtagccaaaccaccttttgtgaggcactccccccggtggaaacagccttccggggacaggaggtgtccgcccgtcttggcacccttggcccctgcccggagccaggcccatgcaggccttggtgcatccatgctgtggaatcgtgagtggatggacggatggacaagtgatgacagcccccgggcggtcagcagggccctggaaagggatcagagcacggtgcagagggtgtttgcctccacctggcgaatccccaagggctccctggaggaggccctgggtgggctgggacttggacagcgaggtgtcatccccagcaaagggacacaggcaagaggctggatgggcgtgggagggggcttcggagagaccatcactttggtctcacaaggccccaccatctccagtgacagtgaaggtgatacctcaccgcctgcccccgaggaggggaggccctggcacaagaaacgtgtgaggtggggccaacttgccctcacctccccagcgcgttccaccaactgcccaaaccaagagagagccgtccactcacagggaagaagaaagccacggggccggggggctgggtgctgggagtggatggtctctcgttctggggaaggtgtggttcctccgggcagccggctgaaagtgggagaaccacagatgggcaggcactgggacggggtgcatgctcccaagcctctctcctgctctggggctgccggggagcctggtcccactttcttctgggaaagaggccttgggggagttccgggtctggggcttccctcgggtccctggcggggccagccgagttcttcctggcgtctggcccccgtggcctgagcccacttcctcttgtgtctcccgcctgtggaccgtccctcgaggttgccctggctccctttgtcccagagcatccccgaaacccccacacagactcagacacccccacccctccgggaagaggccccaggctcctgcctgcccctcggagaccagcagaagggaggtgcagctcactgcctctccttcctgtcgcctccctcccaaaacacttagggagagacagtcaggtaatgaggcacaggcgtgcccctgatgagcatctcagggcaggcagcgaaacaccgataaatgccacactcctgctcctggagagccagcgtcatggatcactgcatccgggggcacctggaacattcccagggaaaataaaacaattatttttgacaacttgcatcaataccatctacattttttctcctgcatggtgctcccctcccccacggcacagccatcccttccacgctcgtctgtgtctttaggaaacagagcctgcaaactcaataagccaattgttagctccatcgatcttatttaagggcctgaaaattcagcagacagcacagtgttaaattattaactaaatgtttccactcgctggggacttgcttttgtaaagaccatctagatgaacaatggcctctttatgaaattaacagccaacaggattggtgggacagggagcagaagtcacgtagtggcccgaggaaggcccccgccatgcccctccggacggggcaagaggagcaaaactgaatgcttcagggtgtgcttccagaggacagagctgccgggggccgccctggaggaagtggaccatgtgcccaggacactctccagctgggcagtggggcgctgaaggccccaggctggggagcgggatgcatgaagcggcaggctgtcaccagccaagtggctggctcagtctaagccttcacttccctgtcgccataatgggatgagcatccacgtagcacaaggatgtcctgggactgaccagaccctccagtgcagagcactcatgccatgaccaacacctggcagagtttacagatgcaactgtcttatcccccgccaggtttgcaccccagctggtcaggggcaggacacagaggcagtgacaggagctcactcatctccctctcgcccagcagaggccaggccatgtgccaggggctggggaatatgcggacactccgggagcttctggtctgttgagggaggcagatgccaggtaaacgtcccctgatgctagtgacccaggatgtccaatagcctccactagtgtgtcaggccagcctggccagtgtggcccaccggggggagccagcaaaagtgaggaagccaggatgtcagaaagcacacacactttcttaatccaaagatcagaaatatcataattcgaaactagcgtaaggcccattcagaccacggtttaaggcgcgcttttcaggcctgctttgctcagccagcaggaggcactggcgaggcacgcccactggcctctgagggcagctccgtgccagcacggctcccccctgctgctgacacactcccctctgaaccccagaaataagcctggagtccagcaggtccctgggggagttcttgagtgtttctgaagtgttgacccactcctgaaaacgtcgagttcctcatgggaagctccagaagagagaaatgaacaaccacatatctggggaagccaccggggcagatgggagcggccaggccacccactaagtcggcggcaccttcctccacgcctgctgtctgcccacagggaaggcagcttgctagatgacgcatccacagcttgtgaacgtgctgaaaattctgtaaacgctgaatggacctccagcccactctatatgaaaaactgcaagagctaacaagggttgagcattttcttcagggccaaccctgttctaggtgttacctgggtatctcattcagctgctacaacaacccacgtggttgactgtccccattttatgactcagagacctgaggccctgggagggtctaggactcacacagtctcccatctagccagtggtggagctgagactcaaaccctggcagcctggcctcagcggccccgcatgtacgatgcgtgatgtagatgataaactgcacacatcgcgagtgtacactgggatgacttttgacgtaactaccaccagcaccaagacagagggtatttccatcgctcctgccatttccagtcagtactccccaagggcaggtggacaaccacctttctgatttccacccccttagatttgtttcacaaatcttcctacaaagagactcacacagtctgtattcttctgtagcttcttccgctcacatcatggtgtgagagtcacccacgccgtcgcgcttgtcagcagctcattctttactctcacagagtagtatctcgtggcgtgaatatgtcacaggttgtttatccattgtcttgtgggtgagcattctgggttgtctctagatttgggctattatgaatatagttgccatgaacattcatgtaccagcatttttgaggacctaagttttcacttatcttgtgtaatttgtcgagaagtaaaattgtggggccgcagggtaagcgtatatctaaatctataagacatcgccaaggtgttttccaaagtgatgagctatgttgcatcccaccagtaacgggcgagtgttctggctgctcagtcctcactggcatggtgttgtcaatcttctatattttaaccattttagtgggtgtgaaatactatctcatcgtggtttcagtaagcattttcctggtgagtaatatgctgagcatattttcacgtcttgtgctcgttagccttgggtgttaactgtgtaactgcttttgtaaaatgttcaagtcttttgtccattttttttaattgaattgtttgtctttttatttgttgatttttagatctctatacattctggatacaagtccttagttatttatatgtattacaaacattttctccagtatttggcctgcctttggtttccttttttgttagtagatcgtgtctgtgtgtgtgtttagtcttttttcagccatttctttccaaattctttttctgttccatttcctgtctcctctctctttctgggcctccaattatacatctatcagatgttgtccacaggccacttgggctctgttcattttgtttcagtacttttcctgtcatttagattggatcatttgtatggctctgtcttcaagttcacctaccctttcttccacaatttccaacttgttattaacagcctgtccagggccttgttcatttcatgcgttgtacttttcacttctagaattctgtctggttcttcattacattttccatttctctggtggtgttccctatctgttcattccttatgattgtcttttcctttaagtctttggacatgtttacaatagctgctctaaagtttttgtctgctaattataacttctaggtcatatcagtgttggtttgtcttgactaatctttcccttgactatgagtcacattttcctgtgtctttgtacatttagtaattttttaaaaattttgctgcattggacattgcagatggtccttggagaaactctggattctgtgatcctcctttgaacagtgttggttcttgttccagcagacctttcagtgactggcttctcaccttgaactccccttgcagaagcttgactatagttggttaggatgtgtctgtttcagtttcactcttagtctcaggatgcaggctttgctctcaaggcatcgagcttctaagcttttaatggcaaaccagagatgttcaccaaggccccctaagtcattgggactcaaattcaagattctctttcccctgctatggtgggcagcagatgaaatccttgctcagctctctcaccttccagctattgccattcactgggctccttgcagtctcccccaagcatgggcggtgcaggaggcagtccaagattctgggcggtggggggggtttggatgcacactggggatcacctgtctgtggccctttcctttccaggatttcctccctcaatttacagctgctttggcaacctccaaccccatcctctgatgcctcaatctgatgagactgcaggtttctgctggatttctagctgccccttgctgagtggactgggtggcagcctcaagggaaaaaccagataattgtggacttcaccatgcagttccttctttcaagggtcaaaccccttcagtttctacctgcttttagtcactctctaatgccttcaaatcttttttaaactttttattaagattatgatagtttacaaccttatgaaatttcagttgtacattattgttagtcatattgtaggtgcaccacttcaccctttgtgccctccccccaccccccctttcccctagtaaccaccaatcaattctctttgtccatatgtttaactcccacctatgagtggagtcatacagagtttttctttctctatctggcttatttcacttaacataataccctcaaggcccatccatgttgttgtgagtgggacaattttatccttttttatggctgagtagtattccattgtatatatatagtaccatatcttctttatccaatcatcagttgatgggcactcaggctgcttccacatcttggctatggtaaataatgctgcaatgaacttaggggtgcatgggacttttggaattgctgatttcaagttctttggatagatacccagtagtgggatggctgggtcatatggtatttctatttttaattttttgagaaatctccatactgttttccacagtggctgcaccagtttgcattcccaccaaaagtgtatgagggttcctttttctccacaatctctccaacatttgtcactttttgttttggttatttttgccattctaacaggtgtaaggtgatatcttagtgtagttttgatttgcatttccctgatgatgagtgatgatgagcatcttttcatgtgtctattggccatccatatatcttctttgtagaaatgtctgttcacgtcccctgcccattttttgattgggttgtttgattttttgttgttgagctgtgtgagttctttatatattatggatattaagcctttgtcggatcgaatccttttataaatattttgtgcagagtgtataattgttatctgcaggagggtgagtccaacacaagctattctaccattactagaatgggaacctccaaatgaccttgttccgtcatcctcgctgcagccctgggaagtactagcacgcacgtcattccctttttacagctggcgatcatgaagttgaaactggttaagtacctcacctgaccacacctgcctggcaggaagaggacttgggtctggaatcccaatgggctgactgcaaaccccatgctcttccagctgttggtcctgaatgtgtcgtatgggcacagcagaacccccgtcccagccacccctcagatctcccctgctgccttccgtagctcagtttttcttggcggccttttcactccacctagagctgctcgttgcccacaccccgggctcccaggagccgacaagagccccactgaggccatgtggctgtgggccagggtctccaccgcgcaggggcagtctgaggggctgcctctgagcatgagtataaaagggggcattgaggggcgggctccaaggagactcggtctccatcagggccccacaatgtgggctttgtaaggcctggcaaggctgagctgagcgggaactttcctctttctcctacggagagattatttaatttaaaaataattgaatgtggaaaatgttggatatggaaactgtttatcaggtttccctctttgtttctctggttggctctgccccacggtgctgagcggcagtgccctccccctgcacgtcgaggtgtctatgctttgagttctgcaccaagccagcgaggggcgccgggtttacagcggatgagcctcaggctgctgacgtggctgctgtggaggagggggctgcggacagtcaggggtcttgggccagacgccgggccagcctggcgggaggtggcatccatccgaatacacgagtccgacatttgggaagtgccgccgttcccccaacgtgccccagccggattctgcatcagtcctcttcagtgcccggtgctccccccaggacaccagaggcttccacacgaccctctcccacatcccttctgtcctggggtctccttcctctcactggtctcccctgtctcctgggggttgtgcaaaaaccatttcctccttgaaatctgtcttgatggccaatcaggtccactgtcttcatcctccgagcaggctcagaacggtgtccactgcccttccggatgcacacacccatgcctcttcctgccgcagctgaaccgaagccaggggtggctcctggcgccccacacagcacccagtcttctttgagatgtgggttggtggctccgtgaggggagggctacctcagacgcgcgcctgtccttggtgtccagccagtgcctggcacatatctgtggactggctgaacacactgaacgtttgatcaatcgtttgagttggctctggagcaagtccttccgattgtttccatgtggctctgccatccctctccctaagtgtcgtgttttcctctgagacagcccatgacatctgtctcagcggttcgtgctgcaacctgatccttcctcaaaagtgctcagcaccgacgatgggggctgacccagactcaggacagcctgggaaacatcttgcaggctcgccgtgacctcctcaaggacctgagaaggtgctcagatggtccagcttccgagtcttgtcctggggaccggaaggagtccaagacagtgtgtggccctgggaaagcaggaagggacgggggacttctcctcgcaggccacttgccctagggttgtgatcttccacgaggttctaccccaggcagacatgcagagagtcaccggggcagccaagagtaagcccagatgctgagagggacctcggcccccattccagatggaaagccaggccaagggagcgagagagagacccctcggagcctctgcatgttctggcacagccactggttccccagcaccagccctggaaggagtccagagctccctgggcctggccaggggggtgacttaggtcccactgacatgtggtgtccatgtgcttctggaaggccagccctccacctctgcacccagggcagaggtctgcatgcagtgggtgctcaaccagtgtctgtggaatgaatgcagggggcagccagggaccagccggggtggcagcacatgaggctagttatggccatctccccctgctggacagggtcagttggtgcaagggcttggggaagtgtgagcctgaatcatgtggaggagtcactgaccattttggtgtgtggattggtgcttctcagacttctggggtgtgaccatggtggacaggtaggtcctgggctaaggaacaggccaagctcggggccccagtgagagcaagctgcagcctgctccaatcccgacctggccctggtccccaccccaaattccccgcacttccccaggtgagcactggcacccgaggcccctgaggcccctgcagcataggggcagccgcctggtcctgcatagcagccactcctggcccatccctgtggctgcggggtcctctgggcatccacagggaaggtgccaggccctgggttcccctgagattctggaccctgggctgtgtctttcagtggctcgtcttgcctaggccagaaccgagctgcagatcaggctactctggcctcgtgcaggactgctgggcagaaggaccctccatggtggcaggaagcagagctttgcccagagcacagagagggcgggagtggagcacagcatggaggggcagaagtggcccccagccccaggcaccagcccagtggcccaggccccacctaagaccctgggcccccaggcgtttttgtacccatttcagtggtgaggatgaggccggctggctggccagggatctccgggtgctgcccggcccccactgccttcagacactgggcctgggccctgagctttctggaacaaacatttgctgcctggctgggttccttttcggtattcgtgcagaggcccagggagaagctgaacggaggagggaggcaagcaggagggacagctgctcggcaccagccctaaaatcctacaatcgcgggccaagggcagggcggcagagcagcgtcttcatcagagcggtgggcatggctttcactgcccacgaggcggcctgagcatgctgggcggtgcagaggggcagtgagcttctgcaggcctcgcctctctggggtgggcccagtcctcgaactcaagaaggccctcttgggaggtgaccttgaaagccgggtgccatcgagccacatgcagggctcctgacaccagcaactaggttcctcacgggacagttgttctgtcccccgctgggggcccacactgcagtgagccacaggagggagcatgcctttggcggggtggtgggggccaggcacctagtgcaggatggtggcaggagaggcctggggctttgtgtgcacgatcactgtagctattattgaattttaggagccagaagtcaatgcgtttctgtacaattctgtcagcgagaaacggcaggccactgctctggcccacccctgcctgtgtcagcggccgatggccgggctgtctggacgcagcattcccagaccctactctctggctcccagcgagggtccccaccccgagggagataagatagagttggtgtcagggaggaggtgcagacggggccgcaccggcacgctgtgagtgataaaaggggcttcagaaaggtgctgacgtggccaccatgcttaatgaggtcatcgcccaagctgctgcccctgataacctggggtcaggcgcccagcaagtggcagcagcggggagcgcggccgtctgtcgccgcccgtcggtgcccagcacccgctcagttcaggcggtcatcctaagcccagcaggcgtccttgtcctgagacaggcaaggggaaaccctgggcccgtgtgctggtggcggagggcagcagaacacagggcagctgtcagccagtggggcgcaggcgtccctcacagccagcccaggcccgccttctcttccctcccaggtcaagggtcagctcctatgtccatggaggtctttgaggctccatggctgagctgtcctgtgagatctcctctgctcggaatgctctcgaagcccgggcaggcccagggctctgcggccagggtctagcttggtgagtgtgcaagcgtgaagctctggcccagacgagatgtatgttagtgcaaagatacgtgcccgggggccagcacggctccctcaggatgccaacagcccaggctatcccagggcctctcaccctggggccatggatggggtggggctccgctgcgagcagggatgggcggaggaagcaggagggcctggtcctgccagtgggtgttgggcacagctcggtttcatcaagggcctcagggtggcgggcagggctcacgtcataggcagcttcctggatggcagccggccctgggtgtccaggggcctcctccaatgtgaaggcagagccaggcagcccagccacgccactgcctccctgggccagagcgctgtgtctgtgaggaatcttcccgatgcagcgcccaatggtttcctcctgggcttcacatttttgtgcccaggtggatggtgcttggccagtcctatcctttctggggcacacagccccagtccttgcagagtccactctaaggaggtgtcagggctcacttcaactcttttcattctgcagcctgggagaaaagcaaatcagaaaaccatttccaaaaatagccctcagagagtgggaactgtctgtgctgctgtgcgtttgcttggccctcctgggtggaggcacaggctggcccactagggaccatggagctggcatgagctgtctcccagacccagaccgggcccccagtgggccctgcgtcttgggagtggtcatccattcgctgggccaggcatggcatggcagggtgcatgggcactggcttgggggtgtctgagcagagtgcctgtgtccccccagggtccactcaggccctgatcagcacctgcttcatcccagccgccctcgcccaccaggcaatgggcagatactgggaacccgggacagggctttccgtggggtggcctgtctggccttttaggtggaaaagaccctactcagggcaggcgttctgaagacccagctggagatttctgacttggctggactatgtctggggagaaaactccgtctagggtccacttggggtcccgggagccctgcagaacctgtctctgaggaacttgagaggaagcaactccaggcctgggcagtgggacccccaaaggggtggtctccactctgcgagggcaccccaccctcccccgtcctgagggtagactgtggaagcccaaagggcttagcgaggtccagggccactggctcccctcccctctggatggtgacagcaggggagcatgcaggaccgcagcccaggagcaccttgacctctgttcagacaggtgggggtgacttttttaatgtgaaaattcagaggaatgattctctgtgtctctgtctctctttctctttctgtctctctgtctctgtctgtctttctgtctctctctcgccattgtctgcctccattctccagcccactttgtgaggttctggaccccgtgggaactcaggctcttactcagcctgagcaggaggccctgcagggctgtgacttggtattgagacccctggggatgaagaggggcaggtgcctgaggggagcccccctgagccagcattggccttgctcagtcccccttccccagaccccgaggacccgaggataaagagcaggatgaccacacactttacgtccaaaggggacactcttgggagaggatgtgtgtggtcgtcctcagggagcacccggtgtgtcggggtgcatccacctcctgtccaccgcagtgctgggctcaggctgcaccccgcgcacggcttctttatttctgcccccagcccggcgctgcccacccctcgcagcctccccacacggggctgtggtctccggcagtggctgcagtttttaattgtttttctcccaatgccccccccccaacaatctctttccccttcactgcctcgccccctccctggctgctccctgcgcgtggggggggggcccggattccggtagcatttccaccctctccgctctatcactttatttgttctcttttcctctccctttcttctccctcccgggcccagcctctgtgattaagcctcaaggtctcctgctgcttttaataggcctgcgggcttgaccatctgatgagaacggcgggcgcagcttgtcccgagtgctcgcgctggaatctcctaatgcacagtattaatcatcactgtcaagccattcaggccctaaatacacccacccagtataaaattataccgaacagaaagaaggctgtgattggcctcatattgaaaacagacattaggtgacccctgcccccacctcgcccccccccccccagtttgagggcccccccagggcctctgcccggtttaaggaacaacagacagctcagggtgtaggtcaggccacatttggtactcataaaagttctgtgtcgtgtgcaaaagtgacgcaaggcagggagggggtggcgtcctccaacgtggggcctgggtgcaagcacagggtgcgcttatgggtgcccaccatgcagacctgtccccaagagtggccggggcttcactgggaccctgcggggccagggtgagggttgtttccagccggcgccccgaggcagaaccagagccaggtgttgagccacaggccccagccatgggggtgaggaggggaaggtggccaccgcgcctgagggctcgccaactggcccccagccagactcccacacccccgggggtcgccctcgtggctgggatctgtctgcttctctctactccctagctgtcactcttcctcttggagtttgtcataaaagttaaccgggtgtgaaatgcgtgttgcgtggggtgtgctaaaagtttacggcagtggctaatctgcctggagtggataatctgctcactgctcaccttcacggcagcacccagggccctgcagtggctggctcagcctggccgcacggcagggagggcagggcccagggcacctctggtcacatcgtggaggtggcccagccctgggtgcgtgtgatgaggccatcggcggccccttgcatttgtcctaggggttgtccaaaatgccattttcaaggatagtcccactggtcagccagtcacactctgtggaagtgtcgagttgaccatggctgaggtcttaatccatgggtgatggcccaggcccctttggcggaaccctccagtcagcaaggcttttcaccacccatcatcgtttcatctgtgtcgtggcccatgcactctgcagagagagaactaggcaatccaagctcaggtttggtgggagtttggggacggccagacacccagggcttgaggccaggcctcccgcctccagtgaccacatgcttctgtgccgccctggcctgcggactccccagccccaccccaaaggtcatcccaactgaagcccccaactgagccaaccacactctgaggagcagccaagagcagctctgcgagcccagaggggtctttccagagactaagagggggcctggggacccacctgcagatggagccccaggctgctcggcccactcaggcctggccaccaggtcctgagagaaggggaagccgtggtgagctcctggctctcggtggctctcctgcgacacctacgcgcggggagctggttcttggtcaagacgcctgtctccccagaatgcgatgatgaaacaccttaaatgcctagaaatccattaaaagtcaggtgacgaggtattctgaattacgtgatattggagggactctaagaatacagaacctttgcgtggccttggcacagcgttcctcacttcactccttggctggcggtgctaggcttggggggtcccctcaagagctgacactggaggagacaacacagggactggcacggaccacagcggtggacgggggacgggggcctcgggggctgcaggagcccagggccaccttccctgtgctgacgtagagagtgtccctggagaagggctgcggctgggtgtggctgggtgtttgcagcgggaaccatctgaacctggcaggagagcaatttcctttcccctttgaacttgctaagtaaactctggctccggagctatcacagagcaagcggggcccaggacgtctgaaatcctgccccacctcccagccggtaccgtccaggatggagaaggcaggaggctatgcgtctgacacccagacgtgcagagacaggcggggatatctcagcagcacaggccgcacgtgcaaagtgtgggggcgcttcgggagggggcgcagcaccccgtgtgcgtggccaggtcgggagaggcagggctggcctgcaccgtgagggtctgcagggccctgtgtagaccagccggaaggggcattccagaacccggggaagcgtgattgggatgagcaggctggagggaggcagctttgcagggagcaccacgggaacaaagcccaggtgcagtgggaccagattctgagaggccttagaggccagatcggggaggtgggagcgagggccacagagcacccagacctgccggtgtgcaatgggcccatggagctcggggccttgcagatgcttctgggctcagggcactgaggcagagaggggctgaggttggaggaggtggaggtgggaggccaccaggcattgggtacaaagcgatagtgcgatggctgcagatggggctttcctccgacccttcctcgcctcggccagctctgccccttggctcaccccacagtcctccggtcgggttaacagcctggctcacccacacgtaccttatttttctatttcttcagcagagtggggagcagcttggcctcagtttctctgtctagcaaactagggttgaactaaacaatccagcagagctttccgggggtgtacgtgatggctacacgtgcaccgaggtctgagtgctggagccctgagcagccccctcccctgccccgcaggacacacagacactacgctctcagtggccaggctgcaaagaaggtggggagcaccaggtgaggagtgcccaggagagaggatgtcacgcccttgcttatggatgcctcccgagaaatagattgcagaggctttctcaacagcagctttgcttaaaggggggtggttcagagggaccctgatttgggagggtgtgtgtcaatgacaataaaaagaagggcagcgttcgccctgctgcagacatgctgggcaccgcactgagctcgctgcacacgtgcattcctgtaaccctcacacctgccccagatggggacgttcggcgcctctcagatcacacagatgagaggccagagcaggaggtgggtgagccgctgcccccgtgaaacagagctggacggggtgccgggttcgcctcctgatctccaggccacctgcccgctctgcccccagctgtctcaggaaatcagggcgccctgccgatcccagacactctcctcctccgacacgcccagccatgagatccgagatgaatgtgggcgaagacactcagcaccagctcctccgggcacctcccgacaagaacaattggcctgtcggtgacaaggtgacagctgccacctacgccttgaatcgagtgcaattttaagggcccaaggcccctccacgttcttctggaaaccgtgccgtttgcttttcccatgactcccatgacggcgttagcaaaacttccctaatcttaagcaaataatttccttgcccacatcagtatgcggcatgcagatggatcgcggccctggcccaagccggcccgccacgtctggtattagtggaattgttccaaagcccccatccctctcggccaagctgacttctctcagctgagaagctaaaggtgagagagccccggagtcagagggagagctccgggcagacgtggacggccatctgctgtatgacatgtggccgccgacccatcagtggtcacaccccattgagggaaacagtgtttggaatcctcattctccagcagggcagcgtggtcagggagaaatccagagggggaatccccctgccccacctcccttccccgtttgcggcatttagaagggaaatcagcagtgcaggcagccctctgagcgcttgctgcgtctgccgtcttggtcaagcctcaggcagccccttgaggaggcactgccgttgcacccattttacagacgagcaggccaagggcaagggaagtgaaatcatgcggacaggtggggcagctggagctggtggagccaggagctagatcgggcgctgccctgactgtccctcgtgcccagacacagaggcagggtcctggaaggtgggtcaccccctgccgagaaagtccccaaaccacagggacgcccatggaacaggcgatgctggccatcctttgtccaggctgctgggacgctggtctccacacaggtgggacggcttcgcatccccttccatgggccttggagcaagcacacggaccagtgctttgcccccacgcagagcctgtgtctcggctgcagcttgctcccccagaccctgagtgggaaccctgggagcccctgaatcccacccagcgagagccgccaggatcctcagccaggcgcagacaagtggcttctgctctgtgaacagccacgcccacgaggccgcctggagggccgatcatgctcgtcagccccaccgcccaagtgctcccactcgctcgggccgtgaacaggcggatcttgtggtcagacccgacggggatccgccttggagaaagagggcccctgccagcctggcagtggcatcctttctgtggctggaactctgctcggttcacagcggcaccttctgagtgagcgaaacactgtcttcaaaggactgtgacgctgcgggcagctggtggagagtcgtgtcatcctcgggtccagccagcccacactcagctggagaagctccttcgcctcccggatccccagcaccagccatggagacgcgatgctagaagtgtggcgggactgagaaaacccaccaagcggcctgggtgggtcccctccatgtggctctcgtgcagaatctcctagagcctggggcctccgcccagccctccttcgccctcccctgggtccaggaagcacagttttccctacgggccgcccggccagtgccgttgggagaggtgcagatggctttcacagggacacctcccccagaccccattcctacgattaacccccttctcgggcggcccactcacatcctcccagaggcccggcagcagtggcagcgagagccttctgcctcagaagcagtgaccttagaccactggttctcaacacaggcctctctgccttccaggggacactggcaatatctggagacatttttggctgtcaccactgggagacagtggatagagtccagggacgctgctcaacaccctccagtgcccaggatggcctcccacagcggagagtgatccagacccaagtgtccccagtggtgggtggaaaagccctgtctgaggctgtggtctcaatagaacgaaaccatgacaagtattaacgctgctgccagaacgtgccagatgcgtgaatctcaccgtgcgctaagatgctgccatcactcgggggacaggggccgtttcccttgaaggcagatgtgtagggcagcacccactggccctcgcacgctgtgtgtgttcccaccccatgcacgtgtgtccattctcagagccaggacagggatggggctggtcaagaccccctcacctgggtcagcagggtcaaaagacacagcacagtggccgttttccaccttgggggtcagtttgttccacgtccccgcctgcagataaaagtcaccataaagcttccatttccacctctcaccggctcaccctccgggagcttaggtaagtagttcctgcaaggggttttctctggacacaaggccttcacccgccctaagagggcactgggggtgagatctgcgcagagtggcagccaggggacgggaggccggagaccctagtggccacaggaaatcgccggccactgacctccactgctgcctgctccagacgcccggccctggcctcgcagcatggctgggatgagggcagaatgcagccctgggctctcccacgcggcctgggaagaggcaaggagtgaggcccctgctgcccccaacccttgctgaaaggtctcgaacatctgcaagtgtgacaacagagtgatgatcttcccggggccgcagaccagatgtggctcccagaaacaaacgtcgccatgcttgttcaagtagcagcggggataatccaccaaacgataggaaatcatgtctgatttccacctgattctgagtctctgcaccgtcaccgtgggcgatgactcatgccaagagaggaggtgagctcacccctaattcaaaccattttgagggaaacggatgctcctcggtacacggaggagaacagaaaatgcagagactcgaacagtggccagcacccctgtggaaagccagagggagcacattagtgccgccactgctgccattgtcatccggggccatccttcagctgattgacttgaagcctccgtttgcccactggagtctttgaatatatgtattttttaaaacctcagaatcatttcttaaatgcagaaaatcgggggcacaaattaaactcatgccacacggcgaaggctggccagccctcctggggaggatgactctctttctacccgggatcacacgaaaacagccactcctgaccctctctctctcaaggcgtgaggctctcccctcctcccggagcttgccgagaagcttccaggaggaggcacaaacagccggacccacgcggcccgggcccgtcaggagcgccagcacagcggccttcccgaggcggggcctgtccgcgccatctgcaagaaatctctatttgcagttacttcagagttcgaatttcaccctccactccacctgtcatcattgtttatcctcatcaacttgcacagctccttctcttcgggccttcttttaaagggtctttagaaaattgttggcaatcttaacttctttccccctggaaaagaaaaaaaaagaacgaaaacgtgtcctcggtgtgactggagagaaatcagcagccagagctcagaagcctggaaaacgagcccccgttaaaagcaacacccttgctaaaaagtgatgtttatttagaagttggcatcagacaagggcttcagacatgtgtcatgtgtaaggtctctcggagaacggacgctgtgtgcgtgtgcttttgtctggacttagacgtgcagcccggcccacgtggagacaggcagcctctgaaggcgcagacagctctttagaggcggcctgccccttgggacggggtcccttcagcggctgacggcgagcaagaagtaacaggcaaacataattaactccatgcgttcccctgagcagggccagcgtcccttgtccctaattgtcacatatttgaaagctctggtaaatgaatcaaacacttcttttatctcatcctccgggacacggcactttaatgacgaccatgtgtgcagagctcacgtgttagggaccgcggacttcagtcccaggagatgttttccgctgcaaccaaaggtggttggcgggacttcaggcaagcgaggccggtcacctctggaagcagcggagtcttctagaacctgcactgctctttgttctcggatgaaagcttgggctctgccgggatagaggccgtcgggctctgcgatgagcgcacgtccctccggtgggtcacaagtccccgatggggaacggaggccaagaacactgcagctggcttgcgtgcatctcggtcacggtggcccccgtctccttgtcctttgggcacccagactgtggatgagcctctggcagaggcgggtcaagcgtgagcgtgggctctgggatgagaggcgtttggagctggacgccatgccgggtcctcactgagtggagggagacttcagccaggctgtgggggcaggggccgcggtgggaggagagacagccctggggcgcagacaggagccgccgagccgtcctgccccagcgcctcgtccctctcaggtcccattgggaccaagctactccatttttccaaagtggaacgtccctggaaagtcacaaaacggtgccagatgcgcgtgtgctgtgctgctgcggggaggaggggctttctgcgcgctggtgttctccaacagagcctcttctctgtgactgagcgaagacactgacaacaatcaccgcagtgtccgcgaaacagggtcccattgagcagcatgaaaagcctttttgaagtgggggacggttttaagagacagcattcctcgtcccacatttcggctctactagggtgggtggacggggccacagactctcattcagaggcctctgcctttgaaccacgagtctgcaccatcccggcctgcaggggctccttcctcctgaccccatttcagcctgcgtgcctcacctgcactcagcacctggtgggagctggaggcaggggtctctgtgtacgaggaagccacctgaagctgtgtgctatccggaggccacactgaagtgtgaccaagaaccttctccaatgttccaggcagctgaggagcccctgcttccaccatgcagaacagcgcaccccccccccaagttgtgtgtggtccatgataatcatcgttttgttaagaaaagaaaaaagctaaaaaagagagagagagatttgtcaaacacaaataacagccacggcatttgtctatggtgcagcccaaattttacccggccacaaccctgaagttcattttgggcatgacacctgccaaaccccggctttcttggagggaagaccgaggaaggaacgcgtcctggactttgggagcaggccccagacgggccaggggaagagtgggatcaaaggagactcagccagtcggtgggaaatgacagggcccaactggccaccgcccagaccagacttctgctggcggccagccagccgccggatgtccatcgggaggcggaggaaagcaacccctcttgactcagtcccacctctcaggcagactttcctccttggagtgtggagtggcctcattttgactctggtgacctcatccttcctgttctggcacaccccccactcacctccagcattcgtagtgcctattgcaaactgaaactctgtggccagaggggacacatggctcgcccgccctgtgcatgggacgcccccaggagtccttcccctgagaacagagaggcaggaagaacagagacacaggcacgttaccagcacaagccgtggatcctgcaaggagagggaaggaattaaacaggagacgtgccgagggcgggggtctggggggctccctggaggaaggagataggcacccacgggggccggacggtcaccagaagaccagcacaggggtcacttgtacaaatgtgactgagaacacaggtcacctcggagggagaaggacactgacggcttgattcaaaactagatgtggatgtggatgtggatgtggacctgatacagacatgggcacagacagacatcgccctcaggaacaggagtgtgtctcttcagagcttgctggaaagcagggatttaaagagcccacgactcgcatcctcattatttcaggaccagtgaaggcccccttgtgactgctcccccaggaacagagagacggatcccggaatctggggggctgctgtgcccatcccaccaatgacttcagggaggatctttctgacccagcaaatccaagtcagtgtggacacagatgacctcccctgggcagctctgagcctcagggaccaaaggcagcaccacagacctacggcccggggcacgaggctgccatcctctcctgacaacatcagcatcagaggggctgcccaccccacgggctggtgcctcaggaattgctgtctgtccactgtcattgtcctttgacaactagtttaaccgccagcgtgaaatggacattcccacgttgtccttaattaaacattctcaggggaggtattcatttctggggaagccctcccaccgcggagggagtcacggggttgatggcccgggaacgctctgagcccaggaatccaggcgagcacggagcggctgggacttccatgtcctcccaggcctcagacgaggctgcttcgcccatggtgaagcggtctgcgatccagcctagatttcataagtataaaattcgtaatattctcactttgccccccaaaaaaggccaccttccaaacaagcctgcacccctgcctcctgcgcagctctgagccggccgcagaggtgcagccatgaggctggcctgacggtcgtcttccaacatgattacggccacctgaggaaacccgcaggacgctggatgttgggaccaaaatccttttgaagagaagggaagaggaggaggagaaaggaggaggagaaaagcccaaaagaaaacaaaacccggatccatgcaggtcccacccccagcacaaaccgctcaaccgaaagcggatgcccccagattagggaccagaggggcccaggcctcctcttgagccagccctggaggtcttcagaaacgtgaatgagccccttcatcttttctacgggccaggggcatgtggcctggaggggagatcaaagtgctgcccctccgccactccccccagcttccgactcaggctcagaacttcaggtgggcgtgtggaagggcgtgggggtcagacccgccccaaacccacccccttttcacagtggcgtggcaggtcatgtgctcaggatgctgttcctcccgtgaggtctggggtatgggcggtttaacggttcctccggggttccttgtttgccaaatagccctgcgcagcagccttcacagagctcaccttgaccttgtgctggagggctgcagataggcctggagacgagctgcagcccctcgaggtgcccagagttggcgctctgggttctaacccagagcccctcctctgataagctcagcaatggctccttctcatttcagcccaggtcgatggagcctcccacatccagaatgtccactgcccggccacgcttccctgccctcctcaccctggcaacagcctggaaatgaccctgtgctcctgcacgtggccccctggtgccagggcaaaattaaaccagactgatggcaacatagttatttgctgcccaggcgattccttccttcccccacccagctgccagggggacaggtgggccttatggaaaccagagcacattttaattatcgcggtccggccaggaggaccagaagactacatatggacccactcttccttgagccccgagaaagacacgttttttgcttcttaaggaagcggcgtgttttagggctgcctaatcctctacgagctccgttcttcccacacctgtgcgtcctgccgtccgaacccacatggagcatccccggccagggctgcaggtggagataaaagggaggaaacgtgcagggggccgcagaataaatccggcttgtctcagtggggaagtgactcagtgggtgaggctgcaggggcgaggaggggtctcctggaagcaacccggagtcagagggctgtgggttcagtgtggttggttcgggtgtacctgagttgagttgcagggttcccccggactcagcaatgaccggaggccctgggatggctgctcaggggcactccggtggggcccactgcagctgcccttctcctctgcatgctcagcccgtcgcagagccacaaacacctccaagcacctactgtgcacactcctgagggtcacaggtagatcagatgcagtcccacccctgagtggggacacacacgcagagcaggcgacccccccggggtcagggagaggaaagtgggtatgcagctctgcttgtgaaggacggagatacaagtgcccctcccacctcttcctcctcagcactgggggaaggctgcccacgccccaaagctccccctccccgaccttgctcagctgtgcacaaggaggggcccctggctggtggcgtccctgaggttgggcgaggagaagaaacactgggggctctgtgccgggcagctggcggacccaaatgtcagccctcatgtcagccacaggccagaggtgtcagggccggcgctgctgctgcacacacaagtcataaatacacctaagtggctgctgtcacttcgccgcggcctcgtttccattctgcacaacccaaggtgattaatgagaattatcattaacgctaatcagagccgtttggaggaggctggggtggaaggaaatgccctttgtgggctgcggagaccactctaaaaggtgtcggttgcatgcgtgtgcatgcttgggggcgcaaacatgcacacgtgcacactcatgcactcacacatgtgcacacacacatgcaatccatgcactcatgcacacaagtgcacacacacgcacacactcacacatgggtcagggctctgaggctggctagcaggagagagtctgggcagatggggaagagccgatgctcagaaatagccctgccctgtcgtgaaaggcaggctcccatgtggccgccccctgccctggcgtgccaacagccggagaacacaggccttgcgcagcccggcccggctggaaggaattctctcccgccgccaccgagcacactcatgacctcacgtgcctggcagttgctcacagactggacaggcttgcccaaaattagaaccagtgccgggaggacaggggctgtgggcaatgctgacacaagggtcagtggagggtacctgtctcctccttcccttggcgcccagccttgaccactgcgctcagccctgggctgcccactgccggcccattatgccatggacgcctctttcccgctcgtctccaaggaagtggccggccaggctgcgcagggggcttccctgccctccgcatcctcgctcagggggccagtgtggcttaaagatacaaagtgtccttttattggaaatatcaattcagaaaggggcgagtagtaagctgagaatatttgggtaaacttgacggcccttaggactgaaaagaccttttgttccctccaagaaaaaggctgtcgcaccaccctgggctggctggagccggtccaggaaaactgaggtcggagcagaaaaatgattagggcgtctcgcgaaagatctttcctctaggtcaaatgtttattattatttgttttatttttttattagaatgtgtggttaacatcctattgcataatatcaggctgtaaccgcgccttcgaacaccgagtcttggtctgccccagtctaccagcgcacaaagggaactttgtggcatttggggggagcaaggggctccgccagcgtggtttcttttgctctgctttgtttgtgagatgtcgaccaggtaaagaaggggtacatgtcttctggtgtctgcactctgacaagcagagggtccctgcagctcggagatctggctgtcacaggggggccacctctccatttgcaatgacaggaggtcgtgcgtgtggatcgcacggcatgcgtgatttgggagggtctcaccccacagagaggaaggtggaatgcaagctcttggctttgcagggcgggtagggatcttgcaagaagctagcttttggctgaccctcaagcccaacgcatcaacagccacacggcttttccggagacactgggacgtgccggggctgccagccccagttcctccctgaaacagctgggcccaagtgtctgttgtccagaaacatgcatacaacccaatttcacggctgtgtctcaacttaccagctccccgaggagactagttactctttaagattcccgtgaaaggacagccctcgagaagccacgagatgaaaatctcaaacctaatttgaatcctcctccacccaaacacatgaaaaggaaaagagctcgtccgacgcaggtcactggaggagaaactgagccgaggtcaagtgagaggtgacttggccaagctgggggatgttttcactcagagttaccccgcccaaatgaagtctaattaccatctgcaagtgagctgttcattgaaacattctgggatctgagttccatgagaatccaaaaatttctccaccagcctctgcagaaaacccgcggatggtgggttttctcatcagtcattcgaggaccccttggcatgctcgtggaacgcagagcacagccttgagaagctgcctccgcggcgagtcatgcccctgtgccgtggccctggacaaagcaggcagtcatcctaagggtgcatctccacgtgccacggcctccgctcagccctccccggcgcacagcaagctggaggccaggacctggggcagcacatccttcccagaggtggctccatgagccgaccccgcagccaggcatcccaaggagtgttgttggggaccccaagcagaagccttccacacagccctgacttggccaaccggggagtccccagtccaagccagcccctgagacgccttgtcaccatcttgtaccaacaaaaggctgaatcccaaaggccagcgttctgactttgtgtgtccattgcctgtggctctaatatctagctcaaggccccaaggagatgaagctttcagagaatttctgcgtccaccatagagtgtacgtgtaaatgtaagatgctgacaaaagtggaagcagtcatgtattttatagtaattacttctgaatttagttttcataatttatagttgatacgacttaaagaattactggtttgagaagcgagggtacttattttaaatattctgattctgttatatttttcactatgtttttaaaaaattaaattggatgttaaatagaaaaaaaaaacttgtcttcaaaacaagacccgtaggaaagaatcaaagtgattatttttacttaaaaataattttaaaatagtcatcatttagagaggaagactagaaaaaaaaaattaaagtaaaagtagagtaatagctgtttagagagtgaaacgaaataaagggtttggggaacattatgcatctggcatacctcagggctgaatgtcctcacagggtcagagaaccttctagaaaagagcctatgccccaacagaggggaggagctgtgtgtgccgaagggaaaccaccagtttggtggacattcctgaccttcgagggctcgctggggacctgcttgccagcttgagctacccaggccttgctgcgtggagttgcagccagcactggccgggcaggcactacatggggtggctctggcagagtctccaggacatagttctccctcaagagccacaccagagaccaggtgtccaacgcctccacccagaggaggtttggggtctccgtcactggagaggggctccacgatcaagccctgggtggctgccactggctaggaagacttcaaaggtaactcatctgtacccatgggtgtcccgactgtctgcggaggagccagagatccagaaagcccaaagtgcaagggagaccactaacggcccctcccccaagaaacaatgctccccccccagccacacccaccccaaggagggaaaggaccatctctgcacacacatggccgctctcttgcgagaactcccatccagtcacctggggaagtgtccccacaggacagcagacgggctagctgccctaaaccgcgctgcacccctttaattaagcaagcagagtgctctcttttcccgtgggttttcattcatatcctgacaacaccagtagaaacagatctgccagaataagtcctacgtgcagctcactcaagggaagaaagtgagtgtgtcctcccctcctttgcctttcttaaatgaaaagagtacaatagcggaacccacaaagaaagatcccgattaaaatgttccaaggctcttaaaacacccccatttaaattctgggtaatcggctgcattgtgctgcccctgggagaggccgtggggttggtgggtgcagctctgaaccatttgtgtcatatgaatgcttgtgattagatttttaataaaccgtttcgattctatttcctggggttgaatagatgtttgaacttggctggtatggacttgaaagggatccaggtaatatcatgggggacacacttttaattcctttaatatatttcgtcaaaggtgactggccgtaggaactggctggaacacggggaaagagggagagctattgaatttaattggttccatttgtcattcaagactgactttgccgcacctctgtctatgaatcatttccaaacttgctgatctgacccaatgtgcccacagcttgggaaggaagccggcttagggtgcccagggagatgtcgcaggctgagcctgcatccaagcgcaggcctggcgtgcagctcaaggcccctcactgagaacctggatggcctctggccaggatgacagagcctgagaactggagaatccaaaagaagcccacatgagccagaaacccaacattgccctgtctcttagtctgatgcactgggagggttggtgcaagagaaaggataggcccaatagatgacagttacagaaccccggggccccgagctccggagcacccggaagaaacagaaatcctaggcggtagaatcgttacaatgagctgttgggaggaaagcacacctcctccctctataagccaaaacaaaggcgcaggcccaggccagcatttttgtggatgcatacctgggcctgctgggggaagaggccagagctggctatttgcaggacatccaaagacaacaccgcctgcaccaggagccctctgccaatgaccatttctgcatgtcaaagagctgggagaagtcctcgctatgggcattggcaaggtatttgttgagattcggcccgctgagatttgagctatttgcgggggctgatctcagaacgtgaggcctgatacggaaggcgatgggtcacggagcttccgcagaccgacacgtcccttggagaaggaagaaggatgggctcggagagactgagtcagccgcagggaaggcactaaacatgacctggctggatccacccagccgcccgccaggccctagtgtgcgctggctcccacagtgctgagctggaggagccggcgcggcctctgccctgggaaacaggatgctgccacacggacctggggggaacatcgcacggtaacgctgaacgcaggacaacagcgccgtaacactggacccgggaaaaattgcaactgatttccagagggcagagcgactaggagggtttgtacctccttcggcagaagccattcaggaatgagaagggaaagggtccagaatgtggagaggggagatgaggagagaaaggcccactgggcaaaggcctacagcagcttgggaggcagtctcagttgtcttgaggcaccttagttcattcgtttgtcccccagacattcactgagcacccactgtctgcctcgctgtgggaatcggcccttgtcatccagaggaagaggaagaaattatgtggataagacaggcaacggcactctgtggtccgtactgtgatgggtggggcgggggctgtgagatccactcagaggggcctacccccatgtgtgggatcaaggcacgcccctgggagaggagtccctgtgttctgccacctttacaactccttgcaggatgttcccttgtatctcacctgggaggtggaggaagccagaggtatctgggcaggttggaaggactgtgctcttcatccatccatccatccatctatccatgatgcatcatctaccccgtcatccatctagccatccatccatccatctactcatccatccacccaaccatccatccatccattcatccatctactcatccatccatccaccctcaaccacccatccaccatctatctgtccatccatccctccatctgtggagacattctgtgagccccgagtgaacactactccttggtgaggctctggaagcttctcttgctcttggctagcatcctcaagtagaggagagatgagaagggcaaccccggccgggaggcatggcactggtccccacagatcatgaaaggggtgagatgggcagccagtgcccactatccagctggtctgcccctttctggctgtgcgacattgggccagttattcacctctgtgtacctcagtttcctcatctacaaatgtggctaccacagtggctgcccctcaggaagatgtgagcatctagcgactatagtgaccgtagtgagacttagcagctggaggagcggtcatcgcagcgtcagcagtctgcagggtggtggtggccagagggacttgcctcctcccgagcaagggtgtggtggagagagccccgggggccaggctctgtggggccacggttctgcacatcgctgacctctccagagagcaggaggatgtctaaacaggaaggggtgcgaagatcccgagcctccctcagcccgatgtgttgggttctctgagaggcgcatctggaagtctcttgcgggtgcagcctgcgttgatccttgtccacagtgatggggacatctgagcaggtctgagggtaaggaggccccaggcctctgagcagcgcctccccaggttgtgggctggaggatgcagaagtgccagccagggggacagggctgctggaggaggccctggggatccttggtgggcaccttggcctggcaattggcttccacacttaggcctttgtccggcaccctgagagtgccggtgggttttggaagcccccacccccgatttgcctcggtttagccattgcgggaattcctcttgcacctgctgtgggggtgccccgttcgccagctctgccgttcgtcaccgtctgtgtgaccagaaaaaggttatgaatgtgacacccccggggggccttgcatcaactcggtagaggaaccgcttcctgtgggcagagccgggctcccacctcaccctgggcaaggacctgccacgagcctttggttttggttgtttgggctttttttccaatctttttttatgatcaaaaaacaaaacaaaagtttctcacttcagaatgtcacagctgaggctgtggtcgtgtgcagccctaggctcagagacttctgctcttggcccagcttcaagtcaaattagggtttgaaaaggtgctggggctgttggcgtttaaccacatcggcccagggagcggggcgggcctgaggccagctgtggggccgggaggggcccgggagtgaaacctgcaagctgcggggacttcacaagccaggccaggatttggggggcttctctgcacccacagcctttacctgtcccaactgtcccgggtgttggcactttggtggcttttcacaactctgcccttccccgagcacctctggtcggcctccggtccaaacaaagcagcaagaccggaagtggacgtttccacccgacaaggagccgaacgcccatgaggctggggtggggctggcctgggcccctccttgctggtgtttgctgggagcccccggtgggaccatgcttctgtatgtgggcaccctcaagctgggtctccattcccccaccacggtgccgggcagcggggacaaggagggcagagcatttacacccacttacacctggattatgtgaggtcagaaacttctccagggacccactctggcaagtggccggccatgtgtgcagccatctcctcggggctcaggtggaagggaagccctgagtctgcagttgcttgctttgcttggcagactcagggccagcccgaaacctttgctttgaccctgttgtccacacagcagctgaaatccggtcttggtgcttgtgagagatgccccagaaaggccacagaagtgggttgggtcgacctggtgtgatggttccgctcgcttccagtggtcgtccctgcaggcaggaccggcacaggttcacggggcctggtggtcagcacagctgggggactcaggactcagcctggcgtggtgtgaagcacccacttgtcctgcactgagagacacaggagatttcagccaggagggagcccaacacaggcatctccacggtgacatcaggccagtcctgggatgtctcccacccagaccatctgcaggaagagggcggagcagggcccaggcccagctgccccgggagctgctgtcaacaagccatcccacttgcttttacttggaactctcggtggcgtgtgacccaccctagtgtcctgggcttggctaggaggacatgactgtgccatcactgtcacctgaggcgggccccactggtgacatacctgccttccctgtgaggatgtgggatggtgttagcactgggcatgtgtcaggagctccggggccagatcctagggtcccagctgtctcctgggggcgaccggcggagtcgctcattcttgctgctccggcgacgtcccccctaagatagagggctgcccgcctcctctagacaaggccctctccacctgcccccagccgaagccggcgagctagcagccgccgcgggcactcaccggcagggagaacttcccattcgaccacaacgcagcacccctttaaagagggcgcatccataaacggggttccccaggacagaagttcagtttaaaaacagcatctccagagaaacaacgtgtgctgtcccctgctcccagcgatgctgtcggcagacacgcgtgtccgctgggagtggctgcagcagccagccaccccacagacggagggtccctggccccccgcccctcagcccctggctcagccgcccgcccccggtctgtgaggaggaccctctcctgctgggactgaggccgggccctgggctctctgcagtgaccgggggcttgctctttggggcaccccaagaaactccaaacacacaggtggacagacagggtcttgaagggaggcacacgggacggagcctgtttcctgagtcccctctcccactcactgccaaggctgctttgtccctttgggggtcctggggcacccaccgcagattcccaatggaccgggggtctgtccttcctgcccacccaggtgacctaggaaagggcccgctctgaccaggaccctcgttgtcctgtggtgggcagagcccccggctggcatgtgtctgaagagcagatgggcaggtgccactggccagggaagcagcaggtgcccgcagcctgtggccagaaatatgtccccacagcatgcgcacactcgagcgttcccaggggtgggcccggggctcccttctgcgtgtcttcaacaacctaccgaggacaaatcgacattcctcacagtgctcctgtgtgcagggagccgtgggcctccgcccagccacaaagcagaagagaggctttttagttccaaaggggaaaatgaggcagaactccagataaccttcatgttatcaaagtgacaattctgcaatttggaggccagaaaggcacctatggttcagagagtccagccccctttcagagcaccctggagggatggccaccaagaccgcctcgaatttgctcagtgacggagtgctcacccctttgtgaggctccccacccacctcatgggctgcccctagagatgtctcagccactggtcctaaggtttcaacagacctctccattgccgacaccctgaggggttgctgtatctgacggagtctgtggttcattcatttatttccctgtagactaaagagcactagtttgggtcacgaatttgcctgtttatttgtgagcggtctgccgacagcccatggccaatgagacaaagtgtagacttgccgtgtggacgagacatgtgcttgggaggaagcccagcccctaaacaccccactcaggcccagagttccgggacgcccctggagtgaggttgcgatcggctcccaccaaggccagtgacctgggtcctgcacaggcagcgtggacgccccacggcctgggtctgcctctcttcacgtccctgtgtgctgttgaccagcccgggacctggagaggcctcaggacgcacgacatcagccccaaactcagctttgctgcatcttacaccccagttttctgcaaagtgcgcagacgctgctgggagagttgccccccttgtgtgtaaccccaacctcgaggaggaaaaaaacaaaagggagaaggaaagcaagaatcaggtggaaggggaggaggggacagagaaggagtgagagctcagctggaaccccagcagctgggagcggcgctgtcgtctgggtcccgcctggcagccagggctgcgcaccccaggagcgccgcggctcccgagggtccgggtattttgtgcgcgatgatctgtgaaccccaggaggctccatggcacccgggtgtctgcatattttgggggagagggtctaaggacactgggagttggagtgacattgggatgtctgaacaccttttggttcatggctttcctcagatcttcgatgatctctgtggctccagaacactggagagcgtgctgtctggggtccggctctgagccgagaggctccactggggaccaacacaggaacaggcctccaggcgtgaacttgaggcatcggccttgctgtggcctcagagtctcccccattgggctcccggcttcctcttgcagcatccccatcctgggctgtgctcttcagtctctgagaccaacaggctctggccctcatccccccacaacacccacaccgcggccctaaaagtccagtggaaaccacggtcagctggggcgtcctaggagcccccactcagcttctttctgacacaagccccagaaaacaccgctctctccatccctctctcttcctgagcttagaagacaggctggcctccaccttggaggaccagctccgtgagttactcctgtttcctagaactagccccctccccgtcacctccctcttgactctgttttctctctctccctccctcccctccccctctttccctcccccctcccttcccctttccgtaccccctcacttcctccccctgcctccccgctgccctccaccacctccctctcagcctaagcaggtaggacccaaaggctgtgaccctcccgccaggactcctgtctgcctggcagcagtgtgaccccatcgcgccagccacatttctccccggtgcttggtcatatcaccaccgagccccataacgagagtcgggacagggatttatgagctcgtccaggctccgtgtccacacccagacagaggtgtttccagaatgcctttcgcccacctccttctgtcacaagaatagactttctgttccagctcctcatggatgccataaattgtaaccacagtgtgtgtgcgtgtgcgtgtgcatgtacgtgtgcctacagacagaagaggcctccagctttgtggccagggcctgtggctctgggtcagctcgtgggtccttaaagaaggggccagggtgaggtgagactgctcccccccgttcccacccactatcataggtggacgccgtacccaggcttctggggagccgagttacagctgcaggctcagggccgagggggagactccagggccgccccctcctgctcacttggaatccattaggcgagctgacgacagatgcttcggcgttgggcctgcgtgcacaccccggacaggcccgcggctggctgtgcccattaacgggagcgcaccagcccagcagagcccacaaatgagaagtgcaggaaacctcaaacgatgggtagaaaaaattgtcaaggctcagagaattcttcatccctttgcttcataactcagcgtttctgacggggggatttacaagccggggagctggcagggtgacaaggggtagggaggcctgcaagcccaagtaggggagagtgggcaggagggcagcttccttccgacgctctgggattcccaagactcagctccaagcagccctgagccaggcttgggagtggggtcaggcaggggagcagacttcagatctggagagagacccagccttgagccctctgcctgctaagccctgcctgtaggagacgccaggggctgggcggcctcctccttccttccctcgctccctccctcgttcgtgccctgagcagaactgtgcacacagcccgggacgggcgctgtgcaggtcagtcgttcctcccccacctacttctaattgtattggcctcctggggccagggctctgagccacagaccaaaccaggcctcaggacctgctgagctggggagggaaggaccttgacccgtctttatggcgttggaaagtctcactccaaaagccgggggagaggaacctgttttctggattcaagtgtacagaatcccgcggttcttggaaccagcatggaggccctccttctggcccttgacctggctctggaccgcgcaacgtgcagggcggcagctgacagcatggctcaggagtggggcagcctgggctgggagcagccctgctcctgggtcctccatctcctgtcctgtaaaatggggcaatgacagcacgtgccccaggggtccacagtaacaagtaaggggcgacactggcacaaagagacctgagtccacgcgggctcatggtgtgaggttcccaaaggccctggctcggccatgcacatgtcccaggaggagagcagagacctgaaaggaaaagaggcgggaggaggcaaatgtctgcagcctgtttctccctttctggagtcagaaatggcacaggttcgtccactccccagacaagaagccaggcctgcaacgtcctgcccacctggccagaagcatgggcccatggccacccctgggagcattcctctggtctgaaacagaaaagcagcagcttctgagcctccggcccctcccactccatcattcaggtgtgcacaggtgggcccaagtgaggcccccatcgcccggccattatgacagacacaggctcagccctctcccagtgcatccgctgctggttcagggcccctgacgcggctgcttctgtcctccttggagaaaggagtcagcagttcacctgacacatctcggaggtggagtttgagtctctttggaaggaaggagttaaccaaaaccgccatcaccccggaagcccagccagggtgtccaccagggagaagggatctcctctcacctcctgggccaccagaaagtaactcccacccgctgaccgtccaaatggtcagaagttgggtgaccaggtctcctgggtcgaggaggagggacgtttcccaggattggccaggaaggagaggctgctggccgaggagtgggaggacagccagagccaggcaagaggtcactgaggatttatggcctccaagccctaggccctggacccaggagggacggggacaggcaggagggcccaggacagtgtctcttgtgggtgagcgatcagccgtctccatgttcagacacacttgagagaactggagatgtttaccctggcacgggaaggcttggaagaacatgggctacgttcacacacctgcagggcagtcgtggggaaaagagagaggcttgttctatgcagacgaaggggggaagttaccggaaggagggtcttcccatccaaaagtgattccccgtgatgcaataagattaacggtagaccaggctgtctgggagcgcgctgtgggggccctgcccacgggagtgccagaaggaggggctccagtgtgaggttgaatgggatgagcctggaaccttccagctgtcctgtgatgacgcagaagctccaggaaagagagggtggcaggccgccactgtgttcagcctcctgtacgtctcctcattcccccccagaacaagtccttcgattacctaaaaggtcaggcatgctctctagggtgacagagagaaaggggacagcagaggaccgccccagcatgtgcatggagctcccctcgctgtgggagacagaggcccttggggagcaagtctacaaggcagaatgcagagtccagccccccagagaatctgcctccggggctcagcccgggaccgccatgtccacatttgaaacctgcacccaggacattctgccgccggccccggccacctggggaagtgtgaggacgggagaggggagggcaggccggggaagacggtggtggagaccggttcagacgggcgatgcccacgagccggagggaagcaggtggttcacagggtggtcggggccctgccggctgcctcagggccacttcctcgtggggctgatttgtcctttcccaaagcacaactgactgctccccacgaccccccgagttgcaccccgatacacacacacacgcacacacacgtgcacacactcatacacacacatatgtgcaaactggatgaaggggagcttccaagcactcaaaacagtagttagatagttagttagttagttagtttagttagttagttagattcttcttccaggagtttcttggggagacaggcctcaagcctgtcatggtgaggcagcacccttgagtcaacaactagtccccaggagaagctggtggcggggaggggaggctgtgatggctcacagcatcctttaccatcttcaccagtcccagacggggccgtgggggtggcgggtggtgagggggagagacccagagagacaggccaacagacacagacacggagaccaacccccgccagcacccatctgtgcagatgctgtgaggtggggaccacagggagctccggcccccgccagtggggtggggtggggtccgtcaggctggtcggcttcactccctccctgtgacagaggaaggtggcgaggctgcctcatcgagtcacgggaccactaaccacagggtttgcttggtgggtttctttggtgggtttcctgcagttggggtgcagaggtgagaggttagtgcctggggaggggcttctcccacgtgggcctgcgctcctccccacccccatcagccagcccagtggccccctggagccagcacagggcaggccgtgcccccttcccccagcattctgagggtctggcattctgagtggctgaggacaggctggctaggacagagagttcctctgctgccatgttgtccctccctccccacctgcccgtctctcgtctccatcctgcccaggccctcggctctctatcttctctttcagaattaggcccatttggttagagtccccccaaaatgaagaactctctgaaccgaggccacttagctcacagccctgttccacatcagcctccagaggggtcagctaagtggagcctcagcgcgtctagaaacttgtcgtttctgggaggggctgccgtgggctcccggaggggtttcagggcgccacagggtgtggatgcccagacggactgaggcaggttctgcacatctcgcagcaaagcagctgcctcctgaaccctcgtttcctccctgatttggaggcaaggcagggcccctggccaattaaaaagtgtccgtggacgtttgcagagcacctgcactgttcacaaagcaccgtgtttctccagccagaagtagcctgctccaggcttccagggatagcctccctcccttcccaggtcctcacacttggtgacagggcactcaggagaccctccagggaataggagccatggtgccctgacttcggcgggtccttctgacactttgcttccatcacccctgctcaccaggacacacctgcccaccaagattcacctgcccatccagacatacctgcccacctggacacacctgttcaccgggacacccctgcgcactaggacacacctgcctgccaggactctcctgaccacctagacagatttactcacctagacacccctgcccaccagggcaccttgcccaccagacatacgtgaccacctggatacccttcctactccaggacctcagtgtggtggtgacctcagcgcccaccccgccatcccctacccctcagcgccggggctgggctgcccaggaagctcgcctaaggtgctgccggcttccaccgttctggattcagagcccccagcaggcactgaacagggcaggcgggaggatgggagggactccacctacagcctccccctcagggcccagcaggcctaagggagtccccctctcagcaggggacagcagtcaggtgtcctcataaacagcctgctcagagtgcacaccctcagaagctctcagcttgtgtgagtcaggggttcagaaaacgacagcaacaacaaagtaactaatgccagtcaagagccgcgtggcctccgagggtgctaagagaggctgggcgtctgcctgtttctgagtgtcaaatggaaactttccacttttaagaaaattaaatgtttttcttactttcagggtgatgtgcacgctggggtgagtgtgcacacacgcatacatgctcgcacatgcacgctcatgcgcgcacactcacacccaggctcgtatgcagccacacaccccaacaacacctttaccttcaagaaggtgttctccagcccagcgccccaactctccttagcagaggctctattctgagactcaagtggcctctgctttgtcacacttggggcggctctactgggggacttttgcatctctggtggcactttttactgccgggccagggcagcgatctgctcggtgagaaagggaagggccatccttccccctctccacagggctccaggaggctcccgggcgtgggtgggctgggtgggccagggccccgcagatgcccggcgggtgggctgttgtccagatgctgaattgccttcctgctctgagcacagacagctgggtttctcgttgggcgaatgcagagagtgctgaccagcacacaggatgtctctgactcttcttggttttctgtcttgccgggcagatctccgaagacctgggcagtgagaagttctgtgtggatgctaatcaggccggggctggcagctggctcaagtacatccgtgtggcatgctcctgcgatgaccagaacctcaccatgtgtcagatcaacgagcaggtaggtccagggtttgcacacgggggatatctgcccttggagttgtcctgggggccttggcattgagacggatgggagtgggggtgtcaaggtgaagggtcagctgcatctctgccgagcactttgccaagcccggtcctgcactgggggcggctatttgacacttggccaacactgagtttccgggagctcagtgacaccccactaatgctccatcgaagcctgcggtcagcggcctcgcccaaggctgactgggcttgagagccaaaagatgagggcttgcgtgaaagaagggagccggagcaggcgggatccgaaactgcagaggtccacattccctacaaacgccactcccacgagagaagtggagaactggacctagccgcttgagaatcgtgtctaaatgactctgtttgaccaagctgtccggaagggtcagtgctggctgtctggaaggctgaagtgtggccccagctgcccagagcccccaggaatcgctatggcagcccgggcagcctgcagagggtactggccttgctttggcacaaaatcctggccctgtgggcgagccgactgatatttttcctgagctatgtggtcctgacttcagccccaggtctcaccagggccaaggataacctttgcttctgctgccagacccccgggggccaaggacctccttcggcacgcctgagcctcgctcactacagcacatctgtgccaccccagggatgcaggcatcactggctctcggtcctcgtggcaggcgtccacacccatgagctgatcaggatgtgggatgccatgcggagtttccgtccaccaagaaggcaacacgtccccagcactgggagctctcagtcgatgtctttaattacaagtttgcaagtggaccccggagactcctcatgctcctgggccaggctcctccggccacaatttcgttctgtgttcccagagcactgggttcctggggagcccaaccctttccatcagagcactttgcttttgcctgtagtatcttgaggttatgcctgagatttcacttgcaaaaaggattccattgctttcaaaaatgtttgcggagaccacaagacaaagggtacattctcgcgtcgcctgtgcaaaccattaacatcggctcgaggctcttgactttagagaaacagtttctcattaagaaatgcagacagcgcacaccagggaggtccctaggacccccataagctgccgggcggctctccaccatctgcatccttctcggccgtggggctcagctggaccaagcagtcctccccggacacctgcgggtgccggacacggtgaagctcgagtctgcctctgctagcgggacgtctgctgctgttagttagaacccaaaagtttgaaagtaacttttatttctgattgcaaaaaatattgcagaaagaaagcaaaaggaatgatcgtgaaaattaggagagaaccttaaaagacactttcccttcttccagatgagagtgaaacgtaagtgaaggagagccggattgcaagcacagacagccctggcagtgcgcttttcttgtagagagcgtgtgtccctggttgtgtgtttgggagccgaggggcagcagaaggaatgcctgggacccgggcccaggactgctcttttgtcagacacggaatagagtaacaaaagagccagattttttagttaagaaataaacgtttgttagactgtatgaaccaaatttaagcagtaacagagatatctttatgggacaagcttttgaaaaagcaaatgagacgtagagtcttctccaagagaagcccgtcttcaagcatcttcgtttcattactaatatctcagggaagcctttatctcagctccacatagagcccaaatggaatggtccgctttgcagcaaactctctttccgtcgtcaggaccaggggacagaaatactttctgctgagatggaataataaatttttaactctcgttgattccatttcgtttaacccactcccaccggccgacgctgtcctgttgccaagctccatcctacaggcagaaacgttgctggctctatcacttaaacccaatgtgccatcaaaactgcctccaactctcctcgcctactggacgtttctctccaacacagcctcctggggggattaattatccccactttagttcagaaactcatggggaacttgagatgtgcatttgagcaaaagcaacaccgaaatggcccaaaaaagggaaattcttccacctggggcctgtcaacccctcgaccctctgaggagacaggagccctgcgcccgcagcgggtgattcctggtgttgcagaccagccgagattccagccaacatgcccgctaagtcccccgtctgaacataatcaactgtgtggccgtggcctccgaaacacacaggagagcgtgagccaccgagaggcgagagtggctggccggtcggcaccgggccgggcggcaccggggaagtcgtcagacacccagcttctccttctaaaacccaactttgcagggtggcccatgtcccggtggttgagtttgcgcactccgcttcagcggcccagggtgtcaccggttcagatcctgggcaaggacatggcaccgctatcaggccatgctgaggccacatcccacatagcacagccagagggacctacgactagaatatacaactgcgtactggggggctttggggagaagaagaagaagaaaaaagaagattggcaacagatgttagctcagggccaatctttaaaaaagtcaaatctcgactttgcatttcaggtagcaaagaaaagggagcaattggtgagacgtattttcctttgaaaaataatgctcataactttaaactataataataatatgaaaaactgctcttacatactcagagactctcaatatttgttgcttgaatgaagtgacttttagaaatgcttttttgaacgggatataagagatttcaagctattttttaaacagtctcccaaaaacggacttgctctctctcgttgggaaggccaccagcctcagcagaaagcattttcgttcccggtcctgaccgctggaaacgcggatgctgcaaagccaccgacgcgactctacatggagctggaataaaaacttccctgaaatattaataatacaacaaagatatctgaagacaggcttctctttgaaaaggaaacattacacgctattaatggggtttctcagatttctataatcccaagagtttctaccaggatttcctggcacttatggacacccccacacccacacgcaaacacacacaaacacacttaagggaacaatttccctttttctctctcacccgcacacacacacccctcctcccagcccagcacgggcgcagagaggaggggatccagattgaagatctgagacccataaaacagtttcaagtttgccagatgggattcaggaggaaagtcatttaagtcagaagagcactaatgttgtcttgaagttcaaggcacatgaccttagccccaatttccagaagacagagggaccgggcaggaacagagtcggggtcccagagagggagttcctgaagccccagctccctcgaggagtgaaggaggggcgctccaaacagcctgggctcccagatgctcctgtgtcttcggagagacccacacatccgtggggcttggaggtcagggactgtcatcaacctcagcctgtcacggcctttacgggtggcccgcatggtgcgtccagatgggccctgggaacagatgcaacccgcccagccgtgggggctctgagcagctggcggctctccagctcatgtcgtgggcactcagggagcccagacgctggcagcacagcacaaggacgactctgtccgtctagccaggcctcgttctccttggagccagactgctggccacgtgcccccctcccagcctcacccagcgtgccctgggacaggcacatgtgctcttctgcagcaatttggggcaagagaagatggatgtgataaaaagtgaaccccagggcttgaaagaattcagcaaatgtccatcaggttccccggaggaccccagcgtgctgcctcctagccgtggaagggcagtcacagaggccctgacggccctccttcctaggcagcggcacctccagttccatgtgaactgggctgtcttttccacagactgggtcacctccatggccatgggaatgcagtgctgcgctcatggccctgtctctgggttctcatctgcgctgcaagaccccctcttggtgccctgtggctctgagggtttcctgccccagcagacctcgcagaggggtgaccagcctgtcactctgcccgctcctccagcacctgcctccaccctcctccccatggctccagatcggggagcccagggcagcagaggccaagatgggccatgaatttcctcctggccccggccccatcccctaaaaggtaactggtttgctacacgcgtggcgcccacagatggtccacagaggtaacacaggctttagggcggcccagctactaggaggaaagcgtccactgcagacagctggggggacccagtcttgctctggggttttggggggatggtcagatcatggccgtagagcctggggtgccttgatttaccttcccacagctcagcctgagcggagggcagggaggagcccacgggactcagctcacttcgggtggcggccccccggccctccttgccgccgtctcctgcttttccttttcgggaactcagacactcagatcatgccggggtggtactttctgcaattaggagtgaatgcgttggtttgaaatgtttcgggagacacagactctgttcctggatttgtttgaatgttgagaaggaaaacaagagcttggggataaagtgcctggtagaggaagcggaggtgggaagaggccagttcacagcgcggctctttctctcggccccacgtccctcaatagggcaaggatgtcggtggcagaatggcacgtggccagtgaggctgaggatggtgtgacatgatgctggggtcacacacatgttggggagcatgacttggggcggcagacaccaccccccccccggtatgcacctcagagaggctgcgggttcacgctgccctccagggctggccgggtgtcaggaaaacctgcacccaccagcacattccgcgggcagagtgcggggctcagcctgagcagagggccggtctccccaaagtggaagcagacagaggctgatcttggccgagcgtgttaagcaaaggcatttcgtgagcagatcagcagactgataaccacgcagcgccatgtcgatgaggccaacgcttgtttctaatgaaaacctggtgcttcccgggccaaggcgcggccgcgggcgcagggcgtgttccttctgcttccctttcctgggccccatgtggacccctctgtccgtcagggctgtgacctgccagatctgccagggctggggctttaaacgaagggcacagacatccgccaccctgctctgacacgtcgcggggacggttccttattccctctgctttcattcctcacaagcttttaaattaaaattctaagtgtattggcaggtttaaatggtagcttccaattagatacaattttcccttgggacatgagatgggcaggggattcgttttagcatttcttcaccggtttacaaaagacttaaaaatacgtcaaatttgttttgagccagaaagccgtcgcattttacagtgccctcttgggagcaaactggccctctttttggcaaagaacaagatacaaattcaaccacgccgtcacaatactggctacaatcatttgtcagaatttatattcttttccttcttgaccaaatgagatactttattagaagaagccatttaagaatgatgaagacaatttaactgtgaccagtaatttatatttatatatgtgtataaataatatccagatccaaaccgtacaactaagaaagagataagtataaagaatttttttttaatttgagctgctagaagatgacagaatctatagcagcaaaacggaaagatttaagcacaagttgcttcagccacgtttgggcagagaggaagggggcaaatgccatggggaacgagccttctcctctctcggccagaggaaaggagtgccggcttgtccctggggcctgcggacggtaccaactccagaaccgctgtcccctgcttctgcccagctctgaaaaaagaaggcaaccagagcaggccagaggaaggttcccgcagtgaagcaacagacaagggcagagagaaataaggacctctgcccgaaccctcagcagctccacgctgtcgctgctggatcctggaacatcagctaggttgttttttaaactccaaatgcacaacccttcagccagtctcagggttccagagtccacaaactttcccggagagagatggcccgcagcttggggacagcaagtagtgaagataaacagtgacacttcccgagtccttgaagcaatgaggcagactgggacccgccaagcctctttggggatcccccaagagacaaaaaatccccagggataaccctcagaccaacatcctttctcaaaaggacaagcaagaggcagaccgggcgaatttacttgtcagcaaccatcttagagaaattcaaattcccctcgccgtgcttctcttttgtgaaaagcccctcacgcgccgtaccaagagccttcacaaatcgagcacaattgaattccaaatccaggatggagagagagcgatgtttttaaagatcagcacccggaaagatatttcacatattttactcaaacactcctttaaagtttctcaaaccagtaaaacggggaaaagaagcagtccggttcactcgatcactttcgtgtttttgagaaaaccgtttgttccagggagcggggcaagggctgcagccagtgctctacccacggcatttagtctgcaatccggtcgcttctatgcagtgtcacggggatggagtttttcatctatttaaatcttaaatttctaagagtaagtaggaatgcaaattgtgaaacttaaaccttcgttttccaaaatagcatctttccttgtgaattgatttgatattaaaaaacaaaatcaacaaacataaagaaccctaagaagaaaaatccgaaactggcagccgttataatttttaaatatttctatctttaagaatattgtgggggggggaatgttcatttttataaaagtctccgtctaaacgaacatggcctgtgctaaatccagcttttctcaggcactttctcttccttttgttgagaacgtttgtcattgatttgtcccagggctccttcatctacatggatggagcacctctgtgtgtctggagagccgtaaaagggcaagaaaacggcaaatgcagttgtaactctgaggagctgttttgggggctgttcacccatgtccactgaagtcatggtcactacaaagagaacctgagatgttgaaacgagggtttaacttcagacccgtctgtttggcagccttatttttaaagctgtgcggagcagacatttatgaggctagtaaactcagctgttgtggcatttcctcaccctcagatcccgaaaccagagggaaaattccagctcagccttgccggcacaacacccacggcctgggacagtttgagtagtgcaggggatgcagctgttcagaaagggtgcaaggacttccatgctgtcacttaagtaaatgcctgttctgggctcctttcctgcagctgagcctggggacgcaagggcatcagagacgagctctctttttccttgaggaacggacttgtctctcccctctcctcaaaactcaaggctcagcgtatccacgtatccacgatgccaacttggccttcgggtctgaatgtgttcgaacaggggcacacaatgaaaggaaggttataaaccgacttcaggaatgtgtaaggggctgcaataataatttattacaggcttgaacggaaggggcccggctggagacagtaaatgcacggtttgtaccgcctcaggcctcagcagctcccaccgaagcacgccctttgtcccgggaagtcgcccgaccccgcggaccaggaacggcggctttctcaacgcgcggccgctgataaggcctttgtgccgttggaaacgggcctgaaaacgcggccgctgtcgtgtggaccacggccggcctggaggacctccgcgccctggcgaacgcggggccagccgagtttgcgaacgtttgcggcgattccaccgcacgcctcgacccctcctccaggtggaggcaggtcgggcacaccccccggccccgccccggggccttgcgattagcactaatagcgattaataccgtctacggctcaaagtccagggggccaccgctgccggcccgccgccctcctgtcctgcgctcgcgcctccgacaggcgcccgggggcggccgtcctgtgggtcccaggccggggagggggcggccgtcctgtgggtcccgggccggggagggggcggccgtcctgtgggtcccgggccggggagggggcgcccggggcggccgtcctgtgggtcccgggcctgggagggggcggccgtcctgtgggtcccgggcaggggagggggcgcccggggcgcacaaatctactcccctcggcccagcgtgctagaccctcccgcggcagctccgggggctgcgtctgcgtccgcggccgcggggagggtcccccgagaggccaggagttctcaaagggcgtaggaggaggagtccggcagcccttgcagcgaggtggagtgtggagaggtcccaggaggtcggggagccgtcggagctgggggggcgtggggggaggggcggggacccgggcagcctcggagtgcgggttccaggaggtctggcggcccttggaggggcttgaaggagagcgtccggcagccctaggagccgaggtggggtgtctcggaagagccggtggcactccggggggtgggggtcctgggaggccgtgtggaccttggagggggtcggagaaggaaggccagcggccttgaggggcgcagaggacggggaggccgaggcgggagcccagcagcccggcccgcgggaggacgcggccgcctcggagccgtgcgcggagccggggacgccaggtcagagcgcgccgccggcctcgcggggcgggggcgcggggcgcagcccgggagggacgctgcgggcgggtccacattgccctctaacggggaccagcagaagccaccgccactccggccgcagtcggacccttctttccccagcctgcgcacatggagcggcggcccgcgggcgctggtggcgcgggcgggcactaagcatgcgcttgctggatgcggtcgcccgctgccaaatgaagtgcggttgtcctcgcgtattaataacccatgactcaccgtccccggacggctccgcggtcatccgggctttgtcagagcgtcagatgcactttgggtccgattttgcaatacagtggctcatctgatttttttaaaaacaattattcagccactgtgtgagtttttaggtaaaatgatacaaaactgagatcttaataacaattggcttgattacagaagttaatatggtccagtttctattccccgttttaaaatctttcattattttagcgacatacctgagaataattgccaagtaaaattgcaaacgagtacagggaaaagaaactttgcacattactagggtctatgtgcatgcgcgcatttttctgctgtgttttctgctgtcctggagtcctgggaataacatccaagtgggcattggagacgctgtggctttctgtgtcttttgagagttcgttggtatcgttcggagttagtagggactggtggtatgtgagggagtccacaactggaccgggcctggggagaggaggagagctcagtcttccagatgtttgggggggcacgggaggtgggcaagctgcgtgcccagctcagtcgggagcatgtccagctgctgctgggaagcctggcctgacccttgaccaggccttccgtgaggaggatgactctggaggagtgaggagggggcacggcctggagaagcctcagccccccggggtctcccagggtagacgaatttgtcttgggtctttctctggcctcccagccagtcctgtggccggagcttcaggctcctggaagctccttggtttttgttaacccaacagagacccaaacacccatgtgccggagtggttggcagtggactggcctgagagagaggcaccccacagtctgacctttccaggtttaagatgcaggattccctggtgtcagccctgctcacgtgtggagtgggtgcccccccaccacctggaggatctgtgtctctggtctccagagagtgcaggggggggtcctaggcaagatgtggcaaaagcaaggaaggccccagttctgtcccaaagtctcccagaactttgcagcctgttccctgggctccatcccagatggcccctgccccgctgaccctctgagttgctggagttttctagatcttggccaccacccccaatgcagacgcttctttttcaggagacacgtgggcaagctatggtgccgctcagagtattgaggccggctcttccaaatttaactccctagacctcagagcctgttccacgctgcagaacccaccctcagcccctccccgctcctccctgctttcaggggtgtccccatcgcaggcccccaactgccgctggcaccaagcctgctgcaggcacacagtcccctctgcctccccctgaggatggcacccacgctcatgtgagactcacagcctggtgtcctggccccggggacctctctctgagttcatggcctctttcataaacattcagattcaccctctagaccccaagccccccacccagaatggcaaggggtgtctcacatacacatcgacaaaaggaacctgagaaccaacccaggccgggctcttggagagagccgctgtccccagtccatccctctgggacgcaccctgcttggggactttgggctaatccttgccgatttaattttgcatcccctgtaccagtatctaaaaactggataacaccaatttaaaagggcctgtagtcattttgagcatattctgcaaacttccgaggggagccccttagcagcaatcacaaggcaggtcaagcggcctgtgggctgggcgtgcacaacgcggctttcagagcaacaaggacggctgcgctcaccctttctctctggtttctccatcctagatttattataaagtcattaaggacattgagccaggagaagagctgctggtgcatgtgaaggaaggtgcctactcgctggggacggtgccccccaatctggatggtaagcccccaccctcaggagcagaggcccaggagctcccaaggcctcccatcccttcatgccacatccctgagctactgcccatggccctgctcagaccccctgcgggtagaaccagcgtcccctctcaaccgaaacacttctctttcccgttccttgggttccatgagctgccccctgaattcaagatagaccctcagaccacatgggtgaatggatgtcgtgctgccttatatctttgtagcggtcgagtcctgggggattcttaaaaactgtcttattatcagtagcagcagcatcttttgctcttcccactagttaaaaaaaaaaaaaacagttttgtgggttcaagacattggcgccctagagctctgtgccacgggagaagtttccctctcgtttactagagggccaagacggcatacacgtcctggaaggtaatacccatctgtagttagcagccaaaagcccaagaggaatgttaagggaaaatttacttagtgagagggaaaatttacttggtgagagggaaaatttacttggtgagagggaaaatgtaaggctgcgggtgggtctcatagtggtatggtgcatcggagctcttccaccaggactgtgggaagcgctctgaatgccctttcctctggattcatccctggtctttgtccagtagacgtgcttctcttccttgtgcgtccacacccacatccttctcaaagggtcttgagaacactccagtctcaaataggctcggatttctgaaatacacatttcttttccttcatccacattgtgaagcaatgcgcccattcgcttggggattctggaacagttgtgagtggcagaggaaagttggttcgaggcacgctgcattggcaacagagggttggccctttaaccacctgggtgccgctgctgagcagatggcgtctttgaacgagacccacaagtggacagcaaagcagtctccccttctagctgtgtccggagctaacaattctgctcacgctggaggaaggggggtggctgatttttcccttcctgtcttgctttccctcccctccaacgtgccaaggatgaatgcttaatgccactaacagcaagtttctctaaggagccactctaatgtcatggcgtggagtggccttggctgggccagggagctgggcatcggagttagcaggaagcctccgagagtcaacacagagatttctgccttaattgacccttttggtccccgggcgtggcctagcattgagggtcctctctgatgcagccatagttcatgttccaaaggcattcaccacagaagcacgattttatgagctgggtcccatccgtgctcttgtctaaagtttggttttaattagaccgtcaagaaaagaaaaataaatggctgagtctgggtaacaggggatggtctcattttccttaaaataattgttgaaaagacctatgaatcttagataacacacgagaactcggaaacataaacccactcaactcaaggagagagctcagcgaggctgtttcatttctgtaacgaaatttctttcttccaaaaggttttaaaaaccttggttccaatttcccctccgaccccggatcctaagtaacctgggtctgcgttttcctaataacgtagccaacgtttgtgggctaattgccttccacacacaatagtccaagagcaaatgtttgacttccacgcactttgctgtcaagatcgttcttctgccccccgacagccaccccccgccccagttaaactctgactcacacagaaagaaaatgcaaaaatgttcctcactaagataattatcataaaaaatcttttatcccggccccggctgaatgcgtcggccccttatcacggcaggaatttggagtggctcttgagcgctcctgagcctccagtctggtagattagcatgtagatgccacataattgtgtccccctcagctctcggaagccattaggcactgatttattttcctcttacatccctgctctgccgtgaaaagttgctgatggaacttcagacggacgagcgaggtgtgcagatttgacaaacgagacgtggactgccacgcgttggtctagtttggtttgggtagaagagagggctctgccccacccctggccccctgcgtgcccccaccggattcccgtggagaggattcagcttgattcacaatttttcgtgcccttgttgggggcagggggctttgccggggacagtgttcaggtgtcgtggaagaaacgaggccctggtttgccagctcgctggggagagagagcccgggtcctgcccctctggggctgcaaacgctctttccaggctgcagcttcgtaaagaaatgcaggtccagtgttccaacaagtggttcagaccaggggaagccactttcagactcattagttccagcgactttttattttgtttttactctttcaatggagaggggaaagatttctctttcaggagaacttcttgctcttcgaagaaaataaagaaaatcatttccattcaaatatgcttttcttttgagaaaggctcaaaatgattaacttttcccctctccaaaaaaggtcaagaggatgtagtgtattgtgtataaagcacgtgagacagacagataatataatataatatgatataacacaatataatataatgtaacagaatataaccaggatgtaacatactcttttagtatttctttaaaatggaatggcttgtgctgggaaaatgaaaaccacgtgggctgtgcgtgtccttgctgcccgcatccagcgtgccccaggtgtgtgttcagtgccagccttacctgcctggaagtccgcaggaacacctgtccacaccccagcaggagtgggcaaggcttagattcgtcctgtccccagctctgtcctccgatcagggcaaacccaggtccatggtgggacggggaacggaggaagagggtgacatttatctccacggttggttccatctcctccaaggcaccctcgaatgccgtcagagtgaaaaatcaggctgaattttcattccaaatccaaatttcacatcaaaactggaaaaataagttggtcagtgttattggatgggtggggttggcattttttcttgtctttgttttcaaatttcgacttctgtggttgttcttagtttggtccagaaagactaaaatacaaattgcatttctaacttaatcatgatgccgtgggagggctccacctcagcctcattccagcccaggacgtcagaataaacccgcaagagcagttttgtggggtctgtgcatccagctctgctctgaaatgggactctcagccctcagctagaaaggaaatccacaccacgcatggtgcatttctcacacccgcagtgggaagtcggggctccactgcccgtttcctggggaccccacagtacaaaggctccagttccacggctctgccacttccgctcagtgctccaggcaggtgactcagtgtcttccagcctcagtttaccaattatgaagaggacgtaaaaatagtccccctgcgtgtgcatgcattgtcgagaggcttagatgcgagaataccgtaaagttctcaggacagtgccagccatgtggaggctcctcagacctcgggcattcacgttattagtcacactatttcacagtagacattttcctgaaaagggtctgcgttctcggtttctattgtaaaggcaggcttcgtggacttgccggctggttattctgtaaagtgcatcatcgctgcccacctcccccatcccgtggatcatatttggggctccaccctggcacctgagcgccggccctgtgccttgatgggttttgttttcgaatcttctctgactttgctctcccgtcggccttggggcccctcgcccctgttccttctgcccagccactctatccgttggtgggaatgccagggtcctgctggccactgtgtttgcaaatgctcgcttaagggtccctggtgtggcgccctctctgtctcctccatccccgagtcctgtggtttgtgtgggccggctgcacaatggactggaaggaggcgagaaggccagcgtctggggagacgttcgggagggagccttccaagtcccagaggcccaagctcaggcccctgcacccactcacggccccagagcagggctgggtgttgtaacctgactccccagacgctgagtgcgagctgttttttcccttcctcttcttgtttttctccaaacaagcactttcttgacagcagactcccatcagagagctggcggggacgcccagcctcaggctgcgtctgcccagcgcaggagcctgccgggcctgccccatccgcacacgtccccaggcaaagacatgaacaaaagtcacatttggtgattcggtgcttgtgaatccgagggtgtcggccagcgcccagggggcttggccaagggcacctagagcagctcctgcagaaaggtccccttgggcgtcagagggtgctgggtgcccaccagtggggatgctgggtgtggccagacacccggagcagtgcctccgcccttaggctcggggaagcttccggtcctggggaggtggccctccctctccccagcccgcagtgcccaggctgcagagctgcccaccctctgcttagcactgggaaacagttaccgtggtctctatggcacaaggaggggccggggcagccccggtcccccttccccccaccgccccacctccttggctcctccaccccttaggtcaggtcctgttggtcagcattcaatccaaagttattaagcctgtgctcagtgctggggggggtcctcgtgcatgtgcacacacacacacacatgctgaaacacatgcacacaaagacacatactcacactcaaacgcacagtcacaggtgtacacagacactcatccacactcacaaaccttcactcttgcacacaaatgtgtgcataccatcccacacacattcacacaagcacacactaacacacgcacacaagtacaatcacataacacctgcacattcacacacaaatacacacacacatattcacgcatgcacaactgcatacacatttacacagacacaccaatgcacacaagtgcatatttgcacacagatgtgcatgcattcactcatatacatatacacacaccaacacatgcacactcaacacctaacacatagtcaaccatgcacacgaacatgtatacttaacatttctgtgcattgctcacctagcacacaaatgtgcacacacatgtgcacagaaacattcacatgtgcaaagtaagcacacacatgaacacacaaaaatgcgcaaatgtacacatgcccaaacactcaggcacacttgcccacagatgtgtgctagtgtgaagatatgcaaacacaaattttccacacttgtagccacatacagaaacacacttgcacattcacacatgtgcacacatacacgtgaatacataaacgcaccaacacaaacatgagggtagaggcacaaaaatgcctacaaattcacacaaggatacatagacacaggtgcaggtaggctcagatcttcacacccatgcgcacacagttgcacacccatgcacaagaagcacacgtgcattcacacattcgctgacaagcacacacatgcacacctttcagttcctttgcgcaagagctttagtgcacgtgttctccagacctgagagggacccggggtccaccccgggcggggatgggacccgctctggggaagagctgctgtcaggcggccgccagcgggggtgccggggaggggcccggaggtcgtcccccgaggaaatttacagctctgggctgattgtttggcttggcacagtggtcaggatgccgcctcgtaaacttagtaatagctgcccctcttttaatttgtttgaccttgacgacaataatttatgatatgcattagagctctgtcgtcttttttctcctaattcataaacaaagcacttggcctggcttccttctccgctcccgagcgggggccttctcaagcccacaacgacctcgacgcagggaggggcccagcgtttctcagtgtttcttttgatgcagacctgggtccctgtggtctccacaccctctgggcagccactcctaggtcggggatgagggcaggaaggaagcaggagggggaacagccagtgggggaggcaggccccacggtgtcccgcaccccctcgcggccctgggccctggggcaggtctcaggctcgcccacccgcctcctgtcctctttcttgcagaggagcccacgttccgctgtgacatgtgtgatgagctcttccagtccaagctggacctgcggcgtcacaagaagtacgcatgcagctcggtgggggccgcgctctaccagggcctgggcgaggagctcaagccggagggcgtgggtggcacgggcagcgacgggcaggcccacgagtgcaaggactgcgagcggatgttcccgaacaagtacaggtgctgcccctcccctccgcctgctcctcttccctcctccctccccaggagccagtctgggacagcagggtcagcggccctgggagcacagagggagcctgagaagggcagaggtcagccctcatccctcagccctgtccatgctggacgcagaggactccagggccagctggagacaagggtcccctcgagcgcggcacacctcaggggcaggtaccccggcccattcatctggcgctggggacagagcaggcccatgagctggcggctggaggccacagtgctcccagctctgaccgtgaaggccaggcctccggtcagagccctcccaccccgggaggatccacaagttgccctgttgagacaccgctggcctggccattctgcacagtgagggctgagctgtggagacagactagagggaagtcaagaaatgagggccagaccccgagacgtgcaggggtcgagccccagcgctacttctgcagtaaagtccgagacaaggacgggggacggcaacaccctgttgcccgggggttcgtggagtggcctggccctccagacacgtgagtgactccttctcagacagtggtcaggagcaggagggggtacccaggagtccccttatccctccaccccaggcaccagcctctgctccccatgggcagcacctgtctgtccagggagacttcacagacatggacacagagtgccaggccagctgtgtgacaactggacatggtcagcctagagcaggcccaagctgccaggcaggattccagggcccggcaggattcaggacctgggatctggggccctggggactggtgagaacccctgggcttcgtggggagcagccaggacacatggtcccagtcctctagggacccgtggccacttttccagtgtgggtccctcctcctgccttcgagaacgtcctccaggacagcctgacaaagtcagagtgaaaccctgtcaggtcactaactggctgtgggactgggggaactgatgccaatccccccacggcttccctggggacgggaaaccctccaccctgaacagatgacggagaaaataagcgtttgggcaaatcccgactgaggcccttggcggtaagcgctgctcaagagggtccgtgttttctttacataaacacataaatcacaattaacaacagggaaaaagatacaatctgtgctgcgcaggaaccaagcgtttctgatctaatggatggctgggcccctaattaacgggccgtgatgtattgctgctctcttcatgaagattaacggtcatacctgggacaggaggagaagcgggctgtcccccgagaggaggggattaggagctgctcgttcgtccaagtgccgcggcctctcccgggcagctgtgccctccccaccccatcacatctcatttggagaccggctcatccaccccggcctgcctccttcatgtgtgcgtggcgggctctcggcctggccaggcggtgtccttgggtttcccaggcctcacccaaccctggggctcatggcagtgtcaaaagcttccagactccttttttcttaggcccaaggttagcttcactgttcacattgtcttgaagagccgctgggaagtgagttgtggtcccgccctgcctctggctgcctgcagggtggtcttggacaagggcctgagccctctctgggcatcggcttctgtccaggtaccggctgcctccccatctgatgtgggaagaccgtttctgcccctccatctccttgccccgtccagagaggaggtgggcagacacgctgcgaggactcgctctgatcccccagcaggacctggtgggggttccgaggggctgctgagaacaggccgtgctgggttcagggcccctcttcccgggtggtgggccccacggtgacccccgtcctctgtgcacagcttggagcagcacatgatcgtgcacacagaggagcgagagtacaagtgtgaccagtgtcccaaggccttcaactggaagtccaacctcatccgccaccagatgtcccacgacagcggcaagcgcttcgaatgtgaaaactgcgtgaaggtaactaacgtatgtgagccccgctccgcgtgctcaccggcccgcctgcgcctgcctcgcccctgctgtaaaaatccagatgaagggtggagagcgagtgagatgcccgggagtcatccggctccttctgtttcccgggaagagaggaagtgtgctgggccacgcagcaggaacgaggcctgagcggcaccagacctgactcccctccagggagggctcctgcccccggccccacccccccgatgtggaaggtgaggctggcaggggtcagcctccccacccacaagccatcccatatcatggcacttcagcttaaaaacgctttctgtgttcctgccttgagcatcctcgcagccacagttctcctgggaactgttattgaggaatcagagtctaggggaggctgggcggctcccctagtgctgtcgggtgggtcacggctccagcagagagctggttggccaggctcagcgtagggggcagaacggcgagtgagagccccatccctcgaccagaactgcctggttagggcagccagaggcgacgggagcccacagtgtccgtctccccaggtgttcacggaccccagcaacctgcagcggcacatccgctcccagcacgtgggcgcgcgggcgcacgcctgccccgactgcgggaagaccttcgccacatcctcgggcctcaagcagcacaagcatatccacagcaccgtcaagcctttcatatgtaagtcgtccccagccgggccgctcatctggggcaggcgctcttggaggcaggcccaggacggactcctcagcctcttgcccgcccctaggactcctgtttgttccaacactcctgctgccatgtgaagccccggccccattgctctctggtgccaccttctcttccttgtaccctctccttttgtgattcggctctcttggtctggcctccctttcccctacaacccgccttggtccccacgcttatccctccagtggccagaggtggcagtgggtggtggggaaggacctgcaggagccagtggcccgccgtcccattcccgccccgccacatgttcatccagcgctgctcaccctcgcagcctttggggaacctggggctgcgtgttcaccgctgtccaggggcaggggttggccataagctgctgggcccctgtccccatggagcttaccccccagtggaggagataggctaagccatataaatgataacagcacatgtgacaggcctcagcctggggggcaggaggctctctaggtgccagggggcggcccagggtggggtggggggccgctgcccctgtgaatacaatgggaggccaggatggcccagcctctgaagacaagcaggagtgatccgagggggcgcctgcagaggaggcgccggacaagggacttccagcacagaggccttgaggccgggatcggtttagggcttgagtgaggagaaggaagaggagaggggtcagacatcacaaggccaggtcccgggcaccttgtgggccagtgcaaggtcacgaccccaattttcagtgggtttggaagccactgaaggttcctctctgcctagcattgtgctgagtcaccattccactccagcattgcgctgagtcatcattcacacgagattgtgctaactcatgtcacacaagttgtgttgagtcatttcacaccaacgttgtcactaagccattcacaccagcgttgcgctgagtcatcgtttcacgcacattatctcacggcatccttacagaaccgtacagagcagggctgcaccatctccagctgacagagagggaaactgaggcttagagacaagtgaccaggcgctggaggccatgctgcggagcctgagttctagctcaaggggcctggctcctgaccccgggtcgtggaccagggcctcttgggctccagcacatcagtgcgagagcaggtgactctgcgagcctggggactgggtcagggtgtcttcaccaatgcgggggctgttcaggtgccagacacctgccccaaagaggccatgccaactggccagtcaagtctttgttgggtagctgggactgtgcccccctcctgcccactgcccccagcccaatgttccaactgtcctcctagaagtgactcagaccataatccacctgggtcactggctcccccaggcgaggaagcaaagccctgcctgtcggcccctcctctagggcagtctttctgctcacccaccacctccttcacctgcaggccctggaaagagagcatctaagcagtggtgtgggcagccccaccgcctggctccaggggcaaggcccactccctctgaggggagctgggaggaggcaagctgggtggcggtggggctgggggtcatgactttgccactggaggtgcatggtgtcctactttccggggatgagcatcccagcgagtccccctatgggagggggtgcgtatgtgtgcaccagatagtagctcagagtttttaagctgagactcaggaaaattgcatgcaggcttaagagctcttgccttgtgggcatgcacacgagctgtgtgaagtctcactgaatcgcgtggatcacatgagttccaggagctgccgttctctggaaggatctgagcccgctgttcccgcagccagcacagcctccccttgtggaggtggagagcttggggcaagctggtcgggtccttgccaggggccagcgctctccgcctgctgccgggctccacactcgccttggggccttggcttttggctaaggggcaaagccaaagagaaacctgcgcttgccaggttgcgtgtgtgaacatttcgttcagagagggtggcacaaattctagaagtcagacgtgaagcacgatcttgcgcatcctgtggcccacgggagggggctgtggtgaggcctggagagagagcgctgcgcacggggcgggggggtacccacatggcacttctaagaggagagcagtcgcttggaggcacacagttgccaccaactagttgaagccggatagtcgctagtgtgcacagactctgcatccacccctgggcggcatgtgggtgcacacacacggcacctgtggatcagcaccaaaacctggctagaagctcccgtgccccctcctcgatgaccagggtggtcaagtgtgtgcatgtgagagagggggtggggtatctgggcgcacaggcaggcagtctcctgtgggcactttccagaaattttggaatgtgtgtgtgtgtgtgtgtgtgtggtgggggaatgtgcatccacacacaacctacaaggaaaagaaactgctgaccgggacaagtactggccagggcccctgtgggtcctggcagggcctgggcggcaccctccatcaggcactggtccccacgtccagcttgctgtgggctgcccaggcctatcccagcccctgcaggcagccacctcactttgccagctctgagccagctgtcagccatcctaggtctgggacttgcctctcccgggacctcagtctcccagaggcctctctgtagccccagcgtttctgcaggaaccccccacccatcatgtgagcaggtggatgggaacggagggttcccaggttccctgtgcccaggcttggctgctggcctgggaggcccctcctgggagctgcctggggtaggagacctgctcacggccagtttccaagctcaggagggcagcggccaccaacaaggcactgacagagccctgctggggccgggggtatgtcacagagccctctggaggctagcagagccacgggggtatttatagaccatccccggggagcagcacctctgcattggcacctcccagaaagggcagatgccgttaaatcaggcagcacgttctccccccagccttgagaacgtcccgccatgccagacgaccaggaaggcagagccgctaatataaatagatgttgtgattaaagaacacgataatctcagggccaaagctcttggctggaatagcgtgacatcaggcagttgaggatttgtggtgagcaagagccacgctgaaatcattgtttccactggggctgcgtttaggaagcaaattgtcctgcagagagagacacacgcagggtgtgtgtgcatgcatgcatgcatgtgtgtgtgtgttcctcactttctggttagaaaaacaagaggagaaagaaagctggcgatttgcagatactccaccacaggccattgggaaaatggcgagacggggtccgctgggcctcgggtgctggtgctgggctctggccggcgctgctgtctcctgtcaggagcagagcagcctgctgcctctgcctcccaatttggggaggtgtcacttttctcaaacctggaaccagcgcctctccgcacagcccaactggggcttccctcccatccccaccttcaggacacccccagacagtgaccgtgcaccaccaggagcgtgttcccaaagcccacactccctgagaagagcccgagtgggctggtcccccggaggcctctgtctcctgggtgtgggtcgcagggcctgggacagagagaggagggagagagttgggatcgttttcaactttagaaagccggagataccctggggggttggtgttttcctctctcccctacggggacagtgtcaccaagtgggcaggcaggtagggccggagtcccaggcgagctgatgcagctgcagacggggctgcatgccatggcgtcacccaacgctgggagcgatttcagaagaccttggaccccaaactgcagacactcttggggaaggagggcgccagcagtggcagcaccccattttccaacagagaaggttgagtcactgtgagacctgcccaagctggcccaaggaccccagggaaggcacagacggtccttgccaatccatgctggtgatcagagggcagcgccagggtagagtgcactcacgtgtgtatgtgtgcacacacgtgcaggcacatggatacagatgtgcgcacacacataaacacttgtgcacacgtgcacaacacacttgcaccaacagtgcaaatgcacacacacatgcatacaccagcatacatatgtacaggcacaagtacatgcacacatgcatgtgttcatgcatacacacgtacaagcacatatatactggcagcacacatacaacatatgcacatgcacacacatgcacggcacacactcatgcatacagacgtgtggctgcagacacacatgcatgtgtgcccaaatacacagatgcaagcacatacaggcacacatatatacacatatgcatgtacatgtgctcacacgaacacatacacacttgcatgtacatgtgcaggcacactcatatacacacacacgtgctcacacacaagcaggggctgctcctgtcaccgaggccagaagcgcccttcatcctgctccccggaaaccacagagtttcccttaatttcatatcgtgttcgtctttgctttctgctgatgttttaggtgaggtctgccacaaatcctacacgcagttctccaacctgtgccggcacaagcggatgcacgccgactgccgcacgcagatcaagtgcaaggactgtgggcagatgttcagcactacctcctccctcaacaagcaccggcgcttctgcgagggcaagaaccattacccgccaggcggcatcttcgccccgggcctgcccctcacccccagccccatgatggacaaggccaagccccctcccaacctcaaccacgccagcctgggcttcaatgagtacttcccctccaggccgcacccagggagcctgcccttctccgcggcgcccccggcgttccccgcgctcacccccggcttcccgggcatcttccctccgtccctgtacccccggccgcccctgctacctcccacgccgctgctcaagagccccctgaaccacacccaggatgccaagctccccagccccctggggaacccggccctacccctcgtctccgccgtcggcaacagcggccagggcgccacggcagcctcggggcccgaggagaagttcgagagccgcctcgaggactcatacgccgagaagctcaagacccggagcagcgacatgtcggacggcagcgacttcgaggacgtcaacaccacgacggggaccgacctggacacgaccacagggacgggctcggacctggacagcgacgtggacagcgaccgcgacaaggccaaggccaagggcaaggcggccgagggcaaggccgagtgtgggggcggcacagctcccccgggggccccgaacagcgtgggcgaggtgcccctcttctgttcccagcactccttcttcccgccacccgacgagcagctgctgacggcgtcgggggccgcgggcgactccatcaaggccatcgcctccatcgcggagaagtacttcgggcccggcttcgtgggcatgcaggagaagaagctgggcgcgctgccctaccactccgtgttccccttccagctcctgcccaacttcccccacgccctctaccccttcacggaccgcacactcacccacaacttgctggtcaaggctgagccaaagtcaccccgggatgccctcaaggtgggcggccccagtgccgagtccccctttgacctcaccaccaaaccaaaagaggccaagcccatcctgcccgtgcccaaggtgcccccggcgcccgcatctggagaggagcagccgctggacctgagcatcggcagccgcgtccgagccagccagaacggagggggccgggagccccgcaagaaccacgtgtatggcgagcgcaggctgggtgccggtgaggggctgcccaaggtgtgcccggcgcagctgccccagcagccgtcgctgcactacgccaagccgtcgcccttcttcatggaccccatctacaggtattaacatgccctgccctcacctgccctcccagaggggccagccatgctggggagctgcgtagggtcagtgtgtgggagaggctgccctcttgtgctcccccaaggttcgtccctctgcctttgctggcttcaggggggtgcctgagctctcgggaggtccttgctgctccagaagacactcctggcttggcccattcctggggtgggcaccaggcgggggccacacttaggaggctggggtaggtcctcccggacatacgcgcttccctttctctgacatccactgggctaccccatgccctgccttgagcacctctggagcctgcgctcagacccaccccagaggccctcccactgcctcccgctgtcttctctggaccgactctgcctcccacacatctcagatccttgctgtttcctctgagcctctctccacctccctgtgtctccgcttccctatggctgcccttgtacacccccgccccggctccctggagccacaactaggggtttctggtggcccaggggaaatcagagaaggaaccactggtccccagaagcagagaggaggaaggaaggaggcaggtttggaggtttgcaaacacttggtgtcatgcctctgacggcccttctggggaggagtgggctaaccccctccccagtgtctgggcagggttagatcagagaggctgcttaggcgggaggggtcagaccctgccagcacacgtcctggaggtgtgcacatgggggtgctcgtgagggcctggaggtgccaggaaaccaacacaggctattgcagcgcatacaggcctacgcctctacgtgagggagggcctgtgcactcgcgtgtgcgtgacggcactggtttgcggggagcacaggagcacgtggccaccttgaggtctccagtgcttgctgttccctgcagggccagccacggcctggggtgggcatgtgggtaaggaggtcacacctgctgaaagtccacatgggagagagtgatgtcagagccaggccgagctgccagggaggggaaggtgggcgagcctcctgctctgcctgccggcccaggatgcccacacgggccctgctctccacgctcgacaccggggccctggccggcctgagtctgcagcccatctgtttcctgctcagtcctgagtcgggaccacggaaaacaaggaaaatgccccgtttgggtgtattttttacaaatccaccatccatagatggtctgagaaggtaggctaggggctcggcagagaggggtggccttcatctgtcaaaggtggcttcccactttttggggcaaatgcgtccatcaaaaagcgcagaagacacttgccagttaacgtaagaatctccgggaccccaggcgaggccaggaaacagctgggatgtgagggaggaggcgcctgtggggcgtgctgcccccgtgcagaggatgggagggagtgactggagactgagggacggaaccttccggccaggcccccagagttggagccagcaggcgtgcgagggtctcagttaacctcctcctgctcctccccggcttctccagccccctcctccgccagctccccgtccagcccgtccccactctcctcttggccctcatccccccctcgtctgcccctgcacttgaccctcagtgcgcccctcccccagcccctctcctccctgctcacggcctggttgagggtcttgggccttttcctggaatctgggggagcaggaagttgagaaccggctgcagggccctctccagtcagagctcatgtgagacccggcgtgtcctgggctctcaccctcagagtttagggtccagcacctgctggaggggctgcctgcaccccgactttgcacgcagacactagcaaaggctgcagtgtggctgagctctgtgtggtctccctgccaccccaaccccacaagataaacacacggggtgaaggcttctctccaagcgtgggtcctgagagtgactctagtgaagctccccacaggccgtcccccgtgagtgaccctgagcgtgtacccacagccctccatcctctcacaccacccggtcggcctccctggccctccaggccctcttgcccccgccccccgatgtggctgctgcgtttgcaggatgggctggggatgaggtgggaatgtggtttatgtggtgacataagccaggcaacttggagaccccgtgtccgctggggtcagctggtgtcagctggccttggttgctcgggagccagcccggtggggctgccacatgactcagaaaaaggcagaagagatggagccaaacctggccctcctcctcccacaagggccccccttctcttggtcccgccccaccaacctgcagtgtctcctcgaaaggccgaatctgacacagtcgcacttggaaagggcctcggtgaaggcctgcagagtgaggtccctcccggccgacttcccacccagtgggtgccgcacctcctggggccacacgtgtggtcctttattttattcctctgggagagaatcttcccaaagtgcaacacgtgcccccccggctcctcccacatctcccgccttctccctcctggaccaggctcggggtctccgtggggtgacctgtggcctcaccctgcaggggatgctcctgcctctggccccctgggtggctgtgctgtcctccttcagccatccccaatcctgctccggtcactgccccagccagtcccctggaggcctgagtgaagggagcacggtgcccagcaaggcccaagagcaaagggcacgggagcatccggccaggattggttcagaggggctgtgtctgtgtgcagagaggcacagcagtccccaaagcgcctgagacgccgggaaatgctcacaaatcgtgctttgtaaagctcaccccccacggggagactgaggcaggcgttgctgggaaacggtgagaggagggatctctgggcgctgcccggccacctcctctgctggttttgtgatgaacagagttgtcaccagcacagagggaaggtcctagacctgtcatctcggcagccatcgcgagtgttaggcagcgcctgcttggctctttccccaggacagaaacctcctggtcactcacgctcgctggtcagcccagtcggccaagggcaggggggacaaggaacagcctcccgtgtgtgccgcgttctgctgcaggcgagctgggatcccaactgtgtaatcccctgggggctgacgggccagtgggcagaggctggtgcgctcagggccttcagagaagattctgggctccggcattaagtgtttgtcacatgcacggtggctgggggcacatgggggacctgaaggggtccagaagtgggcacggcactggggagcgtgggagggaagcagagcaagactgtgggggtgggcagcgggctcggaggctctcctaggtgagcaggtgggcctggccagggccgtcaggtaagacgtggtagctggggagccccagaagctgggccaccgtggcgaggcccccacacctcatgaccagtggggtgcctccctgcccagcgctgggccactccgcggggtatgtaaccctctgtgttgctgtccagcagggtggagaagcggaaggtgacagaccccgtgggagtcctgaaggagaagtacctgcggccgtccccgctgctcttccacccccaggtaagacccagaggggccaggttggcggggcacacctcaggaccctcactctgaggccctctgctctggcctgccccaggctgtgcagggatctttgaggggctggaaccccaaggttctgtccaagcagagccacactcctgaggcgggccttgcgcacggcggccccttgggcgctccgggcctcagctcccccatccaagaggattctctccctgcagaccccacaggagcctgtgaggctggagagaatgagaaaggccaggggaggtgagcagggagggggtccttggcaggaggcagcccgtgggtccctcaaaggcagcaaggaccctggccatggggaggcccactgacccacagcgccagctctaggagggggaatgaacctgagtggggcggagggcagagcaggatggagtgaacgcaagaaatttaaacataaagtgaatagactcgggacgcagccccgaggggtggatgtgagcaggcctttccggggtgtgctgcccttgagggagctcatgagaaaagaccaggagaggaagagcctgagtgaggaggggacagagtgggcatgaggagatggggagcaggagagaaggagggggacctgagttgagggcacagggtcccaggaggtggggggctggaggggcgccgttccccagggccacagagggtggcaggccgtccgccaagactagaatcagggtctaggaaagataacacggaccctgccaagcctgcaggtgtttggtctgcccctccagccacatcctcctgggggtgggcacagaggggtggcagaagtgagggctgagcaccagggccccactgagtctccccgattccctgccctgtgtgcccactgccctcactgtgtggggcccagtgccctcccgggagccacaggctgcccctctctccccgagggtcctggagctgagctgtggggcgggccccaacccagacaccagactgggccattgttctctttcttggtgattcccgccttggacaaacagcccagtttgtgaaaccctctccctgcccaggttcacagctaaacttagcacctcccattgtttcgctggggcctcggagtttggcgaataacttcccctgattttcctcgggatgggctggaaaaagctgacaagccagccaggcgcatcctgtgtttgtgtttgtgggagagctcagccgggaatatctgatcgggcggagcgggccgggcaggcaggacctgcgggcagcctcggtgagacccacccaggcctgaggaagagaggcctggaggggggggccggccacccattgtccctccagggcccacttccctgggccctgccacccccgctgcctgccccctgggcctggagcaggggagtgggcgtctgagatgaagtcactccaaggtcaagccatcactcgggcagaaagcacctgataactgatggtctctacaaagtacagggtcacagctatagaagatgtcagcctgcgctggtcaggtgacaggcagaacaccacaagggccttccgcttctgaggcccaacatgcaggtgtgcagcccccctgggctgcagggtgaggcccttctgcccagctgcgaccgcttaccatctctaggggtcctggggagtctggagtggtggagagttccagggccttccaggctatggctaaagctgagacctgcaggcaagtgaacagcagtctgtgcaccaggccatggagggaccagtgaggggctagacagtggcaagggacatgacaatttccatctgtccatcagcggggacggggagggcagcccacagcggggagcctcatgtgtctgaggagggaacgtgctgccccacagggacagccaagcccgaaggaagcccggccacctagcccgagctggccagcggcccagttggcaggccagggtcccagggctgccttggccattttctactcattaacccctttctggatgtttttattgtaaccaatcggttgttccccaaattgcaaaatatctctgctgctggcacggctgtggcctctgtgtagcatcagcagaaagccctctgtccccaccagcagtcctctgagtgggtacctagaggacagagcctcacggaggtgagggcggggccctggatggtgtggaggggcagggctcgctggaacaccccccatagtgagacatgatgggtttgttcactggtggcccaggctttgctgtgaatgagcaagtaacctcctccgagtatttgccgttaagccttgctcagaggcagcgagcctcagtgagcggccctggcgtgttgctgccagccactcctccaccccctcgccttgcccactcgtgcctgttcttcagatgctagctgcctggggtccaccatggtgaccatggccagtttctcccagtgatcaaggcggttggagaaacacaagctcttcctcccctctccatctccatttggaccatgcctgcccttctgctctgtcctgctccccccatcccttcctctggggtctcccaggtgacacaggctcctgtaaatatttgtttagcaaaggtatagggagagccagctgaggactgggcacagcgggcactctgggacagcccacctccatggggagaaggagagcgtgggaggtactgctgaggcctgtgcagcttcatcagccccgtcccctctccgggccccgagtcccaaggccttgagggacaggcacactctaggattctgatcagaactctgcccacctgggggaaacaactgatgccatgttcccttctcctccctgagcaagtccttggcatccccaaggagcagagaggcagcccctctcgtccatcagatgcactgtgccgtgtgacattggcattatcattttgaacatttggttggcggcatccagtcgagctgttgaattcctttttagggttttgcctgcagttatggcttattgccactaggtggtggtggtgccccaggcccgtggtccatgtcttcacctgagattcaggtagctatcttcatattcaagactcaaagataaaattcttttacccaaactagccaaccaggagtgctggccaggggtcaagcaagtgggtcactgctggctcgtttccaaataaggagatgtgtagtcactcgtgtctaccaaaccaggcagtgaggctggctggtgaaggccagttccagcctagaccctacgctgcaaggggtttccagggaaagacaagagcaggactggccacagaaagcgaaagaatccagcccaatggccaagatgacaccagaagaccgctgattccagtgatggcgtgaggtcccacatggccttgtcctggcctggcccactgatgtgctcggtcatccaggacccctccatgcccctggggggttgagtccacgctctacaagagagagcacggcccaagggagtcccacagcctctcccgctctcatgccaacagatgtcagccatcgagaccatgacagagaagctggaaagcttcgcggccctgaaggcggactcgggcagctccctgcagccccttccccaccaccccttcaacttccggtccccgcccccgacgctctcggaccccatcctcaggaagggcaaggagcgatacacgtgcaggtgagacactgcaagcctctgagggctggtggggcggccctggcgaggtggccgcaggtgccacactgcagctcatgaaccttcgcatccaggaaaaagtgctcaatcaccgagtacagcgtagaggcaataaatgtccattatcagcttgagatcattccccgaaagtgtgcatgtgtgggggacctcccctgaccccacgaagctatgttcatgtgagtgcaagaaaatgcaaccttggcccagccagtcagaattcaggaggcctgaggctgaggcgtgcaggtcaatgtttaaccacagctctgggagtgagcagaaagccctgcactgggaagagaggtgccatgtctgtccccagctacacgggcctgtccaagccagtccgagccagctcgaccaccgctgggctctggagctcagatactgtttagggggctcacagcctgagcgggccggagtaggccaatcagccttctcagctgggccccagggctgaaggagagatagaaatccaggatgctgccacctggccacctgctccccaggtgtgacctcaggccctagaccccgagctggaggaacagctcccaagtcaagggcagtatgcctaacagtctccccacactctccaccccaggtactgtggcaagatcttcccccgatcagcaaacctcacgagacacctgaggacgcacaccggggagcagccgtacaggtaggagccggccggtttgagtcaggggaggcaggctcctggtgcgaccctccccgtcccacccacaggaagggcggcaggtggaagccagcatgcttgcgtcagctcactcctggggcccctccctcctggtccacagccaccctgtgtcctgggatattgtaaagattaagcaggaggtgcctggcgggcttccagaaggctcctgcctgaccacagaggagcacggctggaagcagctgtccaacctgctaccggggtgtcaaccgggcagggggctcagaggaggatcagccagaagctaggcccccaggggtggtctgctggggaagcctcagctcaccccgacccagtccaagtctgttcatctttgacggccttggaggacctaacccaggtggtgagctgggggcgcctaagtcagggggctgtctcagaagaaaaagggctccgtgcaaatgcaggtcccaacccgggcttggggcgtcctgagggattcgctcaggtccccttcactgccctggatggaaaccacgtgtctgctccccacctcgctcaatgcttgggcttctgcctgcagagatgcagccacacagcacgtcagggggtgagagccgtcatcttttgtgttcagaacaagtggaggtgtcctccccatttcaaagatgtggagcccgaggctcagagatgccccatgacttgcccggggtccaagcccacggcttagcattctggctccaggagaagcagatggggtgcccttggtcccttgggccttcatcgggcaggagaacagctgagggtgacaacccggcttttggggaccccgcccagggaggggactgggagtcggcattttaaatgagagccaggtgatggtgactatgaagcaaaccggggaaatgctggttcaggtgtcaggaggtcatcctggcatgtgtctgcgagcagcagctgtgggtaataagtttgccatggtttgaagctagcttcttaggattcctatactctggccacaccaagcttggggtcaccctttggccctggatgtttggtgtgtttccccctccctggggagggcagccgcatggggccctgcacgtgtcgtggtcgccatgcatgcctcactctgagacacaagagtccccactttccaaccaccacagggccacgtcctgtcttggctggacagtgatggttcacaccacttgccagttgtctggtcagtgcccgcagcctctttccaacacacaggcccctctcttccccctgaaaatgagacttagaagttaacaaaggctggcaacagaaagattgatttggtctggtccactccccgaaaacgagctgtatgtttgttctgtaacaagactcgacttctggacagtgttgcgtgttacagcccccagagggtcgggccctgtttatagggaatttaattatcattgatttcttttgctctttccaatctgtgagtttcaacttgtggaacaataattgctttgaaagtttgcattgcaaatcctgactttatctggctcaaggcgacgccatgatgaggagtttggggtctatgactctccttttgacatcactaggggtttctgggctccctctgatcactggaaggatttaggttgaaagaggaaaatgcgcacgtctgtctgcagaaacccgggagctgggcacaggacccccagggagaaggggcccccaccccagcaaagctgattaagaccgcctgcccggtgtgagccccgcccctggccagccgcaagtgcgtgcagaggccagcatgacacaggccctgcgcagccaggcggcagtggggcctctcgtgggctgcccagagtgacgggacagtagaatagatgacactggaaggctcttcccttcgaagtccttgcctccggtcatctagagtcgctcggaatgtgtttcaaagccctgacgtgggaaagaaagacgccttagtgagactggtatgctttctggagggaaagtcaaacctcctgcccggccgtggctggcgagccgtcctcgtccttgtcctcgctcttgtccttggctgatggcttctcctggcttagagatcctcgagtgatgacgctcggctttcttaaagctccagtgtggagggcttgttgagattttggataggagacatgtaaacattgagctgggaaagagggtccaggcctgcttccttgtctcaacctggcagccgtgctcgggggcccctccaggatgctgtgtggcgctgtcttgagtcacactgtggagcgagccttcatgggctgccctgtgggcgcctggcgggacctggggtccccactttcaggacataggcctcttgatctgaagttcatttcctcctgggctcccatcctgtgttaagccaaaacgtccaagccaggtccagctaccctttccgtcaggggctggaggaggccgtatgtgtcctctttctttgtcagttttatttttagggctgtgtttcaaaatctcttctttctttcccttccccccttttacaattaccgaaagcatttttccctggatggctggtgtggccctcctgggtcccagggaggatgctgccgctcgcccaggggccatttgaccctctccagcccttgctgtgtctccaccgtgactggcttcccgggggctgcggcagacacggtcacagcaaggtcccctccaggcctcggctgcacgctgcagtggccagcccactctgggctctcattctcgtcgctttgcaatggcacgttattctagacagcacagccataaatttcaccaggcgcctgtcagcttatttacaacgcagatcccagcggctcagagaaccctgctccctcgcagcctgggctggctgctcccctagatttcaaatggacttctgggccatttatgtagattagaaccaggagagggccgcatgcaggactcgctgcgaccaaatccaatgtgtggctccctcccgaagacacccggccccacggaggttgtagggagagcacctcacacaggagctctgctctctccttctgggaacaagctccttatccagccctgtttctggctcaggattcccaccgctcagagcttatgcggcagctgtttggtgaaatttgtcaaaggctcctgcatttctaaagaaaccaggaacactgtgtgtgcgggcgtgtgcgagcgtgtgtgagggcacatacagactgagggtgggtgcaagctcggcccagggggaaggctgctgagtgggcagaggtggatgcccagagctaggctgagggccagtgtgggctcgtacttggtccttcctgagccattgtcctgcagggcgggatgggagcctgggaccccaggggccggggttctgtaagttcaaggatgtgcctacaggggagtgagatggtgcatgctgggatgtagaagtcacctttctggactgttgtgtggggatcaccccggcatctctgacccggcctcagagagggctcaagcctggccccactcctcctcacagtgcaggcgagaagaacagagcagctggtgcctcccggtattggcttccagggtggtgggtccacatggggccccggaacgtcatgaggctgcgggaggagaggctgtgaccaatgtccagagctcccaggggcctgaggcagctcagtggcccaaggaggagcaggggcccaacaggcacatcctggggaccgaggagcagagaaggccagggttgttcaggaggaacccagggaccgtggcctgaggagcccgtgtgcagatcgctaacagctcttggcccatctgttgcagaggaagccagagttagcctggcctcaggcaaaattggaagcttcaggaactgaaggaatgaggccccagagtcaaactatgagagactggggacaccttttgacagggccctccccattctgtgggactgtcatcaaaccgagtgtcatccggcaggttggggccgggggcaacttggagactgtaagcttcccaggacagagtttcaggaggctgctgccccttcgtgcagggaacgtgcagctgctgggccctggcgacccgtgggggccacagcttcctgcctcagagaggctccgggggacactgagggtccatcgggctccttgggcaggaccaggcctctgaatgtcagatgtcgggcaggcacctcacgtgctgggcagggtggtgctgtgagggccactctcccacggcccctgctggcctggccctggatgggaggccagggctccctgagtgtcctcaggcacgtctccatccatgtgaccccatgcgcgtgaccagtatgctgtgctggagctcggcctcaggcctccagcccaatcggggtgggggtgggggcctcctgtaggaacgccaggctgccatacttcggacaggtccccggcacctggcagccaggcctctctggataaacatcagtctcccacgctcatgccccgctgggactggctgcgtagactctgcctcctgaaggactcgaccagccggggttccgctgcacttgcctcttgtctctctgtggaaacttgttttgtttccaaaataggaaaatacatgagcatatagacaactgtttcaagtgctcacgtgcctccgaggctcagataagcatccctggaggcgttctgaagaccaggtgcgagagtgtgcacacggacacacgtgcacatgcacacacatggaaatatactacacaggtgccagcacccccatgtgcacacacctgtatgcatgcacatgcacacacccacacatgtgcctacatgtgcacactaacacacacgcactgtgtgcatgcacacctgcacatgtgcacacacccacacaattgtgcaactgaaaaccgctgagtcaggcttctcccggctgtgctccagggagtgtgcagagcggcaggagcgcctgtcagtaagccatcacggcctttgcttggatctgccgccaggaggaggcagcgtgcgcgtgtctgcagaccttacaggcgcagggccctctcatccacctgctcccccggctcccgctctccctgtttctggcttgaccatccagctccccagtgcctgggcagtcagtccctggctctcagcggccccactgaaaagaggagaacagcttccctctccccaccctcggggcctctgcgtggtcgtgaagaccgtgcccccatgaggaccccacacctaagggggcgccggtcaggttgtggacgccctagcctgtttatcacaacagtttcctggcagagaaacgctgtgctctgataaactctgaacatgtcacgacaatattccgacaggtgcaactgaccatctggaggaaggagcgtctttttctaattttcccaagtgcgcatgctggctgccagccggtgaccaccacagaggggcacaaagataaactgggcaggcagggctggcacaccctcaaccgaaaggggagggtgtggttgtttgtggtccgtgtagaggaggcggcctgacggggggcagggctggggagcccccccaccttcagggcagttaggaggtgaagagcgcccccagttaatggacggcaagctgaggccccacgtgagacggggcttgcccgaggacccaccagggtgggggccaaagaccaggccagctctcccagtgccggggtgggggtctctgcccggaggaaagttgcaggcaactcctcccgggaaaagcggccagagcatctttgcgtggtgcgcggccgcccccttgaggctgctgggagcatcctgcccttgggctgtggctgccaagcctggggcctccgcactggggggaccagacagcagagaggaagcgaggcgctggggggtccgggtgctacacatccagtggcataggccagagggtccttctgcctacccggggccacattccaacataatcccacccacaggccaagagcgggagagcccctgcttcttggagccgtggctgtttctaggcgcagcttcaccatgaccacctttcaagttgggtgccccagcggcctcagggggtctctgcgaggcctggctgctgtccccaccctgcagtacctggcaccctgggcccagtctctccagggatcctcatgcatccagcccacggcaggcggggtctgggggcccctctgagcctcgtgtgtcttccaggtgtaagtactgcgaccgctccttcagcatctcctccaaccttcagcggcatgtccggaacattcacaacaaggagaagccattcaaatgtcacctgtgtaaccggtgcttcgggcagcagaccaacctggacagacacctcaagaagcacgaacacgagcatgtgccaggtggggccccatcccgggagacccccatgcagacaggccaggcaggcgccacaagaggctgtggtggagttcaagagcagggcagggagaactcgaaagatggggcaactgggagggaggtgaggtggaggagggacggacgaggaagcggacggccgcccctcctctgacgctcccccatccccgcagtgagccagcacgccggggtcctcacgaaccacctggggaccagcgcctcctctcccacctccgagtcggacaaccacgcacttttagacgagaaagaagactcctatttctctgaaatcagaaactttattgccaatagtgagatgaaccaagcgtcaacgcgaacagacaaacggtaagaaacccaccccgggccccagggacggctagactccacagcccgcctctgtggccacacccagagcaggcatctccaccccagcccctgagcaggcacaccccaccccagcaccagcccccaggccacacctggacaggggcctggggcccacagcagagggagaggcccatgggctctgcaacctgtgcccgttcctgccccagctctgccacctggaatctgggggagctttagcaaggggttcctcctccctgcctcagtttccccatctggagaggatggtaagagtaagcacccttggggctggcccggtggcgcagtggtcaagttcgcacattccgctttggtggcctggggttcactggttcagatcctgggtgcagacctacgtactgcttgtcaagccgtgttgtggcaggcgccccacatataaaggagaggaagatgggcacggatgtgagctcagggccagtcttcctcggcaaaaacaggaggattggtggcagatgttagctcagggctaatcttcctcaaaaaagaaaaaagagtaagcacccttgactgatggtgcatgttgcacactcagtgcagcccccggcacgctgcctaaactgcatcaggcgcggttcggcctcgatgtttattaacagacgtgcagggccgtcagccaggtgaggctcccaaacaagaatgctgcccctttaaacccacacacgcaaacacacacacgtacacacacaggtgtgcacacatataggcaggaaaacggcggaattcagattccagagtgtggagggtgttctgcacgatcttgaatgccagtgcttggggtgcagcacttaccctgggcccctggagggacactcagaccccgggaagaggttcttcttgggtgaaagcgggtgctgcctagccagcagagaaccagagatgcctcaaactccaaaagaccccagagatgcaagcccctaattttgcagaagggacatgaggcccagagaagtgggacggcttggctcttggggtgccaacaaagcctggcccagaacccggagccagcgccccagcctcgcgctggccaagggtgagctggacttctgcagccgtcagagcccagatccctccccacccaccctcttcgggcaagtgcggagggtcccccaggagacccggctccccaggagcctgtctgctcctccagggtgggctgcatcttccttgtcatgagtcctcccaggggacagggcagaggccctgggaagatgtgtcatatggataaatgaacggcacttggaccgtttctggtgctgcccagtcctaaatccatgcccgcgggaaggggacctcactcacctgccgggcctcggcttcttcctctatcagagctgggtgggctcacagaggggacggtgggagaggctggctgtgggctgtgcttctcagagcctcacccctgcccggcagtggagggtccccacgggagagcagccctaccccctcttcagcgacaaatgtctcgtttcctcggcctccggcacgtgcacaatggtctcacgggaacctagcccctatcctctggggacaccaagcctgcattcaggcaacgccctgcagctccattcaacagttgtcagctctttgagcaaaaaggcacagatgcagcgcccaggtaccgggccttacagaagccccacaaccgggccacaaggcatgtcctggctataaaactgtcccctagactgtgggtccctgcacccaggacaaagccagacaaagacccgacaaggatttgtcaagggcatcctccttggggtgggcagctgcgcctgggggggcccaggagctcaggagaggcctccacgtgcgagcctgggcagcagacccggctctcagccacagggaggactctccacgagacccaccacagcagaggggctatcccaagggtgaagaggggacagcaccatcctaatccctcgcctttgccaccaccatggctttatccacagccccggggtggggagggggcggtgaggctcagacagaggaggaggctggccccaagtccctcggagagctcagggcagagctgggctgagaacccaggccgtcagacagcctgtgctgctgaggcctcgccctgggagccagggagcctcgcaggagatgcccccacatcattgtcggaagtgggaaccacgtcccggtcgacatgaattaagcaatttgcgtaaggcgataggacgtcagcggctccgttggcagtcaacgttaacatgatccagttctcatccagaagtagtttccttttcttgaccatcagtgagaactcagggcctgccaggcttcctggccccaaaacctcttaagcccctgggctgcattgtgtccccccaaaattgctgcattcaagtccaaattcccaggacctcaaaacgtgacctcatctgggaagaggatcgctgcagatgtaattagttaagatgaggtcacactgggacgggggacccatgtccactaggactggcgtccttatgaaagggggaaatttggacacagacatgcacatggggggaccccgtgtgaagatgaagcagaggtgggctgatgtatctgcaagccgaggatgctagcaggccaccaggagctgggagaggcctggaatgggctctccctcacggcctcagagggagccagccctgcccgcacctggacctcagacctccagcctccagaaggggagaggacggactcctgttgtttaagccccagctgcggtgcttgttacggcagccccaggactctcacgcatgccaggcagccgttcagaggcctggacactgcccccatctttgctgtctgaagcccacgccagcctgcggaccccctactctgtcccagctggacgccccccaagcgcttctgcttgatgcagacctcctaacaaggagcagagggtctgcagggtggggcggggcatgaggatgaagagcgcgtgggccctgctcccaaacagaggggctcacggaaaccacaccgcatgctcaggtgaataaaggacacgtgggctgcaccctgagcataatctgagtctcagcaccggggcccccactgtgctgctcactgggcactcagcatggggctgttctcatgttgccgagagggcgcaggctcccaggtggtaagacttgcctgagttcccccagcccagagccagggagggaggcaggacccgggtcccagcctagctgcctggctccagagccctcactgctggcggacggacatgtgcccagcaaacagcacaggtgaccagagacaagggaagcatgtccgggttcagctgtcctggaaggcaggtcagctgtcagtgtctacatactttctcatgaaaagacaagaaatgaagatgtggaaggcggagagccgggcgatcaggggtctccccagctcagtcgctctgtgcaaggatgcgtcctccacacagctccacacttggggatcggtggtcactcggggaggatgtgccccccaccttgatgcccagagcaggaatcctgtcgcccctgacagtcgtggaccattcccaggggctgtgcaaggtccaggaggagcggggcctgggggctcagaactccgccccgacctgggctcaggaagtgtgggccttgccccccgtccccctccgcctgatggtggacacctgcccaggttgtccccagcttccctgggcccagcccaatgtgacacggctgttagtcaccgacttgcagacagaccaaacaaaggcaggtgggtgggattttctgaaggaagtgagagcgctcaccctcacccagcacgttgtttcttccttttaataacaaggacccagagtggaaaagtctcacccagctggagtgaccctgagggagaggaggtggggtctgggtttcaggacagactccagatggctctgagacaagctcatcccggccggtttccatgattgacaacagcccagccctcactttgcagaacctgggatcccagaccctccatctgcaggggtccctcaccctgggtccccccagtccttgtgtcctcgaggaaggagcaagcctccgtgggcaaacctccactgtgccgagcctccccaagggctcatcttacgtagggctgacaccagctccagtgcccaggaccctttcccgccaggccagctgtggcttcctcccacgacatgcacacctgccctgaacccctcaatggcccggtgtccactccagcataacaagagccctgccgctgaaccggccctgcccatcccctgccacgagttcactgggcatactggtctccttgactcactcactccacagggatggcagcccagggttctcgtgaccgtgaaggcagcctctttttggaggggctgggagagccaaggtgggcggcagtgggggaagagccgagtcgctagccttttggggtccacggccaacttcctcccgtgtcatttcaggccagaagtccaggacctggacggcagctcccagtgtccaggcttggccagcgggaagccagaggatgaagaggaggaggaggaggagctggaggaggacgatgacgacagtctggcggggaagtcccaggacgacactgtgtcccccacgcctgagccccagggtgcctacgaggacgaggacgaggagcctcccacctccctggctgtgggcttcgaccacacccgaaggtgggtgccggcttcacctgctggggtccagccagggagggggcagagcacccccagggtgactcactcttaaacgcccgaggacccgacaccctcccaactgggtccaccctccctcctatcaggggcttctgcttgcaggcctgggtgagtcagggcctgaggaaaggggatcaggggccccagctcacccacacacacacattcacacacacacatactcatgcacacacacacacactttcagggcccggaagcagagtgtgggaggggaaggcatttccccgcactgagaggggtcaggtcctagacccagggccacctctggccctgcagtggagctgcacactgcccacaggcccagggcagtggcgtccagccactagtgggaccatgtgccacaggggctgagcacccatagttttggaagttgtgcctaccagagccacttcccccacgtctcgtctgctcgcctcctacctctggccctggtgatgctgccagcattccaggatcctccaggaggcccttcagggatggcccatgtcctcccaccctgggcccctccggagttgcccagagtctcggcaacgaagccctcccaatctgtcctccgtcccctgaggccaggcgccccgcctgctgcttgccatgtgtgtgcacgtactcaccctctttcgtggccattatctgttgttctgcatccgtatggcttccgtccaggtgtccgtgtgtctgtgtgtctgtctccttgtgcatgtggctcgtggagacacagtggcgtgcatgcaggcacggagccggctgtgggacatcagagaaacggccagaggccagaccagctaacgccaggccctctcccggtcgttggtgcaggtgtgttgaggagcagccgagcggtctgttagctttggagcccgtgccgacttttgggaaggggctggatctccgcagagcagctgaggaagcgtttgaagttaaagatgtgcttaattccaccctagattctgaggctttaaaacagaccctgtacaggcaggctaagaaccaggtaggtacccgcagagcccaccctctccccctcccacggctggcggtgggggcaaccacacctcctccaacagggacagcctcacctctcgctgtggccaccctctgcctgcctgggggacacccaaggccactggtcctcctctgctcattgcctcagctagagagatgccaaatgactgccacttcctggaaagcaggagtggggcagcgagaggctgaaagcaaatgaacgtgagctgccgcccacaccggagctcctccaggagcgctgacagacacatggtcaccgtcctcctgtccccagtgggaggtctccacaagcgggttccagcccccgaggctgcgctggctgcccgttctctgcgttccgtgcgagaatgaccggtagcccaggcttctgtccagggaggctgccctgtcaggtggagctgtgacgggtcagcgagggcaccaggcttggccccacgcacttctcgtctctattccttagtgtgagcggccgcaggaccctgccccctgctgctctaagtctggaagccccgcatctcgttcagcgctctgcagcccagcccgagtctcagcaaaaaccgtctgcatgtctgtcttcgtctttttactaattccgtccagagtgttgactcgggtgggacgagggttgggcttctttgtcacccatgccctgctccaagctgccccaggtctgtcacctcatgccctatggcttgctgcagaaagatgtcctgtggactgtcgaagccacgctctgggctaggaaacacatctgcatgtggctcagaaatgctgcacgtccttttccccgaagcagctctgtgctgtcaccgcagctgtctagagaggagtcccccaggctctccggtgcagccggccccctgccccaccagtcagggacccacttttgttgacgggggtcttaccaataactaccagcaattcccagggccctcccaaagtacacacttgcccataagtttctgctgatcaccaggcagagaaaagtgggggaggggagatgggaaggtgactgcggcggggtgaagcaccccaaagaagctgaaggagccccctgtgcacagaggcccctggctggggccatggcatgctggcgcccctctgcactggcgccagcaggtgccctcctccctccctgctctccactcacatcttcctcttccaggccgggggggcgtcttcactctcccaccccacctggatctgggcaaggctgagtctggccagagggcagtgggagaccaggagggggcaaggacggagccagcctggatctgcaggaccccaacctacacggctcacctgcttgtcctgtgtgtgtgtgttctctcccccactaggcgtatgcaatgatgctgtccctttcggaggatgctcctctccatacctcctcccagagccctctggacgcttggttgaacatctcgggagccacaccggagtccggagcctttaaccccatcaaccacctctgaccagcccaggaggggctggggtcagagtccaagcaaggcgaccgcagggccccccacatcccaacagaaatcccagcttcggaagatggaggcgagagcctcagggagccggcacgcctgggccagggagacccccttgtcacctgcttttgaccactcctcagtgtctgtccccaccccccccatggttcaattctgacttttccaatgaaaactcagagccgggaagtccctgaatcggtcactgagtttcaccgtctctaagacttggtcttgagaacagcgtttgggaacagcacagagcacagccttcccgcacggctccagggctgcctcgcggaaccacccagtgggcgagggtgggattttaatcccaaactgaagccggtgggccactctgggcaccgtgcagcatcttggaatggacagacacagtgctctcaagggggagatggcccaaacggtttttataatgaaaattacgagaataacctttttggtaatcttattgacaacagagtctatttaagcatgtggttttaaaaaatagacagtattttttaaaaaatcaacaaatgacttgcaaattgttttttaaaagtaattttgcattgctttgaaatctcagcttatttgcaaacccaagcgtgcctgagagccggcgctatgtttgggtgtgctctttgttctgtaaataatggagaaattttctatccctgcccgtatttgtaaagccaaggtgattccgggtgcctccgaggcagaaccgaggggcatggggtccgctcctgtgggcagtttcagactgtgcagaggaattctaacatatggtgtcctgggctggcccaaggctgggactgggagatgtgcctgtcattgtgtgacgtgtgattcgtggcccccaccaagtccccccaattgtatggggaaggtcggaacggtggctctggtccccagtgcagaggtgggccaggcacctgatgctcctgcccccatggtctgtgcctcggccaccccactgttcggaaggcaaggcctgctggccacgtccggaggatctccagcccccaccctcactcaccccagtcagatggccaaaaggaaggaagcaggcacccctgtggctgccgtcgggggcacgccacggttcccctgctcctggccgccaagctctagtgcgtcggagaccccggatccaggacacagctgccgcttccactcttcgcacaggtccttggcgggcaggacccggctccagcacggaagggcgtccaggctgaccgagctgccgcaaagagcgaagacagcccgacccacggtcggcgcctcttcggacgggtctgcttctccatcgggtgggcttaaattatttccgtaggggccaatttcaaataataatgttttccctgatggaatttaccttaatctgtatataacttgtaattttttctaattcatttcttttcttattttatttcctccttaacagtatttttggcgttagacattcttattgtgaagaaataatgttaatataagtatccagtgaaggaccaaaaccgtgtaataaggttgtgtgttgtgtgatcgataaccagggagtggggcgatttttaatgtgccaaatacccccgcgtccccccccaacgaatcctgctgtcactgctgcccgttttccagacaatcatgtgtttttgttaaatttgagtttcagttgcatttaagacaggtgttctatttatttcttgtattgtttggaaaaaaaaaaaaaagaataacagcgtcccaacaagaagagaaaaaaatcgcccaagttgcccttaaaaggaagagtgtaagtttaagtacagacagtggcggtgccaccacatcgtgtggatcgggtctcgtggtggacgtctcggcgttcgcccggggcggcggtaggtgatctagcaggcgggtgccccagacgcacagcctggtcgctccaggccaaagacagtcatgtgcgaaggcgctgggaggtccagcctcccactggagacctgcgtggggaggtccccgagggcaccacaaacctgcggatcccagcgaacgggcttcacctctttctcaaacccacacgaggtagtagcaagactgagaaccgccctgtgcctgtgccgaggccgcttacacacgtgtggaggcaggaaagtcgtccctctggggcagccagggagaagctgcatgaggaagagctggtgtggcccatcgaggagcatttgcaaacacagccactcacacctcctccctcggccggagccgccaggtgtggtgtaaggacttccaatttcccttccgtctaaatgggatccctttgaaaagtcaacctcagatacaaacaccccgtttctgtaaacccaaattatatgatttcttctgctaaagattaaggtgtgtgcttttttttgcgatatgactctctcggtctaaaacaggaagtttggactggtggaaacctaagtgtccgttctcggcggttgcagagcagaaaaggggctgcccttcctgtccatcctggatttctctgttgactgccacgtcccgtcattttcggccagtcttcccaagcgttctctcttctcattccttcttttcccctcgtcccctatgagcctcagttgcacattttagtcctatatttttgtcccccagaaaatgactagagcaagcccaatttctcctgaatgttcagccccgcgggccggagcacttgccaagtctgggggagggggctctgcccttggaccccctgacaaagagagcacttatttcctgtgcctggtgcatccatgaaacttggcctcaaagcttgtcattgccccagaccacagcaagctgccctccctgcttcctctctgccccgggctccagcacctggggggcagctcccagctcacacaggtccagcatccttgatgcagatgccaccacgtgctcagagaccccctgggggccaaggagcctgggatgggcatgagaattctgagatcccagcctcggagcacggatgcaacagggcagctggctggctgaccaccgtgtccatgggcacgtggctagggtcgaggctgtaaacccaggtcctactgggccagctttgcccagggccagggtccttcagtccagccttaactgcctcccttttttggaccattgacactcagccccaaatggcaattcagaaggaggcccttccctctgccaccctcagcccccagtccccagtggccctcccccactgtgctctcattcccaggacgatcgggtaggacacacagggctgctgatcctttggccagccagacctttcatgagtgggactcagagctccccaatgggaacacggggactcgcgttcctgcagcctgcctgggaccctggggtgtggatcgacctgagagaccacccagcccacgtggccctcttgtacaaggccaagagtgcagctggtcagtccagaccctgactggcccagcccctctgtggccccagagcttggccctggaaactctgaggacccttggcctgctctgtcctagctgcttcctcagtgagcattcgcaaagcactcgcccgaatacatatcacccgtgttagcctcgaagcctcgaagcactggattgtggacccccctgcccccgtccccttctcctcacccatgtgactgaaatctactgagctgtattcactgtgctgtcctgggggaggggcgaggggcagcgccaggccgggagagcgcgcccctgcactgctgcccggggacagacggggagcccggcacctcgctcccctcccggctggggacgtgaggacacagctgctcctggtcagtggaggtcagccgggtatcaaagccatgaaactgtgtctctgtagcaatgagcgatactgtgactaagacattcttgtgatattccaaggagcgttccttttctcctccattccagtttttttgttttattcttttctgtcactgattcaaattaccacttttggaaaaaaaaatgaaagaaaatataatgaaaaggcagcttgaatttccaaatgtgcaattcacttgtgaacaaagtaattctagaggtttctctccgtgcgttttgcatcgtcactgttctcttcaagacgagggaagctgagccccatgtcagaacacgtcgctgaaagcacacacaggtatatacgcgtgcaggcaccccgacctgtgcagccgcgccatcctctggccgttgttacatctcagatttggtttagtttggtttttgtttttttcttttgaaactgtagagtgttgaagaagaaatgaagagtaaatgtctggttttcatataacgtttaaagaaaccatcaggaaggaggctgacgtgtgtgccatgtcccctgattgtaaattgcttctgttatcagtaaactgtgcatgactcgtgtttagcagtcattatcgtgtctgtttgtgaaatttttatggaaaggaaaattccgtagatgcacttactgactgtgtggttagggtccacgtcctcaagactggagtcctgagctgccgacgcaaaggagacgcagttcttaattacgatggaaattgctgtgggagaagaatgagacaaaaaggcgtaagggctcggaagccgttcagagcgattctgcagatttcatttgatgcttgtgaaaaagacaaatgtacttgtgtagagaaatttctttaagataaagcctaggagaagctgatttgaggttaagctgtagattaggagtgtataccaaatgtaatctttccgatgctacaatgaatttatacatgagattgatatgcaataaatctgtgtgcttttctaa

(19) United States
(12) Patent Application Publication
(10) Pub. No.: US 2025/0297316 A1
(43) Pub. Date: Sep. 25, 2025

(54) Detection of Expression of Markers Useful for Predicting Risk of Catastrophic Injuries in Athletic Animals

(71) Applicant: University of Kentucky Research Foundation, Lexington, KY (US)

(72) Inventors: Allen E. Page, Wilmore, KY (US); David W. Horohov, Lexington, KY (US); Theodore Kalbfleisch, Lexington, KY (US); Emma Adam, Lexington, KY (US); James MacLeod, Lexington, KY (US)

(21) Appl. No.: 19/088,898

(22) Filed: Mar. 24, 2025

Related U.S. Application Data
(60) Provisional application No. 63/569,525, filed on Mar. 25, 2024.

(51) Int. Cl. C12Q 1/6883

(52) U.S. Cl. CPC C12Q 1/6883; C12Q 2600/158

(57) ABSTRACT

The present disclosure relates to methods of detecting gene expression in a biological sample from an animal. More specifically, this disclosure relates to methods of detecting risk for a catastrophic injury in an animal, such as a non-human athletic animal, based on the expression of select genes within a biological sample from the animal, which can be determined using mRNA expression analysis. The select genes include at least one of caveolin-1 (CAV1), caveolae associated protein 1 (CAVIN1), and PR domain containing 16 (PRDM16).

### RELATED APPLICATIONS

This application claims priority from U.S. Provisional Application Ser. No. 63/569,525 filed Mar. 25, 2024, the entire disclosure of which is incorporated herein by this reference.

### REFERENCE TO AN ELECTRONIC SEQUENCE LISTING

The contents of the electronic sequence listing (Page UK 2629 Sequence Listing.xml; Size: 398,413 bytes; and Date of Creation: Mar. 21, 2025) is herein incorporated by reference in its entirety.

### TECHNICAL FIELD

The presently-disclosed subject matter generally relates to gene expression detection in biological samples from animals, such as non-human athletic animals. More specifically, embodiments of the present disclosure relate to methods for identifying risk of catastrophic injury in non-human athletic animals based on gene expression within biological samples obtained from such animals, which can be determined using mRNA expression analysis.

### INTRODUCTION

Despite the work of numerous groups detailing a multitude of risk-factors associated with catastrophic injuries (CI's) in Thoroughbred racehorses across the world,¹⁻¹⁶ the ability to reduce the number of CI's in North America remains a significant challenge.¹⁷ It has been established that many CI's occur in limbs with underlying or pre-existing pathology,¹⁸⁻²⁵ leading to the theory that acute injury is due to the accumulation of mild to moderate damage over time at a rate that exceeds the healing capacity of the affected tissues.⁶ Thus, earlier detection of this damage followed by corrective action could reduce the incidence of fatal and/or career-ending injuries.²⁶

Advanced imaging techniques, such as computed tomography, magnetic resonance imaging, and positron emission tomography, have all been proposed as a possible means for detecting impending CI's.^{19, 27-30} With a recent review providing information specifically on imaging of the fetlock,³¹ more work is urgently needed in this area to better understand and identify risk factor-associated changes. Unfortunately, these approaches may be cost prohibitive if used on a regular, screening basis and/or they may require general anesthesia.

Alternatively, others have suggested using genetic screening or predictive modeling to identify those horses at greatest risk for CI's,^{4, 32-34} though the utility of these approaches have yet to be proven. While the detection of biomarkers for equine injuries has also been explored,³⁵⁻³⁹ their use has not been widely adopted, despite some reported success.^{40, 41}

This overall shift from retrospective examination to a more proactive application of research signals a positive move towards catastrophic injury prevention, especially given recent headlines and negative attention regarding catastrophic injuries in North America.⁴²

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes methods which include obtaining a biological sample from an animal, such as a non-human athletic animal, and detecting expression of a gene or a combination of genes within the biological sample. In particular, the presently-disclosed subject matter includes methods of detecting, within the biological sample, the expression of one or more genes which may serve useful as a potential biomarker for identifying when the animal is at an increased risk of experiencing a catastrophic injury (CI).

In some embodiments of the methods of the present disclosure, the gene(s) detected within the biological sample, which may serve as a potential biomarker indicative of increased risk for CI, includes one or more genes selected from the group consisting of caveolin-1 (CAV1), caveolae associated protein 1 (CAVIN1), and PR domain containing 16 (PRDM16), interleukin 1 receptor antagonist (IL1RN), insulin-like growth factor (IGF-1), matrix metallopeptidase 2 (MMP2), arachidonate 5-Lipoxygenase Activating Protein (ALOX5AP), cluster of differentiation 14 (CD14), interleukin 1 beta (IL-1β), interleukin 6 (IL-6), interleukin 8 (IL-8), interleukin 10 (IL-10), matrix metallopeptidase 1 (MMP1), prostaglandin-endoperoxide synthase 2 (PTGS2), toll-like receptor 4 (TLR4), tumor necrosis factor alpha (TNFα), tumor necrosis factor receptor superfamily member 13B (TNFSF13B), and vascular endothelial growth factor A (VEGFA). In some embodiments, the genes detected within the biological sample is a combination of two or more genes selected from the group consisting of CAV1, CAVIN1, PRDM16, ALOX5AP, IL-6, CD14, IL-18, IL-8, IL-10, MMP1, PTGS2, TLR4, TNFα, TNFSF13B, and VEGFA. In some embodiments, the combination of genes detected within the biological sample includes at least one of the genes selected from the group consisting of CAV1, CAVIN1, and PRDM16.

In some embodiments, the methods of the present disclosure may further comprise identifying the animal from which the biological sample was taken as having a risk of a CI or excluding the animal as having a risk of a CI based on the expression of the one or more genes detected within the bioloigcal sample. In some embodiments, the expression of each gene is identified by comparing the expression of the gene within the biological sample to a baseline calibrator or non-injured population of animals. In some embodiments, changes in the expression of each gene are identified by obtaining a second biological sample from the animal at a time point subsequent to when the first biological sample was obtained and comparing the two samples.

In some embodiments, the biological sample is obtained from whole peripheral blood of the animal. In some embodiments, the biological sample is buffy coat fraction of the whole peripheral blood. In some embodiments, the biological sample is plasma or serum from the whole peripheral blood. In some embodiments, the biological sample is taken from a horse, such as a Thoroughbred racehorse.

In the methods of the present disclosure, detecting the expression of one or more genes serving as a potential biomarker for CI detection is preferably achieved via mRNA expression analysis. In this regard, the expression level of the one or more genes may be determined by the levels of mRNA corresponding to the one or more genes within the biological sample. Certain methods of the present disclosure may thus further comprise extracting mRNA from the biological sample. In some embodiments, mRNA levels corresponding to the one or more genes may be determined by using quantitative polymerase chain reaction (qPCR) to measure cDNA of the mRNA. By virtue of utilizing biological samples taken from the animal and mRNA expression analysis, risk of CI can thus be detected in a more economical, efficient, and non-invasive manner than techniques currently employed within the art.

Further provided in the presently-disclosed subject matter is a kit for detecting gene expression in a biological sample from an animal, such as a non-human athletic animal. The kit includes a primer specific for one or more of the genes referred to herein. In some embodiments, the kit includes a primer specific for each of at least two genes selected from the group consisting of CAV1, CAVIN1, and PRDM16. In some embodiments, the kit further includes a primer specific for at least one additional gene selected from the group consisting of IL1RN, IGF-1, MMP2, ALOX5AP, CD14, IL-1β, IL-6, IL-8, IL-10, MMP1, PTGS2, TLR4, TNFα, TNFSF13B, and VEGFA.

### BRIEF DESCRIPTION OF THE DRAWINGS

The features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1 shows a receiver operating characteristic curve analysis results for a model including three genes from Group A markers (IGF1, MMP2, and IL1RN), a model including three genes from Group B (CAV-1, CAVIN-1, and PRDM16), and a model including all six genes.

FIG. 2 shows a comparison of mRNA expression of the models of FIG. 1 between groups of injured horses compared to non-injured control horses.

FIG. 3 shows a receiver operating characteristic curve analysis results for IGF1, MMP2, CAV-1, CAVIN-1, and PRDM16.

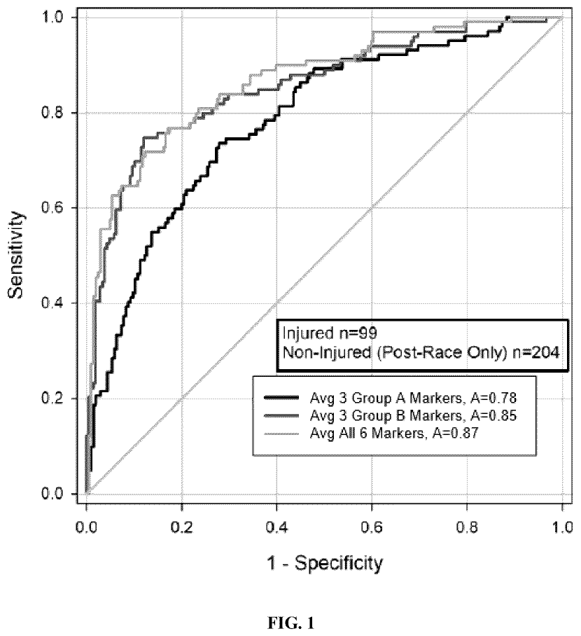

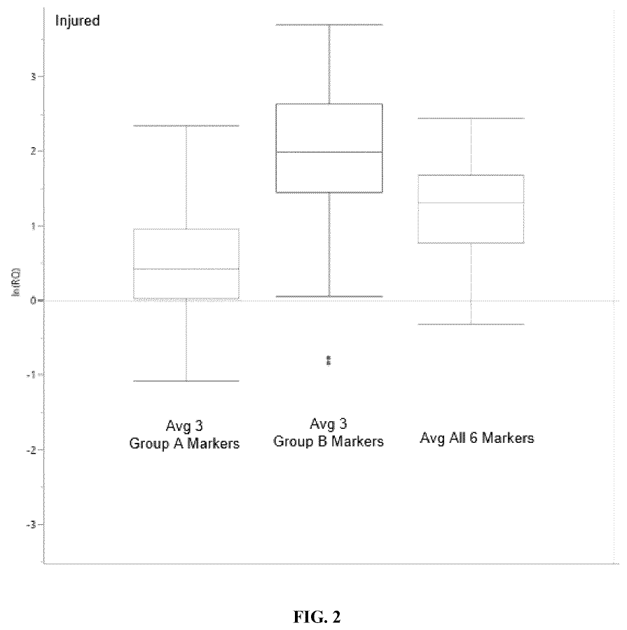

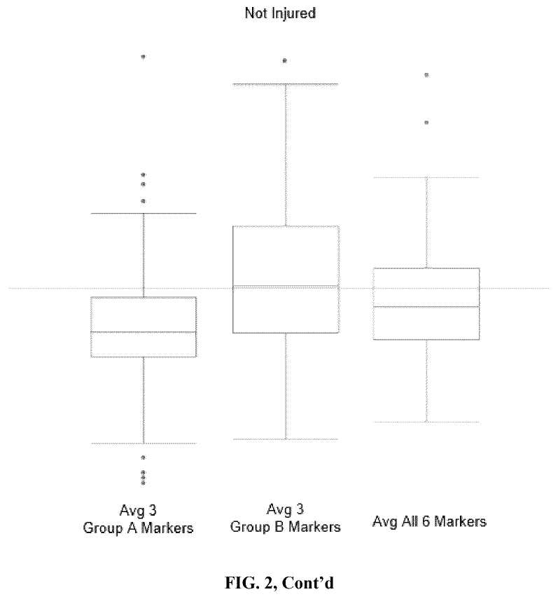

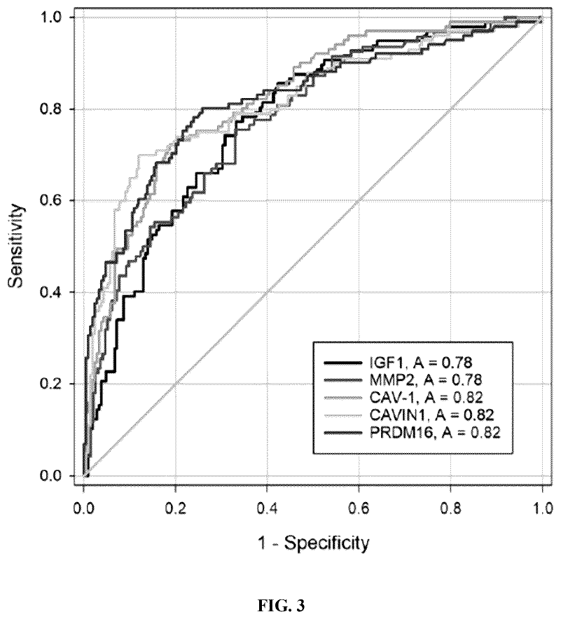

### BRIEF DESCRIPTION OF THE SEQUENCE LISTING

SEQ ID NO: 1 is a nucleotide sequence for CAV1.
SEQ ID NO: 2 is a nucleotide sequence for CAVIN1.
SEQ ID NO: 3 is a nucleotide sequence for PRDM16.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter is illustrated by specific but non-limiting examples throughout this description. The examples may include compilations of data that are representative of data gathered at various times during the course of development and experimentation related to the present invention(s). Each example is provided by way of explanation of the present disclosure and is not a limitation thereon. In fact, it will be apparent to those skilled in the art that various modifications and variations can be made to the teachings of the present disclosure without departing from the scope of the disclosure. For instance, features illustrated or described as part of one embodiment can be used with another embodiment to yield a still further embodiment.

All combinations of methods or process steps as used herein can be performed in any order, unless otherwise specified or clearly implied to the contrary by the context in which the referenced combination is made.

All patents, patent applications, published applications and publications, sequence accession numbers, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUBMB Joint Commission on Biochemical Nomenclature (See, iubmb.qmul.ac.uk/).

Although any methods, devices, and materials similar or equivalent to those described herein can be used in the practice or testing of the presently-disclosed subject matter, representative methods, devices, and materials are described herein.

Some of the polynucleotide and polypeptide sequences disclosed herein are cross-referenced to accession numbers for publicly-accessible databases. The sequences cross-referenced in the database are expressly incorporated by reference as are equivalent and related sequences present in the public databases. Also expressly incorporated herein by reference are all annotations present in the database associated with the sequences disclosed herein. Unless otherwise indicated or apparent, the references to the database are references to the most recent version of the database as of the filing date of this Application.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

As used herein, the term “animal” refers to vertebrate animal that is not a human, and preferably refers to a mammal that is not a human. For example, the term animal can refer, in some embodiments, to a horse, a camel, a dog, an elephant, a pig, a goat, a donkey, and other non-human animals. The term is inclusive of animals of different breeds, for example, a horse is inclusive of a Thoroughbred, Standardbred, Saddlebred, and other breeds of horses, including those classified as cold bloods, warm bloods, and hot bloods.

The term “athletic animal” refers to an animal that participates in an animal sporting event. In some cases, participating in an animal sporting event involves selective breeding, training to prepare for a sporting event, and participation in a sporting event. In some cases, a human is participating with the animal in the animal sporting event. In some cases, a human is not participating with the animal in the animal sporting event. Examples of sporting events include, but are not limited to, racing, polo, jousting, showing, eventing, jumping, dressage, obstacle course, and agility competition.

As used herein, the term “catastrophic injury” refers to a fatal or non-fatal musculoskeletal injury sustained by an athletic animal during racing or training that results in an acute lameness. Such injuries include, but are not limited to, condylar fractures (fractures of the lateral or medial condyle of the third metacarpal or metatarsal bone, also called the cannon bone); fractures of the proximal sesamoid bones, whether involving one or more proximal sesamoid bones; fractures in one or more bones of the carpus (the knee) or tarsus (the hock); rupture of the suspensory apparatus or other tendons or ligaments; P1 (long pastern bone) or P2 (short pastern bone) fractures/sagittal fractures (or any fracture of the distal limb); and any other bony fractures, including those of the scapula, tibia, humerus, pelvis, femur, or stifle joint.

An alternative, less-invasive approach for identifying horses at risk for injury based on research with human athletes regarding exercise-induced, pro-inflammatory cytokine production and its modulation during training⁴³⁻⁴⁵ is contemplated herein. It is believed that exercise-induced inflammation involving high volume/intensity training produces muscle and/or bone trauma resulting in the release of damage associated molecular patterns (DAMPs).⁴⁶ These molecules are released by cells undergoing necrosis and act as endogenous danger signals to promote an inflammatory response.⁴⁷ DAMPs bind to receptors on the surface of dendritic cells, monocytes, macrophages, and other cells leading to the production of pro-inflammatory cytokines such as tumor necrosis factor-alpha (TNF-α), interleukin (IL)-1β, and IL-6.⁴⁸

Post-exercise increases in inflammatory markers are known to occur within several hours of exercise in humans,^{49, 50} and work performed in horses has shown that some of the same markers (TNF-α and IL-1β) exhibit increased expression in circulating leukocytes two or more hours after the completion of exercise.⁵¹⁻⁵⁴ Effective exercise conditioning, on the other hand, leads to a decrease in this inflammatory response and the adoption of an anti-inflammatory state.^{53, 55}

Therefore, it is contemplated that, prior to or during the early post-race period, appropriately conditioned, healthy horses will exhibit reduced expression of inflammatory markers in their peripheral blood. By contrast and based upon the knowledge that CI's occur in limbs with preexisting damage, increased inflammation could be indicative of impending injury or an increased risk for injury.⁵⁶

The present inventors contemplated that athletic animals, such as Thoroughbred racehorses, with a catastrophic injury during training or racing would demonstrate increased inflammatory gene expression at the time of their injury when compared to non-injured control horses. This is based on the known timing of inflammatory mRNA changes in response to exercise^{51, 53} and localized inflammation,⁵⁷ such that samples collected immediately post-injury/post-race will represent the pre-race inflammatory status of the individual horses.

The presently-disclosed subject matter includes a method of detecting gene expression in a biological sample from a non-human athletic animal, which involves obtaining a biological sample from the animal and detecting in the biological sample the expression of at least one gene selected from the group consisting of caveolin-1 (CAV1), caveolae associated protein 1 (CAVIN1), PR domain containing 16 (PRDM16).

In some embodiments, a combination of two or more genes selected from the group consisting of CAV-1, CAVIN-1, and PRDM16 are detected within the biological sample. In some embodiments, the combination of three genes detected in the biological sample includes CAV-1, CAVIN-1, and PRDM16. In some embodiments, the combination of two or more genes detected in the biological sample includes at least one of CAV-1, CAVIN-1, and PRDM16. In one such embodiment, the combination of two or more genes detected in the biological sample further includes at least one additional gene selected from the group consisting of interleukin 1 receptor antagonist (IL1RN), insulin-like growth factor (IGF-1), matrix metallopeptidase 2 (MMP2), arachidonate 5-Lipoxygenase Activating Protein (ALOX5AP), and interleukin 6 (IL-6). In some embodiments, the combination of two or more genes detected in the biological sample includes IL1RN, IGF-1, and MMP2. In some embodiments, the combination of genes detected in the biological sample further includes at least one additional gene selected from the group consisting of cluster of differentiation 14 (CD14), interleukin 1 beta (IL-1β), IL-6, interleukin 8 (IL-8), interleukin 10 (IL-10), matrix metallopeptidase 1 (MMP1), prostaglandin-endoperoxide synthase 2 (PTGS2), toll-like receptor 4 (TLR4), tumor necrosis factor alpha (TNFα), tumor necrosis factor receptor superfamily member 13B (TNFSF13B), and vascular endothelial growth factor A (VEGFA).

In some embodiments, the method of detecting gene expression in a biological sample from a non-human athletic animal, further involves: obtaining an additional (or “second”) biological sample from the animal at a time point subsequent to when the initial (or “first”) biological sample was obtained; detecting in the second biological sample the expression of the same gene(s) detected in the first biological sample; and identifying changes in expression of such gene(s) by comparing the second biological sample to the first biological sample.

In this regard, depending on the gene(s) selected for detection in the first biological sample, at least one of CAV-1, CAVIN-1, and PRDM16 is detected in the second biological sample. In some embodiments, IL1RN, IGF-1, MMP2, ALOX5AP, and IL-6 may also be detected in the second biological sample. In some embodiments, CD14, IL-18, IL-8, IL-10, MMP1, PTGS2, TLR4, TNFα, TNFSF13B, and/or VEGFA may also be detected in the second biological sample. In some embodiments, more than one additional biological sample may be obtained from the animal at a time point subsequent to when the first biological was obtained and compared to the first biological sample to identify changes in gene expression. In this regard, in some embodiments, the method may include detecting changes in gene expression in the animal over various periods of time.

The presently-disclosed subject matter further includes a method of detecting risk for a catastrophic injury in a non-human athletic animal, which involves obtaining a biological sample from the animal; detecting expression of at least one gene in the biological sample; and identifying a risk associated with the animal based on expression of the at least one gene. In some embodiments, the at least one gene detected in the biological sample is selected from the group consisting of: CAV-1, CAVIN-1, and PRDM16. In some embodiments, at least one additional gene is detected in the biological sample, selected from the group consisting of: IL1RN, IGF-1, MMP2, ALOX5AP, IL-6, CD14, IL-1β, IL-6, IL-8, IL-10, MMP1, PTGS2, TLR4, TNFα, TNFSF13B, and VEGFA.

The presently-disclosed subject matter further includes a method of detecting risk for a catastrophic injury in a non-human athletic animal, which comprises obtaining a blood sample from a subject animal; detecting in the blood sample from the subject animal the mRNA expression level of at least one of caveolin-1 (CAV1), caveolae associated protein 1 (CAVIN1), and PR domain containing 16 (PRDM16); and detecting at least one of (i) an increased mRNA expression level of CAV1 in the blood sample obtained from the subject animal relative to a control expression level of CAV1 from the one or more control blood samples obtained from the one or more control animals without catastrophic injury, (ii) an increased mRNA expression level of CAVIN1 in the blood sample obtained from the subject animal relative to a control expression level of CAVIN1 from the one or more control blood samples obtained from the one or more control animals without catastrophic injury, and (iii) an increased mRNA expression level of PRDM16 in the blood sample obtained from the subject animal relative to a control expression level of PRDM16 from the one or more control blood samples obtained from the one or more control animals without catastrophic injury; identifying the subject animal as being at risk for catastrophic injury based on the detection of the at least one of the increased mRNA expression level of CAV1 in the blood sample obtained from the subject animal, the increased mRNA expression level of CAVIN1 in the blood sample obtained from the subject animal, and the increased mRNA expression level of PRDM16 in the blood sample obtained from the subject animal; and treating the subject animal subsequent to being identified as at risk for catastrophic injury; wherein treating the subject animal includes implementing advanced diagnostics to localize potential injury locations.

In some embodiments, the method further comprises detecting in the blood sample from the subject animal the mRNA expression level of at least one of arachidonate 5-lipoxygenase activating protein (ALOX5AP), interleukin 6 (IL-6), cluster of differentiation 14 (CD14), interleukin 1 beta (IL-18), interleukin 8 (IL-8), interleukin 10 (IL-10), matrix metallopeptidase 1 (MMP1), prostaglandin-endoperoxide synthase 2 (PTGS2), toll-like receptor 4 (TLR4), tumor necrosis factor alpha (TNFα), tumor necrosis factor receptor superfamily member 13B (TNFSF13B), and vascular endothelial growth factor A (VEGFA).

In some embodiments of the method, the blood sample from the subject animal is from whole peripheral blood. In some embodiments, the blood sample from the subject animal is plasma or serum from the whole peripheral blood. In some embodiments, the blood sample from the subject animal is a buffy coat fraction of the whole peripheral blood.

In some embodiments, the method further comprises extracting mRNA from the blood sample from the subject animal. In some embodiments, the method further comprises measuring in the extracted mRNA the levels of mRNA corresponding to the at least one of CAV1, CAVIN1, and PRDM16. In some embodiments of the method, quantitative polymerase chain reaction (qPCR) is used to measure the mRNA by measuring cDNA of the mRNA.

In some embodiments of the method, an increased expression level of at least one of CAV1, CAVIN1, and PRDM16 in the blood sample from the subject animal is detected relative to the control expression level of a corresponding gene of the one or more control blood samples obtained from the one or more control animals without catastrophic injury.

In some embodiments of the method, the animal is a horse.

The presently-disclosed subject matter includes a method for detecting biomarker expression indicative of increased risk for catastrophic injury in a non-human athletic animal, which comprises obtaining a first blood sample from a subject animal; detecting in the first blood sample from the subject animal the mRNA expression level of at least one of caveolin-1 (CAV1), caveolae associated protein 1 (CAVIN1), and PR domain containing 16 (PRDM16); obtaining a second blood sample from the subject animal at a time subsequent to when the first blood sample was obtained; and detecting in the second blood sample the mRNA expression level of at least one of CAV1, CAVIN1, and PRDM16; and detecting at least one of (i) an increased level of CAV1 in the second blood sample obtained from the subject animal relative to the expression level of CAV1 in the first blood sample, (ii) an increased level of CAVIN1 in the second blood sample obtained from the subject animal relative to the expression level of CAVIN1 in the first blood sample, and (iii) an increased level of PRDM16 in the second blood sample obtained from the subject animal relative to the expression level of PRDM16 in the first blood sample; identifying the subject animal as being at risk for catastrophic injury based on the detection of the at least one of the increased mRNA expression level of CAV1 in the second blood sample obtained from the subject animal, the increased mRNA expression level of CAVIN1 in the second blood sample obtained from the subject animal, and the increased mRNA expression level of PRDM16 in the second blood sample obtained from the subject animal; treating the subject animal subsequent to being identified as at risk for catastrophic injury; wherein treating the subject animal includes implementing advanced diagnostics to localize potential injury locations.

In some embodiments, the method further comprises detecting in the blood sample from the subject animal the mRNA expression level of at least one of arachidonate 5-lipoxygenase activating protein (ALOX5AP), interleukin 6 (IL-6), cluster of differentiation 14 (CD14), interleukin 1 beta (IL-1β), interleukin 8 (IL-8), interleukin 10 (IL-10), matrix metallopeptidase 1 (MMP1), prostaglandin-endoperoxide synthase 2 (PTGS2), toll-like receptor 4 (TLR4), tumor necrosis factor alpha (TNFα), tumor necrosis factor receptor superfamily member 13B (TNFSF13B), and vascular endothelial growth factor A (VEGFA).

In some embodiments of the method, the blood sample from the subject animal is from whole peripheral blood. In some embodiments of the method, the blood sample from the subject animal is plasma or serum from the whole peripheral blood. In some embodiments of the method, the blood sample from the subject animal is a buffy coat fraction of the whole peripheral blood. In some embodiment, the method further comprises extracting mRNA from the blood sample from the subject animal. In some embodiment, the method further comprises measuring in the extracted mRNA the levels of mRNA corresponding to the at least one of CAV1, CAVIN1, and PRDM16. In some embodiments of the method, quantitative polymerase chain reaction (qPCR) is used to measure the mRNA by measuring cDNA of the mRNA.

In some embodiments of the method, an increased mRNA expression level of at least one of CAV1, CAVIN1, and PRDM16 in the first blood sample is detected relative to the mRNA expression level of an mRNA expression level of a corresponding gene in the second blood sample.

In some embodiments of the method, the animal is a horse.

In some embodiments of the presently-disclosed subject matter qPCR is used to measure expression of a gene of interest by measuring cDNA of the mRNA of the gene that has been extracted from a sample. As will be appreciated by one of ordinary skill in the art, oligonucleotide primers re used when running a PCR reaction. The primers are complementary to a template region of DNA. Methods of designing and synthesizing primers for a nucleotide sequence of interest are well known in the art and there are a number of companies that provide primer synthesis services for qPCR, including, for example, Integrated DNA Technologies, Sigma-Aldrich, Thermo Fisher Scientific, Eurofins Genomics, and Genewiz.

Sequences associated with the various genes identified herein are available in a number of publicly-available databases, including the National Center for Biotechnology Information (NCBI) database. For example, certain sequences of interest have the NCBI accession numbers set forth in Table 1. Of course, sequences for additional isoforms and sequences for additional genes are accessible in the NCBI database, and other publicly available databases, and can be searched, for example, by using the name of the gene of interest.

TABLE 1
Examples of NCBI Accession Number

| | Nucleotide | Amino Acid | |
|---|---|---|---|
| | Sequence | Sequence | mRNA Sequence |
| CAV1 | NC_091687.1 | NP_001107615 | NM_001114143 |
| CAVIN1 | NC_091694 | XP_001494523 | XM_001494473.6 |
| PRDM16 | NC_091685 | XP_070110733 | XM_070254632 |
| ALOX5AP | NC_091700 | XP_070095334 | XM_070239233 |
| IL-6 | NC_091687 | AAC04574 | AF041975 |
| CD14 | NC_091697 | XIF08966 | PQ468778 |
| IL-1β | NC_091698 | NP_001075995 | NM_001082526 |
| IL-8 | AF062377 | AAC16015 | AF062377 |
| IL-10 | NC_091688 | ABZ91980 | EU438771 |
| MMP1 | NC_091690 | NP_001075316 | NM_001081847 |
| PTGS2 | NC_091688 | AAC07911 | AF027334 |
| TLR4 | NC_091708 | XIF08969 | PQ468781 |
| TNFα | NC_091703 | APU51305 | KX345377 |
| TNFSF13B | NC_091700 | NP_001229374 | NM_001242445 |
| VEGFA | NC_091703 | NP_001422835 | NM_001435906 |

In methods disclosed herein, which involve detecting or diagnosing a risk for a catastrophic injury in a non-human athletic animal, a risk of catastrophic injury can be associated with the animal when differential expression shows the gene(s) selected for detection in biological samples exhibiting increased or decreased expression levels. For example, in some embodiments, the animal can be identified as having a risk of catastrophic injury when the gene(s) selected for detection exhibit a decreased or increased expression of the genes as identified in the second column of Table 2A and/or Table 2B. For another example, in some embodiments the animal can be excluded from having a risk of catastrophic injury when the biological sample has substantially the same expression, or an increased or decreased expression of the genes as identified in the third column of the following Table 2A and/or Table 2B.

TABLE 2A
Risk of injury as related to gene expression.

| | Identify a risk | Exclude a risk |
|---|---|---|
| | when differential | when differential |
| | expression shows: | expression shows: |
| caveolin-1 (CAV1) | Increase | Decrease |
| caveolae associated protein 1 | Increase | Decrease |
| (CAVIN1) | | |
| PR domain containing 16 | Increase | Decrease |
| (PRDM16) | | |

TABLE 2B
Risk of injury as related to gene expression.

| | Identify a risk | Exclude a risk |
|---|---|---|
| | when differential | when differential |
| | expression shows: | expression shows: |
| interleukin 1 receptor | Decrease | Increase |
| antagonist (IL1RN) | | |
| insulin-like growth factor | Increase | Decrease |
| (IGF-1) | | |
| matrix metallopeptidase 2 | Increase | Decrease |
| (MMP2) | | |
| arachidonate 5-Lipoxygenase | Decrease | Increase |
| Activating Protein | | |
| (ALOX5AP) | | |
| interleukin 1 beta (IL-1β) | Decrease | Increase |
| interleukin 6 (IL-6) | Decrease | Increase |
| prostaglandin-endoperoxide | Decrease | Increase |
| synthase 2 (PTGS2) | | |
| vascular endothelial growth | Decrease | Increase |
| factor A (VEGFA) | | |
| cluster of differentiation | Increase | Decrease |
| 14 (CD14) | | |
| interleukin 8 (IL-8) | Increase | Decrease |
| interleukin 10 (IL-10) | Increase | Decrease |
| matrix metallopeptidase 1 | Increase | Decrease |
| (MMP1) | | |
| toll-like receptor 4 (TLR4) | Increase | Decrease |
| tumor necrosis factor alpha | Increase | Decrease |
| (TNFα) | | |
| tumor necrosis factor | Increase | Decrease |
| receptor superfamily | | |
| member 13B (TNFSF13B) | | |

The differential expression can be assessed relative to a baseline calibrator or a non-injured population of animals. The “baseline calibrator” for each gene can be the average expression level of that gene in a group of sedentary animals of the same species as the animal from which the biological sample was obtained. The expression in a non-injured population of animals for each gene can be the average expression level of that gene in a group of non-injured animals of the same species as the animal from which the biological sample was obtained.

In embodiments of the methods described herein, when a risk has been identified, the methods can further involve providing treatment to the animal. In some embodiments, treatment of the animal includes implementing advanced diagnostics to localize potential injury locations. In some embodiments treatment of the animal includes suspending the animal from high-intensity exercise. In some embodiments, treatment of the animal may persist until such time as it is determined that the animal is no longer at risk.

In this regard, in some embodiments, the method of detecting risk for a catastrophic injury in a non-human athletic animal involves obtaining an additional (or “second”) biological sample from the animal at a time point subsequent to when the initial (or “first”) biological sample was obtained; detecting in the second biological sample expression of the same gene(s) detected in the first biological sample; and identifying changes in expression of such gene(s) by comparing the second biological sample to the first biological sample. In some embodiments, changes in the expression of the at least one gene in the second biological sample as compared to the first biological sample are identified. Such changes can be indicative of a change in the risk profile for the animal. In this regard, depending on the gene(s) selected for detection in the first biological sample, at least one of CAV-1, CAVIN-1, and PRDM16 is detected in the second biological sample. In some embodiments, IL1RN, IGF-1, MMP2, ALOX5AP, and IL-6 may be detected in the second biological sample. In some embodiments, CD14, IL-1β, IL-8, IL-10, MMP1, PTGS2, TLR4, TNFα, TNFSF13B, and/or VEGFA may be detected in the second biological sample. In some embodiments, more than one additional biological sample may be obtained from the animal at a time point subsequent to when the first biological was obtained and compared to the first biological sample to identify changes in gene expression. In this regard, in some embodiments, the method may include detecting changes in gene expression in the animal over various periods of time.

In some embodiments, the method of detecting risk for a catastrophic injury in a non-human athletic animal involves identifying the animal as having a continued risk and/or having developed a risk when the second biological sample has a decreased or an increased expression relative to a baseline calibrator or a non-injured population of animals for one or more genes as identified in the second column of Table 2A and/or Table 2B, or excluding the animal from having a risk and/or identifying the animal as no longer having a risk when the second biological sample has a decreased or an increased expression relative to a baseline calibrator or a non-injured population of animals for one or more genes as identified in the third column of Table 2A and/or Table 2B.

As noted hereinabove, the methods disclosed herein make use of a biological sample obtained from the animal. Because the methods involve detecting expression of particular genes, the biological sample should be selected to allow for such detection. For example, the biological sample can be selected due to its inherent inclusion of expressed gene products, such as mRNA. In this regard, whole peripheral blood can be selected and used as the biological sample in some embodiments of the methods disclosed herein. Other examples of biological samples which may be selected and used as the biological sample included in the methods disclosed herein include, but are not limited to, plasma, serum, and buffy coat fraction of blood.

In some embodiments of the methods disclosed herein, the method of the presently-disclosed subject matter, a biological sample is obtained from the animal within 1, 2, 3, 4, 5, 6, 7, 8, 9, or 10 days from when the animal is scheduled for training or a race.

In some embodiments of the presently-disclosed subject matter, the method of detecting risk for a catastrophic injury in a non-human athletic animal can further involve determining at least one additional risk factor, including, but not limited to, age of the animal, age of the animal at first race, sex of the animal, distance of first race, distance of most-recent race, average distance of all races, type and condition of track surface for first race, type and condition of track surface for most recent race, most common type and condition of track surface for all races, class of race of first race, class of race of most-recent race, most common class of race, and total number of races.

Some embodiments of the presently-disclosed subject matter also involve extracting mRNA from the biological sample. In some embodiments, the method also involves measuring in the extracted mRNA the levels of mRNA corresponding to the gene(s) selected for detection in the biological sample. In some embodiments, the method also involves using quantitative polymerase chain reaction (qPCR) to measure the mRNA by measuring cDNA of the mRNA.

The presently-disclosed subject matter further includes a kit comprising a primer specific for each of one or more of the genes as disclosed herein. In some embodiments, the kit comprises a primer specific for each of at least two genes selected from the group consisting of CAV-1, CAVIN-1, and PRDM16. In some embodiments, the kit additionally comprises a primer specific for at least one gene selected from the group consisting of IL1RN, IGF-1, MMP2, ALOX5AP, and IL-6. In some embodiments, the kit additionally comprises a primer specific for at least one gene selected from the group consisting of ILRN, IGF-1, and MMP2. In some embodiments, the kit additionally comprises a primer specific for at least one gene selected from the group consisting of CD14, IL-1β, IL-6, IL-8, IL-10, MMP1, PTGS2, TLR4, TNFα, TNFSF13B, and VEGFA.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Carrier, T. K., Estberg, L., Stover, S. M., Gardner, I. A., Johnson, B. J., Read, D. H. and Ardans, A. A. (1998) Association between long periods without high-speed workouts and risk of complete humeral or pelvic fracture in thoroughbred racehorses: 54 cases (1991-1994). J Am Vet Med Assoc 212, 1582-1587.
2. Cohen, N. D., Berry, S. M., Peloso, J. G., Mundy, G. D. and Howard, I. C. (2000) Association of highspeed exercise with racing injury in thoroughbreds. J Am Vet Med Assoc 216, 1273-1278.
3. Cohen, N. D., Peloso, J. G., Mundy, G. D., Fisher, M., Holland, R. E., Little, T. V., Misheff, M. M., Watkins, J. P., Honnas, C. M. and Moyer, W. (1997) Racing-related factors and results of prerace physical inspection and their association with musculoskeletal injuries incurred in thoroughbreds during races. J Am Vet Med Assoc 211, 454-463.
4. Georgopoulos, S. P. and Parkin, T. D. (2017) Risk factors for equine fractures in Thoroughbred flat racing in North America. Prev Vet Med 139, 99-104.
5. Hill, A. E., Gardner, I. A., Carpenter, T. E. and Stover, S. M. (2004) Effects of injury to the suspensory apparatus, exercise, and horseshoe characteristics on the risk of lateral condylar fracture and suspensory apparatus failure in forelimbs of thoroughbred racehorses. Am J Vet Res 65, 1508-1517.
6. Perkins, N. R., Reid, S. W. and Morris, R. S. (2005) Risk factors for musculoskeletal injuries of the lower limbs in Thoroughbred racehorses in New Zealand. N Z Vet J 53, 171-183.
7. Vallance, S. A., Entwistle, R. C., Hitchens, P. L., Gardner, I. A. and Stover, S. M. (2013) Case-control study of high-speed exercise history of Thoroughbred and Quarter Horse racehorses that died related to a complete scapular fracture. Equine Vet J 45, 284-292.
8. Verheyen, K. L., Price, J. S. and Wood, J. L. (2007) Fracture rate in Thoroughbred racehorses is affected by dam age and parity. Vet J 174, 295-301.
9. Anthenill, L. A., Stover, S. M., Gardner, I. A. and Hill, A. E. (2007) Risk factors for proximal sesamoid bone fractures associated with exercise history and horseshoe characteristics in Thoroughbred racehorses. Am J Vet Res 68, 760-771.
10. Anderson, T. M., Mcllwraith, C. W. and Douay, P. (2004) The role of conformation in musculoskeletal problems in the racing Thoroughbred. Equine Vet J 36, 571-575.
11. Parkin, T. D., Clegg, P. D., French, N. P., Proudman, C. J., Riggs, C. M., Singer, E. R., Webbon, P. M. and Morgan, K. L. (2004) Horse-level risk factors for fatal distal limb fracture in racing Thoroughbreds in the UK. Equine Vet J 36, 513-519.
12. Kane, A. J., Stover, S. M., Gardner, I. A., Bock, K. B., Case, J. T., Johnson, B. J., Anderson, M. L., Barr, B. C., Daft, B. M., Kinde, H., Larochelle, D., Moore, J., Mysore, J., Stoltz, J., Woods, L., Read, D. H. and Ardans, A. A. (1998) Hoof size, shape, and balance as possible risk factors for catastrophic musculoskeletal injury of Thoroughbred racehorses. Am J Vet Res 59, 1545-1552.
13. Estberg, L., Stover, S. M., Gardner, I. A., Drake, C. M., Johnson, B. and Ardans, A. (1996) Highspeed exercise history and catastrophic racing fracture in thoroughbreds. Am J Vet Res 57, 15491555.
14. Kane, A. J., Stover, S. M., Gardner, I. A., Case, J. T., Johnson, B. J., Read, D. H. and Ardans, A. A. (1996) Horseshoe characteristics as possible risk factors for fatal musculoskeletal injury of thoroughbred racehorses. Am J Vet Res 57, 1147-1152.
15. Hill, A. E., Gardner, I. A., Carpenter, T. E., Lee, C. M., Hitchens, P. L. and Stover, S. M. (2016) Prevalence, location and symmetry of noncatastrophic ligamentous suspensory apparatus lesions in California Thoroughbred racehorses, and association of these lesions with catastrophic injuries. Equine Vet J 48, 27-32.
16. Kristoffersen, M., Parkin, T. D. and Singer, E. R. (2010) Catastrophic biaxial proximal sesamoid bone fractures in UK Thoroughbred races (1999-2004): horse characteristics and racing history. Equine Vet J 42, 420-424.
17. jockeyclub.com/default.asp?section=Advocacy&area=10.
18. Tranquille, C. A., Parkin, T. D. and Murray, R. C. (2012) Magnetic resonance imaging-detected adaptation and pathology in the distal condyles of the third metacarpus, associated with lateral condylar fracture in Thoroughbred racehorses. Equine Vet J 44, 699-706.
19. Trope, G. D., Ghasem-Zadeh, A., Anderson, G. A., Mackie, E. J. and Whitton, R. C. (2015) Can high-resolution peripheral quantitative computed tomography imaging of subchondral and cortical bone predict condylar fracture in Thoroughbred racehorses? Equine Vet J 47, 428-432.
20. Barr, E. D., Pinchbeck, G. L., Clegg, P. D., Boyde, A. and Riggs, C. M. (2009) Post mortem evaluation of palmar osteochondral disease (traumatic osteochondrosis) of the metacarpo/metatarsophalangeal joint in Thoroughbred racehorses. 41, 366-371.
21. Martig, S., Chen, W., Lee, P. V. and Whitton, R. C. (2014) Bone fatigue and its implications for injuries in racehorses. Equine Vet J 46, 408-415.
22. O'Brien, T., Baker, T. A., Brounts, S. H., Sample, S. J., Markel, M. D., Scollay, M. C., Marquis, P. and Muir, P. (2011) Detection of articular pathology of the distal aspect of the third metacarpal bone in thoroughbred racehorses: comparison of radiography, computed tomography and magnetic resonance imaging. Vet Surg 40, 942-951.
23. Parkin, T. D., Clegg, P. D., French, N. P., Proudman, C. J., Riggs, C. M., Singer, E. R., Webbon, P. M. and Morgan, K. L. (2006) Catastrophic fracture of the lateral condyle of the third metacarpus/metatarsus in UK racehorses-fracture descriptions and pre-existing pathology. Vet J 171, 157-165.
24. Riggs, C. M., Whitehouse, G. H. and Boyde, A. (1999) Pathology of the distal condyles of the third metacarpal and third metatarsal bones of the horse. Equine Vet J 31, 140-148.
25. Scollay, M. C. (2017) Autopsy of the racehorse: the regulator's perspective. Journal of veterinary diagnostic investigation: official publication of the American Association of Veterinary Laboratory Diagnosticians, Inc 29, 383-384.
26. Hesse, K. L. and Verheyen, K. L. (2010) Associations between physiotherapy findings and subsequent diagnosis of pelvic or hindlimb fracture in racing Thoroughbreds. Equine Vet J 42, 234-239.
27. Tranquille, C. A., Murray, R. C. and Parkin, T. D. (2017) Can we use subchondral bone thickness on high-field magnetic resonance images to identify Thoroughbred racehorses at risk of catastrophic lateral condylar fracture? Equine Vet J 49, 167-171.
28. Loughridge, A. B., Hess, A. M., Parkin, T. D. and Kawcak, C. E. (2017) Qualitative assessment of bone density at the distal articulating surface of the third metacarpal in Thoroughbred racehorses with and without condylar fracture. Equine Vet J 49, 172-177.
29. Peloso, J. G., Vogler, J. B., 3rd, Cohen, N. D., Marquis, P. and Hilt, L. (2015) Association of catastrophic biaxial fracture of the proximal sesamoid bones with bony changes of the metacarpophalangeal joint identified by standing magnetic resonance imaging in cadaveric forelimbs of Thoroughbred racehorses. J Am Vet Med Assoc 246, 661-673.
30. Spriet, M., Espinosa-Mur, P., Cissell, D. D., Phillips, K. L., Arino-Estrada, G., Beylin, D., Stepanov, P., Katzman, S. A., Galuppo, L. D., Garcia-Nolen, T., Murphy, B. and Stover, S. M. (2019) 18F-sodium fluoride positron emission tomography of the racing Thoroughbred fetlock: Validation and comparison with other imaging modalities in nine horses. Equine Veterinary Journal 51, 375-383.
31. Denoix, J. M. and Coudry, V. (2020) Clinical insights: Imaging of the equine fetlock in Thoroughbred racehorses: Identification of imaging changes to predict catastrophic injury. Equine Veterinary Journal 52, 342-343.
32. Blott, S. C., Swinburne, J. E., Sibbons, C., Fox-Clipsham, L. Y., Helwegen, M., Hillyer, L., Parkin, T. D. H., Newton, J. R. and Vaudin, M. (2014) A genome-wide association study demonstrates significant genetic variation for fracture risk in Thoroughbred racehorses. BMC Genomics 15, 147.
33. Welsh, C. E., Lewis, T. W., Blott, S. C., Mellor, D. J., Stirk, A. J. and Parkin, T. D. (2014) Estimates of genetic parameters of distal limb fracture and superficial digital flexor tendon injury in UK Thoroughbred racehorses. Vet J 200, 253-256.
34. Tozaki, T., Kusano, K., Ishikawa, Y., Kushiro, A., Nomura, M., Kikuchi, M., Kakoi, H., Hirota, K., Miyake, T., Hill, E. W. and Nagata, S. (2020) A candidate-SNP retrospective cohort study for fracture risk in Japanese Thoroughbred racehorses. Animal Genetics 51, 43-50.
35. Billinghurst, R. C., Brama, P. A., van Weeren, P. R., Knowlton, M. S. and Mcllwraith, C. W. (2003) Significant exercise-related changes in the serum levels of two biomarkers of collagen metabolism in young horses. Osteoarthritis Cartilage 11, 760-769.
36. Brama, P. A., van den Boom, R., DeGroott, J., Kiers, G. H. and van Weeren, P. R. (2004) Collagenase-1 (MMP-1) activity in equine synovial fluid: influence of age, joint pathology, exercise and repeated arthrocentesis. Equine Vet J 36, 34-40.
37. Costa, M. F., Davies, H. M., Anderson, G. A. and Slocombe, R. F. (2011) Effects of two training protocols on Angiotensin I-converting enzyme (ACE) activity in horses. Equine Vet J 43, 466-470.
38. Frisbie, D. D., Al-Sobayil, F., Billinghurst, R. C., Kawcak, C. E. and McIlwraith, C. W. (2008) Changes in synovial fluid and serum biomarkers with exercise and early osteoarthritis in horses. Osteoarthritis Cartilage 16, 1196-1204.
39. Graham, R. J. T. Y., Anderson, J. R., Phelan, M. M., Cillan-Garcia, E., Bladon, B. M. and Taylor, S. E. (2020) Metabolomic analysis of synovial fluid from Thoroughbred racehorses diagnosed with palmar osteochondral disease using magnetic resonance imaging. Equine Veterinary Journal 52, 384-390.
40. Frisbie, D. D., Mc Ilwraith, C. W., Arthur, R. M., Blea, J., Baker, V. A. and Billinghurst, R. C. (2010) Serum biomarker levels for musculoskeletal disease in two- and three-year-old racing Thoroughbred horses: A prospective study of 130 horses. Equine Veterinary Journal 42, 643-651.
41. Turlo, A. J., Cywinska, A. and Frisbie, D. D. (2019) Revisiting predictive biomarkers of musculoskeletal injury in thoroughbred racehorses: longitudinal study in polish population. BMC veterinary research 15, 66.
42. Heleski, C., Stowe, C. J., Fiedler, J., Peterson, M. L., Brady, C., Wickens, C. and MacLeod, J. N. (2020) Thoroughbred Racehorse Welfare through the Lens of Social License to Operate—With an Emphasis on a U. S. Perspective. Sustainability 12.
43. Smith, L. L. (2004) Tissue trauma: the underlying cause of overtraining syndrome? J Strength Cond Res 18, 185-193.
44. Nieman, D. C. and Pedersen, B. K. (1999) Exercise and Immune Function. Sports Medicine 27, 7380.
45. Walsh, N. P., Gleeson, M., Shephard, R. J., Gleeson, M., Woods, J. A., Bishop, N. C., Fleshner, M., Green, C., Pedersen, B. K., Hoffman-Goetz, L., Rogers, C. J., Northoff, H., Abbasi, A. and Simon, P. (2011) Position statement part one: Immune function and exercise. Exercise immunology review 17, 6-63.
46. Huldani, Pattelongi, I., Massi, M. N., Idris, I., Bukhari, A., Wahyu Widodo, A. D. and Achmad, H. (2020) Research Reviews on Effect of Exercise on DAMP's, HMGB1, Proinflammatory Cytokines and Leukocytes. Systematic Reviews in Pharmacy 11, 306-312.
47. Vezzoli, M., Castellani, P., Corna, G., Castiglioni, A., Bosurgi, L., Monno, A., Brunelli, S., Manfredi, A. A., Rubartelli, A. and Rovere-Querini, P. (2011) High-Mobility Group Box 1 Release and Redox Regulation Accompany Regeneration and Remodeling of Skeletal Muscle. Antioxidants & Redox Signaling 15, 2161-2174.
48. Manfredi, A. A., Capobianco, A., Bianchi, M. E. and Rovere-Querini, P. (2009) Regulation of Dendritic-and T-Cell Fate by Injury-Associated Endogenous Signals. 29, 69-86.
49. Ostrowski, K., Rohde, T., Asp, S., Schjerling, P. and Pedersen, B. K. (1999) Pro- and anti-inflammatory cytokine balance in strenuous exercise in humans. The Journal of Physiology 515, 287-291.
50. Philippou, A., Bogdanis, G., Maridaki, M., Halapas, A., Sourla, A. and Koutsilieris, M. (2009) Systemic cytokine response following exercise-induced muscle damage in humans. Clinical chemistry and laboratory medicine 47, 777-782.
51. Liburt, N. R., Adams, A. A., Betancourt, A., Horohov, D. W. and Mckeever, K. H. (2010) Exercise-induced increases in inflammatory cytokines in muscle and blood of horses. Equine Vet J Suppl 42, 280-288.
52. Lehnhard, R., Adams, A., Betancourt, A., Horohov, D., Liburt, N., Streltsova, J., Franke, W. and Mckeever, K. (2010) Phenylbutazone blocks the cytokine response following a high-intensity incremental exercise challenge in horses. Comparative Exercise Physiology 7, 103-108.
53. Horohov, D. W., Sinatra, S. T., Chopra, R. K., Jankowitz, S., Betancourt, A. and Bloomer, R. J. (2012) The Effect of Exercise and Nutritional Supplementation on Proinflammatory Cytokine Expression in Young Racehorses During Training. Journal of Equine Veterinary Science 32, 805-815.
54. Donovan, D. C., Jackson, C. A., Colahan, P. T., Norton, N. and Hurley, D. J. (2007) Exercise-induced alterations in pro-inflammatory cytokines and prostaglandin F2alpha in horses. Vet Immunol Immunopathol 118, 263-269.
55. Page, A. E., Stewart, J. C., Holland, R. E. and Horohov, D. W. (2017) The Impact of Training Regimen on the Inflammatory Response to Exercise in 2-Year-Old Thoroughbreds. Journal of Equine Veterinary Science 58, 78-83.
56. Page, A. E., Stewart, J. C., Scollay, M. C. and Horohov, D. W. (2019) Comparison of pre-race inflammatory marker mRNA expression with race-related parameters in Thoroughbreds. Comparative Exercise Physiology 16, 101-106.
57. Page, A. E., Adam, E., Stewart, J. C., Gonzales, C., Barker, V. and Horohov, D. W. (2020) Alterations of peripheral gene expression in response to lipopolysaccharide-induced synovitis as a model for inflammation in horses. Veterinary Immunology and Immunopathology 225, 110058.
58. Page, A. E., Stewart, J. C., Fielding, C. L. and Horohov, D. W. (2019) The effect of a 160-kilometer competitive endurance ride on inflammatory marker mRNA expression in horses. Journal of Equine Veterinary Science 79, 45-49.
59. Livak, K. J. and Schmittgen, T. D. (2001) Analysis of Relative Gene Expression Data Using Real-Time Quantitative PCR and the 2-ΔΔCT Method. Methods (San Diego, Calif.) 25, 402-408.
60. Breathnach, C. C., Sturgill-Wright, T., Stiltner, J. L., Adams, A. A., Lunn, D. P. and Horohov, D. W. (2006) Foals are interferon gamma-deficient at birth. Vet Immunol Immunopathol 112, 199-209.
61. Vick, M. M., Murphy, B. A., Sessions, D. R., Reedy, S. E., Kennedy, E. L., Horohov, D. W., Cook, R. F. and Fitzgerald, B. P. (2008) Effects of systemic inflammation on insulin sensitivity in horses and inflammatory cytokine expression in adipose tissue. American Journal of Veterinary Research 69, 130-139.
62. Johnson, B. J., Stover, S. M., Daft, B. M., Kinde, H., Read, D. H., Barr, B. C., Anderson, M., Moore, J., Woods, L., Stoltz, J. and Blanchard, P. (1994) Causes of death in racehorses over a 2 year period. Equine Veterinary Journal 26, 327-330.
63. Anthenill, L. A., Stover, S. M., Gardner, I. A., Hill, A. E., Lee, C. M., Anderson, M. L., Barr, B. C., Read, D. H., Johnson, B. J., Woods, L. W., Daft, B. M., Kinde, H., Moore, J. D., Farman, C. A., Odani, J. S., Pesavento, P. A., Uzal, F. A., Case, J. T. and Ardans, A. A. (2006) Association between findings on palmarodorsal radiographic images and detection of a fracture in the proximal sesamoid bones of forelimbs obtained from cadavers of racing Thoroughbreds. American Journal of Veterinary Research 67, 858-868.
64. Hernandez, J., Hawkins, D. L. and Scollay, M. C. (2001) Race-start characteristics and risk of catastrophic musculoskeletal injury in Thoroughbred racehorses. J Am Vet Med Assoc 218, 8386.
65. Estberg, L., Stover, S. M., Gardner, I. A., Johnson, B. J., Jack, R. A., Case, J. T., Ardans, A., Read, D. H., Anderson, M. L., Barr, B. C., Daft, B. M., Kinde, H., Moore, J., Stoltz, J. and Woods, L. (1998) Relationship between race start characteristics and risk of catastrophic injury in thoroughbreds: 78 cases (1992). Journal of the American Veterinary Medical Association 212, 544-549.
66. Cohen, N. D., Mundy, G. D., Peloso, J. G., Carey, V. J. and Amend, N. K. (1999) Results of physical inspection before races and race-related characteristics and their association with musculoskeletal injuries in Thoroughbreds during races. Journal of the American Veterinary Medical Association 215, 654-661.
67. Cohen, N. D., Dresser, B. T., Peloso, J. G., Mundy, G. D. and Woods, A. M. (1999) Frequency of musculoskeletal injuries and risk factors associated with injuries incurred in Quarter Horses during races. J Am Vet Med Assoc 215, 662-669.
68. Heemskerk, V. H., Daemen, M. A. R. C. and Buurman, W. A. (1999) Insulin-like growth factor-1 (IGF-1) and growth hormone (GH) in immunity and inflammation. Cytokine & Growth Factor Reviews 10, 5-14.
69. Majidinia, M., Sadeghpour, A. and Yousefi, B. (2018) The roles of signaling pathways in bone repair and regeneration. Journal of Cellular Physiology 233, 2937-2948.
70. Blumenfeld, I., Srouji, S., Peled, M. and Livne, E. (2002) Metalloproteinases (MMPs-2, -3) are involved in TGF-β and IGF-1-induced bone defect healing in 20-month-old female rats. Archives of Gerontology and Geriatrics 35, 59-69.
71. Lieu, S., Hansen, E., Dedini, R., Behonick, D., Werb, Z., Miclau, T., Marcucio, R. and Colnot, C. (2011) Impaired remodeling phase of fracture repair in the absence of matrix metalloproteinase-2. Disease Models & Mechanisms 4, 203.
72. Nieman, D. C., Henson, D. A., Davis, J. M., Dumke, C. L., Utter, A. C., Murphy, E. A., Pearce, S., Gojanovich, G., McAnulty, S. R. and McAnulty, L. S. (2006) Blood leukocyte mRNA expression for IL-10, IL-1Ra, and IL-8, but not IL-6, increases after exercise. J Interferon Cytokine Res 26, 668674.
73. Anthenill, L. A., Gardner, I. A., Pool, R. R., Garcia, T. C. and Stover, S. M. (2010) Comparison of macrostructural and microstructural bone features in Thoroughbred racehorses with and without midbody fracture of the proximal sesamoid bone. Am J Vet Res 71, 755-765.
74. Peloso, J. G., Mundy, G. D. and Cohen, N. D. (1994) Prevalence of, and factors associated with, musculoskeletal racing injuries of thoroughbreds. J Am Vet Med Assoc 204, 620-626.
75. Parkin, T. D. (2007) Epidemiology of training and racing injuries. Equine Vet J 39, 466-469.
76. Parkin, T. D. (2008) Epidemiology of racetrack injuries in racehorses. The Veterinary clinics of North America. Equine practice 24, 1-19.
77. Georgopoulos, S. P. and Parkin, T. D. (2016) Risk factors associated with fatal injuries in Thoroughbred racehorses competing in flat racing in the United States and Canada. J Am Vet Med Assoc 249, 931-939.
78. Swift, J. and G. M. Coruzzi, A matter of time-How transient transcription factor interactions create dynamic gene regulatory networks. Biochimica et biophysica acta. Gene regulatory mechanisms, 2017. 1860 (1): p. 75-83.
79. Witkowska-Piłaszewicz, O. D., M. Żmigrodzka, A. Winnicka, A. Miśkiewicz, K. Strzelec, and A. Cywińska (2019) Serum amyloid A in equine health and disease, Equine Vet J., 51 (3): 293-298.
80. Page A E, Adam E, Arthur R, Barker V, Franklin F, Friedman R, Grande T, Hardy M, Howard B, Partridge E, Rutledge M, Scollay M, Stewart J C, Vale A, Horohov D W. Expression of select mRNA in Thoroughbreds with catastrophic racing injuries. Equine Vet J. 2022 January; 54 (1): 63-73. doi: 10.1111/evj.13423. Epub 2021 Feb. 15. PMID: 33438228.
81. Beckstett, A. The Horse Managing Editor |May 5, 2015| AAEP Convention, AAEP Convention 2014, Article, MRI to Predict Catastrophic Fetlock Fractures in Racehorses. thehorse.com/111999/mri-to-predict-catastrophic-fetlock-fractures-in-racehorses/82.
82. U.S. Pat. No. 12,180,547 to Page, et al., Detection of Expression of Markers Useful for Predicting Risk of Catastrophic Injuries in Athletic Animals.
83. U.S. Patent Application Publication No. US 2015/0301058 to Schettini, et al., Biomarker compositions and methods.
84. U.S. Pat. No. 10,563,262 to Belli, et al., Biomarkers of traumatic brain injury.
85. U.S. Pat. No. 9,200,324 to Cavet, et al., Biomarkers and methods for measuring and monitoring inflammatory disease activity.
86. International Patent Application Publication No. WO 2011/094815 to Smith, et al., Exercise genotyping.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method of detecting biomarker expression indicative of increased risk for catastrophic injury in a non-human athletic animal, comprising:
(a) obtaining a blood sample from a subject animal;
(b) detecting in the blood sample from the subject animal the mRNA expression level of at least one of caveolin-1 (CAV1), caveolae associated protein 1 (CAVIN1), and PR domain containing 16 (PRDM16); and
(c) detecting at least one of
(i) an increased mRNA expression level of CAV1 in the blood sample obtained from the subject animal relative to a control expression level of CAV1 from the one or more control blood samples obtained from the one or more control animals without catastrophic injury,
(ii) an increased mRNA expression level of CAVIN1 in the blood sample obtained from the subject animal relative to a control expression level of CAVIN1 from the one or more control blood samples obtained from the one or more control animals without catastrophic injury, and
(iii) an increased mRNA expression level of PRDM16 in the blood sample obtained from the subject animal relative to a control expression level of PRDM16 from the one or more control blood samples obtained from the one or more control animals without catastrophic injury;
(d) identifying the subject animal as being at risk for catastrophic injury based on the detection of the at least one of the increased mRNA expression level of CAV1 in the blood sample obtained from the subject animal, the increased mRNA expression level of CAVIN1 in the blood sample obtained from the subject animal, and the increased mRNA expression level of PRDM16 in the blood sample obtained from the subject animal; and
(e) treating the subject animal subsequent to being identified as at risk for catastrophic injury;
wherein treating the subject animal includes implementing advanced diagnostics to localize potential injury locations.

2. The method of claim 1, and further comprising detecting in the blood sample from the subject animal the mRNA expression level of at least one of arachidonate 5-lipoxygenase activating protein (ALOX5AP), interleukin 6 (IL-6), cluster of differentiation 14 (CD14), interleukin 1 beta (IL-1β), interleukin 8 (IL-8), interleukin 10 (IL-10), matrix metallopeptidase 1 (MMP1), prostaglandin-endoperoxide synthase 2 (PTGS2), toll-like receptor 4 (TLR4), tumor necrosis factor alpha (TNFα), tumor necrosis factor receptor superfamily member 13B (TNFSF13B), and vascular endothelial growth factor A (VEGFA).

3. The method of claim 1, wherein the blood sample from the subject animal is from whole peripheral blood.

4. The method of claim 3, wherein the blood sample from the subject animal is plasma or serum from the whole peripheral blood.

5. The method of claim 3, wherein the blood sample from the subject animal is a buffy coat fraction of the whole peripheral blood.

6. The method of claim 1, and further comprising extracting mRNA from the blood sample from the subject animal.

7. The method of claim 6, and further comprising measuring in the extracted mRNA the levels of mRNA corresponding to the at least one of CAV1, CAVIN1, and PRDM16.

8. The method of claim 7, wherein quantitative polymerase chain reaction (qPCR) is used to measure the mRNA by measuring cDNA of the mRNA.

9. The method of claim 1, wherein an increased expression level of at least one of CAV1, CAVIN1, and PRDM16 in the blood sample from the subject animal is detected relative to the control expression level of a corresponding gene of the one or more control blood samples obtained from the one or more control animals without catastrophic injury.

10. The method of claim 1, wherein the animal is a horse.

11. A method for detecting biomarker expression indicative of increased risk for catastrophic injury in a non-human athletic animal, comprising:
(a) obtaining a first blood sample from a subject animal;
(b) detecting in the first blood sample from the subject animal the mRNA expression level of at least one of caveolin-1 (CAV1), caveolae associated protein 1 (CAVIN1), and PR domain containing 16 (PRDM16);
(c) obtaining a second blood sample from the subject animal at a time subsequent to when the first blood sample was obtained; and
(d) detecting in the second blood sample the mRNA expression level of at least one of CAV1, CAVIN1, and PRDM16; and
(e) detecting at least one of
(i) an increased level of CAV1 in the second blood sample obtained from the subject animal relative to the expression level of CAV1 in the first blood sample,
(ii) an increased level of CAVIN1 in the second blood sample obtained from the subject animal relative to the expression level of CAVIN1 in the first blood sample, and
(iii) an increased level of PRDM16 in the second blood sample obtained from the subject animal relative to the expression level of PRDM16 in the first blood sample;
(f) identifying the subject animal as being at risk for catastrophic injury based on the detection of the at least one of the increased mRNA expression level of CAV1 in the second blood sample obtained from the subject animal, the increased mRNA expression level of CAVIN1 in the second blood sample obtained from the subject animal, and the increased mRNA expression level of PRDM16 in the second blood sample obtained from the subject animal;
(g) treating the subject animal subsequent to being identified as at risk for catastrophic injury;
wherein treating the subject animal includes implementing advanced diagnostics to localize potential injury locations.

12. The method of claim 11, and further comprising detecting in the blood sample from the subject animal the mRNA expression level of at least one of arachidonate 5-lipoxygenase activating protein (ALOX5AP), interleukin 6 (IL-6), cluster of differentiation 14 (CD14), interleukin 1 beta (IL-1β), interleukin 8 (IL-8), interleukin 10 (IL-10), matrix metallopeptidase 1 (MMP1), prostaglandin-endoperoxide synthase 2 (PTGS2), toll-like receptor 4 (TLR4), tumor necrosis factor alpha (TNFα), tumor necrosis factor receptor superfamily member 13B (TNFSF13B), and vascular endothelial growth factor A (VEGFA).

13. The method of claim 11, wherein the blood sample from the subject animal is from whole peripheral blood.

14. The method of claim 12, wherein the blood sample from the subject animal is plasma or serum from the whole peripheral blood.

15. The method of claim 12, wherein the blood sample from the subject animal is a buffy coat fraction of the whole peripheral blood.

16. The method of claim 11, and further comprising extracting mRNA from the blood sample from the subject animal.

17. The method of claim 16, and further comprising measuring in the extracted mRNA the levels of mRNA corresponding to the at least one of CAV1, CAVIN1, and PRDM16.

18. The method of claim 17, wherein quantitative polymerase chain reaction (qPCR) is used to measure the mRNA by measuring cDNA of the mRNA.

19. The method of claim 11, wherein an increased mRNA expression level of at least one of CAV1, CAVIN1, and PRDM16 in the first blood sample is detected relative to the mRNA expression level of an mRNA expression level of a corresponding gene in the second blood sample.

20. The method of claim 11, wherein the animal is a horse.
